(19) United States
(12) Patent Application Publication
(10) Pub. No.: US 2005/0234244 A1
(43) Pub. Date: Oct. 20, 2005

(54) Synthesis of COX-2 and FAAH inhibitors

(76) Inventors: Wilmin Bartolini, Amesbury, MA (US); Brian M. Cali, Arlington, MA (US); Barbara Chen, Watertown, MA (US); Yueh-Tyng Chien, Newton, MA (US); Mark G. Currie, Sterling, MA (US); G. Todd Milne, Brookline, MA (US); James Philip Pearson, Cambridge, MA (US); John Jeffrey Talley, Somerville, MA (US); Craig Zimmerman, Topsfield, MA (US)

Correspondence Address: FISH & RICHARDSON PC, P.O. BOX 1022, MINNEAPOLIS, MN, 55440-1022, US

(21) Appl. No.: 10/979,794

(22) Filed: Nov. 1, 2004

Related U.S. Application Data
(63) Continuation-in-part of application No. 10/883,900, filed on Jul. 1, 2004.
(60) Provisional application No. 60/563,589, filed on Apr. 20, 2004.
(60) Provisional application No. 60/570,620, filed on May 13, 2004.
(60) Provisional application No. 60/585,102, filed on Jul. 1, 2004.

(51) Int. Cl. C07D 43/02; C07D 209/18

(52) U.S. Cl. 548/465; 548/494

(57) ABSTRACT

Methods for preparing indoles that are useful COX-2 inhibitors and intermediates useful in such methods are described.

This application is a continuation-in-part of U.S. application Ser. No. 10/883,900, filed Jul. 1, 2004, and under 35 USC §119(e)(1), this application claims the benefit of prior Provisional Application Ser. No. 60/563,589 filed on Apr. 20, 2004; Provisional Application Ser. No. 60/570,620 filed on May 13, 2004; and Provisional Application Ser. No. 60/585,102 filed on Jul. 1, 2004, all of which are hereby incorporated by reference in their entirety.

### BACKGROUND

Cyclooxygenases play an essential role in prostaglandin synthesis. Cyclooxygenase-1 (COX-1) is constitutive and relatively long-lived, whereas cyclooxygenase-2 (COX-2) is inducible and relatively short-lived. COX-1 is thought to be responsible for maintaining basal level prostaglandin production, which is important for normal gastrointestinal and renal function. COX-2 is induced by certain inflammatory agents, hormones, growth factors, cytokines, and other agents. COX-2 plays a significant role in prostaglandin synthesis within inflammatory cells such as macrophages and monocytes, and prostaglandin production associated with COX-2 induction can have a deleterious effect on the body. Thus, to reduce unwanted inflammation and to treat certain other conditions, it can be desirable to inhibit COX-2 activity without significantly inhibiting COX-1 activity.

Many non-steroidal anti-inflammatory drugs (NSAIDs) inhibit both COX-1 and COX-2. These non-selective inhibitors include indomethacin (Shen et al. 1963 J Am Chem Soc 85:4881; 4-chlorobenzoyl-5-methoxy-2-methyl-1H-indole-3-acetic acid). It is desirable to identify NSAIDs that inhibit COX-2 activity, but do not significantly inhibit COX-1 activity at physiological levels where COX-2 activity is significantly inhibited. Such selective inhibitors are expected to have the desirable anti-inflammatory, anti-pyretic, and analgesic properties associated with NSAIDs, while having reduced or no gastrointestinal or renal toxicity.

Subsequent to indomethacin administration, the unchanged parent compound, the desmethyl metabolite (O-desmethylindomethacin), the desbenzoyl metabolite (N-deschlorobenzoylindomethacin) and the desmethy-desbenzoyl metabolite (O-desmethy-N-deschlorobenzoylindomethacin) can be found in plasma in significant amounts (Strachman et al. 1964 J Am Chem Soc 8:799; Helleberg 1981 Clin Pharmacokinet 6:245), all in an unconjugated form (Harman et al. 1964 J Pharmocol Exp Therap 143:215). It has been reported that all three metabolites are devoid of anti-inflammatory activity (Helleberg 1981 Pharmacokine. 6:245 and Duggan et al. 1972 Pharmacol and Exp Ther 181:562), although it has also been reported that the desmethyl metabolite has some ability to inhibit prostaglandin synthesis (Shen et al. 1977 Adv Drug Res 12:90).

Indomethacin derivatives in which the benzoyl group has been replaced by a 4-bromobenzyl group or the acetic acid side chain has been extended exhibit greater selectivity for inhibition of COX-2 relative to COX-1 (Black et al. 1996 Bioorganic & Medicinal Chem Lett 6:725 and Black et al. 1997 Advances in Experimental Medicine and Biology 407:73). In addition, synthesis methodology has been demonstrated for the preparation of indomethacin analogues, some of which do not inhibit cyclooxygenases (Touhey et al. 2002 Eur J Cancer 38:1661).

Many fatty acid amides are known to have analgesic activity. A number of fatty acid amides (e.g., arachidonyl amino acids and anandamide) induce analgesia in animal models of pain (see, for example, Walker et al. 1999 Proc Natl Acad Sci 96:12198, Fride and Mechoulam 1993 Eur J Pharmacol 231:313). Anandamide and certain other fatty acid amides (e.g., N-palmitoyl ethanolamine, N-oleoyl ethanolamide, oleamide, 2-arachidonoylglycerol) are cleaved and inactivated by fatty acid amide hydrolase (FAAH) (Deutsch et al. 2003 Prostaglandins Leukot Essent Fatty Acids 66:201; and Cravatt and Lichtman 2003 Current Opinion in Chemical Biology 7:469).

Inhibition of FAAH is expected to lead to an increase in the level of anandamide and other fatty acid amides. This increase in fatty acid amides may lead to an increase in the nociceptive threshold. Thus, inhibitors of FAAH are useful in the treatment of pain. Such inhibitors might also be useful in the treatment of other disorders that can be treated using fatty acid amides or modulators of cannabinoid receptors (e.g., anxiety, eating disorders, and cardiovascular disorders). NPAA (N-palmitoylethanolamine acid anhydrolase) is a hydrolase that breaks down N-palmitoyl ethanolamine (PEA), a fatty acid amide. PEA is a naturally occurring substrate for the cannabinoid receptor 2 (CB2 receptor). Inhibition of NPAA may lead to increased PEA levels. Accordingly, NPAA inhibitors may be useful in the treatment of inflammation and nociceptive pain control.

In addition, there is evidence (see, e.g., Weber et al. 2004 J. Lipid Res. 45:757) that when FAAH activity is reduced or absent, one of its substrates, anandamide, acts as a substrate for COX-2 that can be converted to a prostamide. Thus, certain prostamides may be elevated in the presence of an FAAH inhibitor. Given that certain prostamides are associated with reduced intraocular pressure and ocular hypotensivity, FAAH inhibitors may be useful agents for treating glaucoma.

### SUMMARY

In one aspect, the invention features a method for preparing a compound having the formula: 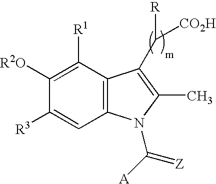
wherein:
each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen; m is 1, 2, 3 or 4; R¹ is H or a halogen; R² is H; a C₁ to C₆ alkyl, optionally independently substituted with one or more halogen; 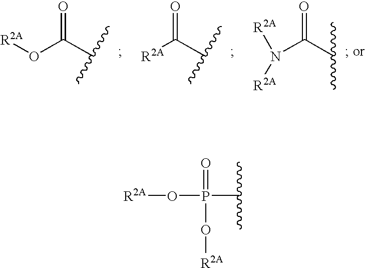
   wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂; R³ is H or a halogen;
A is 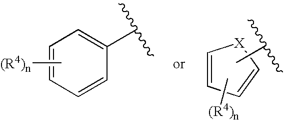
   wherein each R⁴ is independently H, a halogen, —CN, —SH; —OH; a C₁ to C₃ alkyl, optionally, independently substituted with one or more halogen; —OR^{4A}; —SR^{4A}; —SOR^{4A}; —S(O)₂R^{4A}, wherein R^{4A} is a C₁ to C₃ alkyl, optionally independently substituted with one or more halogen; X is S, O, NH or NR⁵, wherein R⁵ is a C₁ to C₁₀ alkyl; n=1,2,3, 4 or 5 when A is 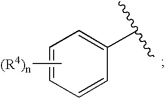
   n=1,2, 3 or 4 when A is 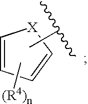
   and Z is O or S, the method including:
   reacting a compound having the formula: 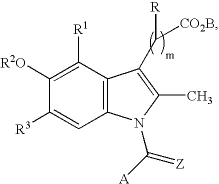
      wherein A, R, R¹, R², R³ and Z are as defined above and B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃ and wherein B is optionally independently substituted with one or more halogen, —OH, —C(O)OH, or —NH₂, with an organic acid or an inorganic acid in the presence of an organic solvent to prepare a compound having the formula:

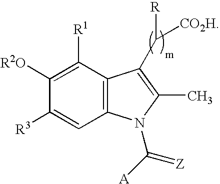

In various embodiments: A is 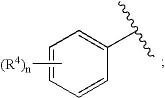
Z is O; m is 1; R is H; R is H and m is 1; B is —CH₂CH₂Si(CH₃)₃; R⁴ is halogen; R⁴ is Cl; n is 1; R¹ is H; and A is

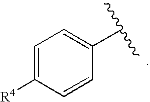

In another aspect, the invention features a method for preparing a compound having the formula: 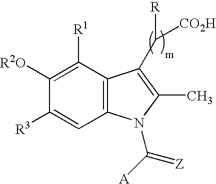
wherein:
each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen; m is 1, 2, 3 or 4; R¹ is H or a halogen; R² is H; a C₁ to C₆ alkyl, optionally independently substituted with one or more halogen; 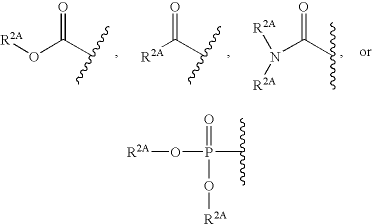
   wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂; R³ is H or a halogen;
A is 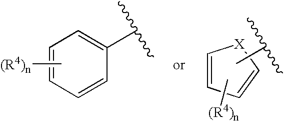
   wherein each R⁴ is independently H, a halogen, —CN, —SH; —OH; a C₁ to C₃ alkyl, optionally, independently substituted with one or more halogen; —OR^{4A}; —SR^{4A}; —SOR^{4A}; —S(O)₂R^{4A}, wherein R^{4A} is a C₁ to C₃ alkyl, optionally independently substituted with one or more halogen; X is S, O, NH or NR⁵, wherein R⁵ is a C₁ to C₁₀ alkyl; n=1, 2, 3, 4 or 5 when A is 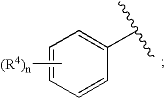
   n=1, 2, 3 or 4 when A is 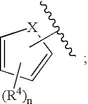
   and Z is O or S;
   the method including:
   (a) reacting a compound having the formula: 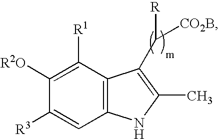
      wherein m, R, R¹, R², and R³ are as defined above, and B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃ and wherein B is optionally independently substituted with one or more halogen, —OH, —C(O)OH, or —NH₂ with a strong base in an organic solvent;
   (b) treating with a compound of the formula 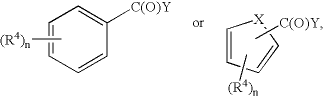
      wherein R⁴, n and X are described above and Y is a suitable leaving group to generate a compound having the formula: 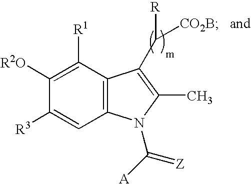
   (c) treating with an organic or inorganic aqueous acid in an organic solvent to prepare a compound having the formula:

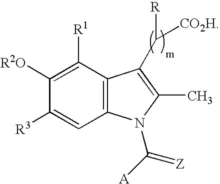

In various embodiments: A is 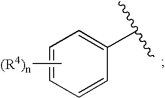
Z is O; m is 1; R is H; R is H and m is 1; B is —CH₂CH₂Si(CH₃)₃; R⁴ is halogen; R⁴ is Cl; n is 1; R¹ is H; A is

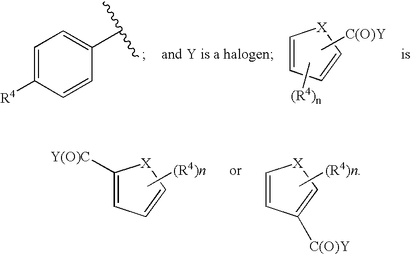

The invention also features useful intermediates such as compounds having the formula: 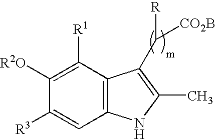
wherein, each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen; m is 1, 2, 3 or 4; R¹ is H or a halogen; R² is H; a C₁ to C₆ alkyl, optionally independently substituted with one or more halogen; 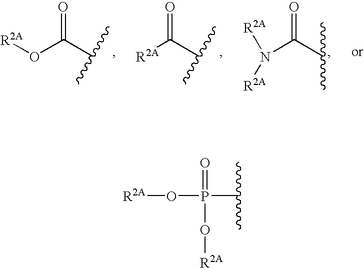
wherein each R^{2A} is independently: H, a C₁ to C₆ alkyl, a C₆ to C₁₀ alkenyl, a C₆ to C₁₀ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂; R³ is H or a halogen; and B is a C₁ to C₆ alkyl, a C₁ to C₆ alkenyl, a C₁ to C₆ alkynyl, aryl, cycloalkyl, or arylalkyl wherein one carbon is optionally replaced by Si(CH₃)₃ and wherein B is optionally independently substituted with one or more halogen, —OH, —C(O)OH, or —NH₂.

In various embodiments: R is H; m is 1; R¹ is H or a halogen; R² is H; a C₁ to C₆ alkyl, optionally independently substituted with one or more halogen; R³ is H or a halogen; and B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃.

In other embodiments: R² is H; a C₁ to C₃ alkyl, optionally independently substituted with one or more halogen; B is —CH₂CH₃, —CH₃, —CH₂C₆H₅, —CH₂CH═CH₂, or —C(CH₃)₃, optionally substituted with one or more halogen; and B is —CH₂CH₂Si(CH₃)₃.

The invention also features a compound having the formula 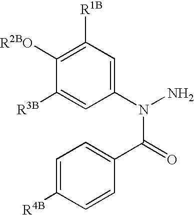
wherein RIB is H or a halogen; R^{2B} is H or a C₁ to C₆ alkyl, optionally independently substituted with one or more halogen; R^{3B} is H or a halogen; and R^{4B} is H or a halogen.

In various embodiments, R^{1B} is H, Cl or F; R^{3B} is H, Cl or F; and R^{4B} is H, Cl or F; and R^{2B} is H or a C₁ to C₃ alkyl.

The invention also features a compound having the formula: 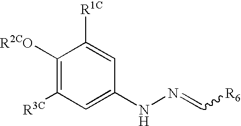
wherein R^{2C} is H or a C₁ to C₆ alkyl, optionally independently substituted with one or more halogen; R^{1C} and R^{3C} are independently H or a halogen; and R⁶ is a C₁ to C₁₀ alkyl, a C₆ to C₂₀ arylalkyl or a C₂ to C₂₀ alkenyl group.

In various embodiments: R^{2C} is methyl or ethyl; R^{1C} and R^{3C} are independently Cl or F; and/or R^{3C} are Cl; R^{1C} and/or R^{3C} are F; and R⁶ is a C₁ to C₁₀ alkyl, optionally substituted with a phenyl.

In another aspect the invention features a method for preparing a compound having the formula: 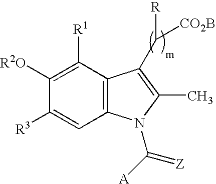
wherein: B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein B is optionally independently substituted with one or more halogen, —OH, —C(O)OH, or —NH₂; R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen; m is 1, 2, 3 or 4; R¹ is H or a halogen; R² is H; a C₁ to C₆ alkyl, optionally independently substituted with one or more halogen; 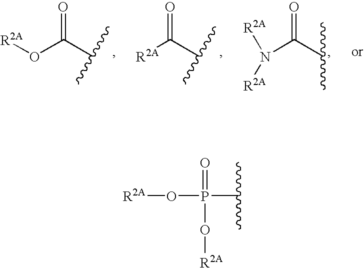
wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂; R³ is H or a halogen; A is 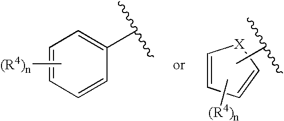
wherein each R⁴ is independently H, a halogen, —CN, —SH; —OH; a C₁-C₃ alkyl, optionally, independently substituted with one or more halogen; —OR^{4A}; —SR^{4A}; —SOR^{4A}; —S(O)₂R^{4A}, wherein R^{4A} is a C₁ to C₃ alkyl, optionally independently substituted with one or more halogen; X is S, O, NH or NR⁵, wherein R⁵ is a C₁ to C₁₀ alkyl; n=1,2,3, 4 or 5 when A is 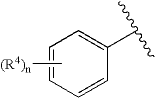
n=1,2, 3 or 4 when A is 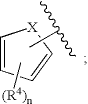
and Z is O or S:
the method including:
(a) reacting a compound having the formula: 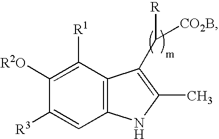
   wherein m, R, R¹, R², B and R³ are as defined above with a strong base in an organic solvent; and (b) adding 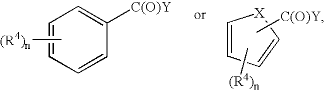
   wherein R⁴, n and X are described above and Y is a suitable leaving group to prepare a compound having the formula:

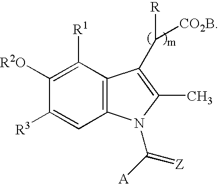

In various embodiments: A is 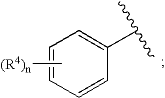
Z is O; m is 1; R is H; and R is H and m is 1;
In certain of the methods, 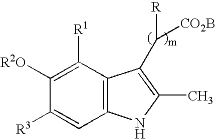
   in which B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃ and wherein B is optionally independently substituted with one or more halogen, —OH, —C(O)OH, or —NH₂; R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen; m is 1, 2, 3 or 4; R¹ is H or a halogen; R² is H; a C₁ to C₆ alkyl, optionally independently substituted with one or more halogen; 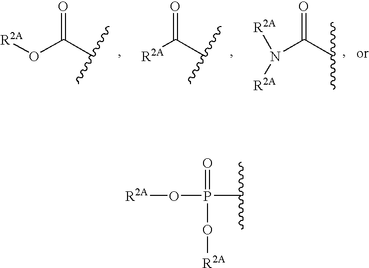
   wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂; and R³ is H or a halogen, is provided by reacting: 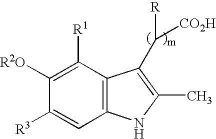
   with BOH, wherein B is as defined above.

In certain methods, 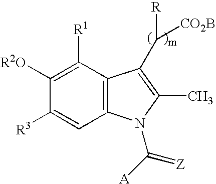
is provided by:
(a) treating 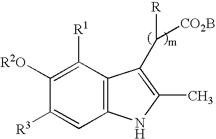
   wherein A, Z, m, R, R¹, R², R³ and B are as defined in claim 1 with a strong base in an organic solvent; and
(b) adding 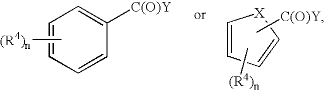
   wherein R⁴, n and X are as defined in claim 1 and Y is a suitable leaving group.

The invention features a method for preparing a compound having the formula: 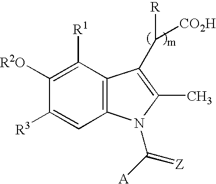
wherein:
each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen; m is 1, 2, 3 or 4; R¹ is H or a halogen; R² is H; a C₁ to C₆ alkyl, optionally independently substituted with one or more halogen; 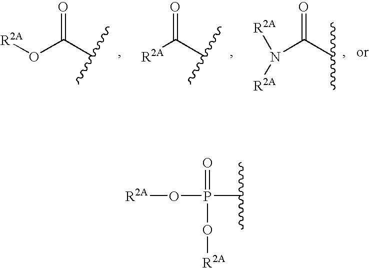
   wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂; R³ is H or a halogen; A is 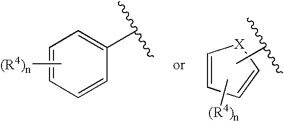
   wherein each R⁴ is independently H, a halogen, —CN, —SH; —OH; a C₁ to C₃ alkyl, optionally, independently substituted with one or more halogen; —OR^{4A}; —SR^{4A}; —SOR^{4A}, —S(O)₂R^{4A}, wherein R^{4A} is a C₁ to C₃ alkyl, optionally independently substituted with one or more halogen; X is S, O, NH or NR⁵, wherein R⁵ is a C₁ to C₁₀ alkyl; n=1, 2, 3, 4 or 5 when A is 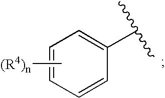
   n=1, 2, 3 or 4 when A is 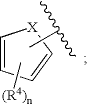
   and Z is O or S;
   the method including:
   (a) reacting 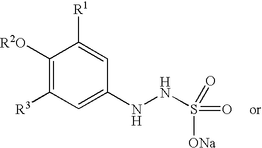 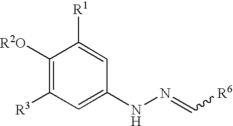
      wherein R¹, R² and R³ are as defined above and R⁶ is a with 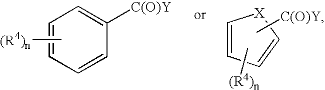
      wherein R⁴, n and X are described above and Y is a suitable leaving group;
   (b) treating with an acid in an alcohol and then neutralizing the acid to yield 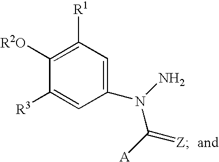
   (c) reacting the resulting compound with 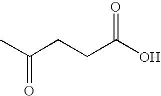
      to produce a compound having the formula:

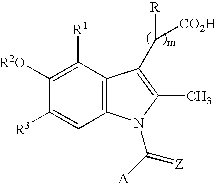

The invention also features a method for preparing a compound having the formula: 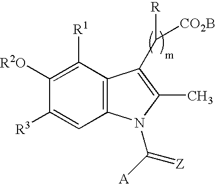
wherein: B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃ and wherein B is optionally independently substituted with one or more halogen, —OH, —C(O)OH, or —NH₂; each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen; m is 1, 2, 3 or 4; R¹ is H or a halogen; R² is H; a C₁ to C₆ alkyl, optionally independently substituted with one or more halogen; 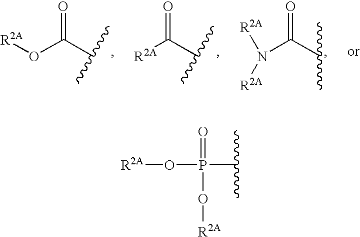
wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂; R³ is H or a halogen; A is 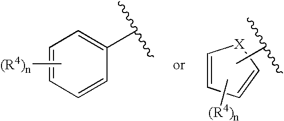
wherein each R⁴ is independently H, a halogen, —CN, —SH; —OH; a C₁ to C₃ alkyl, optionally, independently substituted with one or more halogen; —OR^{4A}; —SR^{4A}; —SOR^{4A}, —S(O)₂R^{4A}, wherein R^{4A} is a C₁ to C₃ alkyl, optionally independently substituted with one or more halogen; X is S, O, NH or NR³, wherein R³ is a C₁ to C₁₀ alkyl; n=1, 2, 3, 4 or 5 when A is 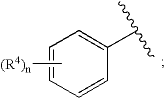
n=1, 2, 3 or 4 when A is 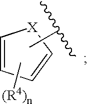
and Z is O or S:
the method including:
(a) reacting 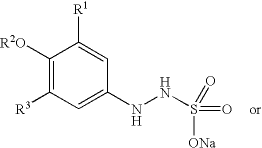 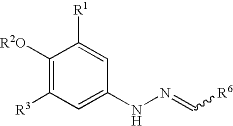
   wherein R¹, R² and R³ are as defined above and R⁶ is a C₁ to C₁₀ alkyl, alkenyl or alkynl with 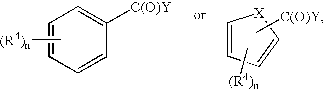
   wherein R⁴, n and X are described above and Y is a suitable leaving group;
(b) treating with an acid in an alcohol and then neutralizing the acid to yield 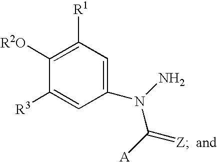
   (c) reacting the resulting compound with 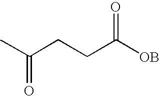
   wherein B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein B is optionally independently substituted with one or more halogen, —OH, —C(O)OH, or —NH₂ to produce a compound having the formula:

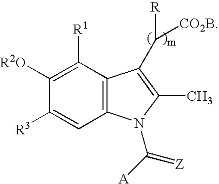

The invention also includes a method for preparing a compound having the formula: 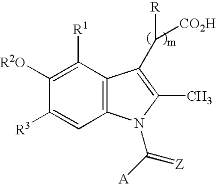
wherein: B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃ and wherein B is optionally independently substituted with one or more halogen, —OH, —C(O)OH, or —NH₂; Each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen; m is 1, 2, 3 or 4; R¹ is H or a halogen; R² is H; a C₁ to C₆ alkyl, optionally independently substituted with one or more halogen; 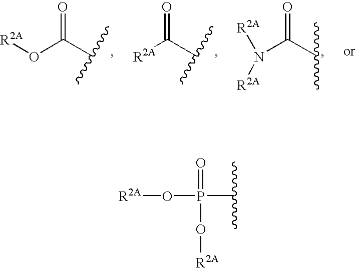
wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂; R³ is H or a halogen; A is 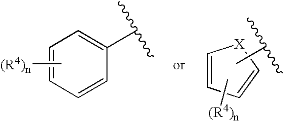
wherein each R⁴ is independently H, a halogen, —CN, —SH; —OH; a C₁ to C₃ alkyl, optionally, independently substituted with one or more halogen; —OR^{4A}; —SR^{4A}; —SOR^{4A}, —S(O)₂R^{4A}, wherein R^{4A} is a C₁ to C₃ alkyl, optionally independently substituted with one or more halogen; X is S, O, NH or NR⁵, R⁵ is a C₁ to C₁₀ alkyl; n=1, 2, 3, 4 or 5 when A is 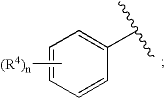
n=1, 2, 3 or 4 when A is 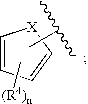
and Z is O or S;
the method including:
(a) reacting 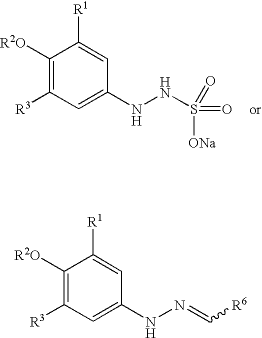
   wherein R¹, R² and R³ are as defined above and R⁶ is a C₁ to C₁₀ alkyl, alkenyl or alkynl with 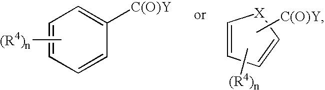
   wherein R⁴, n and X are described above and Y is a suitable leaving group;
(b) treating with an acid in an alcohol and then neutralizing the acid to yield 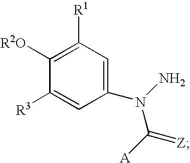
(c) reacting the resulting compound with 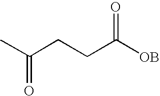
   to produce a compound having the formula: 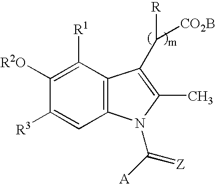
   and treating with an organic or inorganic acid to generate a compound having the formula:

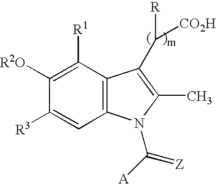

In another aspect, the invention includes a compound having the formula: 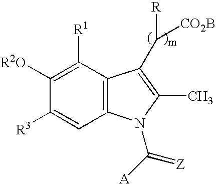
wherein each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen; m is 1, 2, 3 or 4; R¹ is H or a halogen; R² is H; a C₁ to C₆ alkyl, optionally independently substituted with one or more halogen; 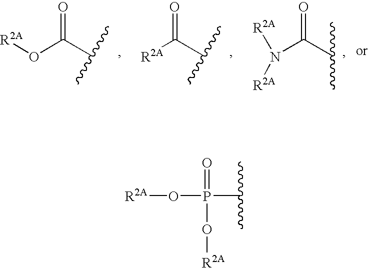
wherein each R^{2A} is independently: H. a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂; R³ is H or a halogen; A is 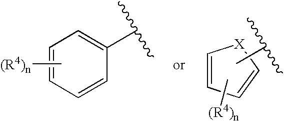
wherein each R⁴ is independently H, a halogen, —CN, —SH; —OH; a C₁ to C₃ alkyl, optionally, independently substituted with one or more halogen; —OR^{4A}; —SR^{4A}; —SOR^{4A}; —S(O)₂R^{4A}, wherein R^{4A} is a C₁ to C₃ alkyl, optionally independently substituted with one or more halogen; X is S, O, NH or NR⁵, wherein R⁵ is a C₁ to C₁₀ alkyl; n=1, 2, 3, 4 or 5 when A is 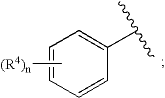
n=1, 2, 3 or 4 when A is 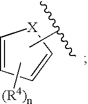
Z is O or S; and B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃ and wherein B is optionally independently substituted with one or more halogen, —OH, —C(O)OH, or —NH₂.

The invention also features a compound having the formula: 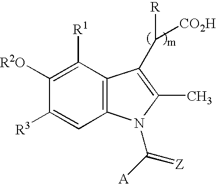
wherein each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen; m is 1, 2, 3 or 4; R¹ is H or a halogen; R² is H; a C₁ to C₆ alkyl, optionally independently substituted with one or more halogen; 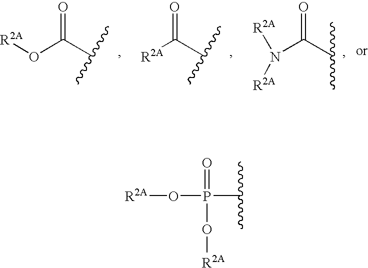
wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂; R³ is H or a halogen; A is 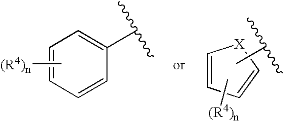
wherein each R⁴ is independently H, a halogen, —CN, —SH; —OH; a C₁ to C₃ alkyl, optionally, independently substituted with one or more halogen; OR^{4A}; SR^{4A}; SOR^{4A}; —S(O)₂R^{4A}, wherein R^{4A} is a C₁ to C₃ alkyl, optionally independently substituted with one or more halogen; X is S, O, NH or NR⁵, wherein R⁵ is a C₁ to C₁₀ alkyl; n=1, 2, 3, 4 or 5 when A is 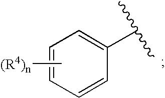
n=1,2, 3 or 4 when A is 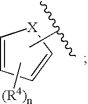
and Z is O or S.

The invention further features; a compound having the formula: 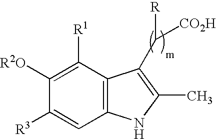
wherein, each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen; m is 1, 2, 3 or 4; R¹ is H or a halogen; R² is H; a C₁ to C₆ alkyl, optionally independently substituted with one or more halogen; 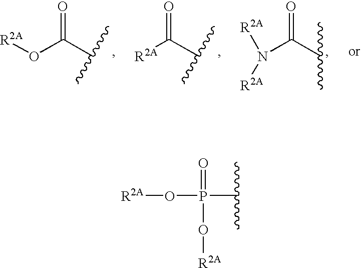
wherein each R^{2A} is independently: H, a C₁ to C₆ alkyl, a C₆ to C₁₀ alkenyl, a C₆ to C₁₀ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂; and R³ is H or a halogen.

Any of the methods described herein for making compounds having the formula: 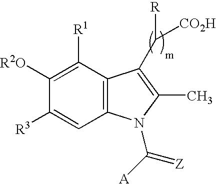
can be used to make any of the following compounds:
({[1-(4-chlorobenzoyl)-5-hydroxy-2-methyl-1H-indol-3-yl]acetyl}amino)acetic acid
3-({[1-(4-chlorobenzoyl)-5-hydroxy-2-methyl-1H-indol-3-yl]acetyl}amino)propanoic acid
3-({[1-(4-chlorobenzoyl)-5-hydroxy-2-methyl-1H-indol-3-yl]acetyl}amino)-2-methylpropanoic acid
3-({[1-(4-chlorobenzoyl)-5-hydroxy-2-methyl-1H-indol-3-yl]acetyl}amino)-3-methylpropanoic acid
2-({[1-(4-chlorobenzoyl)-5-hydroxy-2-methyl-1H-indol-3-yl]acetyl}amino)propanoic acid
2-({[1-(4-chlorobenzoyl)-5-hydroxy-2-methyl-1H-indol-3-yl]acetyl}amino)-2-methylpropanoic acid
(2R)-2-[1-(4-chlorobenzoyl)-5-hydroxy-2-methyl-1H-indol-3-yl]propionic acid,
({[2R)-2-[1-(4-chlorobenzoyl)-5-hydroxy-2-methyl-1H-indol-3-yl]propanoyl}amino)acetic acid
({(2R)-2-[1-(4-chlorobenzoyl)-5-hydroxy-2-methyl-1H-indol-3-yl]propanoyl}amino)-2-methylpropionic acid
({(2R)-2-[1-(4-chlorobenzoyl)-5-hydroxy-2-methyl-1H-indol-3-yl]propanoyl}amino)-2,2-methylbutyric acid
(2R)-3-({[1-(4-chlorobenzoyl)-5-hydroxy-2-methyl-1H-indol-3-yl]propanoyl}amino)propanoic acid
(2R)-3-({[1-(4-chlorobenzoyl)-5-hydroxy-2-methyl-1H-indol-3-yl]propanoyl}amino)-3-methylpropanic acid
(2R)-3-({[1-(4-chlorobenzoyl)-5-hydroxy-2-methyl-1H-indol-3-yl]propanoyl}amino)-2-methylpropanic acid
(2S)-2-[1-(4-chlorobenzoyl)-5-hydroxy-2-methyl-1H-indol-3-yl]propionic acid
({(2S)-2-[1-(4-chlorobenzoyl)-5-hydroxy-2-methyl-1H-indol-3-yl]propanoyl}amino)acetic acid
({(2S)-2-[1-(4-chlorobenzoyl)-5-hydroxy-2-methyl-1H-indol-3-yl]propanoyl}amino)-2-methylpropionic acid
({(2S)-2-[1-(4-chlorobenzoyl)-5-hydroxy-2-methyl-1H-indol-3-yl]propanoyl}amino)-2,2-methylbutyric acid
(2S)-3-({[1-(4-chlorobenzoyl)-5-hydroxy-2-methyl-1H-indol-3-yl]propanoyl}amino)propanoic acid
(2S)-3-({[1-(4-chlorobenzoyl)-5-hydroxy-2-methyl-1H-indol-3-yl]propanoyl}amino)-3-methylpropanic acid
(2S)-3-({[1-(4-chlorobenzoyl)-5-hydroxy-2-methyl-1H-indol-3-yl]propanoyl}amino)-2-methylpropanic acid
(2R,S)-2-[1-(4-chlorobenzoyl)-5-hydroxy-2-methyl-1H-indol-3-yl]propionic acid,
({(2R,S)-2-[1-(4-chlorobenzoyl)-5-hydroxy-2-methyl-1H-indol-3-yl]propanoyl}amino)acetic acid
({(2R,S)-2-[1-(4-chlorobenzoyl)-5-hydroxy-2-methyl-1H-indol-3-yl]propanoyl}amino)-2-methylpropionic acid
({(2R,S)-2-[1-(4-chlorobenzoyl)-5-hydroxy-2-methyl-1H-indol-3-yl]propanoyl}amino)-2,2-methylbutyric acid
(2R,S)-3-({[1-(4-chlorobenzoyl)-5-hydroxy-2-methyl-1H-indol-3-yl]propanoyl}amino)propanoic acid
(2R,S)-3-({[1-(4-chlorobenzoyl)-5-hydroxy-2-methyl-1H-indol-3-yl]propanoyl}amino)-3-methylpropanic acid
(2R,S)-3-({[1-(4-chlorobenzoyl)-5-hydroxy-2-methyl-1H-indol-3-yl]propanoyl}amino)-2-methylpropanic acid
[1-(4-chlorobenzoyl)-6-fluoro-5-hydroxy-2-methyl-1H-indol-3-yl]acetic acid
({[1-(4-chlorobenzoyl)-6-fluoro-5-hydroxy-2-methyl-1H-indol-3-yl]acetyl}amino)acetic acid
3-({[1-(4-chlorobenzoyl)-6-fluoro-5-hydroxy-2-methyl-1H-indol-3-yl]acetyl}amino)propanoic acid
3-({[1-(4-chlorobenzoyl)-6-fluoro-5-hydroxy-2-methyl-1H-indol-3-yl]acetyl}amino)-2-methylpropanoic acid
3-({[1-(4-chlorobenzoyl)-6-fluoro-5-hydroxy-2-methyl-1H-indol-3-yl]acetyl}amino)-3-methylpropanoic acid
2-({[1-(4-chlorobenzoyl)-6-fluoro-5-hydroxy-2-methyl-1H-indol-3-yl]acetyl}amino)propanoic acid
2-({[1-(4-chlorobenzoyl)-6-fluoro-5-hydroxy-2-methyl-1H-indol-3-yl]acetyl}amino)-2-methylpropanoic acid
[1-(4-chlorobenzoyl)-4-fluoro-5-hydroxy-2-methyl-1H-indol-3-yl]acetic acid,
({[1-(4-chlorobenzoyl)-4-fluoro-5-hydroxy-2-methyl-1H-indol-3-yl]acetyl}amino)acetic acid
3-({[1-(4-chlorobenzoyl)-4-fluoro-5-hydroxy-2-methyl-1H-indol-3-yl]acetyl}amino)propanoic acid
3-({[1-(4-chlorobenzoyl)-4-fluoro-5-hydroxy-2-methyl-1H-indol-3-yl]acetyl}amino)-2-methylpropanoic acid
3-({[1-(4-chlorobenzoyl)-4-fluoro-5-hydroxy-2-methyl-1H-indol-3-yl]acetyl}amino)-3-methylpropanoic acid
2-({[1-(4-chlorobenzoyl)-4-fluoro-5-hydroxy-2-methyl-1H-indol-3-yl]acetyl}amino)propanoic acid
2-({[1-(4-chlorobenzoyl)-4-fluoro-5-hydroxy-2-methyl-1H-indol-3-yl]acetyl}amino)-2-methylpropanoic acid
[1-(4-chlorobenzoyl)-4,6-difluoro-5-hydroxy-2-methyl-1H-indol-3-yl]acetic acid
({[1-(4-chlorobenzoyl)-4,6-difluoro-5-hydroxy-2-methyl-1H-indol-3-yl]acetyl}amino)acetic acid
3-({[1-(4-chlorobenzoyl)-4,6-difluoro-5-hydroxy-2-methyl-1H-indol-3-yl]acetyl}amino)propanoic acid
3-({[1-(4-chlorobenzoyl)-4,6-difluoro-5-hydroxy-2-methyl-1H-indol-3-yl]acetyl}amino)-2-methylpropanoic acid
3-({[1-(4-chlorobenzoyl)-4,6-difluoro-5-hydroxy-2-methyl-1H-indol-3-yl]acetyl}amino)-3-methylpropanoic acid
2-({[1-(4-chlorobenzoyl)-4,6-difluoro-5-hydroxy-2-methyl-1H-indol-3-yl]acetyl}amino)propanoic acid
2-({[1-(4-chlorobenzoyl)-4,6-difluoro-5-hydroxy-2-methyl-1H-indol-3-yl]acetyl}amino)-2-methylpropanoic acid
(2R,S)-2[1-(4-chlorobenzoyl)-4-fluoro-5-hydroxy-2-methyl-1H-indol-3-yl]propionic acid
({(2R,S)-2-[1-(4-chlorobenzoyl)-4-fluoro-5-hydroxy-2-methyl-1H-indol-3-yl]propanoyl}amino)acetic acid
({(2R,S)-2-[1-(4-chlorobenzoyl)-4-fluoro-5-hydroxy-2-methyl-1H-indol-3-yl]propanoyl}amino)-2-methylpropionic acid
({(2R,S)-2-[1-(4-chlorobenzoyl)-4-fluoro-5-hydroxy-2-methyl-1H-indol-3-yl]propanoyl}amino)-2,2-methylbutyric acid
(2R,S)-3-({[1-(4-chlorobenzoyl)-4-fluoro-5-hydroxy-2-methyl-1H-indol-3-yl]propanoyl}amino)propanoic acid
(2R,S)-3-({[1-(4-chlorobenzoyl)-4-fluoro-5-hydroxy-2-methyl-1H-indol-3-yl]propanoyl}amino)-3-methylpropanic acid
(2R,S)-3-({[1-(4-chlorobenzoyl)-4-fluoro-5-hydroxy-2-methyl-1H-indol-3-yl]propanoyl}amino)-2-methylpropanic acid
(2R,S)-2[1-(4-chlorobenzoyl)-6-fluoro-5-hydroxy-2-methyl-1H-indol-3-yl]propionic acid
({(2R,S)-2-[1-(4-chlorobenzoyl)-6-fluoro-5-hydroxy-2-methyl-1H-indol-3-yl]propanoyl}amino)acetic acid
({(2R,S)-2-[1-(4-chlorobenzoyl)-6-fluoro-5-hydroxy-2-methyl-1H-indol-3-yl]propanoyl}amino)-2-methylpropionic acid
({(2R,S)-2-[1-(4-chlorobenzoyl)-6-fluoro-5-hydroxy-2-methyl-1H-indol-3-yl]propanoyl}amino)-2,2-methylbutyric acid
(2R,S)-3-({[1-(4-chlorobenzoyl)-6-fluoro-5-hydroxy-2-methyl-1H-indol-3-yl]propanoyl}amino)propanoic acid
(2R,S)-3-({[1-(4-chlorobenzoyl)-6-fluoro-5-hydroxy-2-methyl-1H-indol-3-yl]propanoyl}amino)-3-methylpropanic acid
(2R,S)-3-({[1-(4-chlorobenzoyl)-6-fluoro-5-hydroxy-2-methyl-1H-indol-3-yl]propanoyl}amino)-2-methylpropanic acid
(2R,S)-2[1-(4-chlorobenzoyl)-4,6-difluoro-5-hydroxy-2-methyl-1H-indol-3-yl]propionic acid
({(2R,S)-2-[1-(4-chlorobenzoyl)-4,6-difluoro-5-hydroxy-2-methyl-1H-indol-3-yl]propanoyl}amino)acetic acid
({(2R,S)-2-[1-(4-chlorobenzoyl)-4,6-difluoro-5-hydroxy-2-methyl-1H-indol-3-yl]propanoyl}amino)-2-methylpropionic acid
({(2R,S)-2-[1-(4-chlorobenzoyl)-4,6-difluoro-5-hydroxy-2-methyl-1H-indol-3-yl]propanoyl}amino)-2,2-methylbutyric acid
(2R,S)-3-({[1-(4-chlorobenzoyl)-4,6-difluoro-5-hydroxy-2-methyl-1H-indol-3-yl]propanoyl}amino)propanoic acid
(2R,S)-3-({[1-(4-chlorobenzoyl)-4,6-difluoro-5-hydroxy-2-methyl-1H-indol-3-yl]propanoyl}amino)-3-methylpropanic acid
(2R,S)-3-({[1-(4-chlorobenzoyl)-4,6-difluoro-5-hydroxy-2-methyl-1H-indol-3-yl]propanoyl}amino)-2-methylpropanic acid
[1-(3-chlorobenzoyl)-4,6-difluoro-5-hydroxy-2-methyl-1H-indol-3-yl]acetic acid
({[1-(3-chlorobenzoyl)-4,6-difluoro-5-hydroxy-2-methyl-1H-indol-3-yl]acetyl}amino)acetic acid
3-({[1-(3-chlorobenzoyl)-4,6-difluoro-5-hydroxy-2-methyl-1H-indol-3-yl]acetyl}amino)propanoic acid
3-({[1-(3-chlorobenzoyl)-4,6-difluoro-5-hydroxy-2-methyl-1H-indol-3-yl]acetyl}amino)-2-methylpropanoic acid
3-({[1-(3-chlorobenzoyl)-4,6-difluoro-5-hydroxy-2-methyl-1H-indol-3-yl]acetyl}amino)-3-methylpropanoic acid
2-({[1-(3-chlorobenzoyl)-4,6-difluoro-5-hydroxy-2-methyl-1H-indol-3-yl]acetyl}amino)propanoic acid
2-({[1-(3-chlorobenzoyl)-4,6-difluoro-5-hydroxy-2-methyl-1H-indol-3-yl]acetyl}amino)-2-methylpropanoic acid
[1-(3-chlorobenzoyl)-6-fluoro-5-hydroxy-2-methyl-1H-indol-3-yl]acetic acid
({[1-(3-chlorobenzoyl)-6-fluoro-5-hydroxy-2-methyl-1H-indol-3-yl]acetyl}amino)acetic acid
3-({[1-(3-chlorobenzoyl)-6-fluoro-5-hydroxy-2-methyl-1H-indol-3-yl]acetyl}amino)propanoic acid
3-({[1-(3-chlorobenzoyl)-6-fluoro-5-hydroxy-2-methyl-1H-indol-3-yl]acetyl}amino)-2-methylpropanoic acid
3-({[1-(3-chlorobenzoyl)-6-fluoro-5-hydroxy-2-methyl-1H-indol-3-yl]acetyl}amino)-3-methylpropanoic acid
2-({[1-(3-chlorobenzoyl)-6-fluoro-5-hydroxy-2-methyl-1H-indol-3-yl]acetyl}amino)propanoic acid
2-({[1-(3-chlorobenzoyl)-6-fluoro-5-hydroxy-2-methyl-1H-indol-3-yl]acetyl}amino)-2-methylpropanoic acid
[1-(3-chlorobenzoyl)-4-fluoro-5-hydroxy-2-methyl-1H-indol-3-yl]acetic acid
({[1-(3-chlorobenzoyl)-4-fluoro-5-hydroxy-2-methyl-1H-indol-3-yl]acetyl}amino)acetic acid
3-({[1-(3-chlorobenzoyl)-4-fluoro-5-hydroxy-2-methyl-1H-indol-3-yl]acetyl}amino)propanoic acid
3-({[1-(3-chlorobenzoyl)-4-fluoro-5-hydroxy-2-methyl-1H-indol-3-yl]acetyl}amino)-2-methylpropanoic acid
3-({[1-(3-chlorobenzoyl)-4-fluoro-5-hydroxy-2-methyl-1H-indol-3-yl]acetyl}amino)-3-methylpropanoic acid
2-({[1-(3-chlorobenzoyl)-4-fluoro-5-hydroxy-2-methyl-1H-indol-3-yl]acetyl}amino)propanoic acid
2-({[1-(3-chlorobenzoyl)-4-fluoro-5-hydroxy-2-methyl-1H-indol-3-yl]acetyl}amino)-2-methylpropanoic acid
[1-(3-chlorobenzoyl)-5-hydroxy-2-methyl-1H-indol-3-yl]acetic acid
({[1-(3-chlorobenzoyl)-5-hydroxy-2-methyl-1H-indol-3-yl]acetyl}amino)acetic acid
3-({[1-(3-chlorobenzoyl)-5-hydroxy-2-methyl-1H-indol-3-yl]acetyl}amino)propanoic acid
3-({[1-(3-chlorobenzoyl)-5-hydroxy-2-methyl-1H-indol-3-yl]acetyl}amino)-2-methylpropanoic acid
3-({[1-(3-chlorobenzoyl)-5-hydroxy-2-methyl-1H-indol-3-yl]acetyl}amino)-3-methylpropanoic acid
2-({[1-(3-chlorobenzoyl)-5-hydroxy-2-methyl-1H-indol-3-yl]acetyl}amino)propanoic acid
2-({[1-(3-chlorobenzoyl)-5-hydroxy-2-methyl-1H-indol-3-yl]acetyl}amino)-2-methylpropanoic acid
[1-(3,4-dichlorobenzoyl)-5-hydroxy-2-methyl-1H-indol-3-yl]acetic acid
({[1-(3,4-dichlorobenzoyl)-5-hydroxy-2-methyl-1H-indol-3-yl]acetyl}amino)acetic acid
3-({[1-(3,4-dichlorobenzoyl)-5-hydroxy-2-methyl-1H-indol-3-yl]acetyl}amino)propanoic acid
3-({[1-(3,4-dichlorobenzoyl)-5-hydroxy-2-methyl-1H-indol-3-yl]acetyl}amino)-2-methylpropanoic acid
3-({[1-(3,4-dichlorobenzoyl)-5-hydroxy-2-methyl-1H-indol-3-yl]acetyl}amino)-3-methylpropanoic acid
2-({[1-(3,4-dichlorobenzoyl)-5-hydroxy-2-methyl-1H-indol-3-yl]acetyl}amino)propanoic acid
2-({[1-(3,4-dichlorobenzoyl)-5-hydroxy-2-methyl-1H-indol-3-yl]acetyl}amino)-2-methylpropanoic acid
[1-(3,4-dichlorobenzoyl)-6-fluoro-5-hydroxy-2-methyl-1H-indol-3-yl]acetic acid
({[1-(3,4-dichlorobenzoyl)-6-fluoro-5-hydroxy-2-methyl-1H-indol-3-yl]acetyl}amino)acetic acid
3-({[1-(3,4-dichlorobenzoyl)-6-fluoro-5-hydroxy-2-methyl-1H-indol-3-yl]acetyl}amino)propanoic acid
3-({[1-(3,4-dichlorobenzoyl)-6-fluoro-5-hydroxy-2-methyl-1H-indol-3-yl]acetyl}amino)-2-methylpropanoic acid
3-({[1-(3,4-dichlorobenzoyl)-6-fluoro-5-hydroxy-2-methyl-1H-indol-3-yl]acetyl}amino)-3-methylpropanoic acid
2-({[1-(3,4-dichlorobenzoyl)-6-fluoro-5-hydroxy-2-methyl-1H-indol-3-yl]acetyl}amino)propanoic acid
2-({[1-(3,4-dichlorobenzoyl)-6-fluoro-5-hydroxy-2-methyl-1H-indol-3-yl]acetyl}amino)-2-methylpropanoic acid
[1-(3,4-dichlorobenzoyl)-4-fluoro-5-hydroxy-2-methyl-1H-indol-3-yl]acetic acid
({[1-(3,4-dichlorobenzoyl)-4-fluoro-5-hydroxy-2-methyl-1H-indol-3-yl]acetyl}amino)acetic acid
3-({[1-(3,4-dichlorobenzoyl)-4-fluoro-5-hydroxy-2-methyl-1H-indol-3-yl]acetyl}amino)propanoic acid
3-({[1-(3,4-dichlorobenzoyl)-4-fluoro-5-hydroxy-2-methyl-1H-indol-3-yl]acetyl}amino)-2-methylpropanoic acid
3-({[1-(3,4-dichlorobenzoyl)-4-fluoro-5-hydroxy-2-methyl-1H-indol-3-yl]acetyl}amino)-3-methylpropanoic acid
2-({[1-(3,4-dichlorobenzoyl)-4-fluoro-5-hydroxy-2-methyl-1H-indol-3-yl]acetyl}amino)propanoic acid
2-({[1-(3,4-dichlorobenzoyl)-4-fluoro-5-hydroxy-2-methyl-1H-indol-3-yl]acetyl}amino)-2-methylpropanoic acid
[1-(3,4-dichlorobenzoyl)-4,6-difluoro-5-hydroxy-2-methyl-1H-indol-3-yl]acetic acid
({[1-(3,4-dichlorobenzoyl)-4,6-difluoro-5-hydroxy-2-methyl-1H-indol-3-yl]acetyl}amino)acetic acid
3-({[1-(3,4-dichlorobenzoyl)-4,6-difluoro-5-hydroxy-2-methyl-1H-indol-3-yl]acetyl}amino)propanoic acid
3-({[1-(3,4-dichlorobenzoyl)-4,6-difluoro-5-hydroxy-2-methyl-1H-indol-3-yl]acetyl}amino)-2-methylpropanoic acid
3-({[1-(3,4-dichlorobenzoyl)-4,6-difluoro-5-hydroxy-2-methyl-1H-indol-3-yl]acetyl}amino)-3-methylpropanoic acid
2-({[1-(3,4-dichlorobenzoyl)-4,6-difluoro-5-hydroxy-2-methyl-1H-indol-3-yl]acetyl}amino)propanoic acid
2-({[1-(3,4-dichlorobenzoyl)-4,6-difluoro-5-hydroxy-2-methyl-1H-indol-3-yl]acetyl}amino)-2-methylpropanoic acid
[1-(3,4-difluorobenzoyl)-5-hydroxy-2-methyl-1H-indol-3-yl]acetic acid
({[1-(3,4-difluorobenzoyl)-5-hydroxy-2-methyl-1H-indol-3-yl]acetyl}amino)acetic acid
3-({[1-(3,4-difluorobenzoyl)-5-hydroxy-2-methyl-1H-indol-3-yl]acetyl}amino)propanoic acid
3-({[1-(3,4-difluorobenzoyl)-5-hydroxy-2-methyl-1H-indol-3-yl]acetyl}amino)-2-methylpropanoic acid
3-({[1-(3,4-difluorobenzoyl)-5-hydroxy-2-methyl-1H-indol-3-yl]acetyl}amino)-3-methylpropanoic acid
2-({[1-(3,4-difluorobenzoyl)-5-hydroxy-2-methyl-1H-indol-3-yl]acetyl}amino)propanoic acid
2-({[1-(3,4-difluorobenzoyl)-5-hydroxy-2-methyl-1H-indol-3-yl]acetyl}amino)-2-methylpropanoic acid
[1-(3,4-difluorobenzoyl)-6-fluoro-5-hydroxy-2-methyl-1H-indol-3-yl]acetic acid
({[1-(3,4-difluorobenzoyl)-6-fluoro-5-hydroxy-2-methyl-1H-indol-3-yl]acetyl}amino)acetic acid
3-({[1-(3,4-difluorobenzoyl)-6-fluoro-5-hydroxy-2-methyl-1H-indol-3-yl]acetyl}amino)propanoic acid
3-({[1-(3,4-difluorobenzoyl)-6-fluoro-5-hydroxy-2-methyl-1H-indol-3-yl]acetyl}amino)-2-methylpropanoic acid
3-({[1-(3,4-difluorobenzoyl)-6-fluoro-5-hydroxy-2-methyl-1H-indol-3-yl]acetyl}amino)-3-methylpropanoic acid
2-({[1-(3,4-difluorobenzoyl)-6-fluoro-5-hydroxy-2-methyl-1H-indol-3-yl]acetyl}amino)propanoic acid
2-({[1-(3,4-difluorobenzoyl)-6-fluoro-5-hydroxy-2-methyl-1H-indol-3-yl]acetyl}amino)-2-methylpropanoic acid
[1-(3,4-difluorobenzoyl)-4-fluoro-5-hydroxy-2-methyl-1H-indol-3-yl]acetic acid
({[1-(3,4-difluorobenzoyl)-4-fluoro-5-hydroxy-2-methyl-1H-indol-3-yl]acetyl}amino)acetic acid
3-({[1-(3,4-difluorobenzoyl)-4-fluoro-5-hydroxy-2-methyl-1H-indol-3-yl]acetyl}amino)propanoic acid
3-({[1-(3,4-difluorobenzoyl)-4-fluoro-5-hydroxy-2-methyl-1H-indol-3-yl]acetyl}amino)-2-methylpropanoic acid
3-({[1-(3,4-difluorobenzoyl)-4-fluoro-5-hydroxy-2-methyl-1H-indol-3-yl]acetyl}amino)-3-methylpropanoic acid
2-({[1-(3,4-difluorobenzoyl)-4-fluoro-5-hydroxy-2-methyl-1H-indol-3-yl]acetyl}amino)propanoic acid
2-({[1-(3,4-difluorobenzoyl)-4-fluoro-5-hydroxy-2-methyl-1H-indol-3-yl]acetyl}amino)-2-methylpropanoic acid
[1-(3,4-difluorobenzoyl)-4,6-difluoro-5-hydroxy-2-methyl-1H-indol-3-yl]acetic acid
({[1-(3,4-difluorobenzoyl)-4,6-difluoro-5-hydroxy-2-methyl-1H-indol-3-yl]acetyl}amino)acetic acid
3-({[1-(3,4-difluorobenzoyl)-4,6-difluoro-5-hydroxy-2-methyl-1H-indol-3-yl]acetyl}amino)propanoic acid
3-({[1-(3,4-difluorobenzoyl)-4,6-difluoro-5-hydroxy-2-methyl-1H-indol-3-yl]acetyl}amino)-2-methylpropanoic acid
3-({[1-(3,4-difluorobenzoyl)-4,6-difluoro-5-hydroxy-2-methyl-1H-indol-3-yl]acetyl}amino)-3-methylpropanoic acid
2-({[1-(3,4-difluorobenzoyl)-4,6-difluoro-5-hydroxy-2-methyl-1H-indol-3-yl]acetyl}amino)propanoic acid
2-({[1-(3,4-difluorobenzoyl)-4,6-difluoro-5-hydroxy-2-methyl-1H-indol-3-yl]acetyl}amino)-2-methylpropanoic acid
[1-(4-chloro-3-fluorobenzoyl)—hydroxy-2-methyl-1H-indol-3-yl]acetic acid ([[1-(4-chloro-3-fluorobenzoyl)—hydroxy-2-methyl-1H-indol-3-yl]acetyl}amino)acetic acid
3-({[1-(4-chloro-3-fluorobenzoyl)-5-hydroxy-2-methyl-1H-indol-3-yl]acetyl}amino)propanoic acid
3-({[1-(4-chloro-3-fluorobenzoyl)-5-hydroxy-2-methyl-1H-indol-3-yl]acetyl}amino)-2-methylpropanoic acid
3-({[1-(4-chloro-3-fluorobenzoyl)-5-hydroxy-2-methyl-1H-indol-3-yl]acetyl}amino)-3-methylpropanoic acid
2-({[1-(4-chloro-3-fluorobenzoyl)-5-hydroxy-2-methyl-1H-indol-3-yl]acetyl}amino)propanoic acid
2-({[1-(4-chloro-3-fluorobenzoyl)-5-hydroxy-2-methyl-1H-indol-3-yl]acetyl}amino)-2-methylpropanoic acid
[1-(4-chloro-3-fluorobenzoyl)-6-fluoro-5-hydroxy-2-methyl-1H-indol-3-yl]acetic acid
({[1-(4-chloro-3-fluorobenzoyl)-6-fluoro-5-hydroxy-2-methyl-1H-indol-3-yl]acetyl}amino)acetic acid
3-({[1-(4-chloro-3-fluorobenzoyl)-6-fluoro-5-hydroxy-2-methyl-1H-indol-3-yl]acetyl}amino)propanoic acid
3-({[1-(4-chloro-3-fluorobenzoyl)-6-fluoro-5-hydroxy-2-methyl-1H-indol-3-yl]acetyl}amino)-2-methylpropanoic acid
3-({[1-(4-chloro-3-fluorobenzoyl)-6-fluoro-5-hydroxy-2-methyl-1H-indol-3-yl]acetyl}amino)-3-methylpropanoic acid
2-({[1-(4-chloro-3-fluorobenzoyl)-6-fluoro-5-hydroxy-2-methyl-1H-indol-3-yl]acetyl}amino)propanoic acid
2-({[1-(4-chloro-3-fluorobenzoyl)-6-fluoro-5-hydroxy-2-methyl-1H-indol-3-yl]acetyl}amino)-2-methylpropanoic acid
[1-(4-chloro-3-fluorobenzoyl)-4-fluoro-5-hydroxy-2-methyl-1H-indol-3-yl]acetic acid
({[1-(4-chloro-3-fluorobenzoyl)-4-fluoro-5-hydroxy-2-methyl-1H-indol-3-yl]acetyl}amino)acetic acid
3-({[1-(4-chloro-3-fluorobenzoyl)-4-fluoro-5-hydroxy-2-methyl-1H-indol-3-yl]acetyl}amino)propanoic acid
3-({[1-(4-chloro-3-fluorobenzoyl)-4-fluoro-5-hydroxy-2-methyl-1H-indol-3-yl]acetyl}amino)-2-methylpropanoic acid
3-({[1-(4-chloro-3-fluorobenzoyl)-4-fluoro-5-hydroxy-2-methyl-1H-indol-3-yl]acetyl}amino)-3-methylpropanoic acid
2-({[1-(4-chloro-3-fluorobenzoyl)-4-fluoro-5-hydroxy-2-methyl-1H-indol-3-yl]acetyl}amino)propanoic acid
2-({[1-(4-chloro-3-fluorobenzoyl)-4-fluoro-5-hydroxy-2-methyl-1H-indol-3-yl]acetyl}amino)-2-methylpropanoic acid
[1-(4-chloro-3-fluorobenzoyl)-4,6-difluoro-5-hydroxy-2-methyl-1H-indol-3-yl]acetic acid
({[1-(4-chloro-3-fluorobenzoyl)-4,6-difluoro-5-hydroxy-2-methyl-1H-indol-3-yl]acetyl}amino)acetic acid
3-({[1-(4-chloro-3-fluorobenzoyl)-4,6-difluoro-5-hydroxy-2-methyl-1H-indol-3-yl]acetyl}amino)propanoic acid
3-({[1-(4-chloro-3-fluorobenzoyl)-4,6-difluoro-5-hydroxy-2-methyl-1H-indol-3-yl]acetyl}amino)-2-methylpropanoic acid
3-({[1-(4-chloro-3-fluorobenzoyl)-4,6-difluoro-5-hydroxy-2-methyl 1H-indol-3-yl]acetyl}amino)-3-methylpropanoic acid
2-({[1-(4-chloro-3-fluorobenzoyl)-4,6-difluoro-5-hydroxy-2-methyl-1H-indol-3-yl]acetyl}amino)propanoic acid
2-({[1-(4-chloro-3-fluorobenzoyl)-4,6-difluoro-5-hydroxy-2-methyl-1H-indol-3-yl]acetyl}amino)-2-methylpropanoic acid
[1-(3-fluorobenzoyl)-5-hydroxy-2-methyl-1H-indol-3-yl]acetic acid
({[1-(3-fluorobenzoyl)-5-hydroxy-2-methyl-1H-indol-3-yl]acetyl}amino)acetic acid
3-({[1-(3-fluorobenzoyl)-5-hydroxy-2-methyl-1H-indol-3-yl]acetyl}amino)propanoic acid
3-({[1-(3-fluorobenzoyl)-5-hydroxy-2-methyl-1H-indol-3-yl]acetyl}amino)-2-methylpropanoic acid
3-({[1-(3-fluorobenzoyl)-5-hydroxy-2-methyl-1H-indol-3-yl]acetyl}amino)-3-methylpropanoic acid
2-({[1-(3-fluorobenzoyl)-5-hydroxy-2-methyl-1H-indol-3-yl]acetyl}amino)propanoic acid
2-({[1-(3-fluorobenzoyl)-5-hydroxy-2-methyl-1H-indol-3-yl]acetyl}amino)-2-methylpropanoic acid
[1-(3-fluorobenzoyl)-6-fluoro-5-hydroxy-2-methyl-1H-indol-3-yl]acetic acid
({[1-(3-fluorobenzoyl)-6-fluoro-5-hydroxy-2-methyl-1H-indol-3-yl]acetyl}amino)acetic acid
3-({[1-(3-fluorobenzoyl)-6-fluoro-5-hydroxy-2-methyl-1H-indol-3-yl]acetyl}amino)propanoic acid
3-({[1-(3-fluorobenzoyl)-6-fluoro-5-hydroxy-2-methyl-1H-indol-3-yl]acetyl}amino)-2-methylpropanoic acid
3-({[1-(3-fluorobenzoyl)-6-fluoro-5-hydroxy-2-methyl-1H-indol-3-yl]acetyl}amino)-3-methylpropanoic acid
2-({[1-(3-fluorobenzoyl)-6-fluoro-5-hydroxy-2-methyl-1H-indol-3-yl]acetyl}amino)propanoic acid
2-({[1-(3-fluorobenzoyl)-6-fluoro-5-hydroxy-2-methyl-1H-indol-3-yl]acetyl}amino)-2-methylpropanoic acid
[1-(3-fluorobenzoyl)-4-fluoro-5-hydroxy-2-methyl-1H-indol-3-yl]acetic acid
methyl[1-(3-fluorobenzoyl)-4-fluoro-5-hydroxy-2-methyl-1H-indol-3-yl]acetate
({[1-(3-fluorobenzoyl)-4-fluoro-5-hydroxy-2-methyl-1H-indol-3-yl]acetyl}amino)acetic acid
3-({[1-(3-fluorobenzoyl)-4-fluoro-5-hydroxy-2-methyl-1H-indol-3-yl]acetyl}amino)propanoic acid
3-({[1-(3-fluorobenzoyl)-4-fluoro-5-hydroxy-2-methyl-1H-indol-3-yl]acetyl}amino)-2-methylpropanoic acid
3-({[1-(3-fluorobenzoyl)-4-fluoro-5-hydroxy-2-methyl-1H-indol-3-yl]acetyl}amino)-3-methylpropanoic acid
2-({[1-(3-fluorobenzoyl)-4-fluoro-5-hydroxy-2-methyl-1H-indol-3-yl]acetyl}amino)propanoic acid
2-({[1-(3-fluorobenzoyl)-4-fluoro-5-hydroxy-2-methyl-1H-indol-3-yl]acetyl}amino)-2-methylpropanoic acid
[1-(3-fluorobenzoyl)-4,6-difluoro-5-hydroxy-2-methyl-1H-indol-3-yl]acetic acid
({[1-(3-fluorobenzoyl)-4,6-difluoro-5-hydroxy-2-methyl-1H-indol-3-yl]acetyl}amino)acetic acid
3-({[1-(3-fluorobenzoyl)-4,6-difluoro-5-hydroxy-2-methyl-1H-indol-3-yl]acetyl}amino)propanoic acid
3-({[1-(3-fluorobenzoyl)-4,6-difluoro-5-hydroxy-2-methyl-1H-indol-3-yl]acetyl}amino)-2-methylpropanoic acid
3-({[1-(3-fluorobenzoyl)-4,6-difluoro-5-hydroxy-2-methyl-1H-indol-3-yl]acetyl}amino)-3-methylpropanoic acid
2-({[1-(3-fluorobenzoyl)-4,6-difluoro-5-hydroxy-2-methyl-1H-indol-3-yl]acetyl}amino)propanoic acid
2-({[1-(3-fluorobenzoyl)-4,6-difluoro-5-hydroxy-2-methyl-1H-indol-3-yl]acetyl}amino)-2-methylpropanoic acid
(2R,S)-2[1-(3-chlorobenzoyl)-4,6-difluoro-5-hydroxy-2-methyl-1H-indol-3-yl]propionic acid
({(2R,S)-2-[1-(3-chlorobenzoyl)-4,6-difluoro-5-hydroxy-2-methyl-1H-indol-3-yl]propanoyl}amino)acetic acid
({(2R,S)-2-[1-(3-chlorobenzoyl)-4,6-difluoro-5-hydroxy-2-methyl-1H-indol-3-yl]propanoyl}amino)-2-methylpropionic acid
({(2R,S)-2-[1-(3-chlorobenzoyl)-4,6-difluoro-5-hydroxy-2-methyl-1H-indol-3-yl]propanoyl}amino)-2,2-methylbutyric acid
(2R,S)-3-({[1-(3-chlorobenzoyl)-4,6-difluoro-5-hydroxy-2-methyl-1H-indol-3-yl]propanoyl}amino)propanoic acid
(2R,S)-3-({[1-(3-chlorobenzoyl)-4,6-difluoro-5-hydroxy-2-methyl-1H-indol-3-yl]propanoyl}amino)-3-methylpropanic acid
(2R,S)-3-({[1-(3-chlorobenzoyl)-4,6-difluoro-5-hydroxy-2-methyl-1H-indol-3-yl]propanoyl}amino)-2-methylpropanic acid
(2R,S)-2[1-(3-chlorobenzoyl)-6-fluoro-5-hydroxy-2-methyl-1H-indol-3-yl]propionic acid
({(2R,S)-2-[1-(3-chlorobenzoyl)-6-fluoro-5-hydroxy-2-methyl-1H-indol-3-yl]propanoyl}amino)acetic acid
({(2R,S)-2-[1-(3-chlorobenzoyl)-6-fluoro-5-hydroxy-2-methyl-1H-indol-3-yl]propanoyl}amino)-2-methylpropionic acid
({(2R,S)-2-[1-(3-chlorobenzoyl)-6-fluoro-5-hydroxy-2-methyl-1H-indol-3-yl]propanoyl}amino)-2,2-methylbutyric acid
(2R,S)-3-({[1-(3-chlorobenzoyl)-6-fluoro-5-hydroxy-2-methyl-1H-indol-3-yl]propanoyl}amino)propanoic acid
(2R,S)-3-({[1-(3-chlorobenzoyl)-6-fluoro-5-hydroxy-2-methyl-1H-indol-3-yl]propanoyl}amino)-3-methylpropanic acid
(2R,S)-3-({[1-(3-chlorobenzoyl)-6-fluoro-5-hydroxy-2-methyl-1H-indol-3-yl]propanoyl}amino)-2-methylpropanic acid
(2R,S)-2[1-(3-chlorobenzoyl)-4-fluoro-5-hydroxy-2-methyl-1H-indol-3-yl]propionic acid
({(2R,S)-2-[1-(3-chlorobenzoyl)-4-fluoro-5-hydroxy-2-methyl-1H-indol-3-yl]propanoyl}amino)acetic acid
({(2R,S)-2-[1-(3-chlorobenzoyl)-4-fluoro-5-hydroxy-2-methyl-1H-indol-3-yl]propanoyl}amino)-2-methylpropionic acid
({(2R,S)-2-[1-(3-chlorobenzoyl)-4-fluoro-5-hydroxy-2-methyl-1H-indol-3-yl]propanoyl}amino)-2,2-methylbutyric acid
(2R,S)-3-({[1-(3-chlorobenzoyl)-4-fluoro-5-hydroxy-2-methyl-1H-indol-3-yl]propanoyl}amino)propanoic acid
(2R,S)-3-({[1-(3-chlorobenzoyl)-4-fluoro-5-hydroxy-2-methyl-1H-indol-3-yl]propanoyl}amino)-3-methylpropanic acid
(2R,S)-3-({[1-(3-chlorobenzoyl)-4-fluoro-5-hydroxy-2-methyl-1H-indol-3-yl]propanoyl}amino)-2-methylpropanic acid
(2R,S)-2[1-(3-chlorobenzoyl)-5-hydroxy-2-methyl-1H-indol-3-yl]propionic acid
({(2R,S)-2-[1-(3-chlorobenzoyl)-5-hydroxy-2-methyl-1H-indol-3-yl]propanoyl}amino)acetic acid
({(2R,S)-2-[1-(3-chlorobenzoyl)-5-hydroxy-2-methyl-1H-indol-3-yl]propanoyl}amino)-2-methylpropionic acid
({(2R,S)-2-[1-(3-chlorobenzoyl)-5-hydroxy-2-methyl-1H-indol-3-yl]propanoyl}amino)-2,2-methylbutyric acid
(2R,S)-3-({[1-(3-chlorobenzoyl)-5-hydroxy-2-methyl-1H-indol-3-yl]propanoyl}amino)propanoic acid
(2R,S)-3-({[1-(3-chlorobenzoyl)-5-hydroxy-2-methyl-1H-indol-3-yl]propanoyl}amino)-3-methylpropanic acid
(2R,S)-3-({[1-(3-chlorobenzoyl)-5-hydroxy-2-methyl-1H-indol-3-yl]propanoyl}amino)-2-methylpropanic acid
(2R,S)-2[1-(3,4-dichlorobenzoyl)-5-hydroxy-2-methyl-1H-indol-3-yl]propionic acid
({(2R,S)-2-[1-(3,4-dichlorobenzoyl)-5-hydroxy-2-methyl-1H-indol-3-yl]propanoyl}amino)acetic acid
({(2R,S)-2-[1-(3,4-dichlorobenzoyl)-5-hydroxy-2-methyl-1H-indol-3-yl]propanoyl}amino)-2-methylpropionic acid
({(2R,S)-2-[1-(3,4-dichlorobenzoyl)-5-hydroxy-2-methyl-1H-indol-3-yl]propanoyl}amino)-2,2-methylbutyric acid
(2R,S)-3-({[1-(3,4-dichlorobenzoyl)-5-hydroxy-2-methyl-1H-indol-3-yl]propanoyl}amino)propanoic acid
(2R,S)-3-({[1-(3,4-dichlorobenzoyl)-5-hydroxy-2-methyl-1H-indol-3-yl]propanoyl}amino)-3-methylpropanic acid
(2R,S)-3-({[1-(3,4-dichlorobenzoyl)-5-hydroxy-2-methyl-1H-indol-3-yl]propanoyl}amino)-2-methylpropanic acid
(2R,S)-2[1-(3,4-dichlorobenzoyl)-6-fluoro-5-hydroxy-2-methyl-1H-indol-3-yl]propionic acid
({(2R,S)-2-[1-(3,4-dichlorobenzoyl)-6-fluoro-5-hydroxy-2-methyl-1H-indol-3-yl]propanoyl}amino)acetic acid
({(2R,S)-2-[1-(3,4-dichlorobenzoyl)-6-fluoro-5-hydroxy-2-methyl-1H-indol-3-yl]propanoyl}amino)-2-methylpropionic acid
({(2R,S)-2-[1-(3,4-dichlorobenzoyl)-6-fluoro-5-hydroxy-2-methyl-1H-indol-3-yl]propanoyl}amino)-2,2-methylbutyric acid
(2R,S)-3-({[1-(3,4-dichlorobenzoyl)-6-fluoro-5-hydroxy-2-methyl-1H-indol-3-yl]propanoyl}amino)propanoic acid
(2R,S)-3-({[1-(3,4-dichlorobenzoyl)-6-fluoro-5-hydroxy-2-methyl-1H-indol-3-yl]propanoyl}amino)-3-methylpropanic acid
(2R,S)-3-({[1-(3,4-dichlorobenzoyl)-6-fluoro-5-hydroxy-2-methyl-1H-indol-3-yl]propanoyl}amino)-2-methylpropanic acid
(2R,S)-2[1-(3,4-dichlorobenzoyl)-4-fluoro-5-hydroxy-2-methyl-1H-indol-3-yl]propionic acid
({(2R,S)-2-[1-(3,4-dichlorobenzoyl)-4-fluoro-5-hydroxy-2-methyl-1H-indol-3-yl]propanoyl}amino)acetic acid
({(2R,S)-2-[1-(3,4-dichlorobenzoyl)-4-fluoro-5-hydroxy-2-methyl-1H-indol-3-yl]propanoyl}amino)-2-methylpropionic acid
({(2R,S)-2-[1-(3,4-dichlorobenzoyl)-4-fluoro-5-hydroxy-2-methyl-1H-indol-3-yl]propanoyl}amino)-2,2-methylbutyric acid
(2R,S)-3-({[1-(3,4-dichlorobenzoyl)-4-fluoro-5-hydroxy-2-methyl-1H-indol-3-yl]propanoyl}amino)propanoic acid
(2R,S)-3-({[1-(3,4-dichlorobenzoyl)-4-fluoro-5-hydroxy-2-methyl-1H-indol-3-yl]propanoyl}amino)-3-methylpropanic acid
(2R,S)-3-({[1-(3,4-dichlorobenzoyl)-4-fluoro-5-hydroxy-2-methyl-1H-indol-3-yl]propanoyl}amino)-2-methylpropanic acid
(2R,S)-2[1-(3,4-dichlorobenzoyl)-4,6-difluoro-5-hydroxy-2-methyl-1H-indol-3-yl]propionic acid
({(2R,S)-2-[1-(3,4-dichlorobenzoyl)-4,6-difluoro-5-hydroxy-2-methyl-1H-indol-3-yl]propanoyl}amino)acetic acid
({(2R,S)-2-[1-(3,4-dichlorobenzoyl)-4,6-difluoro-5-hydroxy-2-methyl-1H-indol-3-yl]propanoyl}amino)-2-methylpropionic acid
({(2R,S)-2-[1-(3,4-dichlorobenzoyl)-4,6-difluoro-5-hydroxy-2-methyl-1H-indol-3-yl]propanoyl}amino)-2,2-methylbutyric acid
(2R,S)-3-({[1-(3,4-dichlorobenzoyl)-4,6-difluoro-5-hydroxy-2-methyl-1H-indol-3-yl]propanoyl}amino)propanoic acid
(2R,S)-3-({[1-(3,4-dichlorobenzoyl)-4,6-difluoro-5-hydroxy-2-methyl-1H-indol-3-yl]propanoyl}amino)-3-methylpropanic acid
(2R,S)-3-({[1-(3,4-dichlorobenzoyl)-4,6-difluoro-5-hydroxy-2-methyl-1H-indol-3-yl]propanoyl}amino)-2-methylpropanic acid
(2R,S)-2[1-(3,4-difluorobenzoyl)-5-hydroxy-2-methyl-1H-indol-3-yl]propionic acid
({(2R,S)-2-[1-(3,4-difluorobenzoyl)-5-hydroxy-2-methyl-1H-indol-3-yl]propanoyl}amino)acetic acid
({(2R,S)-2-[1-(3,4-difluorobenzoyl)-5-hydroxy-2-methyl-1H-indol-3-yl]propanoyl}amino)-2-methylpropionic acid
({(2R,S)-2-[1-(3,4-difluorobenzoyl)-5-hydroxy-2-methyl-1H-indol-3-yl]propanoyl}amino)-2,2-methylbutyric acid
(2R,S)-3-({[1-(3,4-difluorobenzoyl)-5-hydroxy-2-methyl-1H-indol-3-yl]propanoyl}amino)propanoic acid
(2R,S)-3-({[1-(3,4-difluorobenzoyl)-5-hydroxy-2-methyl-1H-indol-3-yl]propanoyl}amino)-3-methylpropanic acid
(2R,S)-3-({[1-(3,4-difluorobenzoyl)-5-hydroxy-2-methyl-1H-indol-3-yl]propanoyl}amino)-2-methylpropanic acid
(2R,S)-2[1-(3,4-difluorobenzoyl)-6-fluoro-5-hydroxy-2-methyl-1H-indol-3-yl]propionic acid
({(2R,S)-2-[1-(3,4-difluorobenzoyl)-6-fluoro-5-hydroxy-2-methyl-1H-indol-3-yl]propanoyl}amino)acetic acid
({(2R,S)-2-[1-(3,4-difluorobenzoyl)-6-fluoro-5-hydroxy-2-methyl-1H-indol-3-yl]propanoyl}amino)-2-methylpropionic acid
({(2R,S)-2-[1-(3,4-difluorobenzoyl)-6-fluoro-5-hydroxy-2-methyl-1H-indol-3-yl]propanoyl}amino)-2,2-methylbutyric acid
(2R,S)-3-({[1-(3,4-difluorobenzoyl)-6-fluoro-5-hydroxy-2-methyl-1H-indol-3-yl]propanoyl}amino)propanoic acid
(2R,S)-3-({[1-(3,4-difluorobenzoyl)-6-fluoro-5-hydroxy-2-methyl-1H-indol-3-yl]propanoyl}amino)-3-methylpropanic acid
(2R,S)-3-({[1-(3,4-difluorobenzoyl)-6-fluoro-5-hydroxy-2-methyl-1H-indol-3-yl]propanoyl}amino)-2-methylpropanic acid
(2R,S)-2[1-(3,4-difluorobenzoyl)-4-fluoro-5-hydroxy-2-methyl-1H-indol-3-yl]propionic acid
({(2R,S)-2-[1-(3,4-difluorobenzoyl)-4-fluoro-5-hydroxy-2-methyl-1H-indol-3-yl]propanoyl}amino)acetic acid
({(2R,S)-2-[1-(3,4-difluorobenzoyl)-4-fluoro-5-hydroxy-2-methyl-1H-indol-3-yl]propanoyl}amino)-2-methylpropionic acid
({(2R,S)-2-[1-(3,4-difluorobenzoyl)-4-fluoro-5-hydroxy-2-methyl-1H-indol-3-yl]propanoyl}amino)-2,2-methylbutyric acid
(2R,S)-3-({[1-(3,4-difluorobenzoyl)-4-fluoro-5-hydroxy-2-methyl-1H-indol-3-yl]propanoyl}amino)propanoic acid
(2R,S)-3-({[1-(3,4-difluorobenzoyl)-4-fluoro-5-hydroxy-2-methyl-1H-indol-3-yl]propanoyl}amino)-3-methylpropanic acid
(2R,S)-3-({[1-(3,4-difluorobenzoyl)-4-fluoro-5-hydroxy-2-methyl-1H-indol-3-yl]propanoyl}amino)-2-methylpropanic acid
(2R,S)-2[1-(3,4-difluorobenzoyl)-4,6-difluoro-5-hydroxy-2-methyl-1H-indol-3-yl]propionic acid
({(2R,S)-2-[1-(3,4-difluorobenzoyl)-4,6-difluoro-5-hydroxy-2-methyl-1H-indol-3-yl]propanoyl}amino)acetic acid
({(2R,S)-2-[1-(3,4-difluorobenzoyl)-4,6-difluoro-5-hydroxy-2-methyl-1H-indol-3-yl]propanoyl}amino)-2-methylpropionic acid
({(2R,S)-2-[1-(3,4-difluorobenzoyl)-4,6-difluoro-5-hydroxy-2-methyl-1H-indol-3-yl]propanoyl}amino)-2,2-methylbutyric acid
(2R,S)-3-({[1-(3,4-difluorobenzoyl)-4,6-difluoro-5-hydroxy-2-methyl-1H-indol-3-yl]propanoyl}amino)propanoic acid
(2R,S)-3-({[1-(3,4-difluorobenzoyl)-4,6-difluoro-5-hydroxy-2-methyl-1H-indol-3-yl]propanoyl}amino)-3-methylpropanic acid
(2R,S)-3-({[1-(3,4-difluorobenzoyl)-4,6-difluoro-5-hydroxy-2-methyl-1H-indol-3-yl]propanoyl}amino)-2-methylpropanic acid
(2R,S)-2[1-(4-choro-3-fluorobenzoyl)-5-hydroxy-2-methyl-1H-indol-3-yl]propionic acid
methyl(2R,S)-2-[1-(4-choro-3-fluorobenzoyl)-5-hydroxy-2-methyl-1H-indol-3-({(2R,S)-2-[1-(4-choro-3-fluorobenzoyl)-5-hydroxy-2-methyl-1H-indol-3-yl]propanoyl}amino)acetic acid
({(2R,S)-2-[1-(4-choro-3-fluorobenzoyl)-5-hydroxy-2-methyl-1H-indol-3-yl]propanoyl}amino)-2-methylpropionic acid
({(2R,S)-2-[1-(4-choro-3-fluorobenzoyl)-5-hydroxy-2-methyl-1H-indol-3-yl]propanoyl}amino)-2,2-methylbutyric acid
(2R,S)-3-({[1-(4-choro-3-fluorobenzoyl)-5-hydroxy-2-methyl-1H-indol-3-yl]propanoyl}amino)propanoic acid
(2R,S)-3-({[1-(4-choro-3-fluorobenzoyl)-5-hydroxy-2-methyl-1H-indol-3-yl]propanoyl}amino)-3-methylpropanic acid
(2R,S)-3-({[1-(4-choro-3-fluorobenzoyl)-5-hydroxy-2-methyl-1H-indol-3-yl]propanoyl}amino)-2-methylpropanic acid
(2R,S)-2[1-(4-choro-3-fluorobenzoyl)-6-fluoro-5-hydroxy-2-methyl-1H-indol-3-yl]propionic acid
({(2R,S)-2-[1-(4-choro-3-fluorobenzoyl)-6-fluoro-5-hydroxy-2-methyl-1H-indol-3-yl]propanoyl}amino)acetic acid
({(2R,S)-2-[1-(4-choro-3-fluorobenzoyl)-6-fluoro-5-hydroxy-2-methyl-1H-indol-3-yl]propanoyl}amino)-2-methylpropionic acid
({(2R,S)-2-[1-(4-choro-3-fluorobenzoyl)-6-fluoro-5-hydroxy-2-methyl-1H-indol-3-yl]propanoyl}amino)-2,2-methylbutyric acid
(2R,S)-3-({[1-(4-choro-3-fluorobenzoyl)-6-fluoro-5-hydroxy-2-methyl-1H-indol-3-yl]propanoyl}amino)propanoic acid
(2R,S)-3-({[1-(4-choro-3-fluorobenzoyl)-6-fluoro-5-hydroxy-2-methyl-1H-indol-3-yl]propanoyl}amino)-3-methylpropanic acid
(2R,S)-3-({[1-(4-choro-3-fluorobenzoyl)-6-fluoro-5-hydroxy-2-methyl-1H-indol-3-yl]propanoyl}amino)-2-methylpropanic acid
(2R,S)-2[1-(4-choro-3-fluorobenzoyl)-4-fluoro-5-hydroxy-2-methyl-1H-indol-3-yl]propionic acid
({(2R,S)-2-[1-(4-choro-3-fluorobenzoyl)-4-fluoro-5-hydroxy-2-methyl-1H-indol-3-yl]propanoyl}amino)acetic acid
({(2R,S)-2-[1-(4-choro-3-fluorobenzoyl)-4-fluoro-5-hydroxy-2-methyl-1H-indol-3-yl]propanoyl}amino)-2-methylpropionic acid
({(2R,S)-2-[1-(4-choro-3-fluorobenzoyl)-4-fluoro-5-hydroxy-2-methyl-1H-indol-3-yl]propanoyl}amino)-2,2-methylbutyric acid
(2R,S)-3-({[1-(4-choro-3-fluorobenzoyl)-4-fluoro-5-hydroxy-2-methyl-1H-indol-3-yl]propanoyl}amino)propanoic acid
(2R,S)-3-({[1-(4-choro-3-fluorobenzoyl)-4-fluoro-5-hydroxy-2-methyl-1H-indol-3-yl]propanoyl}amino)-3-methylpropanic acid
(2R,S)-3-({[1-(4-choro-3-fluorobenzoyl)-4-fluoro-5-hydroxy-2-methyl-1H-indol-3-yl]propanoyl}amino)-2-methylpropanic acid
(2R,S)-2[1-(4-choro-3-fluorobenzoyl)-4,6-difluoro-5-hydroxy-2-methyl-1H-indol-3-yl]propionic acid
({(2R,S)-2-[1-(4-choro-3-fluorobenzoyl)-4,6-difluoro-5-hydroxy-2-methyl-1H-indol-3-yl]propanoyl}amino)acetic acid
({(2R,S)-2-[1-(4-choro-3-fluorobenzoyl)-4,6-difluoro-5-hydroxy-2-methyl-1H-indol-3-yl]propanoyl}amino)-2-methylpropionic acid
({(2R,S)-2-[1-(4-choro-3-fluorobenzoyl)-4,6-difluoro-5-hydroxy-2-methyl-1H-indol-3-yl]propanoyl}amino)-2,2-methylbutyric acid
(2R,S)-3-({[1-(4-choro-3-fluorobenzoyl)-4,6-difluoro-5-hydroxy-2-methyl-1H-indol-3-yl]propanoyl}amino)propanoic acid
(2R,S)-3-({[1-(4-choro-3-fluorobenzoyl)-4,6-difluoro-5-hydroxy-2-methyl-1H-indol-3-yl]propanoyl}amino)-3-methylpropanic acid
(2R,S)-3-({[1-(4-choro-3-fluorobenzoyl)-4,6-difluoro-5-hydroxy-2-methyl-1H-indol-3-yl]propanoyl}amino)-2-methylpropanic acid
(2R,S)-2[1-(3-fluorobenzoyl)-5-hydroxy-2-methyl-1H-indol-3-yl]propionic acid
({(2R,S)-2-[1-(3-fluorobenzoyl)-5-hydroxy-2-methyl-1H-indol-3-yl]propanoyl}amino)acetic acid
({(2R,S)-2-[1-(3-fluorobenzoyl)-5-hydroxy-2-methyl-1H-indol-3-yl]propanoyl}amino)-2-methylpropionic acid
({(2R,S)-2-[1-(3-fluorobenzoyl)-5-hydroxy-2-methyl-1H-indol-3-yl]propanoyl}amino)-2,2-methylbutyric acid
(2R,S)-3-({[1-(3-fluorobenzoyl)-5-hydroxy-2-methyl-1H-indol-3-yl]propanoyl}amino)propanoic acid
(2R,S)-3-({[1-(3-fluorobenzoyl)-5-hydroxy-2-methyl-1H-indol-3-yl]propanoyl}amino)-3-methylpropanic acid
(2R,S)-3-({[1-(3-fluorobenzoyl)-5-hydroxy-2-methyl-1H-indol-3-yl]propanoyl}amino)-2-methylpropanic acid
(2R,S)-2[1-(3-fluorobenzoyl)-6-fluoro-5-hydroxy-2-methyl-1H-indol-3-yl]propionic acid
({(2R,S)-2-[1-(3-fluorobenzoyl)-6-fluoro-5-hydroxy-2-methyl-1H-indol-3-yl]propanoyl}amino)acetic acid
({(2R,S)-2-[1-(3-fluorobenzoyl)-6-fluoro-5-hydroxy-2-methyl-1H-indol-3-yl]propanoyl}amino)-2-methylpropionic acid
({(2R,S)-2-[1-(3-fluorobenzoyl)-6-fluoro-5-hydroxy-2-methyl-1H-indol-3-yl]propanoyl}amino)-2,2-methylbutyric acid
(2R,S)-3-({[1-(3-fluorobenzoyl)-6-fluoro-5-hydroxy-2-methyl-1H-indol-3-yl]propanoyl}amino)propanoic acid
(2R,S)-3-({[1-(3-fluorobenzoyl)-6-fluoro-5-hydroxy-2-methyl-1H-indol-3-yl]propanoyl}amino)-3-methylpropanic acid
(2R,S)-3-({[1-(3-fluorobenzoyl)-6-fluoro-5-hydroxy-2-methyl-1H-indol-3-yl]propanoyl}amino)-2-methylpropanic acid
(2R,S)-2[1-(3-fluorobenzoyl)-4-fluoro-5-hydroxy-2-methyl-1H-indol-3-yl]propionic acid
({(2R,S)-2-[1-(3-fluorobenzoyl)-4-fluoro-5-hydroxy-2-methyl-1H-indol-3-yl]propanoyl}amino)acetic acid
({(2R,S)-2-[1-(3-fluorobenzoyl)-4-fluoro-5-hydroxy-2-methyl-1H-indol-3-yl]propanoyl}amino)-2-methylpropionic acid
({(2R,S)-2-[1-(3-fluorobenzoyl)-4-fluoro-5-hydroxy-2-methyl-1H-indol-3-yl]propanoyl}amino)-2,2-methylbutyric acid
(2R,S)-3-({[1-(3-fluorobenzoyl)-4-fluoro-5-hydroxy-2-methyl-1H-indol-3-yl]propanoyl}amino)propanoic acid
(2R,S)-3-({[1-(3-fluorobenzoyl)-4-fluoro-5-hydroxy-2-methyl-1H-indol-3-yl]propanoyl}amino)-3-methylpropanic acid
(2R,S)-3-({[1-(3-fluorobenzoyl)-4-fluoro-5-hydroxy-2-methyl-1H-indol-3-yl]propanoyl}amino)-2-methylpropanic acid
(2R,S)-2[1-(3-fluorobenzoyl)-4,6-difluoro-5-hydroxy-2-methyl-1H-indol-3-yl]propionic acid
({(2R,S)-2-[1-(3-fluorobenzoyl)-4,6-difluoro-5-hydroxy-2-methyl-1H-indol-3-yl]propanoyl}amino)acetic acid
({(2R,S)-2-[1-(3-fluorobenzoyl)-4,6-difluoro-5-hydroxy-2-methyl-1H-indol-3-yl]propanoyl}amino)-2-methylpropionic acid
({(2R,S)-2-[1-(3-fluorobenzoyl)-4,6-difluoro-5-hydroxy-2-methyl-1H-indol-3-yl]propanoyl}amino)-2,2-methylbutyric acid
(2R,S)-3-({[1-(3-fluorobenzoyl)-4,6-difluoro-5-hydroxy-2-methyl-1H-indol-3-yl]propanoyl}amino)propanoic acid
(2R,S)-3-({[1-(3-fluorobenzoyl)-4,6-difluoro-5-hydroxy-2-methyl-1H-indol-3-yl]propanoyl}amino)-3-methylpropanic acid
(2R,S)-3-({[1-(3-fluorobenzoyl)-4,6-difluoro-5-hydroxy-2-methyl-1H-indol-3-yl]propanoyl}amino)-2-methylpropanic acid
[1-(4-trifluoromethoxybenzoyl)-5-hydroxy-2-methyl-1H-indol-3-yl]acetic acid
({[1-(4-trifluoromethoxybenzoyl)-5-hydroxy-2-methyl-1H-indol-3-yl]acetyl}amino)acetic acid
3-({[1-(4-trifluoromethoxybenzoyl)-5-hydroxy-2-methyl-1H-indol-3-yl]acetyl}amino)propanoic acid
3-({[1-(4-trifluoromethoxybenzoyl)-5-hydroxy-2-methyl-1H-indol-3-yl]acetyl}amino)-2-methylpropanoic acid
3-({[1-(4-trifluoromethoxybenzoyl)-5-hydroxy-2-methyl-1H-indol-3-yl]acetyl}amino)-3-methylpropanoic acid
2-({[1-(4-trifluoromethoxybenzoyl)-5-hydroxy-2-methyl-1H-indol-3-yl]acetyl}amino)propanoic acid
2-({[1-(4-trifluoromethoxybenzoyl)-5-hydroxy-2-methyl-1H-indol-3-yl]acetyl}amino)-2-methylpropanoic acid
(2R,S)-2[1-(4-trifluoromethoxybenzoyl)-5-hydroxy-2-methyl-1H-indol-3-yl]propionic acid
({(2R,S)-2-[1-(4-trifluoromethoxybenzoyl)-5-hydroxy-2-methyl-1H-indol-3-yl]propanoyl}amino)acetic acid
({(2R,S)-2-[1-(4-trifluoromethoxybenzoyl)-5-hydroxy-2-methyl-1H-indol-3-yl]propanoyl}amino)-2-methylpropionic acid
({(2R,S)-2-[1-(4-trifluoromethoxybenzoyl)-5-hydroxy-2-methyl-1H-indol-3-yl]propanoyl}amino)-2,2-methylbutyric acid
(2R,S)-3-({[1-(4-trifluoromethoxybenzoyl)-5-hydroxy-2-methyl-1H-indol-3-yl]propanoyl}amino)propanoic acid
(2R,S)-3-({[1-(4-trifluoromethoxybenzoyl)-5-hydroxy-2-methyl-1H-indol-3-yl]propanoyl}amino)-3-methylpropanic acid
(2R,S)-3-({[1-(4-trifluoromethoxybenzoyl)-5-hydroxy-2-methyl-1H-indol-3-yl]propanoyl}amino)-2-methylpropanic acid
[1-(4-trifluoromethoxybenzoyl)-6-fluoro-5-hydroxy-2-methyl-1H-indol-3-yl]acetic acid
({[1-(4-trifluoromethoxybenzoyl)-6-fluoro-5-hydroxy-2-methyl-1H-indol-3-yl]acetyl}amino)acetic acid
3-({[1-(4-trifluoromethoxybenzoyl)-6-fluoro-5-hydroxy-2-methyl-1H-indol-3-yl]acetyl}amino)propanoic acid
3-({[1-(4-trifluoromethoxybenzoyl)-6-fluoro-5-hydroxy-2-methyl-1H-indol-3-yl]acetyl}amino)-2-methylpropanoic acid
3-({[1-(4-trifluoromethoxybenzoyl)-6-fluoro-5-hydroxy-2-methyl-1H-indol-3-yl]acetyl}amino)-3-methylpropanoic acid
2-({[1-(4-trifluoromethoxybenzoyl)-6-fluoro-5-hydroxy-2-methyl-1H-indol-3-yl]acetyl}amino)propanoic acid
2-({[1-(4-trifluoromethoxybenzoyl)-6-fluoro-5-hydroxy-2-methyl-1H-indol-3-yl]acetyl}amino)-2-methylpropanoic acid
(2R,S)-2[1-(4-trifluoromethoxybenzoyl)-6-fluoro-5-hydroxy-2-methyl-1H-indol-3-yl]propionic acid
({(2R,S)-2-[1-(4-trifluoromethoxybenzoyl)-6-fluoro-5-hydroxy-2-methyl-1H-indol-3-yl]propanoyl}amino)acetic acid
({(2R,S)-2-[1-(4-trifluoromethoxybenzoyl)-6-fluoro-5-hydroxy-2-methyl-1H-indol-3-yl]propanoyl}amino)-2-methylpropionic acid
({(2R,S)-2-[1-(4-trifluoromethoxybenzoyl)-6-fluoro-5-hydroxy-2-methyl-1H-indol-3-yl]propanoyl}amino)-2,2-methylbutyric acid
(2R,S)-3-({[1-(4-trifluoromethoxybenzoyl)-6-fluoro-5-hydroxy-2-methyl-1H-indol-3-yl]propanoyl}amino)propanoic acid
(2R,S)-3-({[1-(4-trifluoromethoxybenzoyl)-6-fluoro-5-hydroxy-2-methyl-1H-indol-3-yl]propanoyl}amino)-3-methylpropanic acid
(2R,S)-3-({[1-(4-trifluoromethoxybenzoyl)-6-fluoro-5-hydroxy-2-methyl-1H-indol-3-yl]propanoyl}amino)-2-methylpropanic acid
[1-(4-trifluoromethoxybenzoyl)-4-fluoro-5-hydroxy-2-methyl-1H-indol-3-yl]acetic acid
({[1-(4-trifluoromethoxybenzoyl)-4-fluoro-5-hydroxy-2-methyl-1H-indol-3-yl]acetyl}amino)acetic acid
3-({[1-(4-trifluoromethoxybenzoyl)-4-fluoro-5-hydroxy-2-methyl-1H-indol-3-yl]acetyl}amino)propanoic acid
3-({[1-(4-trifluoromethoxybenzoyl)-4-fluoro-5-hydroxy-2-methyl-1H-indol-3-yl]acetyl}amino)-2-methylpropanoic acid
3-({[1-(4-trifluoromethoxybenzoyl)-4-fluoro-5-hydroxy-2-methyl-1H-indol-3-yl]acetyl}amino)-3-methylpropanoic acid
2-({[1-(4-trifluoromethoxybenzoyl)-4-fluoro-5-hydroxy-2-methyl-1H-indol-3-yl]acetyl}amino)propanoic acid
2-({[1-(4-trifluoromethoxybenzoyl)-4-fluoro-5-hydroxy-2-methyl-1H-indol-3-yl]acetyl}amino)-2-methylpropanoic acid
2-[1-(4-trifluoromethoxybenzoyl)-4-fluoro-5-hydroxy-2-methyl-1H-indol-3-yl]propionic acid
(2R,S)-2[1-(4-trifluoromethoxybenzoyl)-4-fluoro-5-hydroxy-2-methyl-1H-indol-3-yl]propionic acid
({(2R,S)-2-[1-(4-trifluoromethoxybenzoyl)-4-fluoro-5-hydroxy-2-methyl-1H-indol-3-yl]propanoyl}amino)acetic acid
({(2R,S)-2-[1-(4-trifluoromethoxybenzoyl)-4-fluoro-5-hydroxy-2-methyl-1H-indol-3-yl]propanoyl}amino)-2-methylpropionic acid
({(2R,S)-2-[1-(4-trifluoromethoxybenzoyl)-4-fluoro-5-hydroxy-2-methyl-1H-indol-3-yl]propanoyl}amino)-2,2-methylbutyric acid
(2R,S)-3-({[1-(4-trifluoromethoxybenzoyl)-4-fluoro-5-hydroxy-2-methyl-1H-indol-3-yl]propanoyl}amino)propanoic acid
(2R,S)-3-({[1-(4-trifluoromethoxybenzoyl)-4-fluoro-5-hydroxy-2-methyl-1H-indol-3-yl]propanoyl}amino)-3-methylpropanic acid
(2R,S)-3-({[1-(4-trifluoromethoxybenzoyl)-4-fluoro-5-hydroxy-2-methyl-1H-indol-3-yl]propanoyl}amino)-2-methylpropanic acid
[1-(4-trifluoromethoxybenzoyl)-4,6-difluoro-5-hydroxy-2-methyl-1H-indol-3-yl]acetic acid
({[1-(4-trifluoromethoxybenzoyl)-4,6-difluoro-5-hydroxy-2-methyl-1H-indol-3-yl]acetyl}amino)acetic acid
3-({[1-(4-trifluoromethoxybenzoyl)-4,6-difluoro-5-hydroxy-2-methyl-1H-indol-3-yl]acetyl}amino)propanoic acid
3-({[1-(4-trifluoromethoxybenzoyl)-4,6-difluoro-5-hydroxy-2-methyl-1H-indol-3-yl]acetyl}amino)-2-methylpropanoic acid
3-({[1-(4-trifluoromethoxybenzoyl)-4,6-difluoro-5-hydroxy-2-methyl-1H-indol-3-yl]acetyl}amino)-3-methylpropanoic acid
2-({[1-(4-trifluoromethoxybenzoyl)-4,6-difluoro-5-hydroxy-2-methyl-1H-indol-3-yl]acetyl}amino)propanoic acid
2-({[1-(4-trifluoromethoxybenzoyl)-4,6-difluoro-5-hydroxy-2-methyl-1H-indol-3-yl]acetyl}amino)-2-methylpropanoic acid
(2R,S)-2[1-(4-trifluoromethoxybenzoyl)-4,6-difluoro-5-hydroxy-2-methyl-1H-indol-3-yl]propionic acid
({(2R,S)-2-[1-(4-trifluoromethoxybenzoyl)-4,6-difluoro-5-hydroxy-2-methyl-1H-indol-3-yl]propanoyl}amino)acetic acid
({(2R,S)-2-[1-(4-trifluoromethoxybenzoyl)-4,6-difluoro-5-hydroxy-2-methyl-1H-indol-3-yl]propanoyl}amino)-2-methylpropionic acid
({(2R,S)-2-[1-(4-trifluoromethoxybenzoyl)-4,6-difluoro-5-hydroxy-2-methyl-1H-indol-3-yl]propanoyl}amino)-2,2-methylbutyric acid
(2R,S)-3-({[1-(4-trifluoromethoxybenzoyl)-4,6-difluoro-5-hydroxy-2-methyl-1H-indol-3-yl]propanoyl}amino)propanoic acid
(2R,S)-3-({[1-(4-trifluoromethoxybenzoyl)-4,6-difluoro-5-hydroxy-2-methyl-1H-indol-3-yl]propanoyl}amino)-3-methylpropanic acid
(2R,S)-3-({[1-(4-trifluoromethoxybenzoyl)-4,6-difluoro-5-hydroxy-2-methyl-1H-indol-3-yl]propanoyl}amino)-2-methylpropanic acid
{1-[4-(difluoromethoxy)benzoyl]-6-fluoro-5-methoxy-2-methyl-1H-indol-3-yl]acetic acid
({{1-[4-(difluoromethoxy)benzoyl]-6-fluoro-5-methoxy-2-methyl-1H-indol-3-yl]acetyl}amino)acetic acid
3-({{1-[4-(difluoromethoxy)benzoyl]-6-fluoro-5-methoxy-2-methyl-1H-indol-3-yl]acetyl}amino)propanoic acid
3-({{1-[4-(difluoromethoxy)benzoyl]-6-fluoro-5-methoxy-2-methyl-1H-indol-3-yl]acetyl}amino)-2-methylpropanoic acid
3-({{1-[4-(difluoromethoxy)benzoyl]-6-fluoro-5-methoxy-2-methyl-1H-indol-3-yl]acetyl}amino)-3-methylpropanoic acid
2-({{1-[4-(difluoromethoxy)benzoyl]-6-fluoro-5-methoxy-2-methyl-1H-indol-3-yl]acetyl}amino)propanoic acid
2-({{1-[4-(difluoromethoxy)benzoyl]-6-fluoro-5-methoxy-2-methyl-1H-indol-3-yl]acetyl}amino)-2-methylpropanoic acid
1-[4-(difluoromethoxy)benzoyl]-6-fluoro-5-hydroxy-2-methyl-1H-indol-3-yl]acetic acid
({{1-[4-(difluoromethoxy)benzoyl]-6-fluoro-5-hydroxy-2-methyl-1H-indol-3-yl]acetyl}amino)acetic acid
3-({{1-[4-(difluoromethoxy)benzoyl]-6-fluoro-5-hydroxy-2-methyl-1H-indol-3-yl]acetyl}amino)propanoic acid
3-({{1-[4-(difluoromethoxy)benzoyl]-6-fluoro-5-hydroxy-2-methyl-1H-indol-3-yl]acetyl}amino)-2-methylpropanoic acid
3-({{1-[4-(difluoromethoxy)benzoyl]-6-fluoro-5-hydroxy-2-methyl-1H-indol-3-yl]acetyl}amino)-3-methylpropanoic acid
2-({{1-[4-(difluoromethoxy)benzoyl]-6-fluoro-5-hydroxy-2-methyl-1H-indol-3-yl]acetyl}amino)propanoic acid
2-({{1-[4-(difluoromethoxy)benzoyl]-6-fluoro-5-hydroxy-2-methyl-1H-indol-3-yl]acetyl}amino)-2-methylpropanoic acid
1-[4-(difluoromethoxy)benzoyl]-5-methoxy-2-methyl-1H-indol-3-yl]acetic acid
({{1-[4-(difluoromethoxy)benzoyl]-5-methoxy-2-methyl-1H-indol-3-yl]acetyl}amino)acetic acid
3-({{1-[4-(difluoromethoxy)benzoyl]-5-methoxy-2-methyl-1H-indol-3-yl]acetyl}amino)propanoic acid
3-({{1-[4-(difluoromethoxy)benzoyl]-5-methoxy-2-methyl-1H-indol-3-yl]acetyl}amino)-2-methylpropanoic acid
3-({{1-[4-(difluoromethoxy)benzoyl]-5-methoxy-2-methyl-1H-indol-3-yl]acetyl}amino)-3-methylpropanoic acid
2-({{1-[4-(difluoromethoxy)benzoyl]-5-methoxy-2-methyl-1H-indol-3-yl]acetyl}amino)propanoic acid
2-({{1-[4-(difluoromethoxy)benzoyl]-5-methoxy-2-methyl-1H-indol-3-yl]acetyl}amino)-2-methylpropanoic acid
({{1-[4-(difluoromethoxy)benzoyl]-5-hydroxy-2-methyl-1H-indol-3-yl]acetyl}amino)acetic acid
3-({{1-[4-(difluoromethoxy)benzoyl]-5-hydroxy-2-methyl-1H-indol-3-yl]acetyl}amino)propanoic acid
3-({{1-[4-(difluoromethoxy)benzoyl]-5-hydroxy-2-methyl-1H-indol-3-yl]acetyl}amino)-2-methylpropanoic acid
3-({{1-[4-(difluoromethoxy)benzoyl]-5-hydroxy-2-methyl-1H-indol-3-yl]acetyl}amino)-3-methylpropanoic acid
2-({{1-[4-(difluoromethoxy)benzoyl]-5-hydroxy-2-methyl-1H-indol-3-yl]acetyl}amino)propanoic acid
2-({{1-[4-(difluoromethoxy)benzoyl]-5-hydroxy-2-methyl-1H-indol-3-yl]acetyl}amino)-2-methylpropanoic acid
1-[4-chlorobenzoyl]-6-chloro-5-methoxy-2-methyl-1H-indol-3-yl]acetic acid
({1-[4-chlorobenzoyl]-6-chloro-5-methoxy-2-methyl-1H-indol-3-yl]acetyl}amino)acetic acid
3-({{1-[4-chlorobenzoyl]-6-chloro-5-methoxy-2-methyl-1H-indol-3-yl]acetyl}amino)propanoic acid
3-({{1-[4-chlorobenzoyl]-6-chloro-5-methoxy-2-methyl-1H-indol-3-yl]acetyl}amino)-2-methylpropanoic acid
3-({{1-[4-chlorobenzoyl]-6-chloro-5-methoxy-2-methyl-1H-indol-3-yl]acetyl}amino)-3-methylpropanoic acid
2-({{1-[4-chlorobenzoyl]-6-chloro-5-methoxy-2-methyl-1H-indol-3-yl]acetyl}amino)propanoic acid
2-({{1-[4-chlorobenzoyl]-6-chloro-5-methoxy-2-methyl-1H-indol-3-yl]acetyl}amino)-2-methylpropanoic acid
1-[4-chlorobenzoyl]-6-chloro-5-hydroxy-2-methyl-1H-indol-3-yl]acetic acid
({{1-[4-chlorobenzoyl]-6-chloro-5-hydroxy-2-methyl-1H-indol-3-yl]acetyl}amino)acetic acid
3-({{1-[4-chlorobenzoyl]-6-chloro-5-hydroxy-2-methyl-1H-indol-3-yl]acetyl}amino)propanoic acid
3-({{1-[4-chlorobenzoyl]-6-chloro-5-hydroxy-2-methyl-1H-indol-3-yl]acetyl}amino)-2-methylpropanoic acid
3-({{1-[4-chlorobenzoyl]-6-chloro-5-hydroxy-2-methyl-1H-indol-3-yl]acetyl}amino)-3-methylpropanoic acid
2-({{1-[4-chlorobenzoyl]-6-chloro-5-hydroxy-2-methyl-1H-indol-3-yl]acetyl}amino)propanoic acid
2-({{1-[4-chlorobenzoyl]-6-chloro-5-hydroxy-2-methyl-1H-indol-3-yl]acetyl}amino)-2-methylpropanoic acid
1-[4-chlorobenzoyl]-4-chloro-5-methoxy-2-methyl-1H-indol-3-yl]acetic acid
({{1-[4-chlorobenzoyl]-4-chloro-5-methoxy-2-methyl-1H-indol-3-yl]acetyl}amino)acetic acid
3-({{1-[4-chlorobenzoyl]-4-chloro-5-methoxy-2-methyl-1H-indol-3-yl]acetyl}amino)propanoic acid
3-({{1-[4-chlorobenzoyl]-4-chloro-5-methoxy-2-methyl-1H-indol-3-yl]acetyl}amino)-2-methylpropanoic acid
3-({{1-[4-chlorobenzoyl]-4-chloro-5-methoxy-2-methyl-1H-indol-3-yl]acetyl}amino)-3-methylpropanoic acid
2-({{1-[4-chlorobenzoyl]-4-chloro-5-methoxy-2-methyl-1H-indol-3-yl]acetyl}amino)propanoic acid
2-({{1-[4-chlorobenzoyl]-4-chloro-5-methoxy-2-methyl-1H-indol-3-yl]acetyl}amino)-2-methylpropanoic acid
1-[4-chlorobenzoyl]-4-chloro-5-hydroxy-2-methyl-1H-indol-3-yl]acetic acid
({{1-[4-chlorobenzoyl]-4-chloro-5-hydroxy-2-methyl-1H-indol-3-yl]acetyl}amino)acetic acid
3-({{1-[4-chlorobenzoyl]-4-chloro-5-hydroxy-2-methyl-1H-indol-3-yl]acetyl}amino)propanoic acid
3-({{1-[4-chlorobenzoyl]-4-chloro-5-hydroxy-2-methyl-1H-indol-3-yl]acetyl}amino)-2-methylpropanoic acid
3-({{1-[4-chlorobenzoyl]-4-chloro-5-hydroxy-2-methyl-1H-indol-3-yl]acetyl}amino)-3-methylpropanoic acid
2-({{1-[4-chlorobenzoyl]-4-chloro-5-hydroxy-2-methyl-1H-indol-3-yl]acetyl}amino)propanoic acid
2-({{1-[4-chlorobenzoyl]-4-chloro-5-hydroxy-2-methyl-1H-indol-3-yl]acetyl}amino)-2-methylpropanoic acid
[1-(4-trifluoromethoxybenzoyl)-5-methoxy-2-methyl-1H-indol-3-yl]acetic acid
({[1-(4-trifluoromethoxybenzoyl)-5-methoxy-2-methyl-1H-indol-3-yl]acetyl}amino)acetic acid
3-({[1-(4-trifluoromethoxybenzoyl)-5-methoxy-2-methyl-1H-indol-3-yl]acetyl}amino)propanoic acid
3-({[1-(4-trifluoromethoxybenzoyl)-5-methoxy-2-methyl-1H-indol-3-yl]acetyl}amino)-2-methylpropanoic acid
3-({[1-(4-trifluoromethoxybenzoyl)-5-methoxy-2-methyl-1H-indol-3-yl]acetyl}amino)-3-methylpropanoic acid
2-({[1-(4-trifluoromethoxybenzoyl)-5-methoxy-2-methyl-1H-indol-3-yl]acetyl}amino)propanoic acid
2-({[1-(4-trifluoromethoxybenzoyl)-5-methoxy-2-methyl-1H-indol-3-yl]acetyl}amino)-2-methylpropanoic acid
[1-(4-trifluoromethoxybenzoyl)-5-methoxy-6-fluoro-2-methyl-1H-indol-3-yl]acetic acid
({[1-(4-trifluoromethoxybenzoyl)-5-methoxy-6-fluoro-2-methyl-1H-indol-3-yl]acetyl}amino)acetic acid
3-({[1-(4-trifluoromethoxybenzoyl)-5-methoxy-6-fluoro-2-methyl-1H-indol-3-yl]acetyl}amino)propanoic acid
3-({[1-(4-trifluoromethoxybenzoyl)-5-methoxy-6-fluoro-2-methyl-1H-indol-3-yl]acetyl}amino)-2-methylpropanoic acid
3-({[1-(4-trifluoromethoxybenzoyl)-5-methoxy-6-fluoro-2-methyl-1H-indol-3-yl]acetyl}amino)-3-methylpropanoic acid
2-({[1-(4-trifluoromethoxybenzoyl)-5-methoxy-6-fluoro-2-methyl-1H-indol-3-yl]acetyl}amino)propanoic acid
2-({[1-(4-trifluoromethoxybenzoyl)-5-methoxy-6-fluoro-2-methyl-1H-indol-3-yl]acetyl}amino)-2-methylpropanoic acid
[1-(4-trifluoromethylbenzoyl)-5-hydroxy-6-fluoro-2-methyl-1H-indol-3-yl]acetic acid
methyl[1-(4-trifluoromethylbenzoyl)-5-hydroxy-6-fluoro-2-methyl-1H-indol-3-yl]acetate
ethyl[1-(4-trifluoromethylbenzoyl)-5-hydroxy-6-fluoro-2-methyl-1H-indol-3-yl]acetate
propyl[1-(4-trifluoromethylbenzoyl)-5-hydroxy-6-fluoro-2-methyl-1H-indol-3-yl]acetate
butyl[1-(4-trifluoromethylbenzoyl)-5-hydroxy-6-fluoro-2-methyl-1H-indol-3-yl]acetate
sec-butyl[1-(4-trifluoromethylbenzoyl)-5-hydroxy-6-fluoro-2-methyl-1H-indol-3-yl]acetate
(R)sec-butyl[1-(4-trifluoromethylbenzoyl)-5-hydroxy-6-fluoro-2-methyl 1H-indol-3-yl]acetate
(S)sec-butyl[1-(4-trifluoromethylbenzoyl)-5-hydroxy-6-fluoro-2-methyl-1H-indol-3-yl]acetate
isopropyl[1-(4-trifluoromethylbenzoyl)-5-hydroxy-6-fluoro-2-methyl-1H-indol-3-yl]acetate
tert-butyl[1-(4-trifluoromethylbenzoyl)-5-hydroxy-6-fluoro-2-methyl-1H-indol-3-yl]acetate
cyclobutyl[1-(4-trifluoromethylbenzoyl)-5-hydroxy-6-fluoro-2-methyl-1H-indol-3-yl]acetate
cyclopentyl[1-(4-trifluoromethylbenzoyl)-5-hydroxy-6-fluoro-2-methyl-1H-indol-3-yl]acetate
2-[1-(4-trifluoromethylbenzoyl)-5-hydroxy-6-fluoro-2-methyl-1H-indol-3-yl]acetamide
2-[1-(4-trifluoromethylbenzoyl)-5-hydroxy-6-fluoro-2-methyl-1H-indol-3-yl]-N-methylacetamide
2-[1-(4-trifluoromethylbenzoyl)-5-hydroxy-6-fluoro-2-methyl-1H-indol-3-yl]-N-ethylacetamide
2-[1-(4-trifluoromethylbenzoyl)-5-hydroxy-6-fluoro-2-methyl-1H-indol-3-yl]-N-propylacetamide
2-[1-(4-trifluoromethylbenzoyl)-5-hydroxy-6-fluoro-2-methyl-1H-indol-3-yl]-N-isopropylacetamide
2-[1-(4-trifluoromethylbenzoyl)-5-hydroxy-6-fluoro-2-methyl-1H-indol-3-yl]-N-butylacetamide
2-[1-(4-trifluoromethylbenzoyl)-5-hydroxy-6-fluoro-2-methyl-1H-indol-3-yl]-N-sec-butylacetamide
2-[1-(4-trifluoromethylbenzoyl)-5-hydroxy-6-fluoro-2-methyl-1H-indol-3-yl]-N-(2-hydroxyethyl)acetamide
({[1-(4-trifluoromethylbenzoyl)-5-hydroxy-6-fluoro-2-methyl-1H-indol-3-yl]acetyl}amino)acetic acid
3-({[1-(4-trifluoromethylbenzoyl)-5-hydroxy-6-fluoro-2-methyl-1H-indol-3-yl]acetyl}amino)propanoic acid
3-({[1-(4-trifluoromethylbenzoyl)-5-hydroxy-6-fluoro-2-methyl-1H-indol-3-yl]acetyl}amino)-2-methylpropanoic acid
3-({[1-(4-trifluoromethylbenzoyl)-5-hydroxy-6-fluoro-2-methyl-1H-indol-3-yl]acetyl}amino)-3-methylpropanoic acid
2-({[1-(4-trifluoromethylbenzoyl)-5-hydroxy-6-fluoro-2-methyl-1H-indol-3-yl]acetyl}amino)propanoic acid
2-({[1-(4-trifluoromethylbenzoyl)-5-hydroxy-6-fluoro-2-methyl-1H-indol-3-yl]acetyl}amino)-2-methylpropanoic acid
[1-(4-trifluoromethylbenzoyl)-5-methoxy-6-fluoro-2-methyl-1H-indol-3-yl]acetic acid
({[1-(4-trifluoromethylbenzoyl)-5-methoxy-6-fluoro-2-methyl-1H-indol-3-yl]acetyl}amino)acetic acid
3-({[1-(4-trifluoromethylbenzoyl)-5-methoxy-6-fluoro-2-methyl-1H-indol-3-yl]acetyl}amino)propanoic acid
3-({[1-(4-trifluoromethylbenzoyl)-5-methoxy-6-fluoro-2-methyl-1H-indol-3-yl]acetyl}amino)-2-methylpropanoic acid
3-({[1-(4-trifluoromethylbenzoyl)-5-methoxy-6-fluoro-2-methyl-1H-indol-3-yl]acetyl}amino)-3-methylpropanoic acid
2-({[1-(4-trifluoromethylbenzoyl)-5-methoxy-6-fluoro-2-methyl-1H-indol-3-yl]acetyl}amino)propanoic acid
2-({[1-(4-trifluoromethylbenzoyl)-5-methoxy-6-fluoro-2-methyl-1H-indol-3-yl]acetyl}amino)-2-methylpropanoic acid
[1-(4-chlorobenzoyl)-5-hydroxy-6-fluoro-2-methyl-1H-indol-3-yl]acetic acid
({[1-(4-chlorobenzoyl)-5-hydroxy-6-fluoro-2-methyl-1H-indol-3-yl]acetyl}amino)acetic acid
3-({[1-(4-chlorobenzoyl)-5-hydroxy-6-fluoro-2-methyl-1H-indol-3-yl]acetyl}amino)propanoic acid
3-({[1-(4-chlorobenzoyl)-5-hydroxy-6-fluoro-2-methyl-1H-indol-3-yl]acetyl}amino)-2-methylpropanoic acid
3-({[1-(4-chlorobenzoyl)-5-hydroxy-6-fluoro-2-methyl-1H-indol-3-yl]acetyl}amino)-3-methylpropanoic acid
2-({[1-(4-chlorobenzoyl)-5-hydroxy-6-fluoro-2-methyl-1H-indol-3-yl]acetyl}amino)propanoic acid
2-({[1-(4-chlorobenzoyl)-5-hydroxy-6-fluoro-2-methyl-1H-indol-3-yl]acetyl}amino)-2-methylpropanoic acid
[1-(4-fluorobenzoyl)-5-methoxy-6-fluoro-2-methyl-1H-indol-3-yl]acetic acid
({[1-(4-fluorobenzoyl)-5-methoxy-6-fluoro-2-methyl-1H-indol-3-yl]acetyl}amino)acetic acid
3-({[1-(4-fluorobenzoyl)-5-methoxy-6-fluoro-2-methyl-1H-indol-3-yl]acetyl}amino)propanoic acid
3-({[1-(4-fluorobenzoyl)-5-methoxy-6-fluoro-2-methyl-1H-indol-3-yl]acetyl}amino)-2-methylpropanoic acid
3-({[1-(4-fluorobenzoyl)-5-methoxy-6-fluoro-2-methyl-1H-indol-3-yl]acetyl}amino)-3-methylpropanoic acid
2-({[1-(4-fluorobenzoyl)-5-methoxy-6-fluoro-2-methyl-1H-indol-3-yl]acetyl}amino)propanoic acid
2-({[1-(4-fluorobenzoyl)-5-methoxy-6-fluoro-2-methyl-1H-indol-3-yl]acetyl}amino)-2-methylpropanoic acid
[1-(4-fluorobenzoyl)-5-hydroxy-6-fluoro-2-methyl-1H-indol-3-yl]acetic acid
({[1-(4-fluorobenzoyl)-5-hydroxy-6-fluoro-2-methyl-1H-indol-3-yl]acetyl}amino)acetic acid
3-({[1-(4-fluorobenzoyl)-5-hydroxy-6-fluoro-2-methyl-1H-indol-3-yl]acetyl}amino)propanoic acid
3-({[1-(4-fluorobenzoyl)-5-hydroxy-6-fluoro-2-methyl-1H-indol-3-yl]acetyl}amino)-2-methylpropanoic acid
3-({[1-(4-fluorobenzoyl)-5-hydroxy-6-fluoro-2-methyl-1H-indol-3-yl]acetyl}amino)-3-methylpropanoic acid
2-({[1-(4-fluorobenzoyl)-5-hydroxy-6-fluoro-2-methyl-1H-indol-3-yl]acetyl}amino)propanoic acid
2-({[1-(4-fluorobenzoyl)-5-hydroxy-6-fluoro-2-methyl-1H-indol-3-yl]acetyl}amino)-2-methylpropanoic acid
[1-(4-bromobenzoyl)-5-hydroxy-6-fluoro-2-methyl-1H-indol-3-yl]acetic acid methyl[1-(4-bromobenzoyl)-5-hydroxy-6-fluoro-2-methyl-1H-indol-3-yl]acetate
({[1-(4-bromobenzoyl)-5-hydroxy-6-fluoro-2-methyl-1H-indol-3-yl]acetyl}amino)acetic acid
3-({[1-(4-bromobenzoyl)-5-hydroxy-6-fluoro-2-methyl-1H-indol-3-yl]acetyl}amino)propanoic acid
3-({[1-(4-bromobenzoyl)-5-hydroxy-6-fluoro-2-methyl-1H-indol-3-yl]acetyl}amino)-2-methylpropanoic acid
3-({[1-(4-bromobenzoyl)-5-hydroxy-6-fluoro-2-methyl-1H-indol-3-yl]acetyl}amino)-3-methylpropanoic acid
2-({[1-(4-bromobenzoyl)-5-hydroxy-6-fluoro-2-methyl-1H-indol-3-yl]acetyl}amino)propanoic acid
2-({[1-(4-bromobenzoyl)-5-hydroxy-6-fluoro-2-methyl-1H-indol-3-yl]acetyl}amino)-2-methylpropanoic acid
[1-(4-bromobenzoyl)-5-methoxy-6-fluoro-2-methyl-1H-indol-3-yl]acetic acid
({[1-(4-bromobenzoyl)-5-methoxy-6-fluoro-2-methyl-1H-indol-3-yl]acetyl}amino)acetic acid
3-({[1-(4-bromobenzoyl)-5-methoxy-6-fluoro-2-methyl-1H-indol-3-yl]acetyl}amino)propanoic acid
3-({[1-(4-bromobenzoyl)-5-methoxy-6-fluoro-2-methyl-1H-indol-3-yl]acetyl}amino)-2-methylpropanoic acid
3-({[1-(4-bromobenzoyl)-5-methoxy-6-fluoro-2-methyl-1H-indol-3-yl]acetyl}amino)-3-methylpropanoic acid
2-({[1-(4-bromobenzoyl)-5-methoxy-6-fluoro-2-methyl-1H-indol-3-yl]acetyl}amino)propanoic acid
2-({[1-(4-bromobenzoyl)-5-methoxy-6-fluoro-2-methyl-1H-indol-3-yl]acetyl}amino)-2-methylpropanoic acid
[1-(4-bromobenzoyl)-5-hydroxy-2-methyl-1H-indol-3-yl]acetic acid
({[1-(4-bromobenzoyl)-5-hydroxy-2-methyl-1H-indol-3-yl]acetyl}amino)acetic acid
3-({[1-(4-bromobenzoyl)-5-hydroxy-2-methyl-1H-indol-3-yl]acetyl}amino)propanoic acid
3-({[1-(4-bromobenzoyl)-5-hydroxy-2-methyl-1H-indol-3-yl]acetyl}amino)-2-methylpropanoic acid
3-({[1-(4-bromobenzoyl)-5-hydroxy-2-methyl-1H-indol-3-yl]acetyl}amino)-3-methylpropanoic acid
2-({[1-(4-bromobenzoyl)-5-hydroxy-2-methyl-1H-indol-3-yl]acetyl}amino)propanoic acid
2-({[1-(4-bromobenzoyl)-5-hydroxy-2-methyl-1H-indol-3-yl]acetyl}amino)-2-methylpropanoic acid
[1-(4-methylbenzoyl)-5-methoxy-6-fluoro-2-methyl-1H-indol-3-yl]acetic acid
({[1-(4-methylbenzoyl)-5-methoxy-6-fluoro-2-methyl-1H-indol-3-yl]acetyl}amino)acetic acid
3-({[1-(4-methylbenzoyl)-5-methoxy-6-fluoro-2-methyl-1H-indol-3-yl]acetyl}amino)propanoic acid
3-({[1-(4-methylbenzoyl)-5-methoxy-6-fluoro-2-methyl-1H-indol-3-yl]acetyl}amino)-2-methylpropanoic acid
3-({[1-(4-methylbenzoyl)-5-methoxy-6-fluoro-2-methyl-1H-indol-3-yl]acetyl}amino)-3-methylpropanoic acid
2-({[1-(4-methylbenzoyl)-5-methoxy-6-fluoro-2-methyl-1H-indol-3-yl]acetyl}amino)propanoic acid
2-({[1-(4-methylbenzoyl)-5-methoxy-6-fluoro-2-methyl-1H-indol-3-yl]acetyl}amino)-2-methylpropanoic acid
[1-(4-methylbenzoyl)-5-hydroxy-6-fluoro-2-methyl-1H-indol-3-yl]acetic acid
({[1-(4-methylbenzoyl)-5-hydroxy-6-fluoro-2-methyl-1H-indol-3-yl]acetyl}amino)acetic acid
3-({[1-(4-methylbenzoyl)-5-hydroxy-6-fluoro-2-methyl-1H-indol-3-yl]acetyl}amino)propanoic acid
3-({[1-(4-methylbenzoyl)-5-hydroxy-6-fluoro-2-methyl-1H-indol-3-yl]acetyl}amino)-2-methylpropanoic acid
3-({[1-(4-methylbenzoyl)-5-hydroxy-6-fluoro-2-methyl-1H-indol-3-yl]acetyl}amino)-3-methylpropanoic acid
2-({[1-(4-methylbenzoyl)-5-hydroxy-6-fluoro-2-methyl-1H-indol-3-yl]acetyl}amino)propanoic acid
2-({[1-(4-methylbenzoyl)-5-hydroxy-6-fluoro-2-methyl-1H-indol-3-yl]acetyl}amino)-2-methylpropanoic acid
[1-(4-methylthiobenzoyl)-5-methoxy-6-fluoro-2-methyl-1H-indol-3-yl]acetic acid
({[1-(4-methylthiobenzoyl)-5-methoxy-6-fluoro-2-methyl-1H-indol-3-yl]acetyl}amino)acetic acid
3-({[1-(4-methylthiobenzoyl)-5-methoxy-6-fluoro-2-methyl-1H-indol-3-yl]acetyl}amino)propanoic acid
3-({[1-(4-methylthiobenzoyl)-5-methoxy-6-fluoro-2-methyl-1H-indol-3-yl]acetyl}amino)-2-methylpropanoic acid
3-({[1-(4-methylthiobenzoyl)-5-methoxy-6-fluoro-2-methyl-1H-indol-3-yl]acetyl}amino)-3-methylpropanoic acid
2-({[1-(4-methylthiobenzoyl)-5-methoxy-6-fluoro-2-methyl-1H-indol-3-yl]acetyl}amino)propanoic acid
2-({[1-(4-methylthiobenzoyl)-5-methoxy-6-fluoro-2-methyl-1H-indol-3-yl]acetyl}amino)-2-methylpropanoic acid
[1-(4-methylthiobenzoyl)-5-hydroxy-6-fluoro-2-methyl-1H-indol-3-yl]acetic acid
({[1-(4-methylthiobenzoyl)-5-hydroxy-6-fluoro-2-methyl-1H-indol-3-yl]acetyl}amino)acetic acid
3-({[1-(4-methylthiobenzoyl)-5-hydroxy-6-fluoro-2-methyl-1H-indol-3-yl]acetyl}amino)propanoic acid
3-({[1-(4-methylthiobenzoyl)-5-hydroxy-6-fluoro-2-methyl-1H-indol-3-yl]acetyl}amino)-2-methylpropanoic acid
3-({[1-(4-methylthiobenzoyl)-5-hydroxy-6-fluoro-2-methyl-1H-indol-3-yl]acetyl}amino)-3-methylpropanoic acid
2-({[1-(4-methylthiobenzoyl)-5-hydroxy-6-fluoro-2-methyl-1H-indol-3-yl]acetyl}amino)propanoic acid
2-({[1-(4-methylthiobenzoyl)-5-hydroxy-6-fluoro-2-methyl-1H-indol-3-yl]acetyl}amino)-2-methylpropanoic acid
[1-(4-chlorobenzoyl)-5-hydroxy-2-methyl-1H-indol-3-yl]acetic acid
({[1-(4-chlorobenzoyl)-5-hydroxy-2-methyl-1H-indol-3-yl]acetyl}amino)acetic acid
3-({[1-(4-chlorobenzoyl)-5-hydroxy-2-methyl-1H-indol-3-yl]acetyl}amino)propanoic acid
3-({[1-(4-chlorobenzoyl)-5-hydroxy-2-methyl-1H-indol-3-yl]acetyl}amino)-2-methylpropanoic acid
3-({[1-(4-chlorobenzoyl)-5-hydroxy-2-methyl-1H-indol-3-yl]acetyl}amino)-3-methylpropanoic acid
2-({[1-(4-chlorobenzoyl)-5-hydroxy-2-methyl-1H-indol-3-yl]acetyl}amino)propanoic acid
2-({[1-(4-chlorobenzoyl)-5-hydroxy-2-methyl-1H-indol-3-yl]acetyl}amino)-2-methylpropanoic acid
1-[4-fluorobenzoyl]-6-chloro-5-methoxy-2-methyl-1H-indol-3-yl]acetic acid
({{1-[4-fluorobenzoyl]-6-chloro-5-methoxy-2-methyl-1H-indol-3-yl]acetyl}amino)acetic acid
3-({{1-[4-fluorobenzoyl]-6-chloro-5-methoxy-2-methyl-1H-indol-3-yl]acetyl}amino)propanoic acid
3-({{1-[4-fluorobenzoyl]-6-chloro-5-methoxy-2-methyl-1H-indol-3-yl]acetyl}amino)-2-methylpropanoic acid
3-({{1-[4-fluorobenzoyl]-6-chloro-5-methoxy-2-methyl-1H-indol-3-yl]acetyl}amino)-3-methylpropanoic acid
2-({{1-[4-fluorobenzoyl]-6-chloro-5-methoxy-2-methyl-1H-indol-3-yl]acetyl}amino)propanoic acid
2-({{1-[4-fluorobenzoyl]-6-chloro-5-methoxy-2-methyl-1H-indol-3-yl]acetyl}amino)-2-methylpropanoic acid
1-[4-fluorobenzoyl]-6-chloro-5-hydroxy-2-methyl-1H-indol-3-yl]acetic acid
({{1-[4-fluorobenzoyl]-6-chloro-5-hydroxy-2-methyl-1H-indol-3-yl]acetyl}amino)acetic acid
3-({{1-[4-fluorobenzoyl]-6-chloro-5-hydroxy-2-methyl-1H-indol-3-yl]acetyl}amino)propanoic acid
3-({{1-[4-fluorobenzoyl]-6-chloro-5-hydroxy-2-methyl-1H-indol-3-yl]acetyl}amino)-2-methylpropanoic acid
3-({{1-[4-fluorobenzoyl]-6-chloro-5-hydroxy-2-methyl-1H-indol-3-yl]acetyl}amino)-3-methylpropanoic acid
2-({{1-[4-fluorobenzoyl]-6-chloro-5-hydroxy-2-methyl-1H-indol-3-yl]acetyl}amino)propanoic acid
2-({{1-[4-fluorobenzoyl]-6-chloro-5-hydroxy-2-methyl-1H-indol-3-yl]acetyl}amino)-2-methylpropanoic acid
1-[4-bromobenzoyl]-6-chloro-5-methoxy-2-methyl-1H-indol-3-yl]acetic acid
methyl{1-[4-bromobenzoyl]-6-chloro-5-methoxy-2-methyl-1H-indol-3-yl]acetate
({{1-[4-bromobenzoyl]-6-chloro-5-methoxy-2-methyl-1H-indol-3-yl]acetyl}amino)acetic acid
3-({{1-[4-bromobenzoyl]-6-chloro-5-methoxy-2-methyl-1H-indol-3-yl]acetyl}amino)propanoic acid
3-({{1-[4-bromobenzoyl]-6-chloro-5-methoxy-2-methyl-1H-indol-3-yl]acetyl}amino)-2-methylpropanoic acid
3-({{1-[4-bromobenzoyl]-6-chloro-5-methoxy-2-methyl-1H-indol-3-yl]acetyl}amino)-3-methylpropanoic acid
2-({{1-[4-bromobenzoyl]-6-chloro-5-methoxy-2-methyl-1H-indol-3-yl]acetyl}amino)propanoic acid
2-({{1-[4-bromobenzoyl]-6-chloro-5-methoxy-2-methyl-1H-indol-3-yl]acetyl}amino)-2-methylpropanoic acid
1-[4-bromobenzoyl]-6-chloro-5-hydroxy-2-methyl-1H-indol-3-yl]acetic acid
({{1-[4-bromobenzoyl]-6-chloro-5-hydroxy-2-methyl-1H-indol-3-yl]acetyl}amino)acetic acid
3-({{1-[4-bromobenzoyl]-6-chloro-5-hydroxy-2-methyl-1H-indol-3-yl]acetyl}amino)propanoic acid
3-({{1-[4-bromobenzoyl]-6-chloro-5-hydroxy-2-methyl-1H-indol-3-yl]acetyl}amino)-2-methylpropanoic acid
3-({{1-[4-bromobenzoyl]-6-chloro-5-hydroxy-2-methyl-1H-indol-3-yl]acetyl}amino)-3-methylpropanoic acid
2-({{1-[4-bromobenzoyl]-6-chloro-5-hydroxy-2-methyl-1H-indol-3-yl]acetyl}amino)propanoic acid
2-({{1-[4-bromobenzoyl]-6-chloro-5-hydroxy-2-methyl-1H-indol-3-yl]acetyl}amino)-2-methylpropanoic acid
1-[4-trifluoromethylbenzoyl]-6-chloro-5-methoxy-2-methyl-1H-indol-3-yl]acetic acid
({{1-[4-trifluoromethylbenzoyl]-6-chloro-5-methoxy-2-methyl-1H-indol-3-yl]acetyl}amino)acetic acid
3-({{1-[4-trifluoromethylbenzoyl]-6-chloro-5-methoxy-2-methyl-1H-indol-3-yl]acetyl}amino)propanoic acid
3-({{1-[4-trifluoromethylbenzoyl]-6-chloro-5-methoxy-2-methyl-1H-indol-3-yl]acetyl}amino)-2-methylpropanoic acid
3-({{1-[4-trifluoromethylbenzoyl]-6-chloro-5-methoxy-2-methyl-1H-indol-3-yl]acetyl}amino)-3-methylpropanoic acid
2-({{1-[4-trifluoromethylbenzoyl]-6-chloro-5-methoxy-2-methyl-1H-indol-3-yl]acetyl}amino)propanoic acid
2-({{1-[4-trifluoromethylbenzoyl]-6-chloro-5-methoxy-2-methyl-1H-indol-3-yl]acetyl}amino)-2-methylpropanoic acid
1-[4-trifluoromethylbenzoyl]-6-chloro-5-hydroxy-2-methyl-1H-indol-3-yl]acetic acid
({{1-[4-trifluoromethylbenzoyl]-6-chloro-5-hydroxy-2-methyl-1H-indol-3-yl]acetyl}amino)acetic acid
3-({{1-[4-trifluoromethylbenzoyl]-6-chloro-5-hydroxy-2-methyl-1H-indol-3-yl]acetyl}amino)propanoic acid
3-({{1-[4-trifluoromethylbenzoyl]-6-chloro-5-hydroxy-2-methyl-1H-indol-3-yl]acetyl}amino)-2-methylpropanoic acid
3-({{1-[4-trifluoromethylbenzoyl]-6-chloro-5-hydroxy-2-methyl-1H-indol-3-yl]acetyl}amino)-3-methylpropanoic acid
2-({{1-[4-trifluoromethylbenzoyl]-6-chloro-5-hydroxy-2-methyl-1H-indol-3-yl]acetyl}amino)propanoic acid
2-({{1-[4-bromobenzoyl]-6-chloro-5-hydroxy-2-methyl-1H-indol-3-yl]acetyl}amino)-2-methylpropanoic acid
1-[4-trifluoromethoxybenzoyl]-6-chloro-5-methoxy-2-methyl-1H-indol-3-yl]acetic acid
({{1-[4-trifluoromethoxybenzoyl]-6-chloro-5-methoxy-2-methyl-1H-indol-3-yl]acetyl}amino)acetic acid
3-({{1-[4-trifluoromethoxybenzoyl]-6-chloro-5-methoxy-2-methyl-1H-indol-3-yl]acetyl}amino)propanoic acid
3-({{1-[4-trifluoromethoxybenzoyl]-6-chloro-5-methoxy-2-methyl-1H-indol-3-yl]acetyl}amino)-2-methylpropanoic acid
3-({{1-[4-trifluoromethoxybenzoyl]-6-chloro-5-methoxy-2-methyl-1H-indol-3-yl]acetyl}amino)-3-methylpropanoic acid
2-({{1-[4-trifluoromethoxybenzoyl]-6-chloro-5-methoxy-2-methyl-1H-indol-3-yl]acetyl}amino)propanoic acid
2-({{1-[4-trifluoromethoxybenzoyl]-6-chloro-5-methoxy-2-methyl-1H-indol-3-yl]acetyl}amino)-2-methylpropanoic acid
1-[4-trifluoromethoxybenzoyl]-6-chloro-5-hydroxy-2-methyl-1H-indol-3-yl]acetic acid
({{1-[4-trifluoromethoxybenzoyl]-6-chloro-5-hydroxy-2-methyl-1H-indol-3-yl]acetyl}amino)acetic acid
3-({{1-[4-trifluoromethoxybenzoyl]-6-chloro-5-hydroxy-2-methyl-1H-indol-3-yl]acetyl}amino)propanoic acid
3-({{1-[4-trifluoromethoxybenzoyl]-6-chloro-5-hydroxy-2-methyl-1H-indol-3-yl]acetyl}amino)-2-methylpropanoic acid
3-({{1-[4-trifluoromethoxybenzoyl]-6-chloro-5-hydroxy-2-methyl-1H-indol-3-yl]acetyl}amino)-3-methylpropanoic acid
2-({{1-[4-trifluoromethoxybenzoyl]-6-chloro-5-hydroxy-2-methyl-1H-indol-3-yl]acetyl}amino)propanoic acid
2-({{1-[4-bromobenzoyl]-6-chloro-5-hydroxy-2-methyl-1H-indol-3-yl]acetyl}amino)-2-methylpropanoic acid
1-[4-difluoromethoxybenzoyl]-6-chloro-5-methoxy-2-methyl-1H-indol-3-yl]acetic acid
({{1-[4-difluoromethoxybenzoyl]-6-chloro-5-methoxy-2-methyl-1H-indol-3-yl]acetyl}amino)acetic acid
3-({{1-[4-difluoromethoxybenzoyl]-6-chloro-5-methoxy-2-methyl-1H-indol-3-yl]acetyl}amino)propanoic acid
3-({{1-[4-difluoromethoxybenzoyl]-6-chloro-5-methoxy-2-methyl-1H-indol-3-yl]acetyl}amino)-2-methylpropanoic acid
3-({{1-[4-difluoromethoxybenzoyl]-6-chloro-5-methoxy-2-methyl-1H-indol-3-yl]acetyl}amino)-3-methylpropanoic acid
2-({{1-[4-difluoromethoxybenzoyl]-6-chloro-5-methoxy-2-methyl-1H-indol-3-yl]acetyl}amino)propanoic acid
2-({{1-[4-difluoromethoxybenzoyl]-6-chloro-5-methoxy-2-methyl-1H-indol-3-yl]acetyl}amino)-2-methylpropanoic acid
1-[4-difluoromethoxybenzoyl]-6-chloro-5-hydroxy-2-methyl-1H-indol-3-yl]acetic acid
({{1-[4-difluoromethoxybenzoyl]-6-chloro-5-hydroxy-2-methyl-1H-indol-3-yl]acetyl}amino)acetic acid
3-({{1-[4-difluoromethoxybenzoyl]-6-chloro-5-hydroxy-2-methyl-1H-indol-3-yl]acetyl}amino)propanoic acid
3-({{1-[4-difluoromethoxybenzoyl]-6-chloro-5-hydroxy-2-methyl-1H-indol-3-yl]acetyl}amino)-2-methylpropanoic acid
3-({{1-[4-difluoromethoxybenzoyl]-6-chloro-5-hydroxy-2-methyl-1H-indol-3-yl]acetyl}amino)-3-methylpropanoic acid
2-({{1-[4-difluoromethoxybenzoyl]-6-chloro-5-hydroxy-2-methyl-1H-indol-3-yl]acetyl}amino)propanoic acid
2-({{1-[4-bromobenzoyl]-6-chloro-5-hydroxy-2-methyl-1H-indol-3-yl]acetyl}amino)-2-methylpropanoic acid
1-[4-methylthiobenzoyl]-6-chloro-5-methoxy-2-methyl-1H-indol-3-yl]acetic acid
({{1-[4-methylthiobenzoyl]-6-chloro-5-methoxy-2-methyl-1H-indol-3-yl]acetyl}amino)acetic acid
3-({{1-[4-methylthiobenzoyl]-6-chloro-5-methoxy-2-methyl-1H-indol-3-yl]acetyl}amino)propanoic acid
3-({{1-[4-methylthiobenzoyl]-6-chloro-5-methoxy-2-methyl-1H-indol-3-yl]acetyl}amino)-2-methylpropanoic acid
3-({{1-[4-methylthiobenzoyl]-6-chloro-5-methoxy-2-methyl-1H-indol-3-yl]acetyl}amino)-3-methylpropanoic acid
2-({{1-[4-methylthiobenzoyl]-6-chloro-5-methoxy-2-methyl-1H-indol-3-yl]acetyl}amino)propanoic acid
2-({{1-[4-methylthiobenzoyl]-6-chloro-5-methoxy-2-methyl-1H-indol-3-yl]acetyl}amino)-2-methylpropanoic acid
1-[4-methylthiobenzoyl]-6-chloro-5-hydroxy-2-methyl-1H-indol-3-yl]acetic acid
({{1-[4-methylthiobenzoyl]-6-chloro-5-hydroxy-2-methyl-1H-indol-3-yl]acetyl}amino)acetic acid
3-({{1-[4-methylthiobenzoyl]-6-chloro-5-hydroxy-2-methyl-1H-indol-3-yl]acetyl}amino)propanoic acid
3-({{1-[4-methylthiobenzoyl]-6-chloro-5-hydroxy-2-methyl-1H-indol-3-yl]acetyl}amino)-2-methylpropanoic acid
3-({{1-[4-methylthiobenzoyl]-6-chloro-5-hydroxy-2-methyl-1H-indol-3-yl]acetyl}amino)-3-methylpropanoic acid
2-({{1-[4-methylthiobenzoyl]-6-chloro-5-hydroxy-2-methyl-1H-indol-3-yl]acetyl}amino)propanoic acid
2-({{1-[4-methylthiobenzoyl]-6-chloro-5-hydroxy-2-methyl-1H-indol-3-yl]acetyl}amino)-2-methylpropanoic acid
[1-(4-methylbenzoyl)-5-methoxy-6-chloro-2-methyl-1H-indol-3-yl]acetic acid
({[1-(4-methylbenzoyl)-5-methoxy-6-chloro-2-methyl-1H-indol-3-yl]acetyl}amino)acetic acid
3-({[1-(4-methylbenzoyl)-5-methoxy-6-chloro-2-methyl-1H-indol-3-yl]acetyl}amino)propanoic acid
3-({[1-(4-methylbenzoyl)-5-methoxy-6-chloro-2-methyl-1H-indol-3-yl]acetyl}amino)-2-methylpropanoic acid
3-({[1-(4-methylbenzoyl)-5-methoxy-6-chloro-2-methyl-1H-indol-3-yl]acetyl}amino)-3-methylpropanoic acid
2-({[1-(4-methylbenzoyl)-5-methoxy-6-chloro-2-methyl-1H-indol-3-yl]acetyl}amino)propanoic acid
2-({[1-(4-methylbenzoyl)-5-methoxy-6-chloro-2-methyl-1H-indol-3-yl]acetyl}amino)-2-methylpropanoic acid
[1-(4-methylbenzoyl)-5-hydroxy-6-chloro-2-methyl-1H-indol-3-yl]acetic acid
({[1-(4-methylbenzoyl)-5-hydroxy-6-chloro-2-methyl-1H-indol-3-yl]acetyl}amino)acetic acid
3-({[1-(4-methylbenzoyl)-5-hydroxy-6-chloro-2-methyl-1H-indol-3-yl]acetyl}amino)propanoic acid
3-({[1-(4-methylbenzoyl)-5-hydroxy-6-chloro-2-methyl-1H-indol-3-yl]acetyl}amino)-2-methylpropanoic acid
3-({[1-(4-methylbenzoyl)-5-hydroxy-6-chloro-2-methyl-1H-indol-3-yl]acetyl}amino)-3-methylpropanoic acid
2-({[1-(4-methylbenzoyl)-5-hydroxy-6-chloro-2-methyl-1H-indol-3-yl]acetyl}amino)propanoic acid
2-({[1-(4-methylbenzoyl)-5-hydroxy-6-chloro-2-methyl-1H-indol-3-yl]acetyl}amino)-2-methylpropanoic acid
{5-methoxy-2-methyl-1-[4-(trifluoromethoxy)benzoyl]-1H-indol-3-yl}acetic acid
{6-chloro-5-methoxy-2-methyl-1-[4-(trifluoromethoxy)benzoyl]-1H-indol-3-yl}acetic acid.
{6-chloro-5-hydroxy-2-methyl-1-[4-(trifluoromethoxy)benzoyl]-1H-indol-3-yl}acetic acid
{5-hydroxy-2-methyl-1-[4-(trifluoromethoxy)benzoyl]-1H-indol-3-yl}acetic acid
{1-[4-(difluoromethoxy)benzoyl]-5-hydroxy-2-methyl-1H-indol-3-yl}acetic acid
{6-chloro-1-[4-(difluoromethoxy)benzoyl]-5-hydroxy-2-methyl-1H-indol-3-yl}acetic acid
{6-chloro-1-[4-(difluoromethoxy)benzoyl]-5-methoxy-2-methyl-1H-indol-3-yl}acetic acid and
{1-[4-(difluoromethoxy)benzoyl]-5-methoxy-2-methyl-1H-indol-3-yl}acetic acid.

Any of the methods described herein for making compoounds having the formula 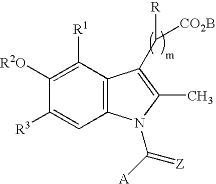
can be used to produce any of the following compounds:
methyl[1-(4-chlorobenzoyl)-5-hydroxy-2-methyl-1H-indol-3-yl]acetate
ethyl[1-(4-chlorobenzoyl)-5-hydroxy-2-methyl-1H-indol-3-yl]acetate
propyl[1-(4-chlorobenzoyl)-5-hydroxy-2-methyl-1H-indol-3-yl]acetate
butyl[1-(4-chlorobenzoyl)-5-hydroxy-2-methyl-1H-indol-3-yl]acetate
sec-butyl[1-(4-chlorobenzoyl)-5-hydroxy-2-methyl-1H-indol-3-yl]acetate
(R)sec-butyl[1-(4-chlorobenzoyl)-5-hydroxy-2-methyl-1H-indol-3-yl]acetate
(S)sec-butyl[1-(4-chlorobenzoyl)-5-hydroxy-2-methyl-1H-indol-3-yl]acetate
isopropyl[1-(4-chlorobenzoyl)-5-hydroxy-2-methyl-1H-indol-3-yl]acetate
tert-butyl[1-(4-chlorobenzoyl)-5-hydroxy-2-methyl-1H-indol-3-yl]acetate
cyclobutyl[1-(4-chlorobenzoyl)-5-hydroxy-2-methyl-1H-indol-3-yl]acetate
cyclopentyl[1-(4-chlorobenzoyl)-5-hydroxy-2-methyl-1H-indol-3-yl]acetate
2-[1-(4-chlorobenzoyl)-5-hydroxy-2-methyl-1H-indol-3-yl]acetamide
2-[1-(4-chlorobenzoyl)-5-hydroxy-2-methyl-1H-indol-3-yl]-N-methylacetamide
2-[1-(4-chlorobenzoyl)-5-hydroxy-2-methyl-1H-indol-3-yl]-N-ethylacetamide
2-[1-(4-chlorobenzoyl)-5-hydroxy-2-methyl-1H-indol-3-yl]-N-propylacetamide
2-[1-(4-chlorobenzoyl)-5-hydroxy-2-methyl-1H-indol-3-yl]-N-isopropylacetamide
2-[1-(4-chlorobenzoyl)-5-hydroxy-2-methyl-1H-indol-3-yl]-N-butylacetamide
2-[1-(4-chlorobenzoyl)-5-hydroxy-2-methyl-1H-indol-3-yl]-N-sec-butylacetamide
2-[1-(4-chlorobenzoyl)-5-hydroxy-2-methyl-1H-indol-3-yl]-N-(2-hydroxyethyl)acetamide
methyl(2R)-2-[1-(4-chlorobenzoyl)-5-hydroxy-2-methyl-1H-indol-3-yl]propanoate
ethyl(2R)-2-[1-(4-chlorobenzoyl)-5-hydroxy-2-methyl-1H-indol-3-yl]propanoate
propyl(2R)-2-[1-(4-chlorobenzoyl)-5-hydroxy-2-methyl-1H-indol-3-yl]propanoate
isopropyl(2R)-2-[1-(4-chlorobenzoyl)-5-hydroxy-2-methyl-1H-indol-3-yl]propanoate
butyl(2R)-2-[1-(4-chlorobenzoyl)-5-hydroxy-2-methyl-1H-indol-3-yl]propanoate
sec-butyl(2R)-2-[1-(4-chlorobenzoyl)-5-hydroxy-2-methyl-1H-indol-3-yl]propanoate
(R)sec-butyl(2R)-2-[1-(4-chlorobenzoyl)-5-hydroxy-2-methyl-1H-indol-3-yl]propanoate
(S)sec-butyl(2R)-2-[1-(4-chlorobenzoyl)-5-hydroxy-2-methyl-1H-indol-3-yl]propanoate
tert-butyl(2R)-2-[1-(4-chlorobenzoyl)-5-hydroxy-2-methyl-1H-indol-3-yl]propanoate
cyclobutyl(2R)-2-[1-(4-chlorobenzoyl)-5-hydroxy-2-methyl-1H-indol-3-yl]propanoate
cyclopentyl(2R)-2-[1-(4-chlorobenzoyl)-5-hydroxy-2-methyl-1H-indol-3-yl]propanoate
(2R)-2-[1-(4-chlorobenzoyl)-5-hydroxy-2-methyl-1H-indol-3-yl]propanamide
(2R)-2-[1-(4-chlorobenzoyl)-5-hydroxy-2-methyl-1H-indol-3-yl]-N-methylpropanamide
(2R)-2-[1-(4-chlorobenzoyl)-5-hydroxy-2-methyl-1H-indol-3-yl]-N-ethylacetamide
(2R)-2-[1-(4-chlorobenzoyl)-5-hydroxy-2-methyl-1H-indol-3-yl]-N-propylacetamide
(2R)-2-[1-(4-chlorobenzoyl)-5-hydroxy-2-methyl-1H-indol-3-yl]-N-isopropylacetamide
(2R)-2-[1-(4-chlorobenzoyl)-5-hydroxy-2-methyl-1H-indol-3-yl]-N-butylacetamide
(2R)-2-[1-(4-chlorobenzoyl)-5-hydroxy-2-methyl-1H-indol-3-yl]-N-sec-butylacetamide
(2R)-2-[1-(4-chlorobenzoyl)-5-hydroxy-2-methyl-1H-indol-3-yl]-N-(hydroxymethyl)propanamide
methyl(2S)-2-[1-(4-chlorobenzoyl)-5-hydroxy-2-methyl-1H-indol-3-yl]propanoate
ethyl(2S)-2-[1-(4-chlorobenzoyl)-5-hydroxy-2-methyl-1H-indol-3-yl]propanoate
propyl(2S)-2-[1-(4-chlorobenzoyl)-5-hydroxy-2-methyl-1H-indol-3-yl]propanoate
isopropyl(2S)-2-[1-(4-chlorobenzoyl)-5-hydroxy-2-methyl-1H-indol-3-yl]propanoate
butyl(2S)-2-[1-(4-chlorobenzoyl)-5-hydroxy-2-methyl-1H-indol-3-yl]propanoate
sec-butyl(2S)-2-[1-(4-chlorobenzoyl)-5-hydroxy-2-methyl-1H-indol-3-yl]propanoate
(R)sec-butyl(2S)-2-[1-(4-chlorobenzoyl)-5-hydroxy-2-methyl-1H-indol-3-yl]propanoate
(S)sec-butyl(2S)-2-[1-(4-chlorobenzoyl)-5-hydroxy-2-methyl-1H-indol-3-yl]propanoate
tert-butyl(2S)-2-[1-(4-chlorobenzoyl)-5-hydroxy-2-methyl-1H-indol-3-yl]propanoate
cyclobutyl(2S)-2-[1-(4-chlorobenzoyl)-5-hydroxy-2-methyl-1H-indol-3-yl]propanoate
cyclopentyl(2S)-2-[1-(4-chlorobenzoyl)-5-hydroxy-2-methyl-1H-indol-3-yl]propanoate
(2S)-2-[1-(4-chlorobenzoyl)-5-hydroxy-2-methyl-1H-indol-3-yl]propanamide
(2S)-2-[1-(4-chlorobenzoyl)-5-hydroxy-2-methyl-1H-indol-3-yl]-N-methylpropanamide
(2S)-2-[1-(4-chlorobenzoyl)-5-hydroxy-2-methyl-1H-indol-3-yl]-N-ethylacetamide
(2S)-2-[1-(4-chlorobenzoyl)-5-hydroxy-2-methyl-1H-indol-3-yl]-N-propylacetamide
(2S)-2-[1-(4-chlorobenzoyl)-5-hydroxy-2-methyl-1H-indol-3-yl]-N-isopropylacetamide
(2S)-2-[1-(4-chlorobenzoyl)-5-hydroxy-2-methyl-1H-indol-3-yl]-N-butylacetamide
(2S)-2-[1-(4-chlorobenzoyl)-5-hydroxy-2-methyl-1H-indol-3-yl]-N-sec-butylacetamide
(2S)-2-[1-(4-chlorobenzoyl)-5-hydroxy-2-methyl-1H-indol-3-yl]-N-(hydroxymethyl)propanamide
methyl(2R,S)-2-[1-(4-chlorobenzoyl)-5-hydroxy-2-methyl-1H-indol-3-yl]propanoate
ethyl(2R,S)-2-[1-(4-chlorobenzoyl)-5-hydroxy-2-methyl-1H-indol-3-yl]propanoate
propyl(2R,S)-2-[1-(4-chlorobenzoyl)-5-hydroxy-2-methyl-1H-indol-3-yl]propanoate
isopropyl(2R,S)-2-[1-(4-chlorobenzoyl)-5-hydroxy-2-methyl-1H-indol-3-yl]propanoate
butyl(2R,S)-2-[1-(4-chlorobenzoyl)-5-hydroxy-2-methyl-1H-indol-3-yl]propanoate
sec-butyl(2R,S)-2-[1-(4-chlorobenzoyl)-5-hydroxy-2-methyl-1H-indol-3-yl]propanoate
(R)sec-butyl(2R,S)-2-[1-(4-chlorobenzoyl)-5-hydroxy-2-methyl-1H-indol-3-yl]propanoate
(S)sec-butyl(2R,S)-2-[1-(4-chlorobenzoyl)-5-hydroxy-2-methyl-1H-indol-3-yl]propanoate
tert-butyl(2R,S)-2-[1-(4-chlorobenzoyl)-5-hydroxy-2-methyl-1H-indol-3-yl]propanoate
cyclobutyl(2R,S)-2-[1-(4-chlorobenzoyl)-5-hydroxy-2-methyl-1H-indol-3-yl]propanoate
cyclopentyl(2R,S)-2-[1-(4-chlorobenzoyl)-5-hydroxy-2-methyl-1H-indol-3-yl]propanoate
(2R,S)-2-[1-(4-chlorobenzoyl)-5-hydroxy-2-methyl-1H-indol-3-yl]propanamide
(2R,S)-2-[1-(4-chlorobenzoyl)-5-hydroxy-2-methyl-1H-indol-3-yl]-N-methylpropanamide
(2R,S)-2-[1-(4-chlorobenzoyl)-5-hydroxy-2-methyl-1H-indol-3-yl]-N-ethylacetamide
(2R,S)-2-[1-(4-chlorobenzoyl)-5-hydroxy-2-methyl-1H-indol-3-yl]-N-propylacetamide
(2R,S)-2-[1-(4-chlorobenzoyl)-5-hydroxy-2-methyl-1H-indol-3-yl]-N-isopropylacetamide
(2R,S)-2-[1-(4-chlorobenzoyl)-5-hydroxy-2-methyl-1H-indol-3-yl]-N-butylacetamide
(2R,S)-2-[1-(4-chlorobenzoyl)-5-hydroxy-2-methyl-1H-indol-3-yl]-N-sec-butylacetamide
(2R,S)-2-[1-(4-chlorobenzoyl)-5-hydroxy-2-methyl-1H-indol-3-yl]-N-(hydroxymethyl)propanamide
methyl[1-(4-chlorobenzoyl)-6-fluoro-5-hydroxy-2-methyl-1H-indol-3-yl]acetate
ethyl[1-(4-chlorobenzoyl)-6-fluoro-5-hydroxy-2-methyl-1H-indol-3-yl]acetate
propyl[1-(4-chlorobenzoyl)-6-fluoro-5-hydroxy-2-methyl-1H-indol-3-yl]acetate
butyl[1-(4-chlorobenzoyl)-6-fluoro-5-hydroxy-2-methyl-1H-indol-3-yl]acetate
sec-butyl[1-(4-chlorobenzoyl)-6-fluoro-5-hydroxy-2-methyl-1H-indol-3-yl]acetate
(R)sec-butyl[1-(4-chlorobenzoyl)-6-fluoro-5-hydroxy-2-methyl-1H-indol-3-yl]acetate
(S)sec-butyl[1-(4-chlorobenzoyl)-6-fluoro-5-hydroxy-2-methyl-1H-indol-3-yl]acetate
isopropyl[1-(4-chlorobenzoyl)-6-fluoro-5-hydroxy-2-methyl-1H-indol-3-yl]acetate
tert-butyl[1-(4-chlorobenzoyl)-6-fluoro-5-hydroxy-2-methyl-1H-indol-3-yl]acetate
cyclobutyl[1-(4-chlorobenzoyl)-6-fluoro-5-hydroxy-2-methyl-1H-indol-3-yl]acetate
cyclopentyl[1-(4-chlorobenzoyl)-6-fluoro-5-hydroxy-2-methyl-1H-indol-3-yl]acetate
2-[1-(4-chlorobenzoyl)-6-fluoro-5-hydroxy-2-methyl-1H-indol-3-yl]acetamide
2-[1-(4-chlorobenzoyl)-6-fluoro-5-hydroxy-2-methyl-1H-indol-3-yl]-N-methylacetamide
2-[1-(4-chlorobenzoyl)-6-fluoro-5-hydroxy-2-methyl-1H-indol-3-yl]-N-ethylacetamide
2-[1-(4-chlorobenzoyl)-6-fluoro-5-hydroxy-2-methyl-1H-indol-3-yl]-N-propylacetamide
2-[1-(4-chlorobenzoyl)-6-fluoro-5-hydroxy-2-methyl-1H-indol-3-yl]-N-isopropylacetamide
2-[1-(4-chlorobenzoyl)-6-fluoro-5-hydroxy-2-methyl-1H-indol-3-yl]-N-butylacetamide
2-[1-(4-chlorobenzoyl)-6-fluoro-5-hydroxy-2-methyl-1H-indol-3-yl]-N-sec-butylacetamide
2-[1-(4-chlorobenzoyl)-6-fluoro-5-hydroxy-2-methyl-1H-indol-3-yl]-N-(2-hydroxyethyl)acetamide
methyl[1-(4-chlorobenzoyl)-4-fluoro-5-hydroxy-2-methyl-1H-indol-3-yl]acetate
ethyl[1-(4-chlorobenzoyl)-4-fluoro-5-hydroxy-2-methyl-1H-indol-3-yl]acetate
propyl[1-(4-chlorobenzoyl)-4-fluoro-5-hydroxy-2-methyl-1H-indol-3-yl]acetate
butyl[1-(4-chlorobenzoyl)-4-fluoro-5-hydroxy-2-methyl-1H-indol-3-yl]acetate
sec-butyl[1-(4-chlorobenzoyl)-4-fluoro-5-hydroxy-2-methyl-1H-indol-3-yl]acetate
(R)sec-butyl[1-(4-chlorobenzoyl)-4-fluoro-5-hydroxy-2-methyl-1H-indol-3-yl]acetate
(S)sec-butyl[1-(4-chlorobenzoyl)-4-fluoro-5-hydroxy-2-methyl-1H-indol-3-yl]acetate
isopropyl[1-(4-chlorobenzoyl)-4-fluoro-5-hydroxy-2-methyl-1H-indol-3-yl]acetate
tert-butyl[1-(4-chlorobenzoyl)-4-fluoro-5-hydroxy-2-methyl-1H-indol-3-yl]acetate
cyclobutyl[1-(4-chlorobenzoyl)-4-fluoro-5-hydroxy-2-methyl-1H-indol-3-yl]acetate
cyclopentyl[1-(4-chlorobenzoyl)-4-fluoro-5-hydroxy-2-methyl-1H-indol-3-yl]acetate
2-[1-(4-chlorobenzoyl)-4-fluoro-5-hydroxy-2-methyl-1H-indol-3-yl]acetamide
2-[1-(4-chlorobenzoyl)-4-fluoro-5-hydroxy-2-methyl-1H-indol-3-yl]-N-methylacetamide
2-[1-(4-chlorobenzoyl)-4-fluoro-5-hydroxy-2-methyl-1H-indol-3-yl]-N-ethylacetamide
2-[1-(4-chlorobenzoyl)-4-fluoro-5-hydroxy-2-methyl-1H-indol-3-yl]-N-propylacetamide
2-[1-(4-chlorobenzoyl)-4-fluoro-5-hydroxy-2-methyl-1H-indol-3-yl]-N-isopropylacetamide
2-[1-(4-chlorobenzoyl)-4-fluoro-5-hydroxy-2-methyl-1H-indol-3-yl]-N-butylacetamide
2-[1-(4-chlorobenzoyl)-4-fluoro-5-hydroxy-2-methyl-1H-indol-3-yl]-N-sec-butylacetamide
2-[1-(4-chlorobenzoyl)-4-fluoro-5-hydroxy-2-methyl-1H-indol-3-yl]-N-(2-hydroxyethyl)acetamide
methyl[1-(4-chlorobenzoyl)-4,6-difluoro-5-hydroxy-2-methyl-1H-indol-3-yl]acetate
ethyl[1-(4-chlorobenzoyl)-4,6-difluoro-5-hydroxy-2-methyl-1H-indol-3-yl]acetate
propyl[1-(4-chlorobenzoyl)-4,6-difluoro-5-hydroxy-2-methyl-1H-indol-3-yl]acetate
butyl[1-(4-chlorobenzoyl)-4,6-difluoro-5-hydroxy-2-methyl-1H-indol-3-yl]acetate
sec-butyl[1-(4-chlorobenzoyl)-4,6-difluoro-5-hydroxy-2-methyl-1H-indol-3-yl]acetate
(R)sec-butyl[1-(4-chlorobenzoyl)-4,6-difluoro-5-hydroxy-2-methyl-1H-indol-3-yl]acetate
(S)sec-butyl[1-(4-chlorobenzoyl)-4,6-difluoro-5-hydroxy-2-methyl-1H-indol-3-yl]acetate
isopropyl[1-(4-chlorobenzoyl)-4,6-difluoro-5-hydroxy-2-methyl-1H-indol-3-yl]acetate
tert-butyl[1-(4-chlorobenzoyl)-4,6-difluoro-5-hydroxy-2-methyl-1H-indol-3-yl]acetate
cyclobutyl[1-(4-chlorobenzoyl)-4,6-difluoro-5-hydroxy-2-methyl-1H-indol-3-yl]acetate
cyclopentyl[1-(4-chlorobenzoyl)-4,6-difluoro-5-hydroxy-2-methyl-1H-indol-3-yl]acetate
2-[1-(4-chlorobenzoyl)-4,6-difluoro-5-hydroxy-2-methyl-1H-indol-3-yl]acetamide
2-[1-(4-chlorobenzoyl)-4,6-difluoro-5-hydroxy-2-methyl-1H-indol-3-yl]-N-methylacetamide
2-[1-(4-chlorobenzoyl)-4,6-difluoro-5-hydroxy-2-methyl-1H-indol-3-yl]-N-ethylacetamide
2-[1-(4-chlorobenzoyl)-4,6-difluoro-5-hydroxy-2-methyl-1H-indol-3-yl]-N-propylacetamide
2-[1-(4-chlorobenzoyl)-4,6-difluoro-5-hydroxy-2-methyl-1H-indol-3-yl]-N-isopropylacetamide
2-[1-(4-chlorobenzoyl)-4,6-difluoro-5-hydroxy-2-methyl-1H-indol-3-yl]-N-butylacetamide
2-[1-(4-chlorobenzoyl)-4,6-difluoro-5-hydroxy-2-methyl-1H-indol-3-yl]-N-sec-butylacetamide
2-[1-(4-chlorobenzoyl)-4,6-difluoro-5-hydroxy-2-methyl-1H-indol-3-yl]-N-(2-hydroxyethyl)acetamide
methyl(2R,S)-2-[1-(4-chlorobenzoyl)-4-fluoro-5-hydroxy-2-methyl-1H-indol-3-yl]propanoate
ethyl(2R,S)-2-[1-(4-chlorobenzoyl)-4-fluoro-5-hydroxy-2-methyl-1H-indol-3-yl]propanoate
propyl(2R,S)-2-[1-(4-chlorobenzoyl)-4-fluoro-5-hydroxy-2-methyl-1H-indol-3-yl]propanoate
isopropyl(2R,S)-2-[1-(4-chlorobenzoyl)-4-fluoro-5-hydroxy-2-methyl-1H-indol-3-yl]propanoate
butyl(2R,S)-2-[1-(4-chlorobenzoyl)-4-fluoro-5-hydroxy-2-methyl-1H-indol-3-yl]propanoate
sec-butyl(2R,S)-2-[1-(4-chlorobenzoyl)-4-fluoro-5-hydroxy-2-methyl-1H-indol-3-yl]propanoate
(R)sec-butyl(2R,S)-2-[1-(4-chlorobenzoyl)-4-fluoro-5-hydroxy-2-methyl-1H-indol-3-yl]propanoate
(S)sec-butyl(2R,S)-2-[1-(4-chlorobenzoyl)-4-fluoro-5-hydroxy-2-methyl-1H-indol-3-yl]propanoate
tert-butyl(2R,S)-2-[1-(4-chlorobenzoyl)-4-fluoro-5-hydroxy-2-methyl-1H-indol-3-yl]propanoate
cyclobutyl(2R,S)-2-[1-(4-chlorobenzoyl)-4-fluoro-5-hydroxy-2-methyl-1H-indol-3-yl]propanoate
cyclopentyl(2R,S)-2-[1-(4-chlorobenzoyl)-4-fluoro-5-hydroxy-2-methyl-1H-indol-3-yl]propanoate
(2R,S)-2-[1-(4-chlorobenzoyl)-4-fluoro-5-hydroxy-2-methyl-1H-indol-3-yl]propanamide
(2R,S)-2-[1-(4-chlorobenzoyl)-4-fluoro-5-hydroxy-2-methyl-1H-indol-3-yl]-N-methylpropanamide
(2R,S)-2-[1-(4-chlorobenzoyl)-4-fluoro-5-hydroxy-2-methyl-1H-indol-3-yl]-N-ethylacetamide
(2R,S)-2-[1-(4-chlorobenzoyl)-4-fluoro-5-hydroxy-2-methyl-1H-indol-3-yl]-N-propylacetamide
(2R,S)-2-[1-(4-chlorobenzoyl)-4-fluoro-5-hydroxy-2-methyl-1H-indol-3-yl]-N-isopropylacetamide
(2R,S)-2-[1-(4-chlorobenzoyl)-4-fluoro-5-hydroxy-2-methyl-1H-indol-3-yl]-N-butylacetamide
(2R,S)-2-[1-(4-chlorobenzoyl)-4-fluoro-5-hydroxy-2-methyl-1H-indol-3-yl]-N-sec-butylacetamide
(2R,S)-2-[1-(4-chlorobenzoyl)-4-fluoro-5-hydroxy-2-methyl-1H-indol-3-yl]-N-(hydroxymethyl)propanamide
methyl(2R,S)-2-[1-(4-chlorobenzoyl)-6-fluoro-5-hydroxy-2-methyl-1H-indol-3-yl]propanoate
ethyl(2R,S)-2-[1-(4-chlorobenzoyl)-6-fluoro-5-hydroxy-2-methyl-1H-indol-3-yl]propanoate
propyl(2R,S)-2-[1-(4-chlorobenzoyl)-6-fluoro-5-hydroxy-2-methyl-1H-indol-3-yl]propanoate
isopropyl(2R,S)-2-[1-(4-chlorobenzoyl)-6-fluoro-5-hydroxy-2-methyl-1H-indol-3-yl]propanoate
butyl(2R,S)-2-[1-(4-chlorobenzoyl)-6-fluoro-5-hydroxy-2-methyl-1H-indol-3-yl]propanoate
sec-butyl(2R,S)-2-[1-(4-chlorobenzoyl)-6-fluoro-5-hydroxy-2-methyl-1H-indol-3-yl]propanoate
(R)sec-butyl(2R,S)-2-[1-(4-chlorobenzoyl)-6-fluoro-5-hydroxy-2-methyl-1H-indol-3-yl]propanoate
(S)sec-butyl(2R¹,S)-2-[1-(4-chlorobenzoyl)-6-fluoro-5-hydroxy-2-methyl-1H-indol-3-yl]propanoate
tert-butyl(2R,S)-2-[1-(4-chlorobenzoyl)-6-fluoro-5-hydroxy-2-methyl-1H-indol-3-yl]propanoate
cyclobutyl(2R,S)-2-[1-(4-chlorobenzoyl)-6-fluoro-5-hydroxy-2-methyl-1H-indol-3-yl]propanoate
cyclopentyl(2R,S)-2-[1-(4-chlorobenzoyl)-6-fluoro-5-hydroxy-2-methyl-1H-indol-3-yl]propanoate
(2R,S)-2-[1-(4-chlorobenzoyl)-6-fluoro-5-hydroxy-2-methyl-1H-indol-3-yl]propanamide
(2R,S)-2-[1-(4-chlorobenzoyl)-6-fluoro-5-hydroxy-2-methyl-1H-indol-3-yl]-N-methylpropanamide
(2R,S)-2-[1-(4-chlorobenzoyl)-6-fluoro-5-hydroxy-2-methyl-1H-indol-3-yl]-N-ethylacetamide
(2R,S)-2-[1-(4-chlorobenzoyl)-6-fluoro-5-hydroxy-2-methyl-1H-indol-3-yl]-N-propylacetamide
(2R,S)-2-[1-(4-chlorobenzoyl)-6-fluoro-5-hydroxy-2-methyl-1H-indol-3-yl]-N-isopropylacetamide
(2R,S)-2-[1-(4-chlorobenzoyl)-6-fluoro-5-hydroxy-2-methyl-1H-indol-3-yl]-N-butylacetamide
(2R,S)-2-[1-(4-chlorobenzoyl)-6-fluoro-5-hydroxy-2-methyl-1H-indol-3-yl]-N-sec-butylacetamide
(2R,S)-2-[1-(4-chlorobenzoyl)-6-fluoro-5-hydroxy-2-methyl-1H-indol-3-yl]-N-(hydroxymethyl)propanamide
methyl(2R,S)-2-[1-(4-chlorobenzoyl)-4,6-difluoro-5-hydroxy-2-methyl-1H-indol-3-yl]propanoate
ethyl(2R,S)-2-[1-(4-chlorobenzoyl)-4,6-difluoro-5-hydroxy-2-methyl-1H-indol-3-yl]propanoate
propyl(2R,S)-2-[1-(4-chlorobenzoyl)-4,6-difluoro-5-hydroxy-2-methyl-1H-indol-3-yl]propanoate
isopropyl(2R,S)-2-[1-(4-chlorobenzoyl)-4,6-difluoro-5-hydroxy-2-methyl-1H-indol-3-yl]propanoate
butyl(2R,S)-2-[1-(4-chlorobenzoyl)-4,6-difluoro-5-hydroxy-2-methyl-1H-indol-3-yl]propanoate
sec-butyl(2R,S)-2-[1-(4-chlorobenzoyl)-4,6-difluoro-5-hydroxy-2-methyl-1H-indol-3-yl]propanoate
(R)sec-butyl(2R,S)-2-[1-(4-chlorobenzoyl)-4,6-difluoro-5-hydroxy-2-methyl-1H-indol-3-yl]propanoate
(S)sec-butyl(2R,S)-2-[1-(4-chlorobenzoyl)-4,6-difluoro-5-hydroxy-2-methyl-1H-indol-3-yl]propanoate
tert-butyl(2R,S)-2-[1-(4-chlorobenzoyl)-4,6-difluoro-5-hydroxy-2-methyl-1H-indol-3-yl]propanoate
cyclobutyl(2R,S)-2-[1-(4-chlorobenzoyl)-4,6-difluoro-5-hydroxy-2-methyl-1H-indol-3-yl]propanoate
cyclopentyl(2R,S)-2-[1-(4-chlorobenzoyl)-4,6-difluoro-5-hydroxy-2-methyl-1H-indol-3-yl]propanoate
(2R,S)-2-[1-(4-chlorobenzoyl)-4,6-difluoro-5-hydroxy-2-methyl-1H-indol-3-yl]propanamide
(2R,S)-2-[1-(4-chlorobenzoyl)-4,6-difluoro-5-hydroxy-2-methyl-1H-indol-3-yl]-N-methylpropanamide
(2R,S)-2-[1-(4-chlorobenzoyl)-4,6-difluoro-5-hydroxy-2-methyl-1H-indol-3-yl]-N-ethylacetamide
(2R,S)-2-[1-(4-chlorobenzoyl)-4,6-difluoro-5-hydroxy-2-methyl-1H-indol-3-yl]-N-propylacetamide
(2R,S)-2-[1-(4-chlorobenzoyl)-4,6-difluoro-5-hydroxy-2-methyl-1H-indol-3-yl]-N-isopropylacetamide
(2R,S)-2-[1-(4-chlorobenzoyl)-4,6-difluoro-5-hydroxy-2-methyl-1H-indol-3-yl]-N-butylacetamide
(2R,S)-2-[1-(4-chlorobenzoyl)-4,6-difluoro-5-hydroxy-2-methyl-1H-indol-3-yl]-N-sec-butylacetamide
(2R,S)-2-[1-(4-chlorobenzoyl)-4,6-difluoro-5-hydroxy-2-methyl-1H-indol-3-yl]-N-(hydroxymethyl)propanamide
methyl[1-(3-chlorobenzoyl)-4,6-difluoro-5-hydroxy-2-methyl-1H-indol-3-yl]acetate
ethyl[1-(3-chlorobenzoyl)-4,6-difluoro-5-hydroxy-2-methyl-1H-indol-3-yl]acetate
propyl[1-(3-chlorobenzoyl)-4,6-difluoro-5-hydroxy-2-methyl-1H-indol-3-yl]acetate
butyl[1-(3-chlorobenzoyl)-4,6-difluoro-5-hydroxy-2-methyl-1H-indol-3-yl]acetate
sec-butyl[1-(3-chlorobenzoyl)-4,6-difluoro-5-hydroxy-2-methyl-1H-indol-3-yl]acetate
(R)sec-butyl[1-(3-chlorobenzoyl)-4,6-difluoro-5-hydroxy-2-methyl-1H-indol-3-yl]acetate
(S)sec-butyl[1-(3-chlorobenzoyl)-4,6-difluoro-5-hydroxy-2-methyl-1H-indol-3-yl]acetate
isopropyl[1-(3-chlorobenzoyl)-4,6-difluoro-5-hydroxy-2-methyl-1H-indol-3-yl]acetate
tert-butyl[1-(3-chlorobenzoyl)-4,6-difluoro-5-hydroxy-2-methyl-1H-indol-3-yl]acetate
cyclobutyl[1-(3-chlorobenzoyl)-4,6-difluoro-5-hydroxy-2-methyl-1H-indol-3-yl]acetate
cyclopentyl[1-(3-chlorobenzoyl)-4,6-difluoro-5-hydroxy-2-methyl-1H-indol-3-yl]acetate
2-[1-(3-chlorobenzoyl)-4,6-difluoro-5-hydroxy-2-methyl-1H-indol-3-yl]acetamide
2-[1-(3-chlorobenzoyl)-4,6-difluoro-5-hydroxy-2-methyl-1H-indol-3-yl]-N-methylacetamide
2-[1-(3-chlorobenzoyl)-4,6-difluoro-5-hydroxy-2-methyl-1H-indol-3-yl]-N-ethylacetamide
2-[1-(3-chlorobenzoyl)-4,6-difluoro-5-hydroxy-2-methyl-1H-indol-3-yl]-N-propylacetamide
2-[1-(3-chlorobenzoyl)-4,6-difluoro-5-hydroxy-2-methyl-1H-indol-3-yl]-N-isopropylacetamide
2-[1-(3-chlorobenzoyl)-4,6-difluoro-5-hydroxy-2-methyl-1H-indol-3-yl]-N-butylacetamide
2-[1-(3-chlorobenzoyl)-4,6-difluoro-5-hydroxy-2-methyl-1H-indol-3-yl]-N-sec-butylacetamide
2-[1-(3-chlorobenzoyl)-4,6-difluoro-5-hydroxy-2-methyl-1H-indol-3-yl]-N-(2-hydroxyethyl)acetamide
methyl[1-(3-chlorobenzoyl)-6-fluoro-5-hydroxy-2-methyl-1H-indol-3-yl]acetate
ethyl[1-(3-chlorobenzoyl)-6-fluoro-5-hydroxy-2-methyl-1H-indol-3-yl]acetate
propyl[1-(3-chlorobenzoyl)-6-fluoro-5-hydroxy-2-methyl-1H-indol-3-yl]acetate
butyl[1-(3-chlorobenzoyl)-6-fluoro-5-hydroxy-2-methyl-1H-indol-3-yl]acetate
sec-butyl[1-(3-chlorobenzoyl)-6-fluoro-5-hydroxy-2-methyl-1H-indol-3-yl]acetate
(R)sec-butyl[1-(3-chlorobenzoyl)-6-fluoro-5-hydroxy-2-methyl-1H-indol-3-yl]acetate
(S)sec-butyl[1-(3-chlorobenzoyl)-6-fluoro-5-hydroxy-2-methyl-1H-indol-3-yl]acetate
isopropyl[1-(3-chlorobenzoyl)-6-fluoro-5-hydroxy-2-methyl-1H-indol-3-yl]acetate
tert-butyl[1-(3-chlorobenzoyl)-6-fluoro-5-hydroxy-2-methyl-1H-indol-3-yl]acetate
cyclobutyl[1-(3-chlorobenzoyl)-6-fluoro-5-hydroxy-2-methyl-1H-indol-3-yl]acetate
cyclopentyl[1-(3-chlorobenzoyl)-6-fluoro-5-hydroxy-2-methyl-1H-indol-3-yl]acetate
2-[1-(3-chlorobenzoyl)-6-fluoro-5-hydroxy-2-methyl-1H-indol-3-yl]acetamide
2-[1-(3-chlorobenzoyl)-6-fluoro-5-hydroxy-2-methyl-1H-indol-3-yl]-N-methylacetamide
2-[1-(3-chlorobenzoyl)-6-fluoro-5-hydroxy-2-methyl-1H-indol-3-yl]-N-ethylacetamide
2-[1-(3-chlorobenzoyl)-6-fluoro-5-hydroxy-2-methyl-1H-indol-3-yl]-N-propylacetamide
2-[1-(3-chlorobenzoyl)-6-fluoro-5-hydroxy-2-methyl-1H-indol-3-yl]-N-isopropylacetamide
2-[1-(3-chlorobenzoyl)-6-fluoro-5-hydroxy-2-methyl-1H-indol-3-yl]-N-butylacetamide
2-[1-(3-chlorobenzoyl)-6-fluoro-5-hydroxy-2-methyl-1H-indol-3-yl]-N-sec-butylacetamide
2-[1-(3-chlorobenzoyl)-6-fluoro-5-hydroxy-2-methyl-1H-indol-3-yl]-N-(2-hydroxyethyl)acetamide
methyl[1-(3-chlorobenzoyl)-4-fluoro-5-hydroxy-2-methyl-1H-indol-3-yl]acetate
ethyl[1-(3-chlorobenzoyl)-4-fluoro-5-hydroxy-2-methyl-1H-indol-3-yl]acetate
propyl[1-(3-chlorobenzoyl)-4-fluoro-5-hydroxy-2-methyl-1H-indol-3-yl]acetate
butyl[1-(3-chlorobenzoyl)-4-fluoro-5-hydroxy-2-methyl-1H-indol-3-yl]acetate
sec-butyl[1-(3-chlorobenzoyl)-4-fluoro-5-hydroxy-2-methyl-1H-indol-3-yl]acetate
(R)sec-butyl[1-(3-chlorobenzoyl)-4-fluoro-5-hydroxy-2-methyl-1H-indol-3-yl]acetate
(S)sec-butyl[1-(3-chlorobenzoyl)-4-fluoro-5-hydroxy-2-methyl-1H-indol-3-yl]acetate
isopropyl[1-(3-chlorobenzoyl)-4-fluoro-5-hydroxy-2-methyl-1H-indol-3-yl]acetate
tert-butyl[1-(3-chlorobenzoyl)-4-fluoro-5-hydroxy-2-methyl-1H-indol-3-yl]acetate
cyclobutyl[1-(3-chlorobenzoyl)-4-fluoro-5-hydroxy-2-methyl-1H-indol-3-yl]acetate
cyclopentyl[1-(3-chlorobenzoyl)-4-fluoro-5-hydroxy-2-methyl-1H-indol-3-yl]acetate
2-[1-(3-chlorobenzoyl)-4-fluoro-5-hydroxy-2-methyl-1H-indol-3-yl]acetamide
2-[1-(3-chlorobenzoyl)-4-fluoro-5-hydroxy-2-methyl-1H-indol-3-yl]-N-methylacetamide
2-[1-(3-chlorobenzoyl)-4-fluoro-5-hydroxy-2-methyl-1H-indol-3-yl]-N-ethylacetamide
2-[1-(3-chlorobenzoyl)-4-fluoro-5-hydroxy-2-methyl-1H-indol-3-yl]-N-propylacetamide
2-[1-(3-chlorobenzoyl)-4-fluoro-5-hydroxy-2-methyl-1H-indol-3-yl]-N-isopropylacetamide
2-[1-(3-chlorobenzoyl)-4-fluoro-5-hydroxy-2-methyl-1H-indol-3-yl]-N-butylacetamide
2-[1-(3-chlorobenzoyl)-4-fluoro-5-hydroxy-2-methyl-1H-indol-3-yl]-N-sec-butylacetamide
2-[1-(3-chlorobenzoyl)-4-fluoro-5-hydroxy-2-methyl-1H-indol-3-yl]-N-(2-hydroxyethyl)acetamide
methyl[1-(3-chlorobenzoyl)-5-hydroxy-2-methyl-1H-indol-3-yl]acetate
ethyl[1-(3-chlorobenzoyl)-5-hydroxy-2-methyl-1H-indol-3-yl]acetate
propyl[1-(3-chlorobenzoyl)-5-hydroxy-2-methyl-1H-indol-3-yl]acetate
butyl[1-(3-chlorobenzoyl)-5-hydroxy-2-methyl-1H-indol-3-yl]acetate
sec-butyl[1-(3-chlorobenzoyl)-5-hydroxy-2-methyl-1H-indol-3-yl]acetate
(R)sec-butyl[1-(3-chlorobenzoyl)-5-hydroxy-2-methyl-1H-indol-3-yl]acetate
(S)sec-butyl[1-(3-chlorobenzoyl)-5-hydroxy-2-methyl-1H-indol-3-yl]acetate
isopropyl[1-(3-chlorobenzoyl)-5-hydroxy-2-methyl-1H-indol-3-yl]acetate
tert-butyl[1-(3-chlorobenzoyl)-5-hydroxy-2-methyl-1H-indol-3-yl]acetate
cyclobutyl[1-(3-chlorobenzoyl)-5-hydroxy-2-methyl-1H-indol-3-yl]acetate
cyclopentyl[1-(3-chlorobenzoyl)-5-hydroxy-2-methyl-1H-indol-3-yl]acetate
2-[1-(3-chlorobenzoyl)-5-hydroxy-2-methyl-1H-indol-3-yl]acetamide
2-[1-(3-chlorobenzoyl)-5-hydroxy-2-methyl-1H-indol-3-yl]-N-methylacetamide
2-[1-(3-chlorobenzoyl)-5-hydroxy-2-methyl-1H-indol-3-yl]-N-ethylacetamide
2-[1-(3-chlorobenzoyl)-5-hydroxy-2-methyl-1H-indol-3-yl]-N-propylacetamide
2-[1-(3-chlorobenzoyl)-5-hydroxy-2-methyl-1H-indol-3-yl]-N-isopropylacetamide
2-[1-(3-chlorobenzoyl)-5-hydroxy-2-methyl-1H-indol-3-yl]-N-butylacetamide
2-[1-(3-chlorobenzoyl)-5-hydroxy-2-methyl-1H-indol-3-yl]-N-sec-butylacetamide
2-[1-(3-chlorobenzoyl)-5-hydroxy-2-methyl-1H-indol-3-yl]-N-(2-hydroxyethyl)acetamide
methyl[1-(3,4-dichlorobenzoyl)-5-hydroxy-2-methyl-1H-indol-3-yl]acetate
ethyl[1-(3,4-dichlorobenzoyl)-5-hydroxy-2-methyl-1H-indol-3-yl]acetate
propyl[1-(3,4-dichlorobenzoyl)-5-hydroxy-2-methyl-1H-indol-3-yl]acetate
butyl[1-(3,4-dichlorobenzoyl)-5-hydroxy-2-methyl-1H-indol-3-yl]acetate
sec-butyl[1-(3,4-dichlorobenzoyl)-5-hydroxy-2-methyl-1H-indol-3-yl]acetate
(R)sec-butyl[1-(3,4-dichlorobenzoyl)-5-hydroxy-2-methyl-1H-indol-3-yl]acetate
(S)sec-butyl[1-(3,4-dichlorobenzoyl)-5-hydroxy-2-methyl-1H-indol-3-yl]acetate
isopropyl[1-(3,4-dichlorobenzoyl)-5-hydroxy-2-methyl-1H-indol-3-yl]acetate
tert-butyl[1-(3,4-dichlorobenzoyl)-5-hydroxy-2-methyl-1H-indol-3-yl]acetate
cyclobutyl[1-(3,4-dichlorobenzoyl)-5-hydroxy-2-methyl-1H-indol-3-yl]acetate
cyclopentyl[1-(3,4-dichlorobenzoyl)-5-hydroxy-2-methyl-1H-indol-3-yl]acetate
2-[1-(3,4-dichlorobenzoyl)-5-hydroxy-2-methyl-1H-indol-3-yl]acetamide
2-[1-(3,4-dichlorobenzoyl)-5-hydroxy-2-methyl-1H-indol-3-yl]-N-methylacetamide
2-[1-(3,4-dichlorobenzoyl)-5-hydroxy-2-methyl-1H-indol-3-yl]-N-ethylacetamide
2-[1-(3,4-dichlorobenzoyl)-5-hydroxy-2-methyl-1H-indol-3-yl]-N-propylacetamide
2-[1-(3,4-dichlorobenzoyl)-5-hydroxy-2-methyl-1H-indol-3-yl]-N-isopropylacetamide
2-[1-(3,4-dichlorobenzoyl)-5-hydroxy-2-methyl-1H-indol-3-yl]-N-butylacetamide
2-[1-(3,4-dichlorobenzoyl)-5-hydroxy-2-methyl-1H-indol-3-yl]-N-sec-butylacetamide
2-[1-(3,4-dichlorobenzoyl)-5-hydroxy-2-methyl-1H-indol-3-yl]-N-(2-hydroxyethyl)acetamide
methyl[1-(3,4-dichlorobenzoyl)-6-fluoro-5-hydroxy-2-methyl-1H-indol-3-yl]acetate
ethyl[1-(3,4-dichlorobenzoyl)-6-fluoro-5-hydroxy-2-methyl-1H-indol-3-yl]acetate
propyl[1-(3,4-dichlorobenzoyl)-6-fluoro-5-hydroxy-2-methyl-1H-indol-3-yl]acetate
butyl[1-(3,4-dichlorobenzoyl)-6-fluoro-5-hydroxy-2-methyl-1H-indol-3-yl]acetate
sec-butyl[1-(3,4-dichlorobenzoyl)-6-fluoro-5-hydroxy-2-methyl-1H-indol-3-yl]acetate
(R)sec-butyl[1-(3,4-dichlorobenzoyl)-6-fluoro-5-hydroxy-2-methyl-1H-indol-3-yl]acetate
(S)sec-butyl[1-(3,4-dichlorobenzoyl)-6-fluoro-5-hydroxy-2-methyl-1H-indol-3-yl]acetate
isopropyl[1-(3,4-dichlorobenzoyl)-6-fluoro-5-hydroxy-2-methyl-1H-indol-3-yl]acetate
tert-butyl[1-(3,4-dichlorobenzoyl)-6-fluoro-5-hydroxy-2-methyl-1H-indol-3-yl]acetate
cyclobutyl[1-(3,4-dichlorobenzoyl)-6-fluoro-5-hydroxy-2-methyl-1H-indol-3-yl]acetate
cyclopentyl[1-(3,4-dichlorobenzoyl)-6-fluoro-5-hydroxy-2-methyl-1H-indol-3-yl]acetate
2-[1-(3,4-dichlorobenzoyl)-6-fluoro-5-hydroxy-2-methyl-1H-indol-3-yl]acetamide
2-[1-(3,4-dichlorobenzoyl)-6-fluoro-5-hydroxy-2-methyl-1H-indol-3-yl]-N-methylacetamide
2-[1-(3,4-dichlorobenzoyl)-6-fluoro-5-hydroxy-2-methyl-1H-indol-3-yl]-N-ethylacetamide
2-[1-(3,4-dichlorobenzoyl)-6-fluoro-5-hydroxy-2-methyl-1H-indol-3-yl]-N-propylacetamide
2-[1-(3,4-dichlorobenzoyl)-6-fluoro-5-hydroxy-2-methyl-1H-indol-3-yl]-N-isopropylacetamide
2-[1-(3,4-dichlorobenzoyl)-6-fluoro-5-hydroxy-2-methyl-1H-indol-3-yl]-N-butylacetamide
2-[1-(3,4-dichlorobenzoyl)-6-fluoro-5-hydroxy-2-methyl-1H-indol-3-yl]-N-sec-butylacetamide
2-[1-(3,4-dichlorobenzoyl)-6-fluoro-5-hydroxy-2-methyl-1H-indol-3-yl]-N-(2-hydroxyethyl)acetamide
methyl[1-(3,4-dichlorobenzoyl)-4-fluoro-5-hydroxy-2-methyl-1H-indol-3-yl]acetate
ethyl[1-(3,4-dichlorobenzoyl)-4-fluoro-5-hydroxy-2-methyl-1H-indol-3-yl]acetate
propyl[1-(3,4-dichlorobenzoyl)-4-fluoro-5-hydroxy-2-methyl-1H-indol-3-yl]acetate
butyl[1-(3,4-dichlorobenzoyl)-4-fluoro-5-hydroxy-2-methyl-1H-indol-3-yl]acetate
sec-butyl[1-(3,4-dichlorobenzoyl)-4-fluoro-5-hydroxy-2-methyl-1H-indol-3-yl]acetate
(R)sec-butyl[1-(3,4-dichlorobenzoyl)-4-fluoro-5-hydroxy-2-methyl-1H-indol-3-yl]acetate
(S)sec-butyl[1-(3,4-dichlorobenzoyl)-4-fluoro-5-hydroxy-2-methyl-1H-indol-3-yl]acetate
isopropyl[1-(3,4-dichlorobenzoyl)-4-fluoro-5-hydroxy-2-methyl-1H-indol-3-yl]acetate
tert-butyl[1-(3,4-dichlorobenzoyl)-4-fluoro-5-hydroxy-2-methyl-1H-indol-3-yl]acetate
cyclobutyl[1-(3,4-dichlorobenzoyl)-4-fluoro-5-hydroxy-2-methyl-1H-indol-3-yl]acetate
cyclopentyl[1-(3,4-dichlorobenzoyl)-4-fluoro-5-hydroxy-2-methyl-1H-indol-3-yl]acetate
2-[1-(3,4-dichlorobenzoyl)-4-fluoro-5-hydroxy-2-methyl-1H-indol-3-yl]acetamide
2-[1-(3,4-dichlorobenzoyl)-4-fluoro-5-hydroxy-2-methyl-1H-indol-3-yl]-N-methylacetamide
2-[1-(3,4-dichlorobenzoyl)-4-fluoro-5-hydroxy-2-methyl-1H-indol-3-yl]-N-ethylacetamide
2-[1-(3,4-dichlorobenzoyl)-4-fluoro-5-hydroxy-2-methyl-1H-indol-3-yl]-N-propylacetamide
2-[1-(3,4-dichlorobenzoyl)-4-fluoro-5-hydroxy-2-methyl-1H-indol-3-yl]-N-isopropylacetamide
2-[1-(3,4-dichlorobenzoyl)-4-fluoro-5-hydroxy-2-methyl-1H-indol-3-yl]-N-butylacetamide
2-[1-(3,4-dichlorobenzoyl)-4-fluoro-5-hydroxy-2-methyl-1H-indol-3-yl]-N-sec-butylacetamide
2-[1-(3,4-dichlorobenzoyl)-4-fluoro-5-hydroxy-2-methyl-1H-indol-3-yl]-N-(2-hydroxyethyl)acetamide
methyl[1-(3,4-dichlorobenzoyl)-4,6-difluoro-5-hydroxy-2-methyl-1H-indol-3-yl]acetate
ethyl[1-(3,4-dichlorobenzoyl)-4,6-difluoro-5-hydroxy-2-methyl-1H-indol-3-yl]acetate
propyl[1-(3,4-dichlorobenzoyl)-4,6-difluoro-5-hydroxy-2-methyl-1H-indol-3-yl]acetate
butyl[1-(3,4-dichlorobenzoyl)-4,6-difluoro-5-hydroxy-2-methyl-1H-indol-3-yl]acetate
sec-butyl[1-(3,4-dichlorobenzoyl)-4,6-difluoro-5-hydroxy-2-methyl-1H-indol-3-yl]acetate
(R)sec-butyl[1-(3,4-dichlorobenzoyl)-4,6-difluoro-5-hydroxy-2-methyl-1H-indol-3-yl]acetate
(S)sec-butyl[1-(3,4-dichlorobenzoyl)-4,6-difluoro-5-hydroxy-2-methyl-1H-indol-3-yl]acetate
isopropyl[1-(3,4-dichlorobenzoyl)-4,6-difluoro-5-hydroxy-2-methyl-1H-indol-3-yl]acetate
tert-butyl[1-(3,4-dichlorobenzoyl)-4,6-difluoro-5-hydroxy-2-methyl-1H-indol-3-yl]acetate
cyclobutyl[1-(3,4-dichlorobenzoyl)-4,6-difluoro-5-hydroxy-2-methyl-1H-indol-3-yl]acetate
cyclopentyl[1-(3,4-dichlorobenzoyl)-4,6-difluoro-5-hydroxy-2-methyl-1H-indol-3-yl]acetate
2-[1-(3,4-dichlorobenzoyl)-4,6-difluoro-5-hydroxy-2-methyl-1H-indol-3-yl]acetamide
2-[1-(3,4-dichlorobenzoyl)-4,6-difluoro-1-hydroxy-2-methyl-1H-indol-3-yl]-N-methylacetamide
2-[1-(3,4-dichlorobenzoyl)-4,6-difluoro-5-hydroxy-2-methyl-1H-indol-3-yl]-N-ethylacetamide
2-[1-(3,4-dichlorobenzoyl)-4,6-difluoro-5-hydroxy-2-methyl-1H-indol-3-yl]-N-propylacetamide
2-[1-(3,4-dichlorobenzoyl)-4,6-difluoro-5-hydroxy-2-methyl-1H-indol-3-yl]-N-isopropylacetamide
2-[1-(3,4-dichlorobenzoyl)-4,6-difluoro-5-hydroxy-2-methyl-1H-indol-3-yl]-N-butylacetamide
2-[1-(3,4-dichlorobenzoyl)-4,6-difluoro-5-hydroxy-2-methyl-1H-indol-3-yl]-N-sec-butylacetamide
2-[1-(3,4-dichlorobenzoyl)-4,6-difluoro-5-hydroxy-2-methyl-1H-indol-3-yl]-N-(2-hydroxyethyl)acetamide
methyl[1-(3,4-difluorobenzoyl)-5-hydroxy-2-methyl-1H-indol-3-yl]acetate
ethyl[1-(3,4-difluorobenzoyl)-5-hydroxy-2-methyl-1H-indol-3-yl]acetate
propyl[1-(3,4-difluorobenzoyl)-5-hydroxy-2-methyl-1H-indol-3-yl]acetate
butyl[1-(3,4-difluorobenzoyl)-5-hydroxy-2-methyl-1H-indol-3-yl]acetate
sec-butyl[1-(3,4-difluorobenzoyl)-5-hydroxy-2-methyl-1H-indol-3-yl]acetate
(R)sec-butyl[1-(3,4-difluorobenzoyl)-5-hydroxy-2-methyl-1H-indol-3-yl]acetate
(S)sec-butyl[1-(3,4-difluorobenzoyl)-5-hydroxy-2-methyl-1H-indol-3-yl]acetate
isopropyl[1-(3,4-difluorobenzoyl)-5-hydroxy-2-methyl-1H-indol-3-yl]acetate
tert-butyl[1-(3,4-difluorobenzoyl)-5-hydroxy-2-methyl-1H-indol-3-yl]acetate
cyclobutyl[1-(3,4-difluorobenzoyl)-5-hydroxy-2-methyl-1H-indol-3-yl]acetate
cyclopentyl[1-(3,4-difluorobenzoyl)-5-hydroxy-2-methyl-1H-indol-3-yl]acetate
2-[1-(3,4-difluorobenzoyl)-5-hydroxy-2-methyl-1H-indol-3-yl]acetamide
2-[1-(3,4-difluorobenzoyl)-5-hydroxy-2-methyl-1H-indol-3-yl]-N-methylacetamide
2-[1-(3,4-difluorobenzoyl)-5-hydroxy-2-methyl-1H-indol-3-yl]-N-ethylacetamide
2-[1-(3,4-difluorobenzoyl)-5-hydroxy-2-methyl-1H-indol-3-yl]-N-propylacetamide
2-[1-(3,4-difluorobenzoyl)-5-hydroxy-2-methyl-1H-indol-3-yl]-N-isopropylacetamide
2-[1-(3,4-difluorobenzoyl)-5-hydroxy-2-methyl-1H-indol-3-yl]-N-butylacetamide
2-[1-(3,4-difluorobenzoyl)-5-hydroxy-2-methyl-1H-indol-3-yl]-N-sec-butylacetamide
2-[1-(3,4-difluorobenzoyl)-5-hydroxy-2-methyl-1H-indol-3-yl]-N-(2-hydroxyethyl)acetamide
methyl[1-(3,4-difluorobenzoyl)-6-fluoro-5-hydroxy-2-methyl-1H-indol-3-yl]acetate
ethyl[1-(3,4-difluorobenzoyl)-6-fluoro-5-hydroxy-2-methyl-1H-indol-3-yl]acetate
propyl[1-(3,4-difluorobenzoyl)-6-fluoro-5-hydroxy-2-methyl-1H-indol-3-yl]acetate
butyl[1-(3,4-difluorobenzoyl)-6-fluoro-5-hydroxy-2-methyl-1H-indol-3-yl]acetate
sec-butyl[1-(3,4-difluorobenzoyl)-6-fluoro-5-hydroxy-2-methyl-1H-indol-3-yl]acetate
(R)sec-butyl[1-(3,4-difluorobenzoyl)-6-fluoro-5-hydroxy-2-methyl-1H-indol-3-yl]acetate
(S)sec-butyl[1-(3,4-difluorobenzoyl)-6-fluoro-5-hydroxy-2-methyl-1H-indol-3-yl]acetate
isopropyl[1-(3,4-difluorobenzoyl)-6-fluoro-5-hydroxy-2-methyl-1H-indol-3-yl]acetate
tert-butyl[1-(3,4-difluorobenzoyl)-6-fluoro-5-hydroxy-2-methyl-1H-indol-3-yl]acetate
cyclobutyl[1-(3,4-difluorobenzoyl)-6-fluoro-5-hydroxy-2-methyl-1H-indol-3-yl]acetate
cyclopentyl[1-(3,4-difluorobenzoyl)-6-fluoro-5-hydroxy-2-methyl-1H-indol-3-yl]acetate
2-[1-(3,4-difluorobenzoyl)-6-fluoro-5-hydroxy-2-methyl-1H-indol-3-yl]acetamide
2-[1-(3,4-difluorobenzoyl)-6-fluoro-5-hydroxy-2-methyl-1H-indol-3-yl]-N-methylacetamide
2-[1-(3,4-difluorobenzoyl)-6-fluoro-5-hydroxy-2-methyl-1H-indol-3-yl]-N-ethylacetamide
2-[1-(3,4-difluorobenzoyl)-6-fluoro-5-hydroxy-2-methyl-1H-indol-3-yl]-N-propylacetamide
2-[1-(3,4-difluorobenzoyl)-6-fluoro-5-hydroxy-2-methyl-1H-indol-3-yl]-N-isopropylacetamide
2-[1-(3,4-difluorobenzoyl)-6-fluoro-5-hydroxy-2-methyl-1H-indol-3-yl]-N-butylacetamide
2-[1-(3,4-difluorobenzoyl)-6-fluoro-5-hydroxy-2-methyl-1H-indol-3-yl]-N-sec-butylacetamide
2-[1-(3,4-difluorobenzoyl)-6-fluoro-5-hydroxy-2-methyl-1H-indol-3-yl]-N-(2-hydroxyethyl)acetamide
methyl[1-(3,4-difluorobenzoyl)-4-fluoro-5-hydroxy-2-methyl-1H-indol-3-yl]acetate
ethyl[1-(3,4-difluorobenzoyl)-4-fluoro-5-hydroxy-2-methyl-1H-indol-3-yl]acetate
propyl[1-(3,4-difluorobenzoyl)-4-fluoro-5-hydroxy-2-methyl-1H-indol-3-yl]acetate
butyl[1-(3,4-difluorobenzoyl)-4-fluoro-5-hydroxy-2-methyl-1H-indol-3-yl]acetate
sec-butyl[1-(3,4-difluorobenzoyl)-4-fluoro-5-hydroxy-2-methyl-1H-indol-3-yl]acetate
(R)sec-butyl[1-(3,4-difluorobenzoyl)-4-fluoro-5-hydroxy-2-methyl-1H-indol-3-yl]acetate
(S)sec-butyl[1-(3,4-difluorobenzoyl)-4-fluoro-5-hydroxy-2-methyl-1H-indol-3-yl]acetate
isopropyl[1-(3,4-difluorobenzoyl)-4-fluoro-5-hydroxy-2-methyl-1H-indol-3-yl]acetate
tert-butyl[1-(3,4-difluorobenzoyl)-4-fluoro-5-hydroxy-2-methyl-1H-indol-3-yl]acetate
cyclobutyl[1-(3,4-difluorobenzoyl)-4-fluoro-5-hydroxy-2-methyl-1H-indol-3-yl]acetate
cyclopentyl[1-(3,4-difluorobenzoyl)-4-fluoro-5-hydroxy-2-methyl-1H-indol-3-yl]acetate
2-[1-(3,4-difluorobenzoyl)-4-fluoro-5-hydroxy-2-methyl-1H-indol-3-yl]acetamide
2-[1-(3,4-difluorobenzoyl)-4-fluoro-5-hydroxy-2-methyl-1H-indol-3-yl]-N-methylacetamide
2-[1-(3,4-difluorobenzoyl)-4-fluoro-5-hydroxy-2-methyl-1H-indol-3-yl]-N-ethylacetamide
2-[1-(3,4-difluorobenzoyl)-4-fluoro-5-hydroxy-2-methyl-1H-indol-3-yl]-N-propylacetamide
2-[1-(3,4-difluorobenzoyl)-4-fluoro-5-hydroxy-2-methyl-1H-indol-3-yl]-N-isopropylacetamide
2-[1-(3,4-difluorobenzoyl)-4-fluoro-5-hydroxy-2-methyl-1H-indol-3-yl]-N-butylacetamide
2-[1-(3,4-difluorobenzoyl)-4-fluoro-5-hydroxy-2-methyl-1H-indol-3-yl]-N-sec-butylacetamide
2-[1-(3,4-difluorobenzoyl)-4-fluoro-5-hydroxy-2-methyl-1H-indol-3-yl]-N-(2-hydroxyethyl)acetamide
methyl[1-(3,4-difluorobenzoyl)-4,6-difluoro-5-hydroxy-2-methyl-1H-indol-3-yl]acetate
ethyl[1-(3,4-difluorobenzoyl)-4,6-difluoro-5-hydroxy-2-methyl-1H-indol-3-yl]acetate
propyl[1-(3,4-difluorobenzoyl)-4,6-difluoro-5-hydroxy-2-methyl-1H-indol-3-yl]acetate
butyl[1-(3,4-difluorobenzoyl)-4,6-difluoro-5-hydroxy-2-methyl-1H-indol-3-yl]acetate
sec-butyl[1-(3,4-difluorobenzoyl)-4,6-difluoro-5-hydroxy-2-methyl-1H-indol-3-yl]acetate
(R)sec-butyl[1-(3,4-difluorobenzoyl)-4,6-difluoro-5-hydroxy-2-methyl-1H-indol-3-yl]acetate
(S)sec-butyl[1-(3,4-difluorobenzoyl)-4,6-difluoro-5-hydroxy-2-methyl-1H-indol-3-yl]acetate
isopropyl[1-(3,4-difluorobenzoyl)-4,6-difluoro-5-hydroxy-2-methyl-1H-indol-3-yl]acetate
tert-butyl[1-(3,4-difluorobenzoyl)-4,6-difluoro-5-hydroxy-2-methyl-1H-indol-3-yl]acetate
cyclobutyl[1-(3,4-difluorobenzoyl)-4,6-difluoro-5-hydroxy-2-methyl-1H-indol-3-yl]acetate
cyclopentyl[1-(3,4-difluorobenzoyl)-4,6-difluoro-5-hydroxy-2-methyl-1H-indol-3-yl]acetate
2-[1-(3,4-difluorobenzoyl)-4,6-difluoro-5-hydroxy-2-methyl-1H-indol-3-yl]acetamide
2-[1-(3,4-difluorobenzoyl)-4,6-difluoro-5-hydroxy-2-methyl-1H-indol-3-yl]-N-methylacetamide
2-[1-(3,4-difluorobenzoyl)-4,6-difluoro-5-hydroxy-2-methyl-1H-indol-3-yl]-N-ethylacetamide
2-[1-(3,4-difluorobenzoyl)-4,6-difluoro-5-hydroxy-2-methyl-1H-indol-3-yl]-N-propylacetamide
2-[1-(3,4-difluorobenzoyl)-4,6-difluoro-5-hydroxy-2-methyl-1H-indol-3-yl]-N-isopropylacetamide
2-[1-(3,4-difluorobenzoyl)-4,6-difluoro-5-hydroxy-2-methyl-1H-indol-3-yl]-N-butylacetamide
2-[1-(3,4-difluorobenzoyl)-4,6-difluoro-5-hydroxy-2-methyl-1H-indol-3-yl]-N-sec-butylacetamide
2-[1-(3,4-difluorobenzoyl)-4,6-difluoro-5-hydroxy-2-methyl-1H-indol-3-yl]-N-(2-hydroxyethyl)acetamide
methyl[1-(4-chloro-3-fluorobenzoyl)-5-hydroxy-2-methyl-1H-indol-3-yl]acetate
ethyl[1-(4-chloro-3-fluorobenzoyl)-5-hydroxy-2-methyl-1H-indol-3-yl]acetate
propyl[1-(4-chloro-3-fluorobenzoyl)-5-hydroxy-2-methyl-1H-indol-3-yl]acetate
butyl[1-(4-chloro-3-fluorobenzoyl)-5-hydroxy-2-methyl-1H-indol-3-yl]acetate
sec-butyl[1-(4-chloro-3-fluorobenzoyl)-5-hydroxy-2-methyl-1H-indol-3-yl]acetate
(R)sec-butyl[1-(4-chloro-3-fluorobenzoyl)-5-hydroxy-2-methyl-1H-indol-3-yl]acetate
(S)sec-butyl[1-(4-chloro-3-fluorobenzoyl)-5-hydroxy-2-methyl-1H-indol-3-yl]acetate
isopropyl[1-(4-chloro-3-fluorobenzoyl)-5-hydroxy-2-methyl-1H-indol-3-yl]acetate
tert-butyl[1-(4-chloro-3-fluorobenzoyl)-5-hydroxy-2-methyl-1H-indol-3-yl]acetate
cyclobutyl[1-(4-chloro-3-fluorobenzoyl)-5-hydroxy-2-methyl-1H-indol-3-yl]acetate
cyclopentyl[1-(4-chloro-3-fluorobenzoyl)-5-hydroxy-2-methyl-1H-indol-3-yl]acetate
2-[1-(4-chloro-3-fluorobenzoyl)-5-hydroxy-2-methyl-1H-indol-3-yl]acetamide
2-[1-(4-chloro-3-fluorobenzoyl)-5-hydroxy-2-methyl-1H-indol-3-yl]-N-methylacetamide
2-[1-(4-chloro-3-fluorobenzoyl)-5-hydroxy-2-methyl-1H-indol-3-yl]-N-ethylacetamide
2-[1-(4-chloro-3-fluorobenzoyl)-5-hydroxy-2-methyl-1H-indol-3-yl]-N-propylacetamide
2-[1-(4-chloro-3-fluorobenzoyl)-5-hydroxy-2-methyl-1H-indol-3-yl]-N-isopropylacetamide
2-[1-(4-chloro-3-fluorobenzoyl)-5-hydroxy-2-methyl-1H-indol-3-yl]-N-butylacetamide
2-[1-(4-chloro-3-fluorobenzoyl)-5-hydroxy-2-methyl-1H-indol-3-yl]-N-sec-butylacetamide
2-[1-(4-chloro-3-fluorobenzoyl)-5-hydroxy-2-methyl-1H-indol-3-yl]-N-(2-hydroxyethyl)acetamide
methyl[1-(4-chloro-3-fluorobenzoyl)-6-fluoro-5-hydroxy-2-methyl-1H-indol-3-yl]acetate
ethyl[1-(4-chloro-3-fluorobenzoyl)-6-fluoro-5-hydroxy-2-methyl-1H-indol-3-yl]acetate
propyl[1-(4-chloro-3-fluorobenzoyl)-6-fluoro-5-hydroxy-2-methyl-1H-indol-3-yl]acetate
butyl[1-(4-chloro-3-fluorobenzoyl)-6-fluoro-5-hydroxy-2-methyl-1H-indol-3-yl]acetate
sec-butyl[1-(4-chloro-3-fluorobenzoyl)-6-fluoro-5-hydroxy-2-methyl-1H-indol-3-yl]acetate
(R)sec-butyl[1-(4-chloro-3-fluorobenzoyl)-6-fluoro-5-hydroxy-2-methyl-1H-indol-3-yl]acetate
(S)sec-butyl[1-(4-chloro-3-fluorobenzoyl)-6-fluoro-5-hydroxy-2-methyl-1H-indol-3-yl]acetate
isopropyl[1-(4-chloro-3-fluorobenzoyl)-6-fluoro-5-hydroxy-2-methyl-1H-indol-3-yl]acetate
tert-butyl[1-(4-chloro-3-fluorobenzoyl)-6-fluoro-5-hydroxy-2-methyl-1H-indol-3-yl]acetate
cyclobutyl[1-(4-chloro-3-fluorobenzoyl)-6-fluoro-5-hydroxy-2-methyl-1H-indol-3-yl]acetate
cyclopentyl[1-(4-chloro-3-fluorobenzoyl)-6-fluoro-5-hydroxy-2-methyl-1H-indol-3-yl]acetate
2-[1-(4-chloro-3-fluorobenzoyl)-6-fluoro-5-hydroxy-2-methyl-1H-indol-3-yl]acetamide
2-[1-(4-chloro-3-fluorobenzoyl)-6-fluoro-5-hydroxy-2-methyl-1H-indol-3-yl]-N-methylacetamide
2-[1-(4-chloro-3-fluorobenzoyl)-6-fluoro-5-hydroxy-2-methyl-1H-indol-3-yl]-N-ethylacetamide
2-[1-(4-chloro-3-fluorobenzoyl)-6-fluoro-5-hydroxy-2-methyl-1H-indol-3-yl]-N-propylacetamide
2-[1-(4-chloro-3-fluorobenzoyl)-6-fluoro-5-hydroxy-2-methyl-1H-indol-3-yl]-N-isopropylacetamide
2-[1-(4-chloro-3-fluorobenzoyl)-6-fluoro-5-hydroxy-2-methyl-1H-indol-3-yl]-N-butylacetamide
2-[1-(4-chloro-3-fluorobenzoyl)-6-fluoro-5-hydroxy-2-methyl-1H-indol-3-yl]-N-sec-butylacetamide
2-[1-(4-chloro-3-fluorobenzoyl)-6-fluoro-5-hydroxy-2-methyl-1H-indol-3-yl]-N-(2-hydroxyethyl)acetamide
methyl[1-(4-chloro-3-fluorobenzoyl)-4-fluoro-5-hydroxy-2-methyl-1H-indol-3-yl]acetate
ethyl[1-(4-chloro-3-fluorobenzoyl)-4-fluoro-5-hydroxy-2-methyl-1H-indol-3-yl]acetate
propyl[1-(4-chloro-3-fluorobenzoyl)-4-fluoro-5-hydroxy-2-methyl-1H-indol-3-yl]acetate
butyl[1-(4-chloro-3-fluorobenzoyl)-4-fluoro-5-hydroxy-2-methyl-1H-indol-3-yl]acetate
sec-butyl[1-(4-chloro-3-fluorobenzoyl)-4-fluoro-5-hydroxy-2-methyl-1H-indol-3-yl]acetate
(R)sec-butyl[1-(4-chloro-3-fluorobenzoyl)-4-fluoro-5-hydroxy-2-methyl-1H-indol-3-yl]acetate
(S)sec-butyl[1-(4-chloro-3-fluorobenzoyl)-4-fluoro-5-hydroxy-2-methyl-1H-indol-3-yl]acetate
isopropyl[1-(4-chloro-3-fluorobenzoyl)-4-fluoro-5-hydroxy-2-methyl-1H-indol-3-yl]acetate
tert-butyl[1-(4-chloro-3-fluorobenzoyl)-4-fluoro-5-hydroxy-2-methyl-1H-indol-3-yl]acetate
cyclobutyl[1-(4-chloro-3-fluorobenzoyl)-4-fluoro-5-hydroxy-2-methyl-1H-indol-3-yl]acetate
cyclopentyl[1-(4-chloro-3-fluorobenzoyl)-4-fluoro-5-hydroxy-2-methyl-1H-indol-3-yl]acetate
2-[1-(4-chloro-3-fluorobenzoyl)-4-fluoro-5-hydroxy-2-methyl-1H-indol-3-yl]acetamide
2-[1-(4-chloro-3-fluorobenzoyl)-4-fluoro-5-hydroxy-2-methyl-1H-indol-3-yl]-N-methylacetamide
2-[1-(4-chloro-3-fluorobenzoyl)-4-fluoro-5-hydroxy-2-methyl-1H-indol-3-yl]-N-ethylacetamide
2-[1-(4-chloro-3-fluorobenzoyl)-4-fluoro-5-hydroxy-2-methyl-1H-indol-3-yl]-N-propylacetamide
2-[1-(4-chloro-3-fluorobenzoyl)-4-fluoro-5-hydroxy-2-methyl-1H-indol-3-yl]-N-isopropylacetamide
2-[1-(4-chloro-3-fluorobenzoyl)-4-fluoro-5-hydroxy-2-methyl-1H-indol-3-yl]-N-butylacetamide
2-[1-(4-chloro-3-fluorobenzoyl)-4-fluoro-5-hydroxy-2-methyl-1H-indol-3-yl]-N-sec-butylacetamide
2-[1-(4-chloro-3-fluorobenzoyl)-4-fluoro-5-hydroxy-2-methyl-1H-indol-3-yl]-N-(2-hydroxyethyl)acetamide
methyl[1-(4-chloro-3-fluorobenzoyl)-4,6-difluoro-5-hydroxy-2-methyl-1H-indol-3-yl]acetate
ethyl[1-(4-chloro-3-fluorobenzoyl)-4,6-difluoro-5-hydroxy-2-methyl-1H-indol-3-yl]acetate
propyl[1-(4-chloro-3-fluorobenzoyl)-4,6-difluoro-5-hydroxy-2-methyl-1H-indol-3-yl]acetate
butyl[1-(4-chloro-3-fluorobenzoyl)-4,6-difluoro-5-hydroxy-2-methyl-1H-indol-3-yl]acetate
sec-butyl[1-(4-chloro-3-fluorobenzoyl)-4,6-difluoro-5-hydroxy-2-methyl-1H-indol-3-yl]acetate
(R)sec-butyl[1-(4-chloro-3-fluorobenzoyl)-4,6-difluoro-5-hydroxy-2-methyl-1H-indol-3-yl]acetate
(S)sec-butyl[1-(4-chloro-3-fluorobenzoyl)-4,6-difluoro-5-hydroxy-2-methyl-1H-indol-3-yl]acetate
isopropyl[1-(4-chloro-3-fluorobenzoyl)-4,6-difluoro-5-hydroxy-2-methyl-1H-indol-3-yl]acetate
tert-butyl[1-(4-chloro-3-fluorobenzoyl)-4,6-difluoro-5-hydroxy-2-methyl-1H-indol-3-yl]acetate
cyclobutyl[1-(4-chloro-3-fluorobenzoyl)-4,6-difluoro-5-hydroxy-2-methyl-1H-indol-3-yl]acetate
cyclopentyl[1-(4-chloro-3-fluorobenzoyl)-4,6-difluoro-5-hydroxy-2-methyl-1H-indol-3-yl]acetate
2-[1-(4-chloro-3-fluorobenzoyl)-4,6-difluoro-5-hydroxy-2-methyl-1H-indol-3-yl]acetamide
2-[1-(4-chloro-3-fluorobenzoyl)-4,6-difluoro-5-hydroxy-2-methyl-1H-indol-3-yl]-N-methylacetamide
2-[1-(4-chloro-3-fluorobenzoyl)-4,6-difluoro-5-hydroxy-2-methyl-1H-indol-3-yl]-N-ethylacetamide
2-[1-(4-chloro-3-fluorobenzoyl)-4,6-difluoro-5-hydroxy-2-methyl-1H-indol-3-yl]-N-propylacetamide
2-[1-(4-chloro-3-fluorobenzoyl)-4,6-difluoro-5-hydroxy-2-methyl-1H-indol-3-yl]-N-isopropylacetamide
2-[1-(4-chloro-3-fluorobenzoyl)-4,6-difluoro-5-hydroxy-2-methyl-1H-indol-3-yl]-N-butylacetamide
2-[1-(4-chloro-3-fluorobenzoyl)-4,6-difluoro-5-hydroxy-2-methyl-1H-indol-3-yl]-N-sec-butylacetamide
2-[1-(4-chloro-3-fluorobenzoyl)-4,6-difluoro-5-hydroxy-2-methyl-1H-indol-3-yl]-N-(2-hydroxyethyl)acetamide
methyl[1-(3-fluorobenzoyl)-5-hydroxy-2-methyl-1H-indol-3-yl]acetate
ethyl[1-(3-fluorobenzoyl)-5-hydroxy-2-methyl-1H-indol-3-yl]acetate
propyl[1-(3-fluorobenzoyl)-5-hydroxy-2-methyl-1H-indol-3-yl]acetate
butyl[1-(3-fluorobenzoyl)-5-hydroxy-2-methyl-1H-indol-3-yl]acetate
sec-butyl[1-(3-fluorobenzoyl)-5-hydroxy-2-methyl-1H-indol-3-yl]acetate
(R)sec-butyl[1-(3-fluorobenzoyl)-5-hydroxy-2-methyl-1H-indol-3-yl]acetate
(S)sec-butyl[1-(3-fluorobenzoyl)-5-hydroxy-2-methyl-1H-indol-3-yl]acetate
isopropyl[1-(3-fluorobenzoyl)-5-hydroxy-2-methyl-1H-indol-3-yl]acetate
tert-butyl[1-(3-fluorobenzoyl)-5-hydroxy-2-methyl-1H-indol-3-yl]acetate
cyclobutyl[1-(3-fluorobenzoyl)-5-hydroxy-2-methyl-1H-indol-3-yl]acetate
cyclopentyl[1-(3-fluorobenzoyl)-5-hydroxy-2-methyl-1H-indol-3-yl]acetate
2-[1-(3-fluorobenzoyl)-5-hydroxy-2-methyl-1H-indol-3-yl]acetamide
2-[1-(3-fluorobenzoyl)-5-hydroxy-2-methyl-1H-indol-3-yl]-N-methylacetamide
2-[1-(3-fluorobenzoyl)-5-hydroxy-2-methyl-1H-indol-3-yl]-N-ethylacetamide
2-[1-(3-fluorobenzoyl)-5-hydroxy-2-methyl-1H-indol-3-yl]-N-propylacetamide
2-[1-(3-fluorobenzoyl)-5-hydroxy-2-methyl-1H-indol-3-yl]-N-isopropylacetamide
2-[1-(3-fluorobenzoyl)-5-hydroxy-2-methyl-1H-indol-3-yl]-N-butylacetamide
2-[1-(3-fluorobenzoyl)-5-hydroxy-2-methyl-1H-indol-3-yl]-N-sec-butylacetamide
2-[1-(3-fluorobenzoyl)-5-hydroxy-2-methyl-1H-indol-3-yl]-N-(2-hydroxyethyl)acetamide
methyl[1-(3-fluorobenzoyl)-6-fluoro-5-hydroxy-2-methyl-1H-indol-3-yl]acetate
ethyl[1-(3-fluorobenzoyl)-6-fluoro-5-hydroxy-2-methyl-1H-indol-3-yl]acetate
propyl[1-(3-fluorobenzoyl)-6-fluoro-5-hydroxy-2-methyl-1H-indol-3-yl]acetate
butyl[1-(3-fluorobenzoyl)-6-fluoro-5-hydroxy-2-methyl-1H-indol-3-yl]acetate
sec-butyl[1-(3-fluorobenzoyl)-6-fluoro-5-hydroxy-2-methyl-1H-indol-3-yl]acetate
(R)sec-butyl[1-(3-fluorobenzoyl)-6-fluoro-5-hydroxy-2-methyl-1H-indol-3-yl]acetate
(S)sec-butyl[1-(3-fluorobenzoyl)-6-fluoro-5-hydroxy-2-methyl-1H-indol-3-yl]acetate
isopropyl[1-(3-fluorobenzoyl)-6-fluoro-5-hydroxy-2-methyl-1H-indol-3-yl]acetate
tert-butyl[1-(3-fluorobenzoyl)-6-fluoro-5-hydroxy-2-methyl-1H-indol-3-yl]acetate
cyclobutyl[1-(3-fluorobenzoyl)-6-fluoro-5-hydroxy-2-methyl-1H-indol-3-yl]acetate
cyclopentyl[1-(3-fluorobenzoyl)-6-fluoro-5-hydroxy-2-methyl-1H-indol-3-yl]acetate
2-[1-(3-fluorobenzoyl)-6-fluoro-5-hydroxy-2-methyl-1H-indol-3-yl]acetamide
2-[1-(3-fluorobenzoyl)-6-fluoro-5-hydroxy-2-methyl-1H-indol-3-yl]-N-methylacetamide
2-[1-(3-fluorobenzoyl)-6-fluoro-5-hydroxy-2-methyl-1H-indol-3-yl]-N-ethylacetamide
2-[1-(3-fluorobenzoyl)-6-fluoro-5-hydroxy-2-methyl-1H-indol-3-yl]-N-propylacetamide
2-[1-(3-fluorobenzoyl)-6-fluoro-5-hydroxy-2-methyl-1H-indol-3-yl]-N-isopropylacetamide
2-[1-(3-fluorobenzoyl)-6-fluoro-5-hydroxy-2-methyl-1H-indol-3-yl]-N-butylacetamide
2-[1-(3-fluorobenzoyl)-6-fluoro-5-hydroxy-2-methyl-1H-indol-3-yl]-N-sec-butylacetamide
2-[1-(3-fluorobenzoyl)-6-fluoro-5-hydroxy-2-methyl-1H-indol-3-yl]-N-(2-hydroxyethyl)acetamide
methyl[1-(3-fluorobenzoyl)-4-fluoro-5-hydroxy-2-methyl-1H-indol-3-yl]acetate
ethyl[1-(3-fluorobenzoyl)-4-fluoro-5-hydroxy-2-methyl-1H-indol-3-yl]acetate
propyl[1-(3-fluorobenzoyl)-4-fluoro-5-hydroxy-2-methyl-1H-indol-3-yl]acetate
butyl[1-(3-fluorobenzoyl)-4-fluoro-5-hydroxy-2-methyl-1H-indol-3-yl]acetate
sec-butyl[1-(3-fluorobenzoyl)-4-fluoro-5-hydroxy-2-methyl-1H-indol-3-yl]acetate
(R)sec-butyl[1-(3-fluorobenzoyl)-4-fluoro-5-hydroxy-2-methyl-1H-indol-3-yl]acetate
(S)sec-butyl[1-(3-fluorobenzoyl)-4-fluoro-5-hydroxy-2-methyl-1H-indol-3-yl]acetate
isopropyl[1-(3-fluorobenzoyl)-4-fluoro-5-hydroxy-2-methyl-1H-indol-3-yl]acetate
tert-butyl[1-(3-fluorobenzoyl)-4-fluoro-5-hydroxy-2-methyl-1H-indol-3-yl]acetate
cyclobutyl[1-(3-fluorobenzoyl)-4-fluoro-5-hydroxy-2-methyl-1H-indol-3-yl]acetate
cyclopentyl[1-(3-fluorobenzoyl)-4-fluoro-5-hydroxy-2-methyl-1H-indol-3-yl]acetate
2-[1-(3-fluorobenzoyl)-4-fluoro-5-hydroxy-2-methyl-1H-indol-3-yl]acetamide
2-[1-(3-fluorobenzoyl)-4-fluoro-5-hydroxy-2-methyl-1H-indol-3-yl]-N-methylacetamide
2-[1-(3-fluorobenzoyl)-4-fluoro-5-hydroxy-2-methyl-1H-indol-3-yl]-N-ethylacetamide
2-[1-(3-fluorobenzoyl)-4-fluoro-5-hydroxy-2-methyl-1H-indol-3-yl]-N-propylacetamide
2-[1-(3-fluorobenzoyl)-4-fluoro-5-hydroxy-2-methyl-1H-indol-3-yl]-N-isopropylacetamide
2-[1-(3-fluorobenzoyl)-4-fluoro-5-hydroxy-2-methyl-1H-indol-3-yl]-N-butylacetamide
2-[1-(3-fluorobenzoyl)-4-fluoro-5-hydroxy-2-methyl-1H-indol-3-yl]-N-sec-butylacetamide
2-[1-(3-fluorobenzoyl)-4-fluoro-5-hydroxy-2-methyl-1H-indol-3-yl]-N-(2-hydroxyethyl)acetamide
methyl[1-(3-fluorobenzoyl)-4,6-difluoro-5-hydroxy-2-methyl-1H-indol-3-yl]acetate
ethyl[1-(3-fluorobenzoyl)-4,6-difluoro-5-hydroxy-2-methyl-1H-indol-3-yl]acetate
propyl[1-(3-fluorobenzoyl)-4,6-difluoro-5-hydroxy-2-methyl-1H-indol-3-yl]acetate
butyl[1-(3-fluorobenzoyl)-4,6-difluoro-5-hydroxy-2-methyl-1H-indol-3-yl]acetate
sec-butyl[1-(3-fluorobenzoyl)-4,6-difluoro-5-hydroxy-2-methyl-1H-indol-3-yl]acetate
(R)sec-butyl[1-(3-fluorobenzoyl)-4,6-difluoro-5-hydroxy-2-methyl-1H-indol-3-yl]acetate
(S)sec-butyl[1-(3-fluorobenzoyl)-4,6-difluoro-5-hydroxy-2-methyl-1H-indol-3-yl]acetate
isopropyl[1-(3-fluorobenzoyl)-4,6-difluoro-5-hydroxy-2-methyl-1H-indol-3-yl]acetate
tert-butyl[1-(3-fluorobenzoyl)-4,6-difluoro-5-hydroxy-2-methyl-1H-indol-3-yl]acetate
cyclobutyl[1-(3-fluorobenzoyl)-4,6-difluoro-5-hydroxy-2-methyl-1H-indol-3-yl]acetate
cyclopentyl[1-(3-fluorobenzoyl)-4,6-difluoro-5-hydroxy-2-methyl-1H-indol-3-yl]acetate
2-[1-(3-fluorobenzoyl)-4,6-difluoro-5-hydroxy-2-methyl-1H-indol-3-yl]acetamide
2-[1-(3-fluorobenzoyl)-4,6-difluoro-5-hydroxy-2-methyl-1H-indol-3-yl]-N-methylacetamide
2-[1-(3-fluorobenzoyl)-4,6-difluoro-5-hydroxy-2-methyl-1H-indol-3-yl]-N-ethylacetamide
2-[1-(3-fluorobenzoyl)-4,6-difluoro-5-hydroxy-2-methyl-1H-indol-3-yl]-N-propylacetamide
2-[1-(3-fluorobenzoyl)-4,6-difluoro-5-hydroxy-2-methyl-1H-indol-3-yl]-N-isopropylacetamide
2-[1-(3-fluorobenzoyl)-4,6-difluoro-5-hydroxy-2-methyl-1H-indol-3-yl]-N-butylacetamide
2-[1-(3-fluorobenzoyl)-4,6-difluoro-5-hydroxy-2-methyl-1H-indol-3-yl]-N-sec-butylacetamide
2-[1-(3-fluorobenzoyl)-4,6-difluoro-5-hydroxy-2-methyl-1H-indol-3-yl]-N-(2-hydroxyethyl)acetamide
methyl(2R,S)-2-[1-(3-chlorobenzoyl)-4,6-difluoro-5-hydroxy-2-methyl-1H-indol-3-yl]propanoate
ethyl(2R,S)-2-[1-(3-chlorobenzoyl)-4,6-difluoro-5-hydroxy-2-methyl-1H-indol-3-yl]propanoate
propyl(2R,S)-2-[1-(3-chlorobenzoyl)-4,6-difluoro-5-hydroxy-2-methyl-1H-indol-3-yl]propanoate
isopropyl(2R,S)-2-[1-(3-chlorobenzoyl)-4,6-difluoro-5-hydroxy-2-methyl-1H-indol-3-yl]propanoate
butyl(2R,S)-2-[1-(3-chlorobenzoyl)-4,6-difluoro-5-hydroxy-2-methyl-1H-indol-3-yl]propanoate
sec-butyl(2R,S)-2-[1-(3-chlorobenzoyl)-4,6-difluoro-5-hydroxy-2-methyl-1H-indol-3-yl]propanoate
(R)sec-butyl(2R,S)-2-[1-(3-chlorobenzoyl)-4,6-difluoro-5-hydroxy-2-methyl-1H-indol-3-yl]propanoate
(S)sec-butyl(2R,S)-2-[1-(3-chlorobenzoyl)-4,6-difluoro-5-hydroxy-2-methyl-1H-indol-3-yl]propanoate
tert-butyl(2R,S)-2-[1-(3-chlorobenzoyl)-4,6-difluoro-5-hydroxy-2-methyl-1H-indol-3-yl]propanoate
cyclobutyl(2R,S)-2-[1-(3-chlorobenzoyl)-4,6-difluoro-5-hydroxy-2-methyl-1H-indol-3-yl]propanoate
cyclopentyl(2R,S)-2-[1-(3-chlorobenzoyl)-4,6-difluoro-5-hydroxy-2-methyl-1H-indol-3-yl]propanoate
(2R,S)-2-[1-(3-chlorobenzoyl)-4,6-difluoro-5-hydroxy-2-methyl-1H-indol-3-yl]propanamide
(2R,S)-2-[1-(3-chlorobenzoyl)-4,6-difluoro-5-hydroxy-2-methyl-1H-indol-3-yl]-N-methylpropanamide
(2R,S)-2-[1-(3-chlorobenzoyl)-4,6-difluoro-5-hydroxy-2-methyl-1H-indol-3-yl]-N-ethylacetamide
(2R,S)-2-[1-(3-chlorobenzoyl)-4,6-difluoro-5-hydroxy-2-methyl-1H-indol-3-yl]-N-propylacetamide
(2R,S)-2-[1-(3-chlorobenzoyl)-4,6-difluoro-5-hydroxy-2-methyl-1H-indol-3-yl]-N-isopropylacetamide
(2R,S)-2-[1-(3-chlorobenzoyl)-4,6-difluoro-5-hydroxy-2-methyl-1H-indol-3-yl]-N-butylacetamide
(2R,S)-2-[1-(3-chlorobenzoyl)-4,6-difluoro-5-hydroxy-2-methyl-1H-indol-3-yl]-N-sec-butylacetamide
(2R,S)-2-[1-(3-chlorobenzoyl)-4,6-difluoro-5-hydroxy-2-methyl-1H-indol-3-yl]-N-(hydroxymethyl)propanamide
methyl(2R,S)-2-[1-(3-chlorobenzoyl)-6-fluoro-5-hydroxy-2-methyl-1H-indol-3-yl]propanoate
ethyl(2R,S)-2-[1-(3-chlorobenzoyl)-6-fluoro-5-hydroxy-2-methyl-1H-indol-3-yl]propanoate
propyl(2R,S)-2-[1-(3-chlorobenzoyl)-6-fluoro-5-hydroxy-2-methyl-1H-indol-3-yl]propanoate
isopropyl(2R,S)-2-[1-(3-chlorobenzoyl)-6-fluoro-5-hydroxy-2-methyl-1H-indol-3-yl]propanoate
butyl(2R,S)-2-[1-(3-chlorobenzoyl)-6-fluoro-5-hydroxy-2-methyl-1H-indol-3-yl]propanoate
sec-butyl(2R,S)-2-[1-(3-chlorobenzoyl)-6-fluoro-5-hydroxy-2-methyl-1H-indol-3-yl]propanoate
(R)sec-butyl(2R,S)-2-[1-(3-chlorobenzoyl)-6-fluoro-5-hydroxy-2-methyl-1H-indol-3-yl]propanoate
(S)sec-butyl(2R,S)-2-[1-(3-chlorobenzoyl)-6-fluoro-5-hydroxy-2-methyl-1H-indol-3-yl]propanoate
tert-butyl(2R,S)-2-[1-(3-chlorobenzoyl)-6-fluoro-5-hydroxy-2-methyl-1H-indol-3-yl]propanoate
cyclobutyl(2R,S)-2-[1-(3-chlorobenzoyl)-6-fluoro-5-hydroxy-2-methyl-1H-indol-3-yl]propanoate
cyclopentyl(2R,S)-2-[1-(3-chlorobenzoyl)-6-fluoro-5-hydroxy-2-methyl-1H-indol-3-yl]propanoate
(2R,S)-2-[1-(3-chlorobenzoyl)-6-fluoro-5-hydroxy-2-methyl-1H-indol-3-yl]propanamide
(2R,S)-2-[1-(3-chlorobenzoyl)-6-fluoro-5-hydroxy-2-methyl-1H-indol-3-yl]-N-methylpropanamide
(2R,S)-2-[1-(3-chlorobenzoyl)-6-fluoro-5-hydroxy-2-methyl-1H-indol-3-yl]-N-ethylacetamide
(2R,S)-2-[1-(3-chlorobenzoyl)-6-fluoro-5-hydroxy-2-methyl-1H-indol-3-yl]-N-propylacetamide
(2R,S)-2-[1-(3-chlorobenzoyl)-6-fluoro-5-hydroxy-2-methyl-1H-indol-3-yl]-N-isopropylacetamide
(2R,S)-2-[1-(3-chlorobenzoyl)-6-fluoro-5-hydroxy-2-methyl-1H-indol-3-yl]-N-butylacetamide
(2R,S)-2-[1-(3-chlorobenzoyl)-6-fluoro-5-hydroxy-2-methyl-1H-indol-3-yl]-N-sec-butylacetamide
(2R,S)-2-[1-(3-chlorobenzoyl)-6-fluoro-5-hydroxy-2-methyl-1H-indol-3-yl]-N-(hydroxymethyl)propanamide
methyl(2R,S)-2-[1-(3-chlorobenzoyl)-4-fluoro-5-hydroxy-2-methyl-1H-indol-3-yl]propanoate
ethyl(2R,S)-2-[1-(3-chlorobenzoyl)-4-fluoro-5-hydroxy-2-methyl-1H-indol-3-yl]propanoate
propyl(2R,S)-2-[1-(3-chlorobenzoyl)-4-fluoro-5-hydroxy-2-methyl-1H-indol-3-yl]propanoate
isopropyl(2R,S)-2-[1-(3-chlorobenzoyl)-4-fluoro-5-hydroxy-2-methyl-1H-indol-3-yl]propanoate
butyl(2R,S)-2-[1-(3-chlorobenzoyl)-4-fluoro-5-hydroxy-2-methyl-1H-indol-3-yl]propanoate
sec-butyl(2R,S)-2-[1-(3-chlorobenzoyl)-4-fluoro-5-hydroxy-2-methyl-1H-indol-3-yl]propanoate
(R)sec-butyl(2R,S)-2-[1-(3-chlorobenzoyl)-4-fluoro-5-hydroxy-2-methyl-1H-indol-3-yl]propanoate
(S)sec-butyl(2R¹⁰,S)-2-[1-(3-chlorobenzoyl)-4-fluoro-5-hydroxy-2-methyl-1H-indol-3-yl]propanoate
tert-butyl(2R,S)-2-[1-(3-chlorobenzoyl)-4-fluoro-5-hydroxy-2-methyl-1H-indol-3-yl]propanoate
cyclobutyl(2R¹,S)-2-[1-(3-chlorobenzoyl)-4-fluoro-5-hydroxy-2-methyl-1H-indol-3-yl]propanoate
cyclopentyl(2R,S)-2-[1-(3-chlorobenzoyl)-4-fluoro-5-hydroxy-2-methyl-1H-indol-3-yl]propanoate
(2R,S)-2-[1-(3-chlorobenzoyl)-4-fluoro-5-hydroxy-2-methyl-1H-indol-3-yl]propanamide
(2R,S)-2-[1-(3-chlorobenzoyl)-4-fluoro-5-hydroxy-2-methyl-1H-indol-3-yl]-N-methylpropanamide
(2R,S)-2-[1-(3-chlorobenzoyl)-4-fluoro-5-hydroxy-2-methyl-1H-indol-3-yl]-N-ethylacetamide
(2R,S)-2-[1-(3-chlorobenzoyl)-4-fluoro-5-hydroxy-2-methyl-1H-indol-3-yl]-N-propylacetamide
(2R,S)-2-[1-(3-chlorobenzoyl)-4-fluoro-5-hydroxy-2-methyl-1H-indol-3-yl]-N-isopropylacetamide
(2R,S)-2-[1-(3-chlorobenzoyl)-4-fluoro-5-hydroxy-2-methyl-1H-indol-3-yl]-N-butylacetamide
(2R,S)-2-[1-(3-chlorobenzoyl)-4-fluoro-5-hydroxy-2-methyl-1H-indol-3-yl]-N-sec-butylacetamide
(2R,S)-2-[1-(3-chlorobenzoyl)-4-fluoro-5-hydroxy-2-methyl-1H-indol-3-yl]-N-(hydroxymethyl)propanamide
methyl(2R,S)-2-[1-(3-chlorobenzoyl)-5-hydroxy-2-methyl-1H-indol-3-yl]propanoate
ethyl(2R,S)-2-[1-(3-chlorobenzoyl)-5-hydroxy-2-methyl-1H-indol-3-yl]propanoate
propyl(2R,S)-2-[1-(3-chlorobenzoyl)-5-hydroxy-2-methyl-1H-indol-3-yl]propanoate
isopropyl(2R,S)-2-[1-(3-chlorobenzoyl)-5-hydroxy-2-methyl-1H-indol-3-yl]propanoate
butyl(2R,S)-2-[1-(3-chlorobenzoyl)-5-hydroxy-2-methyl-1H-indol-3-yl]propanoate
sec-butyl(2R,S)-2-[1-(3-chlorobenzoyl)-5-hydroxy-2-methyl-1H-indol-3-yl]propanoate
(R)sec-butyl(2R,S)-2-[1-(3-chlorobenzoyl)-5-hydroxy-2-methyl-1H-indol-3-yl]propanoate
(S)sec-butyl(2R,S)-2-[1-(3-chlorobenzoyl)-5-hydroxy-2-methyl-1H-indol-3-yl]propanoate
tert-butyl(2R,S)-2-[1-(3-chlorobenzoyl)-5-hydroxy-2-methyl-1H-indol-3-yl]propanoate
cyclobutyl(2R,S)-2-[1-(3-chlorobenzoyl)-5-hydroxy-2-methyl-1H-indol-3-yl]propanoate
cyclopentyl(2R,S)-2-[1-(3-chlorobenzoyl)-5-hydroxy-2-methyl-1H-indol-3-yl]propanoate
(2R,S)-2-[1-(3-chlorobenzoyl)-5-hydroxy-2-methyl-1H-indol-3-yl]propanamide
(2R,S)-2-[1-(3-chlorobenzoyl)-5-hydroxy-2-methyl-1H-indol-3-yl]-N-methylpropanamide
(2R,S)-2-[1-(3-chlorobenzoyl)-5-hydroxy-2-methyl-1H-indol-3-yl]-N-ethylacetamide
(2R,S)-2-[1-(3-chlorobenzoyl)-5-hydroxy-2-methyl-1H-indol-3-yl]-N-propylacetamide
(2R,S)-2-[1-(3-chlorobenzoyl)-5-hydroxy-2-methyl-1H-indol-3-yl]-N-isopropylacetamide
(2R,S)-2-[1-(3-chlorobenzoyl)-5-hydroxy-2-methyl-1H-indol-3-yl]-N-butylacetamide
(2R,S)-2-[1-(3-chlorobenzoyl)-5-hydroxy-2-methyl-1H-indol-3-yl]-N-sec-butylacetamide
(2R,S)-2-[1-(3-chlorobenzoyl)-5-hydroxy-2-methyl-1H-indol-3-yl]-N-(hydroxymethyl)propanamide
methyl(2R,S)-2-[1-(3,4-dichlorobenzoyl)-5-hydroxy-2-methyl-1H-indol-3-yl]propanoate
ethyl(2R,S)-2-[1-(3,4-dichlorobenzoyl)-5-hydroxy-2-methyl-1H-indol-3-yl]propanoate
propyl(2R,S)-2-[1-(3,4-dichlorobenzoyl)-5-hydroxy-2-methyl-1H-indol-3-yl]propanoate
isopropyl(2R,S)-2-[1-(3,4-dichlorobenzoyl)-5-hydroxy-2-methyl-1H-indol-3-yl]propanoate
butyl(2R,S)-2-[1-(3,4-dichlorobenzoyl)-5-hydroxy-2-methyl-1H-indol-3-yl]propanoate
sec-butyl(2R,S)-2-[1-(3,4-dichlorobenzoyl)-5-hydroxy-2-methyl-1H-indol-3-yl]propanoate
(R)sec-butyl(2R,S)-2-[1-(3,4-dichlorobenzoyl)-5-hydroxy-2-methyl-1H-indol-3-yl]propanoate
(S)sec-butyl(2R,S)-2-[1-(3,4-dichlorobenzoyl)-5-hydroxy-2-methyl-1H-indol-3-yl]propanoate
tert-butyl(2R,S)-2-[1-(3,4-dichlorobenzoyl)-5-hydroxy-2-methyl-1H-indol-3-yl]propanoate
cyclobutyl(2R,S)-2-[1-(3,4-dichlorobenzoyl)-5-hydroxy-2-methyl-1H-indol-3-yl]propanoate
cyclopentyl(2R,S)-2-[1-(3,4-dichlorobenzoyl)-5-hydroxy-2-methyl-1H-indol-3-yl]propanoate
(2R,S)-2-[1-(3,4-dichlorobenzoyl)-5-hydroxy-2-methyl-1H-indol-3-yl]propanamide
(2R,S)-2-[1-(3,4-dichlorobenzoyl)-5-hydroxy-2-methyl-1H-indol-3-yl]-N-methylpropanamide
(2R,S)-2-[1-(3,4-dichlorobenzoyl)-5-hydroxy-2-methyl-1H-indol-3-yl]-N-ethylacetamide
(2R,S)-2-[1-(3,4-dichlorobenzoyl)-5-hydroxy-2-methyl-1H-indol-3-yl]-N-propylacetamide
(2R,S)-2-[1-(3,4-dichlorobenzoyl)-5-hydroxy-2-methyl-1H-indol-3-yl]-N-isopropylacetamide
(2R,S)-2-[1-(3,4-dichlorobenzoyl)-5-hydroxy-2-methyl-1H-indol-3-yl]-N-butylacetamide
(2R,S)-2-[1-(3,4-dichlorobenzoyl)-5-hydroxy-2-methyl-1H-indol-3-yl]-N-sec-butylacetamide
(2R,S)-2-[1-(3,4-dichlorobenzoyl)-5-hydroxy-2-methyl-1H-indol-3-yl]-N-(hydroxymethyl)propanamide
methyl(2R,S)-2-[1-(3,4-dichlorobenzoyl)-6-fluoro-5-hydroxy-2-methyl-1H-indol-3-yl]propanoate
ethyl(2R,S)-2-[1-(3,4-dichlorobenzoyl)-6-fluoro-5-hydroxy-2-methyl-1H-indol-3-yl]propanoate
propyl(2R,S)-2-[1-(3,4-dichlorobenzoyl)-6-fluoro-5-hydroxy-2-methyl-1H-indol-3-yl]propanoate
isopropyl(2R,S)-2-[1-(3,4-dichlorobenzoyl)-6-fluoro-5-hydroxy-2-methyl-1H-indol-3-yl]propanoate
butyl(2R,S)-2-[1-(3,4-dichlorobenzoyl)-6-fluoro-5-hydroxy-2-methyl-1H-indol-3-yl]propanoate
sec-butyl(2R,S)-2-[1-(3,4-dichlorobenzoyl)-6-fluoro-5-hydroxy-2-methyl-1H-indol-3-yl]propanoate
(R)sec-butyl(2R,S)-2-[1-(3,4-dichlorobenzoyl)-6-fluoro-5-hydroxy-2-methyl-1H-indol-3-yl]propanoate
(S)sec-butyl(2R,S)-2-[1-(3,4-dichlorobenzoyl)-6-fluoro-5-hydroxy-2-methyl-1H-indol-3-yl]propanoate
tert-butyl(2R,S)-2-[1-(3,4-dichlorobenzoyl)-6-fluoro-5-hydroxy-2-methyl-1H-indol-3-yl]propanoate
cyclobutyl(2R,S)-2-[1-(3,4-dichlorobenzoyl)-6-fluoro-5-hydroxy-2-methyl-1H-indol-3-yl]propanoate
cyclopentyl(2R,S)-2-[1-(3,4-dichlorobenzoyl)-6-fluoro-5-hydroxy-2-methyl-1H-indol-3-yl]propanoate
(2R,S)-2-[1-(3,4-dichlorobenzoyl)-6-fluoro-5-hydroxy-2-methyl-1H-indol-3-yl]propanamide
(2R,S)-2-[1-(3,4-dichlorobenzoyl)-6-fluoro-5-hydroxy-2-methyl-1H-indol-3-yl]-N-methylpropanamide
(2R,S)-2-[1-(3,4-dichlorobenzoyl)-6-fluoro-5-hydroxy-2-methyl-1H-indol-3-yl]-N-ethylacetamide
(2R,S)-2-[1-(3,4-dichlorobenzoyl)-6-fluoro-5-hydroxy-2-methyl-1H-indol-3-yl]-N-propylacetamide
(2R,S)-2-[1-(3,4-dichlorobenzoyl)-6-fluoro-5-hydroxy-2-methyl-1H-indol-3-yl]-N-isopropylacetamide
(2R,S)-2-[1-(3,4-dichlorobenzoyl)-6-fluoro-5-hydroxy-2-methyl-1H-indol-3-yl]-N-butylacetamide
(2R,S)-2-[1-(3,4-dichlorobenzoyl)-6-fluoro-5-hydroxy-2-methyl-1H-indol-3-yl]-N-sec-butylacetamide
(2R,S)-2-[1-(3,4-dichlorobenzoyl)-6-fluoro-5-hydroxy-2-methyl-1H-indol-3-yl]-N-(hydroxymethyl)propanamide
methyl(2R,S)-2-[1-(3,4-dichlorobenzoyl)-4-fluoro-5-hydroxy-2-methyl-1H-indol-3-yl]propanoate
ethyl(2R,S)-2-[1-(3,4-dichlorobenzoyl)-4-fluoro-5-hydroxy-2-methyl-1H-indol-3-yl]propanoate
propyl(2R,S)-2-[1-(3,4-dichlorobenzoyl)-4-fluoro-5-hydroxy-2-methyl-1H-indol-3-yl]propanoate
isopropyl(2R,S)-2-[1-(3,4-dichlorobenzoyl)-4-fluoro-5-hydroxy-2-methyl-1H-indol-3-yl]propanoate
butyl(2R,S)-2-[1-(3,4-dichlorobenzoyl)-4-fluoro-5-hydroxy-2-methyl-1H-indol-3-yl]propanoate
sec-butyl(2R,S)-2-[1-(3,4-dichlorobenzoyl)-4-fluoro-5-hydroxy-2-methyl-1H-indol-3-yl]propanoate
(R)sec-butyl(2R,S)-2-[1-(3,4-dichlorobenzoyl)-4-fluoro-5-hydroxy-2-methyl-1H-indol-3-yl]propanoate
(S)sec-butyl(2R,S)-2-[1-(3,4-dichlorobenzoyl)-4-fluoro-5-hydroxy-2-methyl-1H-indol-3-yl]propanoate
tert-butyl(2R,S)-2-[1-(3,4-dichlorobenzoyl)-4-fluoro-5-hydroxy-2-methyl-1H-indol-3-yl]propanoate
cyclobutyl(2R,S)-2-[1-(3,4-dichlorobenzoyl)-4-fluoro-5-hydroxy-2-methyl-1H-indol-3-yl]propanoate
cyclopentyl(2R,S)-2-[1-(3,4-dichlorobenzoyl)-4-fluoro-5-hydroxy-2-methyl-1H-indol-3-yl]propanoate
(2R,S)-2-[1-(3,4-dichlorobenzoyl)-4-fluoro-5-hydroxy-2-methyl-1H-indol-3-yl]propanamide
(2R,S)-2-[1-(3,4-dichlorobenzoyl)-4-fluoro-5-hydroxy-2-methyl-1H-indol-3-yl]-N-methylpropanamide
(2R,S)-2-[1-(3,4-dichlorobenzoyl)-4-fluoro-5-hydroxy-2-methyl-1H-indol-3-yl]-N-ethylacetamide
(2R,S)-2-[1-(3,4-dichlorobenzoyl)-4-fluoro-5-hydroxy-2-methyl-1H-indol-3-yl]-N-propylacetamide
(2R,S)-2-[1-(3,4-dichlorobenzoyl)-4-fluoro-5-hydroxy-2-methyl-1H-indol-3-yl]-N-isopropylacetamide
(2R,S)-2-[1-(3,4-dichlorobenzoyl)-4-fluoro-5-hydroxy-2-methyl-1H-indol-3-yl]-N-butylacetamide
(2R,S)-2-[1-(3,4-dichlorobenzoyl)-4-fluoro-5-hydroxy-2-methyl-1H-indol-3-yl]-N-sec-butylacetamide
(2R,S)-2-[1-(3,4-dichlorobenzoyl)-4-fluoro-5-hydroxy-2-methyl-1H-indol-3-yl]-N-(hydroxymethyl)propanamide
methyl(2R,S)-2-[1-(3,4-dichlorobenzoyl)-4,6-difluoro-5-hydroxy-2-methyl-1H-indol-3-yl]propanoate
ethyl(2R,S)-2-[1-(3,4-dichlorobenzoyl)-4,6-difluoro-5-hydroxy-2-methyl-1H-indol-3-yl]propanoate
propyl(2R,S)-2-[1-(3,4-dichlorobenzoyl)-4,6-difluoro-5-hydroxy-2-methyl-1H-indol-3-yl]propanoate
isopropyl(2R,S)-2-[1-(3,4-dichlorobenzoyl)-4,6-difluoro-5-hydroxy-2-methyl-1H-indol-3-yl]propanoate
butyl(2R,S)-2-[1-(3,4-dichlorobenzoyl)-4,6-difluoro-5-hydroxy-2-methyl-1H-indol-3-yl]propanoate
sec-butyl(2R,S)-2-[1-(3,4-dichlorobenzoyl)-4,6-difluoro-5-hydroxy-2-methyl-1H-indol-3-yl]propanoate
(R)sec-butyl(2R,S)-2-[1-(3,4-dichlorobenzoyl)-4,6-difluoro-5-hydroxy-2-methyl-1H-indol-3-yl]propanoate
(S)sec-butyl(2R,S)-2-[1-(3,4-dichlorobenzoyl)-4,6-difluoro-5-hydroxy-2-methyl-1H-indol-3-yl]propanoate
tert-butyl(2R,S)-2-[1-(3,4-dichlorobenzoyl)-4,6-difluoro-5-hydroxy-2-methyl-1H-indol-3-yl]propanoate
cyclobutyl(2R,S)-2-[1-(3,4-dichlorobenzoyl)-4,6-difluoro-5-hydroxy-2-methyl-1H-indol-3-yl]propanoate
cyclopentyl(2R,S)-2-[1-(3,4-dichlorobenzoyl)-4,6-difluoro-5-hydroxy-2-methyl-1H-indol-3-yl]propanoate
(2R,S)-2-[1-(3,4-dichlorobenzoyl)-4,6-difluoro-5-hydroxy-2-methyl-1H-indol-3-yl]propanamide
(2R,S)-2-[1-(3,4-dichlorobenzoyl)-4,6-difluoro-5-hydroxy-2-methyl-1H-indol-3-yl]-N-methylpropanamide
(2R,S)-2-[1-(3,4-dichlorobenzoyl)-4,6-difluoro-5-hydroxy-2-methyl-1H-indol-3-yl]-N-ethylacetamide
(2R,S)-2-[1-(3,4-dichlorobenzoyl)-4,6-difluoro-5-hydroxy-2-methyl-1H-indol-3-yl]-Ns propylacetamide
(2R,S)-2-[1-(3,4-dichlorobenzoyl)-4,6-difluoro-5-hydroxy-2-methyl-1H-indol-3-yl]-N-isopropylacetamide
(2R,S)-2-[1-(3,4-dichlorobenzoyl)-4,6-difluoro-5-hydroxy-2-methyl-1H-indol-3-yl]-N-butylacetamide
(2R,S)-2-[1-(3,4-dichlorobenzoyl)-4,6-difluoro-5-hydroxy-2-methyl-1H-indol-3-yl]-N-sec-butylacetamide
(2R,S)-2-[1-(3,4-dichlorobenzoyl)-4,6-difluoro-5-hydroxy-2-methyl-1H-indol-3-yl]-N-(hydroxymethyl)propanamide
methyl(2R,S)-2-[1-(3,4-difluorobenzoyl)-5-hydroxy-2-methyl-1H-indol-3-yl]propanoate
ethyl(2R,S)-2-[1-(3,4-difluorobenzoyl)-5-hydroxy-2-methyl-1H-indol-3-yl]propanoate
propyl(2R,S)-2-[1-(3,4-difluorobenzoyl)-5-hydroxy-2-methyl-1H-indol-3-yl]propanoate
isopropyl(2R,S)-2-[1-(3,4-difluorobenzoyl)-5-hydroxy-2-methyl-1H-indol-3-yl]propanoate
butyl(2R,S)-2-[1-(3,4-difluorobenzoyl)-5-hydroxy-2-methyl-1H-indol-3-yl]propanoate
sec-butyl(2R,S)-2-[1-(3,4-difluorobenzoyl)-5-hydroxy-2-methyl-1H-indol-3-yl]propanoate
(R)sec-butyl(2R,S)-2-[1-(3,4-difluorobenzoyl)-5-hydroxy-2-methyl-1H-indol-3-yl]propanoate
(S)sec-butyl(2R,S)-2-[1-(3,4-difluorobenzoyl)-5-hydroxy-2-methyl-1H-indol-3-yl]propanoate
tert-butyl(2R,S)-2-[1-(3,4-difluorobenzoyl)-5-hydroxy-2-methyl-1H-indol-3-yl]propanoate
cyclobutyl(2R,S)-2-[1-(3,4-difluorobenzoyl)-5-hydroxy-2-methyl-1H-indol-3-yl]propanoate
cyclopentyl(2R,S)-2-[1-(3,4-difluorobenzoyl)-5-hydroxy-2-methyl-1H-indol-3-yl]propanoate
(2R,S)-2-[1-(3,4-difluorobenzoyl)-5-hydroxy-2-methyl-1H-indol-3-yl]propanamide
(2R,S)-2-[1-(3,4-difluorobenzoyl)-5-hydroxy-2-methyl-1H-indol-3-yl]-N-methylpropanamide
(2R,S)-2-[1-(3,4-difluorobenzoyl)-5-hydroxy-2-methyl-1H-indol-3-yl]-N-ethylacetamide
(2R,S)-2-[1-(3,4-difluorobenzoyl)-5-hydroxy-2-methyl-1H-indol-3-yl]-N-propylacetamide
(2R,S)-2-[1-(3,4-difluorobenzoyl)-5-hydroxy-2-methyl-1H-indol-3-yl]-N-isopropylacetamide
(2R,S)-2-[1-(3,4-difluorobenzoyl)-5-hydroxy-2-methyl-1H-indol-3-yl]-N-butylacetamide
(2R,S)-2-[1-(3,4-difluorobenzoyl)-5-hydroxy-2-methyl-1H-indol-3-yl]-N-sec-butylacetamide
(2R,S)-2-[1-(3,4-difluorobenzoyl)-5-hydroxy-2-methyl-1H-indol-3-yl]-N-(hydroxymethyl)propanamide
methyl(2R,S)-2-[1-(3,4-difluorobenzoyl)-6-fluoro-5-hydroxy-2-methyl-1H-indol-3-yl]propanoate
ethyl(2R,S)-2-[1-(3,4-difluorobenzoyl)-6-fluoro-5-hydroxy-2-methyl-1H-indol-3-yl]propanoate
propyl(2R,S)-2-[1-(3,4-difluorobenzoyl)-6-fluoro-5-hydroxy-2-methyl-1H-indol-3-yl]propanoate
isopropyl(2R,S)-2-[1-(3,4-difluorobenzoyl)-6-fluoro-5-hydroxy-2-methyl-1H-indol-3-yl]propanoate
butyl(2R¹⁰,S)-2-[1-(3,4-difluorobenzoyl)-6-fluoro-5-hydroxy-2-methyl-1H-indol-3-yl]propanoate
sec-butyl(2R,S)-2-[1-(3,4-difluorobenzoyl)-6-fluoro-5-hydroxy-2-methyl-1H-indol-3-yl]propanoate
(R)sec-butyl(2R,S)-2-[1-(3,4-difluorobenzoyl)-6-fluoro-5-hydroxy-2-methyl-1H-indol-3-yl]propanoate
(S)sec-butyl(2R,S)-2-[1-(3,4-difluorobenzoyl)-6-fluoro-5-hydroxy-2-methyl-1H-indol-3-yl]propanoate
tert-butyl(2R,S)-2-[1-(3,4-difluorobenzoyl)-6-fluoro-5-hydroxy-2-methyl-1H-indol-3-yl]propanoate
cyclobutyl(2R,S)-2-[1-(3,4-difluorobenzoyl)-6-fluoro-5-hydroxy-2-methyl-1H-indol-3-yl]propanoate
cyclopentyl(2R,S)-2-[1-(3,4-difluorobenzoyl)-6-fluoro-5-hydroxy-2-methyl-1H-indol-3-yl]propanoate
(2R,S)-2-[1-(3,4-difluorobenzoyl)-6-fluoro-5-hydroxy-2-methyl-1H-indol-3-yl]propanamide
(2R,S)-2-[1-(3,4-difluorobenzoyl)-6-fluoro-5-hydroxy-2-methyl-1H-indol-3-yl]-N-methylpropanamide
(2R,S)-2-[1-(3,4-difluorobenzoyl)-6-fluoro-5-hydroxy-2-methyl-1H-indol-3-yl]-N-ethylacetamide
(2R,S)-2-[1-(3,4-difluorobenzoyl)-6-fluoro-5-hydroxy-2-methyl-1H-indol-3-yl]-N-propylacetamide
(2R,S)-2-[1-(3,4-difluorobenzoyl)-6-fluoro-5-hydroxy-2-methyl-1H-indol-3-yl]-N-isopropylacetamide
(2R,S)-2-[1-(3,4-difluorobenzoyl)-6-fluoro-5-hydroxy-2-methyl-1H-indol-3-yl]-N-butylacetamide
(2R,S)-2-[1-(3,4-difluorobenzoyl)-6-fluoro-5-hydroxy-2-methyl-1H-indol-3-yl]-N-sec-butylacetamide
(2R,S)-2-[1-(3,4-difluorobenzoyl)-6-fluoro-5-hydroxy-2-methyl-1H-indol-3-yl]-N-(hydroxymethyl)propanamide
methyl(2R,S)-2-[1-(3,4-difluorobenzoyl)-4-fluoro-5-hydroxy-2-methyl-1H-indol-3-yl]propanoate
ethyl(2R,S)-2-[1-(3,4-difluorobenzoyl)-4-fluoro-5-hydroxy-2-methyl-1H-indol-3-yl]propanoate
propyl(2R,S)-2-[1-(3,4-difluorobenzoyl)-4-fluoro-5-hydroxy-2-methyl-1H-indol-3-yl]propanoate
isopropyl(2R,S)-2-[1-(3,4-difluorobenzoyl)-4-fluoro-5-hydroxy-2-methyl-1H-indol-3-yl]propanoate
butyl(2R,S)-2-[1-(3,4-difluorobenzoyl)-4-fluoro-5-hydroxy-2-methyl-1H-indol-3-yl]propanoate
sec-butyl(2R,S)-2-[1-(3,4-difluorobenzoyl)-4-fluoro-5-hydroxy-2-methyl-1H-indol-3-yl]propanoate
(R)sec-butyl(2R,S)-2-[1-(3,4-difluorobenzoyl)-4-fluoro-5-hydroxy-2-methyl-1H-indol-3-yl]propanoate
(S)sec-butyl(2R,S)-2-[1-(3,4-difluorobenzoyl)-4-fluoro-5-hydroxy-2-methyl-1H-indol-3-yl]propanoate
tert-butyl(2R,S)-2-[1-(3,4-difluorobenzoyl)-4-fluoro-5-hydroxy-2-methyl-1H-indol-3-yl]propanoate
cyclobutyl(2R,S)-2-[1-(3,4-difluorobenzoyl)-4-fluoro-5-hydroxy-2-methyl-1H-indol-3-yl]propanoate
cyclopentyl(2R,S)-2-[1-(3,4-difluorobenzoyl)-4-fluoro-5-hydroxy-2-methyl-1H-indol-3-yl]propanoate
(2R,S)-2-[1-(3,4-difluorobenzoyl)-4-fluoro-5-hydroxy-2-methyl-1H-indol-3-yl]propanamide
(2R,S)-2-[1-(3,4-difluorobenzoyl)-4-fluoro-5-hydroxy-2-methyl-1H-indol-3-yl]-N-methylpropanamide
(2R,S)-2-[1-(3,4-difluorobenzoyl)-4-fluoro-5-hydroxy-2-methyl-1H-indol-3-yl]-N-ethylacetamide
(2R,S)-2-[1-(3,4-difluorobenzoyl)-4-fluoro-5-hydroxy-2-methyl-1H-indol-3-yl]-N-propylacetamide
(2R,S)-2-[1-(3,4-difluorobenzoyl)-4-fluoro-5-hydroxy-2-methyl-1H-indol-3-yl]-N-isopropylacetamide
(2R,S)-2-[1-(3,4-difluorobenzoyl)-4-fluoro-5-hydroxy-2-methyl-1H-indol-3-yl]-N-butylacetamide
(2R,S)-2-[1-(3,4-difluorobenzoyl)-4-fluoro-5-hydroxy-2-methyl-1H-indol-3-yl]-N-sec-butylacetamide
(2R,S)-2-[1-(3,4-difluorobenzoyl)-4-fluoro-5-hydroxy-2-methyl-1H-indol-3-yl]-N-(hydroxymethyl)propanamide
methyl(2R,S)-2-[1-(3,4-difluorobenzoyl)-4,6-difluoro-5-hydroxy-2-methyl-1H-indol-3-yl]propanoate
ethyl(2R,S)-2-[1-(3,4-difluorobenzoyl)-4,6-difluoro-5-hydroxy-2-methyl-1H-indol-3-yl]propanoate
propyl(2R,S)-2-[1-(3,4-difluorobenzoyl)-4,6-difluoro-5-hydroxy-2-methyl-1H-indol-3-yl]propanoate
isopropyl(2R,S)-2-[1-(3,4-difluorobenzoyl)-4,6-difluoro-5-hydroxy-2-methyl-1H-indol-3-yl]propanoate
butyl(2R,S)-2-[1-(3,4-difluorobenzoyl)-4,6-difluoro-5-hydroxy-2-methyl-1H-indol-3-yl]propanoate
sec-butyl(2R,S)-2-[1-(3,4-difluorobenzoyl)-4,6-difluoro-5-hydroxy-2-methyl-1H-indol-3-yl]propanoate
(R)sec-butyl(2R,S)-2-[1-(3,4-difluorobenzoyl)-4,6-difluoro-5-hydroxy-2-methyl-1H-indol-3-yl]propanoate
(S)sec-butyl(2R,S)-2-[1-(3,4-difluorobenzoyl)-4,6-difluoro-5-hydroxy-2-methyl-1H-indol-3-yl]propanoate
tert-butyl(2R,S)-2-[1-(3,4-difluorobenzoyl)-4,6-difluoro-5-hydroxy-2-methyl-1H-indol-3-yl]propanoate
cyclobutyl(2R,S)-2-[1-(3,4-difluorobenzoyl)-4,6-difluoro-5-hydroxy-2-methyl-1H-indol-3-yl]propanoate
cyclopentyl(2R,S)-2-[1-(3,4-difluorobenzoyl)-4,6-difluoro-5-hydroxy-2-methyl-1H-indol-3-yl]propanoate
(2R,S)-2-[1-(3,4-difluorobenzoyl)-4,6-difluoro-5-hydroxy-2-methyl-1H-indol-3-yl]propanamide
(2R,S)-2-[1-(3,4-difluorobenzoyl)-4,6-difluoro-5-hydroxy-2-methyl-1H-indol-3-yl]-N-methylpropanamide
(2R,S)-2-[1-(3,4-difluorobenzoyl)-4,6-difluoro-5-hydroxy-2-methyl-1H-indol-3-yl]-N-ethylacetamide
(2R,S)-2-[1-(3,4-difluorobenzoyl)-4,6-difluoro-5-hydroxy-2-methyl-1H-indol-3-yl]-N-propylacetamide
(2R,S)-2-[1-(3,4-difluorobenzoyl)-4,6-difluoro-5-hydroxy-2-methyl-1H-indol-3-yl]-N-isopropylacetamide
(2R,S)-2-[1-(3,4-difluorobenzoyl)-4,6-difluoro-5-hydroxy-2-methyl-1H-indol-3-yl]-N-butylacetamide
(2R,S)-2-[1-(3,4-difluorobenzoyl)-4,6-difluoro-5-hydroxy-2-methyl-1H-indol-3-yl]-N-sec-butylacetamide
(2R,S)-2-[1-(3,4-difluorobenzoyl)-4,6-difluoro-5-hydroxy-2-methyl-1H-indol-3-yl]-N-(hydroxymethyl)propanamide
methyl(2R,S)-2-[1-(4-choro-3-fluorobenzoyl)-5-hydroxy-2-methyl-1H-indol-3-yl]propanoate
ethyl(2R,S)-2-[1-(4-choro-3-fluorobenzoyl)-5-hydroxy-2-methyl-1H-indol-3-yl]propanoate
propyl(2R,S)-2-[1-(4-choro-3-fluorobenzoyl)-5-hydroxy-2-methyl-1H-indol-3-yl]propanoate
isopropyl(2R,S)-2-[1-(4-choro-3-fluorobenzoyl)-5-hydroxy-2-methyl-1H-indol-3-yl]propanoate
butyl(2R,S)-2-[1-(4-choro-3-fluorobenzoyl)-5-hydroxy-2-methyl-1H-indol-3-yl]propanoate
sec-butyl(2R,S)-2-[1-(4-choro-3-fluorobenzoyl)-5-hydroxy-2-methyl-1H-indol-3-yl]propanoate
(R)sec-butyl(2R,S)-2-[1-(4-choro-3-fluorobenzoyl)-5-hydroxy-2-methyl-1H-indol-3-yl]propanoate
(S)sec-butyl(2R,S)-2-[1-(4-choro-3-fluorobenzoyl)-5-hydroxy-2-methyl-1H-indol-3-yl]propanoate
tert-butyl(2R,S)-2-[1-(4-choro-3-fluorobenzoyl)-5-hydroxy-2-methyl-1H-indol-3-yl]propanoate
cyclobutyl(2R,S)-2-[1-(4-choro-3-fluorobenzoyl)-5-hydroxy-2-methyl-1H-indol-3-yl]propanoate
cyclopentyl(2R,S)-2-[1-(4-choro-3-fluorobenzoyl)-5-hydroxy-2-methyl-1H-indol-3-yl]propanoate
(2R,S)-2-[1-(4-choro-3-fluorobenzoyl)-5-hydroxy-2-methyl-1H-indol-3-yl]propanamide
(2R,S)-2-[1-(4-choro-3-fluorobenzoyl)-5-hydroxy-2-methyl-1H-indol-3-yl]-N-methylpropanamide
(2R,S)-2-[1-(4-choro-3-fluorobenzoyl)-5-hydroxy-2-methyl-1H-indol-3-yl]-N-ethylacetamide
(2R,S)-2-[1-(4-choro-3-fluorobenzoyl)-5-hydroxy-2-methyl-1H-indol-3-yl]-N-propylacetamide
(2R,S)-2-[1-(4-choro-3-fluorobenzoyl)-5-hydroxy-2-methyl-1H-indol-3-yl]-N-isopropylacetamide
(2R,S)-2-[1-(4-choro-3-fluorobenzoyl)-5-hydroxy-2-methyl-1H-indol-3-yl]-N-butylacetamide
(2R,S)-2-[1-(4-choro-3-fluorobenzoyl)-5-hydroxy-2-methyl-1H-indol-3-yl]-N-sec-butylacetamide
(2R,S)-2-[1-(4-choro-3-fluorobenzoyl)-5-hydroxy-2-methyl-1H-indol-3-yl]-N-(hydroxymethyl)propanamide
methyl(2R,S)-2-[1-(4-choro-3-fluorobenzoyl)-6-fluoro-5-hydroxy-2-methyl-1H-indol-3-yl]propanoate
ethyl(2R,S)-2-[1-(4-choro-3-fluorobenzoyl)-6-fluoro-5-hydroxy-2-methyl-1H-indol-3-yl]propanoate
propyl(2R,S)-2-[1-(4-choro-3-fluorobenzoyl)-6-fluoro-5-hydroxy-2-methyl-1H-indol-3-yl]propanoate
isopropyl(2R,S)-2-[1-(4-choro-3-fluorobenzoyl)-6-fluoro-5-hydroxy-2-methyl-1H-indol-3-yl]propanoate
butyl(2R,S)-2-[1-(4-choro-3-fluorobenzoyl)-6-fluoro-5-hydroxy-2-methyl 1H-indol-3-yl]propanoate
sec-butyl(2R,S)-2-[1-(4-choro-3-fluorobenzoyl)-6-fluoro-5-hydroxy-2-methyl-1H-indol-3-yl) propanoate
(R)sec-butyl(2R,S)-2-[1-(4-choro-3-fluorobenzoyl)-6-fluoro-5-hydroxy-2-methyl-1H-indol-3-yl]propanoate
(S)sec-butyl(2R,S)-2-[1-(4-choro-3-fluorobenzoyl)-6-fluoro-5-hydroxy-2-methyl-1H-indol-3-yl]propanoate
tert-butyl(2R,S)-2-[1-(4-choro-3-fluorobenzoyl)-6-fluoro-5-hydroxy-2-methyl-1H-indol-3-yl]propanoate
cyclobutyl(2R,S)-2-[1-(4-choro-3-fluorobenzoyl)-6-fluoro-5-hydroxy-2-methyl-1H-indol-3-yl]propanoate
cyclopentyl(2R,S)-2-[1-(4-choro-3-fluorobenzoyl)-6-fluoro-5-hydroxy-2-methyl-1H-indol-3-yl]propanoate
(2R,S)-2-[1-(4-choro-3-fluorobenzoyl)-6-fluoro-5-hydroxy-2-methyl-1H-indol-3-yl]propanamide
(2R,S)-2-[1-(4-choro-3-fluorobenzoyl)-6-fluoro-5-hydroxy-2-methyl-1H-indol-3-yl]-N-methylpropanamide
(2R,S)-2-[1-(4-choro-3-fluorobenzoyl)-6-fluoro-5-hydroxy-2-methyl-1H-indol-3-yl]-N-ethylacetamide
(2R,S)-2-[1-(4-choro-3-fluorobenzoyl)-6-fluoro-5-hydroxy-2-methyl-1H-indol-3-yl]-N-propylacetamide
(2R,S)-2-[1-(4-choro-3-fluorobenzoyl)-6-fluoro-5-hydroxy-2-methyl-1H-indol-3-yl]-N-isopropylacetamide
(2R,S)-2-[1-(4-choro-3-fluorobenzoyl)-6-fluoro-5-hydroxy-2-methyl-1H-indol-3-yl]-N-butylacetamide
(2R,S)-2-[1-(4-choro-3-fluorobenzoyl)-6-fluoro-5-hydroxy-2-methyl-1H-indol-3-yl]-N-sec-butylacetamide
(2R,S)-2-[1-(4-choro-3-fluorobenzoyl)-6-fluoro-5-hydroxy-2-methyl-1H-indol-3-yl]-N-(hydroxymethyl)propanamide
methyl(2R,S)-2-[1-(4-choro-3-fluorobenzoyl)-4-fluoro-5-hydroxy-2-methyl-1H-indol-3-yl]propanoate
ethyl(2R,S)-2-[1-(4-choro-3-fluorobenzoyl)-4-fluoro-5-hydroxy-2-methyl-1H-indol-3-yl]propanoate
propyl(2R,S)-2-[1-(4-choro-3-fluorobenzoyl)-4-fluoro-5-hydroxy-2-methyl-1H-indol-3-yl]propanoate
isopropyl(2R,S)-2-[1-(4-choro-3-fluorobenzoyl)-4-fluoro-5-hydroxy-2-methyl-1H-indol-3-yl]propanoate
butyl(2R,S)-2-[1-(4-choro-3-fluorobenzoyl)-4-fluoro-5-hydroxy-2-methyl-1H-indol-3-yl]propanoate
sec-butyl(2R,S)-2-[1-(4-choro-3-fluorobenzoyl)-4-fluoro-5-hydroxy-2-methyl-1H-indol-3-yl]propanoate
(R)sec-butyl(2R,S)-2-[1-(4-choro-3-fluorobenzoyl)-4-fluoro-5-hydroxy-2-methyl-1H-indol-3-yl]propanoate
(S)sec-butyl(2R,S)-2-[1-(4-choro-3-fluorobenzoyl)-4-fluoro-5-hydroxy-2-methyl-1H-indol-3-yl]propanoate
tert-butyl(2R,S)-2-[1-(4-choro-3-fluorobenzoyl)-4-fluoro-5-hydroxy-2-methyl-1H-indol-3-yl]propanoate
cyclobutyl(2R,S)-2-[1-(4-choro-3-fluorobenzoyl)-4-fluoro-5-hydroxy-2-methyl-1H-indol-3-yl]propanoate
cyclopentyl(2R,S)-2-[1-(4-choro-3-fluorobenzoyl)-4-fluoro-5-hydroxy-2-methyl-1H-indol-3-yl]propanoate
(2R,S)-2-[1-(4-choro-3-fluorobenzoyl)-4-fluoro-5-hydroxy-2-methyl-1H-indol-3-yl]propanamide
(2R,S)-2-[1-(4-choro-3-fluorobenzoyl)-4-fluoro-5-hydroxy-2-methyl-1H-indol-3-yl]-N-methylpropanamide
(2R,S)-2-[1-(4-choro-3-fluorobenzoyl)-4-fluoro-5-hydroxy-2-methyl-1H-indol-3-yl]-N-ethylacetamide
(2R,S)-2-[1-(4-choro-3-fluorobenzoyl)-4-fluoro-5-hydroxy-2-methyl-1H-indol-3-yl]-N-propylacetamide
(2R,S)-2-[1-(4-choro-3-fluorobenzoyl)-4-fluoro-5-hydroxy-2-methyl-1H-indol-3-yl]-N-isopropylacetamide
(2R,S)-2-[1-(4-choro-3-fluorobenzoyl)-4-fluoro-5-hydroxy-2-methyl-1H-indol-3-yl]-N-butylacetamide
(2R,S)-2-[1-(4-choro-3-fluorobenzoyl)-4-fluoro-5-hydroxy-2-methyl-1H-indol-3-yl]-N-sec-butylacetamide
(2R,S)-2-[1-(4-choro-3-fluorobenzoyl)-4-fluoro-5-hydroxy-2-methyl-1H-indol-3-yl]-N-(hydroxymethyl)propanamide
methyl(2R,S)-2-[1-(4-choro-3-fluorobenzoyl)-4,6-difluoro-5-hydroxy-2-methyl-1H-indol-3-yl]propanoate
ethyl(2R,S)-2-[1-(4-choro-3-fluorobenzoyl)-4,6-difluoro-5-hydroxy-2-methyl-1H-indol-3-yl]propanoate
propyl(2R,S)-2-[1-(4-choro-3-fluorobenzoyl)-4,6-difluoro-5-hydroxy-2-methyl-1H-indol-3-yl]propanoate
isopropyl(2R,S)-2-[1-(4-choro-3-fluorobenzoyl)-4,6-difluoro-5-hydroxy-2-methyl-1H-indol-3-yl]propanoate
butyl(2R,S)-2-[1-(4-choro-3-fluorobenzoyl)-4,6-difluoro-5-hydroxy-2-methyl-1H-indol-3-yl]propanoate
sec-butyl(2R,S)-2-[1-(4-choro-3-fluorobenzoyl)-4,6-difluoro-5-hydroxy-2-methyl-1H-indol-3-yl]propanoate
(R)sec-butyl(2R,S)-2-[1-(4-choro-3-fluorobenzoyl)-4,6-difluoro-5-hydroxy-2-methyl-1H-indol-3-yl]propanoate
(S)sec-butyl(2R,S)-2-[1-(4-choro-3-fluorobenzoyl)-4,6-difluoro-5-hydroxy-2-methyl-1H-indol-3-yl]propanoate
tert-butyl(2R,S)-2-[1-(4-choro-3-fluorobenzoyl)-4,6-difluoro-5-hydroxy-2-methyl-1H-indol-3-yl]propanoate
cyclobutyl(2R,S)-2-[1-(4-choro-3-fluorobenzoyl)-4,6-difluoro-5-hydroxy-2-methyl-1H-indol-3-yl]propanoate
cyclopentyl(2R,S)-2-[1-(4-choro-3-fluorobenzoyl)-4,6-difluoro-5-hydroxy-2-methyl-1H-indol-3-yl]propanoate
(2R,S)-2-[1-(4-choro-3-fluorobenzoyl)-4,6-difluoro-5-hydroxy-2-methyl-1H-indol-3-yl]propanamide
(2R,S)-2-[1-(4-choro-3-fluorobenzoyl)-4,6-difluoro-5-hydroxy-2-methyl-1H-indol-3-yl]-N-methylpropanamide
(2R,S)-2-[1-(4-choro-3-fluorobenzoyl)-4,6-difluoro-5-hydroxy-2-methyl-1H-indol-3-yl]-N-ethylacetamide
(2R,S)-2-[1-(4-choro-3-fluorobenzoyl)-4,6-difluoro-5-hydroxy-2-methyl-1H-indol-3-yl]-N-propylacetamide
(2R,S)-2-[1-(4-choro-3-fluorobenzoyl)-4,6-difluoro-5-hydroxy-2-methyl-1H-indol-3-yl]-N-isopropylacetamide
(2R,S)-2-[1-(4-choro-3-fluorobenzoyl)-4,6-difluoro-5-hydroxy-2-methyl-1H-indol-3-yl]-N-butylacetamide
(2R,S)-2-[1-(4-choro-3-fluorobenzoyl)-4,6-difluoro-5-hydroxy-2-methyl-1H-indol-3-yl]-N-sec-butylacetamide
(2R,S)-2-[1-(4-choro-3-fluorobenzoyl)-4,6-difluoro-5-hydroxy-2-methyl-1H-indol-3-yl]-N-(hydroxymethyl)propanamide
methyl(2R,S)-2-[1-(3-fluorobenzoyl)-5-hydroxy-2-methyl-1H-indol-3-yl]propanoate
ethyl(2R,S)-2-[1-(3-fluorobenzoyl)-5-hydroxy-2-methyl-1H-indol-3-yl]propanoate
propyl(2R,S)-2-[1-(3-fluorobenzoyl)-5-hydroxy-2-methyl-1H-indol-3-yl]propanoate
isopropyl(2R,S)-2-[1-(3-fluorobenzoyl)-5-hydroxy-2-methyl-1H-indol-3-yl]propanoate
butyl(2R,S)-2-[1-(3-fluorobenzoyl)-5-hydroxy-2-methyl-1H-indol-3-yl]propanoate
sec-butyl(2R,S)-2-[1-(3-fluorobenzoyl)-5-hydroxy-2-methyl-1H-indol-3-yl]propanoate
(R)sec-butyl(2R,S)-2-[1-(3-fluorobenzoyl)-5-hydroxy-2-methyl-1H-indol-3-yl]propanoate
(S)sec-butyl(2R,S)-2-[1-(3-fluorobenzoyl)-5-hydroxy-2-methyl-1H-indol-3-yl]propanoate
tert-butyl(2R,S)-2-[1-(3-fluorobenzoyl)-5-hydroxy-2-methyl-1H-indol-3-yl]propanoate
cyclobutyl(2R,S)-2-[1-(3-fluorobenzoyl)-5-hydroxy-2-methyl-1H-indol-3-yl]propanoate
cyclopentyl(2R,S)-2-[1-(3-fluorobenzoyl)-5-hydroxy-2-methyl-1H-indol-3-yl]propanoate
(2R,S)-2-[1-(3-fluorobenzoyl)-5-hydroxy-2-methyl-1H-indol-3-yl]propanamide
(2R,S)-2-[1-(3-fluorobenzoyl)-5-hydroxy-2-methyl-1H-indol-3-yl]-N-methylpropanamide
(2R,S)-2-[1-(3-fluorobenzoyl)-5-hydroxy-2-methyl-1H-indol-3-yl]-N-ethylacetamide
(2R,S)-2-[1-(3-fluorobenzoyl)-5-hydroxy-2-methyl-1H-indol-3-yl]-N-propylacetamide
(2R,S)-2-[1-(3-fluorobenzoyl)-5-hydroxy-2-methyl-1H-indol-3-yl]-N-isopropylacetamide
(2R,S)-2-[1-(3-fluorobenzoyl)-5-hydroxy-2-methyl-1H-indol-3-yl]-N-butylacetamide
(2R,S)-2-[1-(3-fluorobenzoyl)-5-hydroxy-2-methyl-1H-indol-3-yl]-N-sec-butylacetamide
(2R,S)-2-[1-(3-fluorobenzoyl)-5-hydroxy-2-methyl-1H-indol-3-yl]-N-(hydroxymethyl)propanamide
methyl(2R,S)-2-[1-(3-fluorobenzoyl)-6-fluoro-5-hydroxy-2-methyl-1H-indol-3-yl]propanoate
ethyl(2R,S)-2-[1-(3-fluorobenzoyl)-6-fluoro-5-hydroxy-2-methyl-1H-indol-3-yl]propanoate
propyl(2R,S)-2-[1-(3-fluorobenzoyl)-6-fluoro-5-hydroxy-2-methyl-1H-indol-3-yl]propanoate
isopropyl(2R,S)-2-[1-(3-fluorobenzoyl)-6-fluoro-5-hydroxy-2-methyl-1H-indol-3-yl]propanoate
butyl(2R,S)-2-[1-(3-fluorobenzoyl)-6-fluoro-5-hydroxy-2-methyl-1H-indol-3-yl]propanoate
sec-butyl(2R,S)-2-[1-(3-fluorobenzoyl)-6-fluoro-5-hydroxy-2-methyl-1H-indol-3-yl]propanoate
(R)sec-butyl(2R,S)-2-[1-(3-fluorobenzoyl)-6-fluoro-5-hydroxy-2-methyl-1H-indol-3-yl]propanoate
(S)sec-butyl(2R,S)-2-[1-(3-fluorobenzoyl)-6-fluoro-5-hydroxy-2-methyl-1H-indol-3-yl]propanoate
tert-butyl(2R,S)-2-[1-(3-fluorobenzoyl)-6-fluoro-5-hydroxy-2-methyl-1H-indol-3-yl]propanoate
cyclobutyl(2R,S)-2-[1-(3-fluorobenzoyl)-6-fluoro-5-hydroxy-2-methyl-1H-indol-3-yl]propanoate
cyclopentyl(2R,S)-2-[1-(3-fluorobenzoyl)-6-fluoro-5-hydroxy-2-methyl-1H-indol-3-yl]propanoate
(2R,S)-2-[1-(3-fluorobenzoyl)-6-fluoro-5-hydroxy-2-methyl-1H-indol-3-yl]propanamide
(2R,S)-2-[1-(3-fluorobenzoyl)-6-fluoro-5-hydroxy-2-methyl-1H-indol-3-yl]-N-methylpropanamide
(2R,S)-2-[1-(3-fluorobenzoyl)-6-fluoro-5-hydroxy-2-methyl-1H-indol-3-yl]-N-ethylacetamide
(2R,S)-2-[1-(3-fluorobenzoyl)-6-fluoro-5-hydroxy-2-methyl-1H-indol-3-yl]-N-propylacetamide
(2R,S)-2-[1-(3-fluorobenzoyl)-6-fluoro-5-hydroxy-2-methyl-1H-indol-3-yl]-N-isopropylacetamide
(2R,S)-2-[1-(3-fluorobenzoyl)-6-fluoro-5-hydroxy-2-methyl-1H-indol-3-yl]-N-butylacetamide
(2R,S)-2-[1-(3-fluorobenzoyl)-6-fluoro-5-hydroxy-2-methyl-1H-indol-3-yl]-N-sec-butylacetamide
(2R,S)-2-[1-(3-fluorobenzoyl)-6-fluoro-5-hydroxy-2-methyl-1H-indol-3-yl]-N-(hydroxymethyl)propanamide
methyl(2R,S)-2-[1-(3-fluorobenzoyl)-4-fluoro-5-hydroxy-2-methyl-1H-indol-3-yl]propanoate
ethyl(2R,S)-2-[1-(3-fluorobenzoyl)-4-fluoro-5-hydroxy-2-methyl-1H-indol-3-yl]propanoate
propyl(2R,S)-2-[1-(3-fluorobenzoyl)-4-fluoro-5-hydroxy-2-methyl-1H-indol-3-yl]propanoate
isopropyl(2R,S)-2-[1-(3-fluorobenzoyl)-4-fluoro-5-hydroxy-2-methyl-1H-indol-3-yl]propanoate
butyl(2R,S)-2-[1-(3-fluorobenzoyl)-4-fluoro-5-hydroxy-2-methyl-1H-indol-3-yl]propanoate
sec-butyl(2R,S)-2-[1-(3-fluorobenzoyl)-4-fluoro-5-hydroxy-2-methyl-1H-indol-3-yl]propanoate
(R)sec-butyl(2R,S)-2-[1-(3-fluorobenzoyl)-4-fluoro-5-hydroxy-2-methyl-1H-indol-3-yl]propanoate
(S)sec-butyl(2R,S)-2-[1-(3-fluorobenzoyl)-4-fluoro-5-hydroxy-2-methyl-1H-indol-3-yl]propanoate
tert-butyl(2R,S)-2-[1-(3-fluorobenzoyl)-4-fluoro-5-hydroxy-2-methyl-1H-indol-3-yl]propanoate
cyclobutyl(2R,S)-2-[1-(3-fluorobenzoyl)-4-fluoro-5-hydroxy-2-methyl-1H-indol-3-yl]propanoate
cyclopentyl(2R,S)-2-[1-(3-fluorobenzoyl)-4-fluoro-5-hydroxy-2-methyl-1H-indol-3-yl]propanoate
(2R,S)-2-[1-(3-fluorobenzoyl)-4-fluoro-5-hydroxy-2-methyl-1H-indol-3-yl]propanamide
(2R,S)-2-[1-(3-fluorobenzoyl)-4-fluoro-5-hydroxy-2-methyl-1H-indol-3-yl]-N-methylpropanamide
(2R,S)-2-[1-(3-fluorobenzoyl)-4-fluoro-5-hydroxy-2-methyl-1H-indol-3-yl]-N-ethylacetamide
(2R,S)-2-[1-(3-fluorobenzoyl)-4-fluoro-5-hydroxy-2-methyl-1H-indol-3-yl]-N-propylacetamide
(2R,S)-2-[1-(3-fluorobenzoyl)-4-fluoro-5-hydroxy-2-methyl-1H-indol-3-yl]-N-isopropylacetamide
(2R,S)-2-[1-(3-fluorobenzoyl)-4-fluoro-5-hydroxy-2-methyl-1H-indol-3-yl]-N-butylacetamide
(2R,S)-2-[1-(3-fluorobenzoyl)-4-fluoro-5-hydroxy-2-methyl-1H-indol-3-yl]-N-sec-butylacetamide
(2R,S)-2-[1-(3-fluorobenzoyl)-4-fluoro-5-hydroxy-2-methyl-1H-indol-3-yl]-N-(hydroxymethyl)propanamide
methyl(2R,S)-2-[1-(3-fluorobenzoyl)-4,6-difluoro-5-hydroxy-2-methyl-1H-indol-3-yl]propanoate
ethyl(2R,S)-2-[1-(3-fluorobenzoyl)-4,6-difluoro-5-hydroxy-2-methyl-1H-indol-3-yl]propanoate
propyl(2R,S)-2-[1-(3-fluorobenzoyl)-4,6-difluoro-5-hydroxy-2-methyl-1H-indol-3-yl]propanoate
isopropyl(2R,S)-2-[1-(3-fluorobenzoyl)-4,6-difluoro-5-hydroxy-2-methyl-1H-indol-3-yl]propanoate
butyl(2R,S)-2-[1-(3-fluorobenzoyl)-4,6-difluoro-5-hydroxy-2-methyl-1H-indol-3-yl]propanoate
sec-butyl(2R,S)-2-[1-(3-fluorobenzoyl)-4,6-difluoro-5-hydroxy-2-methyl-1H-indol-3-yl]propanoate
(R)sec-butyl(2R,S)-2-[1-(3-fluorobenzoyl)-4,6-difluoro-5-hydroxy-2-methyl-1H-indol-3-yl]propanoate
(S)sec-butyl(2R,S)-2-[1-(3-fluorobenzoyl)-4,6-difluoro-5-hydroxy-2-methyl-1H-indol-3-yl]propanoate
tert-butyl(2R,S)-2-[1-(3-fluorobenzoyl)-4,6-difluoro-5-hydroxy-2-methyl-1H-indol-3-yl]propanoate
cyclobutyl(2R,S)-2-[1-(3-fluorobenzoyl)-4,6-difluoro-5-hydroxy-2-methyl-1H-indol-3-yl]propanoate
cyclopentyl(2R,S)-2-[1-(3-fluorobenzoyl)-4,6-difluoro-5-hydroxy-2-methyl-1H-indol-3-yl]propanoate
(2R,S)-2-[1-(3-fluorobenzoyl)-4,6-difluoro-5-hydroxy-2-methyl-1H-indol-3-yl]propanamide
(2R,S)-2-[1-(3-fluorobenzoyl)-4,6-difluoro-5-hydroxy-2-methyl-1H-indol-3-yl]-N-methylpropanamide
(2R,S)-2-[1-(3-fluorobenzoyl)-4,6-difluoro-5-hydroxy-2-methyl-1H-indol-3-yl]-N-ethylacetamide
(2R,S)-2-[1-(3-fluorobenzoyl)-4,6-difluoro-5-hydroxy-2-methyl-1H-indol-3-yl]-N-propylacetamide
(2R,S)-2-[1-(3-fluorobenzoyl)-4,6-difluoro-5-hydroxy-2-methyl-1H-indol-3-yl]-N-isopropylacetamide
(2R,S)-2-[1-(3-fluorobenzoyl)-4,6-difluoro-5-hydroxy-2-methyl-1H-indol-3-yl]-N-butylacetamide
(2R,S)-2-[1-(3-fluorobenzoyl)-4,6-difluoro-5-hydroxy-2-methyl-1H-indol-3-yl]-N-sec-butylacetamide
(2R,S)-2-[1-(3-fluorobenzoyl)-4,6-difluoro-5-hydroxy-2-methyl-1H-indol-3-yl]-N-(hydroxymethyl)propanamide
methyl[1-(4-trifluoromethoxybenzoyl)-5-hydroxy-2-methyl-1H-indol-3-yl]acetate
ethyl[1-(4-trifluoromethoxybenzoyl)-5-hydroxy-2-methyl-1H-indol-3-yl]acetate
propyl[1-(4-trifluoromethoxybenzoyl)-5-hydroxy-2-methyl-1H-indol-3-yl]acetate
butyl[1-(4-trifluoromethoxybenzoyl)-5-hydroxy-2-methyl-1H-indol-3-yl]acetate
sec-butyl[1-(4-trifluoromethoxybenzoyl)-5-hydroxy-2-methyl-1H-indol-3-yl]acetate
(R)sec-butyl[1-(4-trifluoromethoxybenzoyl)-5-hydroxy-2-methyl-1H-indol-3-yl]acetate
(S)sec-butyl[1-(4-trifluoromethoxybenzoyl)-5-hydroxy-2-methyl-1H-indol-3-yl]acetate
isopropyl[1-(4-trifluoromethoxybenzoyl)-5-hydroxy-2-methyl-1H-indol-3-yl]acetate
tert-butyl[1-(4-trifluoromethoxybenzoyl)-5-hydroxy-2-methyl-1H-indol-3-yl]acetate
cyclobutyl[1-(4-trifluoromethoxybenzoyl)-5-hydroxy-2-methyl-1H-indol-3-yl]acetate
cyclopentyl[1-(4-trifluoromethoxybenzoyl)-5-hydroxy-2-methyl-1H-indol-3-yl]acetate
2-[1-(4-trifluoromethoxybenzoyl)-5-hydroxy-2-methyl-1H-indol-3-yl]acetamide
2-[1-(4-trifluoromethoxybenzoyl)-5-hydroxy-2-methyl-1H-indol-3-yl]-N-methylacetamide
2-[1-(4-trifluoromethoxybenzoyl)-5-hydroxy-2-methyl-1H-indol-3-yl]-N-ethylacetamide
2-[1-(4-trifluoromethoxybenzoyl)-5-hydroxy-2-methyl-1H-indol-3-yl]-N-propylacetamide
2-[1-(4-trifluoromethoxybenzoyl)-5-hydroxy-2-methyl-1H-indol-3-yl]-N-isopropylacetamide
2-[1-(4-trifluoromethoxybenzoyl)-5-hydroxy-2-methyl-1H-indol-3-yl]-N-butylacetamide
2-[1-(4-trifluoromethoxybenzoyl)-5-hydroxy-2-methyl-1H-indol-3-yl]-N-sec-butylacetamide
2-[1-(4-trifluoromethoxybenzoyl)-5-hydroxy-2-methyl-1H-indol-3-yl]-N-(2-hydroxyethyl)acetamide
methyl(2R,S)-2-[1-(4-trifluoromethoxybenzoyl)-5-hydroxy-2-methyl-1H-indol-3-yl]propanoate
ethyl(2R,S)-2-[1-(4-trifluoromethoxybenzoyl)-5-hydroxy-2-methyl-1H-indol-3-yl]propanoate
propyl(2R,S)-2-[1-(4-trifluoromethoxybenzoyl)-5-hydroxy-2-methyl-1H-indol-3-yl]propanoate
isopropyl(2R,S)-2-[1-(4-trifluoromethoxybenzoyl)-5-hydroxy-2-methyl-1H-indol-3-yl]propanoate
butyl(2R,S)-2-[1-(4-trifluoromethoxybenzoyl)-5-hydroxy-2-methyl-1H-indol-3-yl]propanoate
sec-butyl(2R,S)-2-[1-(4-trifluoromethoxybenzoyl)-5-hydroxy-2-methyl-1H-indol-3-yl]propanoate
(R)sec-butyl(2R,S)-2-[1-(4-trifluoromethoxybenzoyl)-5-hydroxy-2-methyl-1H-indol-3-yl]propanoate
(S)sec-butyl(2R,S)-2-[1-(4-trifluoromethoxybenzoyl)-5-hydroxy-2-methyl-1H-indol-3-yl]propanoate
tert-butyl(2R,S)-2-[1-(4-trifluoromethoxybenzoyl)-5-hydroxy-2-methyl-1H-indol-3-yl]propanoate
cyclobutyl(2R,S)-2-[1-(4-trifluoromethoxybenzoyl)-5-hydroxy-2-methyl-1H-indol-3-yl]propanoate
cyclopentyl(2R,S)-2-[1-(4-trifluoromethoxybenzoyl)-5-hydroxy-2-methyl-1H-indol-3-yl]propanoate
(2R,S)-2-[1-(4-trifluoromethoxybenzoyl)-5-hydroxy-2-methyl-1H-indol-3-yl]propanamide
(2R,S)-2-[1-(4-trifluoromethoxybenzoyl)-5-hydroxy-2-methyl-1H-indol-3-yl]-N-methylpropanamide
(2R,S)-2-[1-(4-trifluoromethoxybenzoyl)-5-hydroxy-2-methyl-1H-indol-3-yl]-N-ethylacetamide
(2R,S)-2-[1-(4-trifluoromethoxybenzoyl)-5-hydroxy-2-methyl-1H-indol-3-yl]-N-propylacetamide
(2R,S)-2-[1-(4-trifluoromethoxybenzoyl)-5-hydroxy-2-methyl-1H-indol-3-yl]-N-isopropylacetamide
(2R,S)-2-[1-(4-trifluoromethoxybenzoyl)-5-hydroxy-2-methyl-1H-indol-3-yl]-N-butylacetamide
(2R,S)-2-[1-(4-trifluoromethoxybenzoyl)-5-hydroxy-2-methyl-1H-indol-3-yl]-N-sec-butylacetamide
(2R,S)-2-[1-(4-trifluoromethoxybenzoyl)-5-hydroxy-2-methyl-1H-indol-3-yl]-N-(hydroxymethyl)propanamide
methyl[1-(4-trifluoromethoxybenzoyl)-6-fluoro-5-hydroxy-2-methyl-1H-indol-3-yl]acetate
ethyl[1-(4-trifluoromethoxybenzoyl)-6-fluoro-5-hydroxy-2-methyl-1H-indol-3-yl]acetate
propyl[1-(4-trifluoromethoxybenzoyl)-6-fluoro-5-hydroxy-2-methyl-1H-indol-3-yl]acetate
butyl[1-(4-trifluoromethoxybenzoyl)-6-fluoro-5-hydroxy-2-methyl-1H-indol-3-yl]acetate
sec-butyl[1-(4-trifluoromethoxybenzoyl)-6-fluoro-5-hydroxy-2-methyl-1H-indol-3-yl]acetate
(R)sec-butyl[1-(4-trifluoromethoxybenzoyl)-6-fluoro-5-hydroxy-2-methyl-1H-indol-3-yl]acetate
(S)sec-butyl[1-(4-trifluoromethoxybenzoyl)-6-fluoro-5-hydroxy-2-methyl-1H-indol-3-yl]acetate
isopropyl[1-(4-trifluoromethoxybenzoyl)-6-fluoro-5-hydroxy-2-methyl-1H-indol-3-yl]acetate
tert-butyl[1-(4-trifluoromethoxybenzoyl)-6-fluoro-5-hydroxy-2-methyl-1H-indol-3-yl]acetate
cyclobutyl[1-(4-trifluoromethoxybenzoyl)-6-fluoro-5-hydroxy-2-methyl-1H-indol-3-yl]acetate
cyclopentyl[1-(4-trifluoromethoxybenzoyl)-6-fluoro-5-hydroxy-2-methyl-1H-indol-3-yl]acetate
2-[1-(4-trifluoromethoxybenzoyl)-6-fluoro-5-hydroxy-2-methyl-1H-indol-3-yl]acetamide
2-[1-(4-trifluoromethoxybenzoyl)-6-fluoro-5-hydroxy-2-methyl-1H-indol-3-yl]-N-methylacetamide
2-[1-(4-trifluoromethoxybenzoyl)-6-fluoro-5-hydroxy-2-methyl-1H-indol-3-yl]-N-ethylacetamide
2-[1-(4-trifluoromethoxybenzoyl)-6-fluoro-5-hydroxy-2-methyl-1H-indol-3-yl]-N-propylacetamide
2-[1-(4-trifluoromethoxybenzoyl)-6-fluoro-5-hydroxy-2-methyl-1H-indol-3-yl]-N-isopropylacetamide
2-[1-(4-trifluoromethoxybenzoyl)-6-fluoro-5-hydroxy-2-methyl-1H-indol-3-yl]-N-butylacetamide
2-[1-(4-trifluoromethoxybenzoyl)-6-fluoro-5-hydroxy-2-methyl-1H-indol-3-yl]-N-sec-butylacetamide
2-[1-(4-trifluoromethoxybenzoyl)-6-fluoro-5-hydroxy-2-methyl-1H-indol-3-yl]-N-(2-hydroxyethyl)acetamide
methyl(2R,S)-2-[1-(4-trifluoromethoxybenzoyl)-6-fluoro-5-hydroxy-2-methyl-1H-indol-3-yl]propanoate
ethyl(2R,S)-2-[1-(4-trifluoromethoxybenzoyl)-6-fluoro-5-hydroxy-2-methyl-1H-indol-3-yl]propanoate
propyl(2R,S)-2-[1-(4-trifluoromethoxybenzoyl)-6-fluoro-5-hydroxy-2-methyl-1H-indol-3-yl]propanoate
isopropyl(2R,S)-2-[1-(4-trifluoromethoxybenzoyl)-6-fluoro-5-hydroxy-2-methyl-1H-indol-3-yl]propanoate
butyl(2R,S)-2-[1-(4-trifluoromethoxybenzoyl)-6-fluoro-5-hydroxy-2-methyl-1H-indol-3-yl]propanoate
sec-butyl(2R,S)-2-[1-(4-trifluoromethoxybenzoyl)-6-fluoro-5-hydroxy-2-methyl-1H-indol-3-yl]propanoate
(R)sec-butyl(2R,S)-2-[1-(4-trifluoromethoxybenzoyl)-6-fluoro-5-hydroxy-2-methyl-1H-indol-3-yl]propanoate
(S)sec-butyl(2R,S)-2-[1-(4-trifluoromethoxybenzoyl)-6-fluoro-5-hydroxy-2-methyl-1H-indol-3-yl]propanoate
tert-butyl(2R,S)-2-[1-(4-trifluoromethoxybenzoyl)-6-fluoro-5-hydroxy-2-methyl-1H-indol-3-yl]propanoate
cyclobutyl(2R,S)-2-[1-(4-trifluoromethoxybenzoyl)-6-fluoro-5-hydroxy-2-methyl-1H-indol-3-yl]propanoate
cyclopentyl(2R,S)-2-[1-(4-trifluoromethoxybenzoyl)-6-fluoro-5-hydroxy-2-methyl-1H-indol-3-yl]propanoate
(2R,S)-2-[1-(4-trifluoromethoxybenzoyl)-6-fluoro-5-hydroxy-2-methyl-1H-indol-3-yl]propanamide
(2R,S)-2-[1-(4-trifluoromethoxybenzoyl)-6-fluoro-5-hydroxy-2-methyl-1H-indol-3-yl]-N-methylpropanamide
(2R,S)-2-[1-(4-trifluoromethoxybenzoyl)-6-fluoro-5-hydroxy-2-methyl-1H-indol-3-yl]-N-ethylacetamide
(2R,S)-2-[1-(4-trifluoromethoxybenzoyl)-6-fluoro-5-hydroxy-2-methyl-1H-indol-3-yl]-N-propylacetamide
(2R,S)-2-[1-(4-trifluoromethoxybenzoyl)-6-fluoro-5-hydroxy-2-methyl-1H-indol-3-yl]-N-isopropylacetamide
(2R,S)-2-[1-(4-trifluoromethoxybenzoyl)-6-fluoro-5-hydroxy-2-methyl-1H-indol-3-yl]-N-butylacetamide
(2R,S)-2-[1-(4-trifluoromethoxybenzoyl)-6-fluoro-5-hydroxy-2-methyl-1H-indol-3-yl]-N-sec-butylacetamide
(2R,S)-2-[1-(4-trifluoromethoxybenzoyl)-6-fluoro-5-hydroxy-2-methyl-1H-indol-3-yl]-N-(hydroxymethyl)propanamide
methyl[1-(4-trifluoromethoxybenzoyl)-4-fluoro-5-hydroxy-2-methyl-1H-indol-3-yl]acetate
ethyl[1-(4-trifluoromethoxybenzoyl)-4-fluoro-5-hydroxy-2-methyl-1H-indol-3-yl]acetate
propyl[1-(4-trifluoromethoxybenzoyl)-4-fluoro-5-hydroxy-2-methyl-1H-indol-3-yl]acetate
butyl[1-(4-trifluoromethoxybenzoyl)-4-fluoro-5-hydroxy-2-methyl-1H-indol-3-yl]acetate
sec-butyl[1-(4-trifluoromethoxybenzoyl)-4-fluoro-5-hydroxy-2-methyl-1H-indol-3-yl]acetate
(R)sec-butyl[1-(4-trifluoromethoxybenzoyl)-4-fluoro-5-hydroxy-2-methyl-1H-indol-3-yl]acetate
(S)sec-butyl[1-(4-trifluoromethoxybenzoyl)-4-fluoro-5-hydroxy-2-methyl-1H-indol-3-yl]acetate
isopropyl[1-(4-trifluoromethoxybenzoyl)-4-fluoro-5-hydroxy-2-methyl-1H-indol-3-yl]acetate
tert-butyl[1-(4-trifluoromethoxybenzoyl)-4-fluoro-5-hydroxy-2-methyl-1H-indol-3-yl]acetate
cyclobutyl[1-(4-trifluoromethoxybenzoyl)-4-fluoro-5-hydroxy-2-methyl-1H-indol-3-yl]acetate
cyclopentyl[1-(4-trifluoromethoxybenzoyl)-4-fluoro-5-hydroxy-2-methyl-1H-indol-3-yl]acetate
2-[1-(4-trifluoromethoxybenzoyl)-4-fluoro-5-hydroxy-2-methyl-1H-indol-3-yl]acetamide
2-[1-(4-trifluoromethoxybenzoyl)-4-fluoro-5-hydroxy-2-methyl-1H-indol-3-yl]-N-methylacetamide
2-[1-(4-trifluoromethoxybenzoyl)-4-fluoro-5-hydroxy-2-methyl-1H-indol-3-yl]-N-ethylacetamide
2-[1-(4-trifluoromethoxybenzoyl)-4-fluoro-5-hydroxy-2-methyl-1H-indol-3-yl]-N-propylacetamide
2-[1-(4-trifluoromethoxybenzoyl)-4-fluoro-5-hydroxy-2-methyl-1H-indol-3-yl]-N-isopropylacetamide
2-[1-(4-trifluoromethoxybenzoyl)-4-fluoro-5-hydroxy-2-methyl-1H-indol-3-yl]-N-butylacetamide
2-[1-(4-trifluoromethoxybenzoyl)-4-fluoro-5-hydroxy-2-methyl-1H-indol-3-yl]-N-sec-butylacetamide
2-[1-(4-trifluoromethoxybenzoyl)-4-fluoro-5-hydroxy-2-methyl-1H-indol-3-yl]-N-(2-hydroxyethyl)acetamide
methyl(2R,S)-2-[1-(4-trifluoromethoxybenzoyl)-4-fluoro-5-hydroxy-2-methyl-1H-indol-3-yl]propanoate
ethyl(2R,S)-2-[1-(4-trifluoromethoxybenzoyl)-4-fluoro-5-hydroxy-2-methyl-1H-indol-3-yl]propanoate
propyl(2R,S)-2-[1-(4-trifluoromethoxybenzoyl)-4-fluoro-5-hydroxy-2-methyl-1H-indol-3-yl]propanoate
isopropyl(2R,S)-2-[1-(4-trifluoromethoxybenzoyl)-4-fluoro-5-hydroxy-2-methyl-1H-indol-3-yl]propanoate
butyl(2R,S)-2-[1-(4-trifluoromethoxybenzoyl)-4-fluoro-5-hydroxy-2-methyl-1H-indol-3-yl]propanoate
sec-butyl(2R,S)-2-[1-(4-trifluoromethoxybenzoyl)-4-fluoro-5-hydroxy-2-methyl-1H-indol-3-yl]propanoate
(R)sec-butyl(2R,S)-2-[1-(4-trifluoromethoxybenzoyl)-4-fluoro-5-hydroxy-2-methyl-1H-indol-3-yl]propanoate
(S)sec-butyl(2R,S)-2-[1-(4-trifluoromethoxybenzoyl)-4-fluoro-5-hydroxy-2-methyl-1H-indol-3-yl]propanoate
tert-butyl(2R,S)-2-[1-(4-trifluoromethoxybenzoyl)-4-fluoro-5-hydroxy-2-methyl-1H-indol-3-yl]propanoate
cyclobutyl(2R,S)-2-[1-(4-trifluoromethoxybenzoyl)-4-fluoro-5-hydroxy-2-methyl-1H-indol-3-yl]propanoate
cyclopentyl(2R,S)-2-[1-(4-trifluoromethoxybenzoyl)-4-fluoro-5-hydroxy-2-methyl-1H-indol-3-yl]propanoate
(2R,S)-2-[1-(4-trifluoromethoxybenzoyl)-4-fluoro-5-hydroxy-2-methyl-1H-indol-3-yl]propanamide
(2R,S)-2-[1-(4-trifluoromethoxybenzoyl)-4-fluoro-5-hydroxy-2-methyl-1H-indol-3-yl]-N-methylpropanamide
(2R,S)-2-[1-(4-trifluoromethoxybenzoyl)-4-fluoro-5-hydroxy-2-methyl-1H-indol-3-yl]-N-ethylacetamide
(2R,S)-2-[1-(4-trifluoromethoxybenzoyl)-4-fluoro-5-hydroxy-2-methyl-1H-indol-3-yl]-N-propylacetamide
(2R,S)-2-[1-(4-trifluoromethoxybenzoyl)-4-fluoro-5-hydroxy-2-methyl-1H-indol-3-yl]-N-isopropylacetamide
(2R,S)-2-[1-(4-trifluoromethoxybenzoyl)-4-fluoro-5-hydroxy-2-methyl-1H-indol-3-yl]-N-butylacetamide
(2R,S)-2-[1-(4-trifluoromethoxybenzoyl)-4-fluoro-5-hydroxy-2-methyl-1H-indol-3-yl]-N-sec-butylacetamide
(2R,S)-2-[1-(4-trifluoromethoxybenzoyl)-4-fluoro-5-hydroxy-2-methyl-1H-indol-3-yl]-N-(hydroxymethyl)propanamide
methyl[1-(4-trifluoromethoxybenzoyl)-4,6-difluoro-5-hydroxy-2-methyl-1H-indol-3-yl]acetate
ethyl[1-(4-trifluoromethoxybenzoyl)-4,6-difluoro-5-hydroxy-2-methyl-1H-indol-3-yl]acetate
propyl[1-(4-trifluoromethoxybenzoyl)-4,6-difluoro-5-hydroxy-2-methyl-1H-indol-3-yl]acetate
butyl[1-(4-trifluoromethoxybenzoyl)-4,6-difluoro-5-hydroxy-2-methyl-1H-indol-3-yl]acetate
sec-butyl[1-(4-trifluoromethoxybenzoyl)-4,6-difluoro-5-hydroxy-2-methyl-1H-indol-3-yl]acetate
(R)sec-butyl[1-(4-trifluoromethoxybenzoyl)-4,6-difluoro-5-hydroxy-2-methyl-1H-indol-3-yl]acetate
(S)sec-butyl[1-(4-trifluoromethoxybenzoyl)-4,6-difluoro-5-hydroxy-2-methyl-1H-indol-3-yl]acetate
isopropyl[1-(4-trifluoromethoxybenzoyl)-4,6-difluoro-5-hydroxy-2-methyl-1H-indol-3-yl]acetate
tert-butyl[1-(4-trifluoromethoxybenzoyl)-4,6-difluoro-5-hydroxy-2-methyl-1H-indol-3-yl]acetate
cyclobutyl[1-(4-trifluoromethoxybenzoyl)-4,6-difluoro-5-hydroxy-2-methyl-1H-indol-3-yl]acetate
cyclopentyl[1-(4-trifluoromethoxybenzoyl)-4,6-difluoro-5-hydroxy-2-methyl-1H-indol-3-yl]acetate
2-[1-(4-trifluoromethoxybenzoyl)-4,6-difluoro-5-hydroxy-2-methyl-1H-indol-3-yl]acetamide
2-[1-(4-trifluoromethoxybenzoyl)-4,6-difluoro-5-hydroxy-2-methyl-1H-indol-3-yl]-N-methylacetamide
2-[1-(4-trifluoromethoxybenzoyl)-4,6-difluoro-5-hydroxy-2-methyl-1H-indol-3-yl]-N-ethylacetamide
2-[1-(4-trifluoromethoxybenzoyl)-4,6-difluoro-5-hydroxy-2-methyl-1H-indol-3-yl]-N-propylacetamide
2-[1-(4-trifluoromethoxybenzoyl)-4,6-difluoro-5-hydroxy-2-methyl-1H-indol-3-yl]-N-isopropylacetamide
2-[1-(4-trifluoromethoxybenzoyl)-4,6-difluoro-5-hydroxy-2-methyl-1H-indol-3-yl]-N-butylacetamide
2-[1-(4-trifluoromethoxybenzoyl)-4,6-difluoro-5-hydroxy-2-methyl-1H-indol-3-yl]-N-sec-butylacetamide
2-[1-(4-trifluoromethoxybenzoyl)-4,6-difluoro-5-hydroxy-2-methyl-1H-indol-3-yl]-N-(2-hydroxyethyl)acetamide
methyl(2R,S)-2-[1-(4-trifluoromethoxybenzoyl)-4,6-difluoro-5-hydroxy-2-methyl-1H-indol-3-yl]propanoate
ethyl(2R,S)-2-[1-(4-trifluoromethoxybenzoyl)-4,6-difluoro-5-hydroxy-2-methyl-1H-indol-3-yl]propanoate
propyl(2R,S)-2-[1-(4-trifluoromethoxybenzoyl)-4,6-difluoro-5-hydroxy-2-methyl-1H-indol-3-yl]propanoate
isopropyl(2R,S)-2-[1-(4-trifluoromethoxybenzoyl)-4,6-difluoro-5-hydroxy-2-methyl-1H-indol-3-yl]propanoate
butyl(2R,S)-2-[1-(4-trifluoromethoxybenzoyl)-4,6-difluoro-5-hydroxy-2-methyl-1H-indol-3-yl]propanoate
sec-butyl(2R,S)-2-[1-(4-trifluoromethoxybenzoyl)-4,6-difluoro-5-hydroxy-2-methyl-1H-indol-3-yl]propanoate
(R)sec-butyl(2R,S)-2-[1-(4-trifluoromethoxybenzoyl)-4,6-difluoro-5-hydroxy-2-methyl-1H-indol-3-yl]propanoate
(S)sec-butyl(2R,S)-2-[1-(4-trifluoromethoxybenzoyl)-4,6-difluoro-5-hydroxy-2-methyl-1H-indol-3-yl]propanoate
tert-butyl(2R,S)-2-[1-(4-trifluoromethoxybenzoyl)-4,6-difluoro-5-hydroxy-2-methyl-1H-indol-3-yl]propanoate
cyclobutyl(2R,S)-2-[1-(4-trifluoromethoxybenzoyl)-4,6-difluoro-5-hydroxy-2-methyl-1H-indol-3-yl]propanoate
cyclopentyl(2R,S)-2-[1-(4-trifluoromethoxybenzoyl)-4,6-difluoro-5-hydroxy-2-methyl-1H-indol-3-yl]propanoate
(2R,S)-2-[1-(4-trifluoromethoxybenzoyl)-4,6-difluoro-5-hydroxy-2-methyl-1H-indol-3-yl]propanamide
(2R,S)-2-[1-(4-trifluoromethoxybenzoyl)-4,6-difluoro-5-hydroxy-2-methyl-1H-indol-3-yl]-N-methylpropanamide
(2R,S)-2-[1-(4-trifluoromethoxybenzoyl)-4,6-difluoro-5-hydroxy-2-methyl-1H-indol-3-yl]-N-ethylacetamide
(2R,S)-2-[1-(4-trifluoromethoxybenzoyl)-4,6-difluoro-5-hydroxy-2-methyl-1H-indol-3-yl]-N-propylacetamide
(2R,S)-2-[1-(4-trifluoromethoxybenzoyl)-4,6-difluoro-5-hydroxy-2-methyl-1H-indol-3-yl]-N-isopropylacetamide
(2R,S)-2-[1-(4-trifluoromethoxybenzoyl)-4,6-difluoro-5-hydroxy-2-methyl-1H-indol-3-yl]-N-butylacetamide
(2R,S)-2-[1-(4-trifluoromethoxybenzoyl)-4,6-difluoro-5-hydroxy-2-methyl-1H-indol-3-yl]-N-sec-butylacetamide
(2R,S)-2-[1-(4-trifluoromethoxybenzoyl)-4,6-difluoro-5-hydroxy-2-methyl-1H-indol-3-yl]-N-(hydroxymethyl)propanamide
methyl{1-[4-(difluoromethoxy)benzoyl]-6-fluoro-5-methoxy-2-methyl-1H-indol-3-yl]acetate
ethyl{1-[4-(difluoromethoxy)benzoyl]-6-fluoro-5-methoxy-2-methyl-1H-indol-3-yl]acetate
propyl{1-[4-(difluoromethoxy)benzoyl]-6-fluoro-5-methoxy-2-methyl-1H-indol-3-yl]acetate
butyl{1-[4-(difluoromethoxy)benzoyl]-6-fluoro-5-methoxy-2-methyl-1H-indol-3-yl]acetate
sec-butyl{1-[4-(difluoromethoxy)benzoyl]-6-fluoro-5-methoxy-2-methyl-1H-indol-3-yl]acetate
(R)sec-butyl{1-[4-(difluoromethoxy)benzoyl]-6-fluoro-5-methoxy-2-methyl-1H-indol-3-yl]acetate
(S)sec-butyl{1-[4-(difluoromethoxy)benzoyl]-6-fluoro-5-methoxy-2-methyl-1H-indol-3-yl]acetate
isopropyl{1-[4-(difluoromethoxy)benzoyl]-6-fluoro-5-methoxy-2-methyl-1H-indol-3-yl]acetate
tert-butyl{1-[4-(difluoromethoxy)benzoyl]-6-fluoro-5-methoxy-2-methyl-1H-indol-3-yl]acetate
cyclobutyl{1-[4-(difluoromethoxy)benzoyl]-6-fluoro-5-methoxy-2-methyl-1H-indol-3-yl]acetate
cyclopentyl{1-[4-(difluoromethoxy)benzoyl]-6-fluoro-5-methoxy-2-methyl-1H-indol-3-yl]acetate
2-{1-[4-(difluoromethoxy)benzoyl]-6-fluoro-5-methoxy-2-methyl-1H-indol-3-yl]acetamide
2-{1-[4-(difluoromethoxy)benzoyl]-6-fluoro-5-methoxy-2-methyl-1H-indol-3-yl]-N-methylacetamide
2-{1-[4-(difluoromethoxy)benzoyl]-6-fluoro-5-methoxy-2-methyl-1H-indol-3-yl]-N-ethylacetamide
2-{1-[4-(difluoromethoxy)benzoyl]-6-fluoro-5-methoxy-2-methyl-1H-indol-3-yl]-N-propylacetamide
2-{1-[4-(difluoromethoxy)benzoyl]-6-fluoro-5-methoxy-2-methyl-1H-indol-3-yl]-N-isopropylacetamide
2-{1-[4-(difluoromethoxy)benzoyl]-6-fluoro-5-methoxy-2-methyl-1H-indol-3-yl]-N-butylacetamide
2-{1-[4-(difluoromethoxy)benzoyl]-6-fluoro-5-methoxy-2-methyl-1H-indol-3-yl]-N-sec-butylacetamide
2-{1-[4-(difluoromethoxy)benzoyl]-6-fluoro-5-methoxy-2-methyl-1H-indol-3-yl]-N-(2-hydroxyethyl)acetamide
methyl{1-[4-(difluoromethoxy)benzoyl]-6-fluoro-5-hydroxy-2-methyl-1H-indol-3-yl]acetate
ethyl{1-[4-(difluoromethoxy)benzoyl]-6-fluoro-5-hydroxy-2-methyl-1H-indol-3-yl]acetate
propyl{1-[4-(difluoromethoxy)benzoyl]-6-fluoro-5-hydroxy-2-methyl-1H-indol-3-yl]acetate
butyl{1-[4-(difluoromethoxy)benzoyl]-6-fluoro-5-hydroxy-2-methyl-1H-indol-3-yl]acetate
sec-butyl{1-[4-(difluoromethoxy)benzoyl]-6-fluoro-5-hydroxy-2-methyl-1H-indol-3-yl]acetate
(R)sec-butyl{1-[4-(difluoromethoxy)benzoyl]-6-fluoro-5-hydroxy-2-methyl-1H-indol-3-yl]acetate
(S)sec-butyl{1-[4-(difluoromethoxy)benzoyl]-6-fluoro-5-hydroxy-2-methyl-1H-indol-3-yl]acetate
isopropyl{1-[4-(difluoromethoxy)benzoyl]-6-fluoro-5-hydroxy-2-methyl-1H-indol-3-yl]acetate
tert-butyl{1-[4-(difluoromethoxy)benzoyl]-6-fluoro-5-hydroxy-2-methyl-1H-indol-3-yl]acetate
cyclobutyl{1-[4-(difluoromethoxy)benzoyl]-6-fluoro-5-hydroxy-2-methyl-1H-indol-3-yl]acetate
cyclopentyl{1-[4-(difluoromethoxy)benzoyl]-6-fluoro-5-hydroxy-2-methyl-1H-indol-3-yl]acetate
2-{1-[4-(difluoromethoxy)benzoyl]-6-fluoro-5-hydroxy-2-methyl-1H-indol-3-yl]acetamide
2-{1-[4-(difluoromethoxy)benzoyl]-6-fluoro-5-hydroxy-2-methyl-1H-indol-3-yl]-N-methylacetamide
2-{1-[4-(difluoromethoxy)benzoyl]-6-fluoro-5-hydroxy-2-methyl-1H-indol-3-yl]-N-ethylacetamide
2-{1-[4-(difluoromethoxy)benzoyl]-6-fluoro-5-hydroxy-2-methyl-1H-indol-3-yl]-N-propylacetamide
2-{1-[4-(difluoromethoxy)benzoyl]-6-fluoro-5-hydroxy-2-methyl-1H-indol-3-yl]-N-isopropylacetamide
2-{1-[4-(difluoromethoxy)benzoyl]-6-fluoro-5-hydroxy-2-methyl-1H-indol-3-yl]-N-butylacetamide
2-{1-[4-(difluoromethoxy)benzoyl]-6-fluoro-5-hydroxy-2-methyl-1H-indol-3-yl]-N-sec-butylacetamide
2-{1-[4-(difluoromethoxy)benzoyl]-6-fluoro-5-hydroxy-2-methyl-1H-indol-3-yl]-N-(2-hydroxyethyl)acetamide
methyl{1-[4-(difluoromethoxy)benzoyl]-5-methoxy-2-methyl-1H-indol-3-yl]acetate
ethyl{1-[4-(difluoromethoxy)benzoyl]-5-methoxy-2-methyl-1H-indol-3-yl]acetate
propyl{1-[4-(difluoromethoxy)benzoyl]-5-methoxy-2-methyl-1H-indol-3-yl]acetate
butyl{1-[4-(difluoromethoxy)benzoyl]-5-methoxy-2-methyl-1H-indol-3-yl]acetate
sec-butyl{1-[4-(difluoromethoxy)benzoyl]-5-methoxy-2-methyl-1H-indol-3-yl]acetate
(R)sec-butyl{1-[4-(difluoromethoxy)benzoyl]-5-methoxy-2-methyl-1H-indol-3-yl]acetate
(S)sec-butyl{1-[4-(difluoromethoxy)benzoyl]-5-methoxy-2-methyl-1H-indol-3-yl]acetate
isopropyl{1-[4-(difluoromethoxy)benzoyl]-5-methoxy-2-methyl-1H-indol-3-yl]acetate
tert-butyl{1-[4-(difluoromethoxy)benzoyl]-5-methoxy-2-methyl-1H-indol-3-yl]acetate
cyclobutyl{1-[4-(difluoromethoxy)benzoyl]-5-methoxy-2-methyl-1H-indol-3-yl]acetate
cyclopentyl{1-[4-(difluoromethoxy)benzoyl]-5-methoxy-2-methyl-1H-indol-3-yl]acetate
2-{1-[4-(difluoromethoxy)benzoyl]-5-methoxy-2-methyl-1H-indol-3-yl]acetamide
2-{1-[4-(difluoromethoxy)benzoyl]-5-methoxy-2-methyl-1H-indol-3-yl]-N-methylacetamide
2-{1-[4-(difluoromethoxy)benzoyl]-5-methoxy-2-methyl-1H-indol-3-yl]-N-ethylacetamide
2-{1-[4-(difluoromethoxy)benzoyl]-5-methoxy-2-methyl-1H-indol-3-yl]-N-propylacetamide
2-{1-[4-(difluoromethoxy)benzoyl]-5-methoxy-2-methyl-1H-indol-3-yl]-N-isopropylacetamide
2-{1-[4-(difluoromethoxy)benzoyl]-5-methoxy-2-methyl-1H-indol-3-yl]-N-butylacetamide
2-{1-[4-(difluoromethoxy)benzoyl]-5-methoxy-2-methyl-1H-indol-3-yl]-N-sec-butylacetamide
2-{1-[4-(difluoromethoxy)benzoyl]-5-methoxy-2-methyl-1H-indol-3-yl]-N-(2-hydroxyethyl)acetamide
methyl{1-[4-(difluoromethoxy)benzoyl]-5-hydroxy-2-methyl-1H-indol-3-yl]acetate
ethyl{1-[4-(difluoromethoxy)benzoyl]-5-hydroxy-2-methyl-1H-indol-3-yl]acetate
propyl{1-[4-(difluoromethoxy)benzoyl]-5-hydroxy-2-methyl-1H-indol-3-yl]acetate
butyl{1-[4-(difluoromethoxy)benzoyl]-5-hydroxy-2-methyl-1H-indol-3-yl]acetate
sec-butyl{1-[4-(difluoromethoxy)benzoyl]-5-hydroxy-2-methyl-1H-indol-3-yl]acetate
(R)sec-butyl{1-[4-(difluoromethoxy)benzoyl]-5-hydroxy-2-methyl-1H-indol-3-yl]acetate
(S)sec-butyl{1-[4-(difluoromethoxy)benzoyl]-5-hydroxy-2-methyl-1H-indol-3-yl]acetate
isopropyl{1-[4-(difluoromethoxy)benzoyl]-5-hydroxy-2-methyl-1H-indol-3-yl]acetate
tert-butyl{1-[4-(difluoromethoxy)benzoyl]-5-hydroxy-2-methyl-1H-indol-3-yl]acetate
cyclobutyl{1-[4-(difluoromethoxy)benzoyl]-5-hydroxy-2-methyl-1H-indol-3-yl]acetate
cyclopentyl{1-[4-(difluoromethoxy)benzoyl]-5-hydroxy-2-methyl-1H-indol-3-yl]acetate
2-{1-[4-(difluoromethoxy)benzoyl)-5-hydroxy-2-methyl-1H-indol-3-yl]acetamide
2-{1-[4-(difluoromethoxy)benzoyl]-5-hydroxy-2-methyl-1H-indol-3-yl]-N-methylacetamide
2-{1-[4-(difluoromethoxy)benzoyl]-5-hydroxy-2-methyl-1H-indol-3-yl]-N-ethylacetamide
2-{1-[4-(difluoromethoxy)benzoyl]-5-hydroxy-2-methyl-1H-indol-3-yl]-N-propylacetamide
2-{1-[4-(difluoromethoxy)benzoyl]-5-hydroxy-2-methyl-1H-indol-3-yl]-N-isopropylacetamide
2-{1-[4-(difluoromethoxy)benzoyl)-5-hydroxy-2-methyl-1H-indol-3-yl]-N-butylacetamide
2-{1-[4-(difluoromethoxy)benzoyl]-5-hydroxy-2-methyl-1H-indol-3-yl]-N-sec-butylacetamide
2-{1-[4-(difluoromethoxy)benzoyl]-5-hydroxy-2-methyl-1H-indol-3-yl]-N-(2-hydroxyethyl)acetamide
methyl{1-[4-chlorobenzoyl]-6-chloro-5-methoxy-2-methyl-1H-indol-3-yl]acetate
ethyl{1-[4-chlorobenzoyl]-6-chloro-5-methoxy-2-methyl-1H-indol-3-yl]acetate
propyl{1-[4-chlorobenzoyl]-6-chloro-5-methoxy-2-methyl-1H-indol-3-yl]acetate
butyl{1-[4-chlorobenzoyl]-6-chloro-5-methoxy-2-methyl-1H-indol-3-yl]acetate
sec-butyl{1-[4-chlorobenzoyl]-6-chloro-5-methoxy-2-methyl-1H-indol-3-yl]acetate
(R)sec-butyl{1-[4-chlorobenzoyl]-6-chloro-5-methoxy-2-methyl-1H-indol-3-yl]acetate
(S)sec-butyl{1-[4-chlorobenzoyl]-6-chloro-5-methoxy-2-methyl-1H-indol-3-yl]acetate
isopropyl{1-[4-chlorobenzoyl]-6-chloro-5-methoxy-2-methyl-1H-indol-3-yl]acetate
tert-butyl{1-[4-chlorobenzoyl]-6-chloro-5-methoxy-2-methyl-1H-indol-3-yl]acetate
cyclobutyl{1-[4-chlorobenzoyl]-6-chloro-5-methoxy-2-methyl-1H-indol-3-yl]acetate
cyclopentyl{1-[4-chlorobenzoyl]-6-chloro-5-methoxy-2-methyl-1H-indol-3-yl]acetate
2-{1-[4-chlorobenzoyl]-6-chloro-5-methoxy-2-methyl-1H-indol-3-yl]acetamide
2-{1-[4-chlorobenzoyl]-6-chloro-5-methoxy-2-methyl-1H-indol-3-yl]-N-methylacetamide
2-{1-[4-chlorobenzoyl]-6-chloro-5-methoxy-2-methyl-1H-indol-3-yl]-N-ethylacetamide
2-{1-[4-chlorobenzoyl]-6-chloro-5-methoxy-2-methyl-1H-indol-3-yl]-N-propylacetamide
2-{1-[4-chlorobenzoyl]-6-chloro-5-methoxy-2-methyl-1H-indol-3-yl]-N-isopropylacetamide
2-{1-[4-chlorobenzoyl]-6-chloro-5-methoxy-2-methyl-1H-indol-3-yl]-N-butylacetamide
2-{1-[4-chlorobenzoyl]-6-chloro-5-methoxy-2-methyl-1H-indol-3-yl]-N-sec-butylacetamide
2-{1-[4-chlorobenzoyl]-6-chloro-5-methoxy-2-methyl-1H-indol-3-yl]-N-(2-hydroxyethyl)acetamide
methyl{1-[4-chlorobenzoyl]-6-chloro-5-hydroxy-2-methyl-1H-indol-3-yl]acetate
ethyl{1-[4-chlorobenzoyl]-6-chloro-5-hydroxy-2-methyl-1H-indol-3-yl]acetate
propyl{1-[4-chlorobenzoyl]-6-chloro-5-hydroxy-2-methyl-1H-indol-3-yl]acetate
butyl{1-[4-chlorobenzoyl]-6-chloro-5-hydroxy-2-methyl-1H-indol-3-yl]acetate
sec-butyl{1-[4-chlorobenzoyl]-6-chloro-5-hydroxy-2-methyl-1H-indol-3-yl]acetate
(R)sec-butyl{1-[4-chlorobenzoyl]-6-chloro-5-hydroxy-2-methyl-1H-indol-3-yl]acetate
(S)sec-butyl{1-[4-chlorobenzoyl]-6-chloro-5-hydroxy-2-methyl-1H-indol-3-yl]acetate
isopropyl{1-[4-chlorobenzoyl]-6-chloro-5-hydroxy-2-methyl-1H-indol-3-yl]acetate
tert-butyl{1-[4-chlorobenzoyl]-6-chloro-5-hydroxy-2-methyl-1H-indol-3-yl]acetate
cyclobutyl{1-[4-chlorobenzoyl]-6-chloro-5-hydroxy-2-methyl-1H-indol-3-yl]acetate
cyclopentyl{1-[4-chlorobenzoyl]-6-chloro-5-hydroxy-2-methyl-1H-indol-3-yl]acetate
2-{1-[4-chlorobenzoyl]-6-chloro-5-hydroxy-2-methyl-1H-indol-3-yl]acetamide
2-{1-[4-chlorobenzoyl]-6-chloro-5-hydroxy-2-methyl-1H-indol-3-yl]-N-methylacetamide
2-{1-[4-chlorobenzoyl]-6-chloro-5-hydroxy-2-methyl-1H-indol-3-yl]-N-ethylacetamide
2-{1-[4-chlorobenzoyl]-6-chloro-5-hydroxy-2-methyl-1H-indol-3-yl]-N-propylacetamide
2-{1-[4-chlorobenzoyl]-6-chloro-5-hydroxy-2-methyl-1H-indol-3-yl]-N-isopropylacetamide
2-{1-[4-chlorobenzoyl]-6-chloro-5-hydroxy-2-methyl-1H-indol-3-yl]-N-butylacetamide
2-{1-[4-chlorobenzoyl]-6-chloro-5-hydroxy-2-methyl-1H-indol-3-yl]-N-sec-butylacetamide
2-{1-[4-chlorobenzoyl]-6-chloro-5-hydroxy-2-methyl-1H-indol-3-yl]-N-(2-hydroxyethyl)acetamide
methyl{1-[4-chlorobenzoyl]-4-chloro-5-methoxy-2-methyl-1H-indol-3-yl]acetate
ethyl{1-[4-chlorobenzoyl]-4-chloro-5-methoxy-2-methyl-1H-indol-3-yl]acetate
propyl{1-[4-chlorobenzoyl]-4-chloro-5-methoxy-2-methyl-1H-indol-3-yl]acetate
butyl{1-[4-chlorobenzoyl]-4-chloro-5-methoxy-2-methyl-1H-indol-3-yl]acetate
sec-butyl{1-[4-chlorobenzoyl]-4-chloro-5-methoxy-2-methyl-1H-indol-3-yl]acetate
(R)sec-butyl{1-[4-chlorobenzoyl]-4-chloro-5-methoxy-2-methyl-1H-indol-3-yl]acetate
(S)sec-butyl{1-[4-chlorobenzoyl]-4-chloro-5-methoxy-2-methyl-1H-indol-3-yl]acetate
isopropyl{1-[4-chlorobenzoyl]-4-chloro-5-methoxy-2-methyl-1H-indol-3-yl]acetate
tert-butyl{1-[4-chlorobenzoyl]-4-chloro-5-methoxy-2-methyl-1H-indol-3-yl]acetate
cyclobutyl{1-[4-chlorobenzoyl]-4-chloro-5-methoxy-2-methyl-1H-indol-3-yl]acetate
cyclopentyl{1-[4-chlorobenzoyl]-4-chloro-5-methoxy-2-methyl-1H-indol-3-yl]acetate
2-{1-[4-chlorobenzoyl]-4-chloro-5-methoxy-2-methyl-1H-indol-3-yl]acetamide
2-{1-[4-chlorobenzoyl]-4-chloro-5-methoxy-2-methyl-1H-indol-3-yl]-N-methylacetamide
2-{1-[4-chlorobenzoyl]-4-chloro-5-methoxy-2-methyl-1H-indol-3-yl]-N-ethylacetamide
2-{1-[4-chlorobenzoyl]-4-chloro-5-methoxy-2-methyl-1H-indol-3-yl]-N-propylacetamide
2-{1-[4-chlorobenzoyl)-4-chloro-5-methoxy-2-methyl-1H-indol-3-yl]-N-isopropylacetamide
2-{1-[4-chlorobenzoyl]-4-chloro-5-methoxy-2-methyl-1H-indol-3-yl]-N-butylacetamide
2-{1-[4-chlorobenzoyl]-4-chloro-5-methoxy-2-methyl-1H-indol-3-yl]-N-sec-butylacetamide
2-{1-[4-chlorobenzoyl]-4-chloro-5-methoxy-2-methyl-1H-indol-3-yl]-N-(2-hydroxyethyl)acetamide
methyl{1-[4-chlorobenzoyl]-4-chloro-5-hydroxy-2-methyl-1H-indol-3-yl]acetate
ethyl{1-[4-chlorobenzoyl]-4-chloro-5-hydroxy-2-methyl-1H-indol-3-yl]acetate
propyl{1-[4-chlorobenzoyl]-4-chloro-5-hydroxy-2-methyl-1H-indol-3-yl]acetate
butyl{1-[4-chlorobenzoyl]-4-chloro-5-hydroxy-2-methyl-1H-indol-3-yl]acetate
sec-butyl{1-[4-chlorobenzoyl]-4-chloro-5-hydroxy-2-methyl-1H-indol-3-yl]acetate
(R)sec-butyl{1-[4-chlorobenzoyl]-4-chloro-5-hydroxy-2-methyl-1H-indol-3-yl]acetate
(S)sec-butyl{1-[4-chlorobenzoyl]-4-chloro-5-hydroxy-2-methyl-1H-indol-3-yl]acetate
isopropyl{1-[4-chlorobenzoyl]-4-chloro-5-hydroxy-2-methyl-1H-indol-3-yl]acetate
tert-butyl{1-[4-chlorobenzoyl]-4-chloro-5-hydroxy-2-methyl-1H-indol-3-yl]acetate
cyclobutyl{1-[4-chlorobenzoyl]-4-chloro-5-hydroxy-2-methyl-1H-indol-3-yl]acetate
cyclopentyl{1-[4-chlorobenzoyl]-4-chloro-5-hydroxy-2-methyl-1H-indol-3-yl]acetate
2-{1-[4-chlorobenzoyl]-4-chloro-5-hydroxy-2-methyl-1H-indol-3-yl]acetamide
2-{1-[4-chlorobenzoyl]-4-chloro-5-hydroxy-2-methyl-1H-indol-3-yl]-N-methylacetamide
2-{1-[4-chlorobenzoyl]-4-chloro-5-hydroxy-2-methyl-1H-indol-3-yl]-N-ethylacetamide
2-{1-[4-chlorobenzoyl]-4-chloro-5-hydroxy-2-methyl-1H-indol-3-yl]-N-propylacetamide
2-{1-[4-chlorobenzoyl]-4-chloro-5-hydroxy-2-methyl-1H-indol-3-yl]-N-isopropylacetamide
2-{1-[4-chlorobenzoyl]-4-chloro-5-hydroxy-2-methyl-1H-indol-3-yl]-N-butylacetamide
2-{1-[4-chlorobenzoyl]-4-chloro-5-hydroxy-2-methyl-1H-indol-3-yl]-N-sec-butylacetamide
2-{1-[4-chlorobenzoyl]-4-chloro-5-hydroxy-2-methyl-1H-indol-3-yl]-N-(2-hydroxyethyl)acetamide
methyl[1-(4-trifluoromethoxybenzoyl)-5-methoxy-2-methyl-1H-indol-3-yl]acetate
ethyl[1-(4-trifluoromethoxybenzoyl)-5-methoxy-2-methyl-1H-indol-3-yl]acetate
propyl[1-(4-trifluoromethoxybenzoyl)-5-methoxy-2-methyl-1H-indol-3-yl]acetate
butyl[1-(4-trifluoromethoxybenzoyl)-5-methoxy-2-methyl-1H-indol-3-yl]acetate
sec-butyl[1-(4-trifluoromethoxybenzoyl)-5-methoxy-2-methyl-1H-indol-3-yl]acetate
(R)sec-butyl[1-(4-trifluoromethoxybenzoyl)-5-methoxy-2-methyl-1H-indol-3-yl]acetate
(S)sec-butyl[1-(4-trifluoromethoxybenzoyl)-5-methoxy-2-methyl-1H-indol-3-yl]acetate
isopropyl[1-(4-trifluoromethoxybenzoyl)-5-methoxy-2-methyl-1H-indol-3-yl]acetate
tert-butyl[1-(4-trifluoromethoxybenzoyl)-5-methoxy-2-methyl-1H-indol-3-yl]acetate
cyclobutyl[1-(4-trifluoromethoxybenzoyl)-5-methoxy-2-methyl-1H-indol-3-yl]acetate
cyclopentyl[1-(4-trifluoromethoxybenzoyl)-5-methoxy-2-methyl-1H-indol-3-yl]acetate
2-[1-(4-trifluoromethoxybenzoyl)-5-methoxy-2-methyl-1H-indol-3-yl]acetamide
2-[1-(4-trifluoromethoxybenzoyl)-5-methoxy-2-methyl-1H-indol-3-yl]-N-methylacetamide
2-[1-(4-trifluoromethoxybenzoyl)-5-methoxy-2-methyl-1H-indol-3-yl]-N-ethylacetamide
2-[1-(4-trifluoromethoxybenzoyl)-5-methoxy-2-methyl-1H-indol-3-yl]-N-propylacetamide
2-[1-(4-trifluoromethoxybenzoyl)-5-methoxy-2-methyl-1H-indol-3-yl]-N-isopropylacetamide
2-[1-(4-trifluoromethoxybenzoyl)-5-methoxy-2-methyl-1H-indol-3-yl]-N-butylacetamide
2-[1-(4-trifluoromethoxybenzoyl)-5-methoxy-2-methyl-1H-indol-3-yl]-N-sec-butylacetamide
2-[1-(4-trifluoromethoxybenzoyl)-5-methoxy-2-methyl-1H-indol-3-yl]-N-(2-hydroxyethyl)acetamide
methyl[1-(4-trifluoromethoxybenzoyl)-5-methoxy-6-fluoro-2-methyl-1H-indol-3-yl]acetate
ethyl[1-(4-trifluoromethoxybenzoyl)-5-methoxy-6-fluoro-2-methyl-1H-indol-3-yl]acetate
propyl[1-(4-trifluoromethoxybenzoyl)-5-methoxy-6-fluoro-2-methyl-1H-indol-3-yl]acetate
butyl[1-(4-trifluoromethoxybenzoyl)-5-methoxy-6-fluoro-2-methyl-1H-indol-3-yl]acetate
sec-butyl[1-(4-trifluoromethoxybenzoyl)-5-methoxy-6-fluoro-2-methyl-1H-indol-3-yl]acetate
(R)sec-butyl[1-(4-trifluoromethoxybenzoyl)-5-methoxy-6-fluoro-2-methyl-1H-indol-3-yl]acetate
(S)sec-butyl[1-(4-trifluoromethoxybenzoyl)-5-methoxy-6-fluoro-2-methyl-1H-indol-3-yl]acetate
isopropyl[1-(4-trifluoromethoxybenzoyl)-5-methoxy-6-fluoro-2-methyl-1H-indol-3-yl]acetate
tert-butyl[1-(4-trifluoromethoxybenzoyl)-5-methoxy-6-fluoro-2-methyl-1H-indol-3-yl]acetate
cyclobutyl[1-(4-trifluoromethoxybenzoyl)-5-methoxy-6-fluoro-2-methyl-1H-indol-3-yl]acetate
cyclopentyl[1-(4-trifluoromethoxybenzoyl)-5-methoxy-6-fluoro-2-methyl-1H-indol-3-yl]acetate
2-[1-(4-trifluoromethoxybenzoyl)-5-methoxy-6-fluoro-2-methyl-1H-indol-3-yl]acetamide
2-[1-(4-trifluoromethoxybenzoyl)-5-methoxy-6-fluoro-2-methyl-1H-indol-3-yl]-N-methylacetamide
2-[1-(4-trifluoromethoxybenzoyl)-5-methoxy-6-fluoro-2-methyl-1H-indol-3-yl]-N-ethylacetamide
2-[1-(4-trifluoromethoxybenzoyl)-5-methoxy-6-fluoro-2-methyl-1H-indol-3-yl]-N-propylacetamide
2-[1-(4-trifluoromethoxybenzoyl)-5-methoxy-6-fluoro-2-methyl-1H-indol-3-yl]-N-isopropylacetamide
2-[1-(4-trifluoromethoxybenzoyl)-5-methoxy-6-fluoro-2-methyl-1H-indol-3-yl]-N-butylacetamide
2-[1-(4-trifluoromethoxybenzoyl)-5-methoxy-6-fluoro-2-methyl-1H-indol-3-yl]-N-sec-butylacetamide
2-[1-(4-trifluoromethoxybenzoyl)-5-methoxy-6-fluoro-2-methyl-1H-indol-3-yl]-N-(2-hydroxyethyl)acetamide
methyl[1-(4-trifluoromethylbenzoyl)-5-hydroxy-6-fluoro-2-methyl-1H-indol-3-yl]acetate
ethyl[1-(4-trifluoromethylbenzoyl)-5-hydroxy-6-fluoro-2-methyl-1H-indol-3-yl]acetate
propyl[1-(4-trifluoromethylbenzoyl)-5-hydroxy-6-fluoro-2-methyl-1H-indol-3-yl]acetate
butyl[1-(4-trifluoromethylbenzoyl)-5-hydroxy-6-fluoro-2-methyl-1H-indol-3-yl]acetate
sec-butyl[1-(4-trifluoromethylbenzoyl)-5-hydroxy-6-fluoro-2-methyl-1H-indol-3-yl]acetate
(R)sec-butyl[1-(4-trifluoromethylbenzoyl)-5-hydroxy-6-fluoro-2-methyl-1H-indol-3-yl]acetate
(S)sec-butyl[1-(4-trifluoromethylbenzoyl)-5-hydroxy-6-fluoro-2-methyl-1H-indol-3-yl]acetate
isopropyl[1-(4-trifluoromethylbenzoyl)-5-hydroxy-6-fluoro-2-methyl-1H-indol-3-yl]acetate
tert-butyl[1-(4-trifluoromethylbenzoyl)-5-hydroxy-6-fluoro-2-methyl-1H-indol-3-yl]acetate
cyclobutyl[1-(4-trifluoromethylbenzoyl)-5-hydroxy-6-fluoro-2-methyl-1H-indol-3-yl]acetate
cyclopentyl[1-(4-trifluoromethylbenzoyl)-5-hydroxy-6-fluoro-2-methyl-1H-indol-3-yl]acetate
2-[1-(4-trifluoromethylbenzoyl)-5-hydroxy-6-fluoro-2-methyl-1H-indol-3-yl]acetamide
2-[1-(4-trifluoromethylbenzoyl)-5-hydroxy-6-fluoro-2-methyl-1H-indol-3-yl]-N-methylacetamide
2-[1-(4-trifluoromethylbenzoyl)-5-hydroxy-6-fluoro-2-methyl-1H-indol-3-yl]-N-ethylacetamide
2-[1-(4-trifluoromethylbenzoyl)-5-hydroxy-6-fluoro-2-methyl-1H-indol-3-yl]-N-propylacetamide
2-[1-(4-trifluoromethylbenzoyl)-5-hydroxy-6-fluoro-2-methyl-1H-indol-3-yl]-N-isopropylacetamide
2-[1-(4-trifluoromethylbenzoyl)-5-hydroxy-6-fluoro-2-methyl-1H-indol-3-yl]-N-butylacetamide
2-[1-(4-trifluoromethylbenzoyl)-5-hydroxy-6-fluoro-2-methyl-1H-indol-3-yl]-N-sec-butylacetamide
2-[1-(4-trifluoromethylbenzoyl)-5-hydroxy-6-fluoro-2-methyl-1H-indol-3-yl]-N-(2-hydroxyethyl)acetamide
methyl[1-(4-trifluoromethylbenzoyl)-5-methoxy-6-fluoro-2-methyl-1H-indol-3-yl]acetate
ethyl[1-(4-trifluoromethylbenzoyl)-5-methoxy-6-fluoro-2-methyl-1H-indol-3-yl]acetate
propyl[1-(4-trifluoromethylbenzoyl)-5-methoxy-6-fluoro-2-methyl-1H-indol-3-yl]acetate
butyl[1-(4-trifluoromethylbenzoyl)-5-methoxy-6-fluoro-2-methyl-1H-indol-3-yl]acetate
sec-butyl[1-(4-trifluoromethylbenzoyl)-5-methoxy-6-fluoro-2-methyl-1H-indol-3-yl]acetate
(R)sec-butyl[1-(4-trifluoromethylbenzoyl)-5-methoxy-6-fluoro-2-methyl-1H-indol-3-yl]acetate
(S)sec-butyl[1-(4-trifluoromethylbenzoyl)-5-methoxy-6-fluoro-2-methyl-1H-indol-3-yl]acetate
isopropyl[1-(4-trifluoromethylbenzoyl)-5-methoxy-6-fluoro-2-methyl-1H-indol-3-yl]acetate
tert-butyl[1-(4-trifluoromethylbenzoyl)-5-methoxy-6-fluoro-2-methyl-1H-indol-3-yl]acetate
cyclobutyl[1-(4-trifluoromethylbenzoyl)-5-methoxy-6-fluoro-2-methyl-1H-indol-3-yl]acetate
cyclopentyl[1-(4-trifluoromethylbenzoyl)-5-methoxy-6-fluoro-2-methyl-1H-indol-3-yl]acetate
2-[1-(4-trifluoromethylbenzoyl)-5-methoxy-6-fluoro-2-methyl-1H-indol-3-yl]acetamide
2-[1-(4-trifluoromethylbenzoyl)-5-methoxy-6-fluoro-2-methyl-1H-indol-3-yl]-N-methylacetamide
2-[1-(4-trifluoromethylbenzoyl)-5-methoxy-6-fluoro-2-methyl-1H-indol-3-yl]-N-ethylacetamide
2-[1-(4-trifluoromethylbenzoyl)-5-methoxy-6-fluoro-2-methyl-1H-indol-3-yl]-N-propylacetamide
2-[1-(4-trifluoromethylbenzoyl)-5-methoxy-6-fluoro-2-methyl-1H-indol-3-yl]-N-isopropylacetamide
2-[1-(4-trifluoromethylbenzoyl)-5-methoxy-6-fluoro-2-methyl-1H-indol-3-yl]-N-butylacetamide
2-[1-(4-trifluoromethylbenzoyl)-5-methoxy-6-fluoro-2-methyl-1H-indol-3-yl]-N-sec-butylacetamide
2-[1-(4-trifluoromethylbenzoyl)-5-methoxy-6-fluoro-2-methyl-1H-indol-3-yl]-N-(2-hydroxyethyl)acetamide
methyl[1-(4-chlorobenzoyl)-5-hydroxy-6-fluoro-2-methyl-1H-indol-3-yl]acetate
ethyl[1-(4-chlorobenzoyl)-5-hydroxy-6-fluoro-2-methyl-1H-indol-3-yl]acetate
propyl[1-(4-chlorobenzoyl)-5-hydroxy-6-fluoro-2-methyl-1H-indol-3-yl]acetate
butyl[1-(4-chlorobenzoyl)-5-hydroxy-6-fluoro-2-methyl-1H-indol-3-yl]acetate
sec-butyl[1-(4-chlorobenzoyl)-5-hydroxy-6-fluoro-2-methyl-1H-indol-3-yl]acetate
(R)sec-butyl[1-(4-chlorobenzoyl)-5-hydroxy-6-fluoro-2-methyl-1H-indol-3-yl]acetate
(S)sec-butyl[1-(4-chlorobenzoyl)-5-hydroxy-6-fluoro-2-methyl-1H-indol-3-yl]acetate
isopropyl[1-(4-chlorobenzoyl)-5-hydroxy-6-fluoro-2-methyl-1H-indol-3-yl]acetate
tert-butyl[1-(4-chlorobenzoyl)-5-hydroxy-6-fluoro-2-methyl-1H-indol-3-yl]acetate
cyclobutyl[1-(4-chlorobenzoyl)-5-hydroxy-6-fluoro-2-methyl-1H-indol-3-yl]acetate
cyclopentyl[1-(4-chlorobenzoyl)-5-hydroxy-6-fluoro-2-methyl-1H-indol-3-yl]acetate
2-[1-(4-chlorobenzoyl)-5-hydroxy-6-fluoro-2-methyl-1H-indol-3-yl]acetamide
2-[1-(4-chlorobenzoyl)-5-hydroxy-6-fluoro-2-methyl-1H-indol-3-yl]-N-methylacetamide
2-[1-(4-chlorobenzoyl)-5-hydroxy-6-fluoro-2-methyl-1H-indol-3-yl]-N-ethylacetamide
2-[1-(4-chlorobenzoyl)-5-hydroxy-6-fluoro-2-methyl-1H-indol-3-yl]-N-propylacetamide
2-[1-(4-chlorobenzoyl)-5-hydroxy-6-fluoro-2-methyl-1H-indol-3-yl]-N-isopropylacetamide
2-[1-(4-chlorobenzoyl)-5-hydroxy-6-fluoro-2-methyl-1H-indol-3-yl]-N-butylacetamide
2-[1-(4-chlorobenzoyl)-5-hydroxy-6-fluoro-2-methyl-1H-indol-3-yl]-N-sec-butylacetamide
2-[1-(4-chlorobenzoyl)-5-hydroxy-6-fluoro-2-methyl-1H-indol-3-yl]-N-(2-hydroxyethyl)acetamide
methyl[1-(4-fluorobenzoyl)-5-methoxy-6-fluoro-2-methyl-1H-indol-3-yl]acetate
ethyl[1-(4-fluorobenzoyl)-5-methoxy-6-fluoro-2-methyl-1H-indol-3-yl]acetate
propyl[1-(4-fluorobenzoyl)-5-methoxy-6-fluoro-2-methyl-1H-indol-3-yl]acetate
butyl[1-(4-fluorobenzoyl)-5-methoxy-6-fluoro-2-methyl-1H-indol-3-yl]acetate
sec-butyl[1-(4-fluorobenzoyl)-5-methoxy-6-fluoro-2-methyl-1H-indol-3-yl]acetate
(R)sec-butyl[1-(4-fluorobenzoyl)-5-methoxy-6-fluoro-2-methyl-1H-indol-3-yl]acetate
(S)sec-butyl[1-(4-fluorobenzoyl)-5-methoxy-6-fluoro-2-methyl-1H-indol-3-yl]acetate
isopropyl[1-(4-fluorobenzoyl)-5-methoxy-6-fluoro-2-methyl-1H-indol-3-yl]acetate
tert-butyl[1-(4-fluorobenzoyl)-5-methoxy-6-fluoro-2-methyl-1H-indol-3-yl]acetate
cyclobutyl[1-(4-fluorobenzoyl)-5-methoxy-6-fluoro-2-methyl-1H-indol-3-yl]acetate
cyclopentyl[1-(4-fluorobenzoyl)-5-methoxy-6-fluoro-2-methyl-1H-indol-3-yl]acetate
2-[1-(4-fluorobenzoyl)-5-methoxy-6-fluoro-2-methyl-1H-indol-3-yl]acetamide
2-[1-(4-fluorobenzoyl)-5-methoxy-6-fluoro-2-methyl-1H-indol-3-yl]-N-methylacetamide
2-[1-(4-fluorobenzoyl)-5-methoxy-6-fluoro-2-methyl-1H-indol-3-yl]-N-ethylacetamide
2-[1-(4-fluorobenzoyl)-5-methoxy-6-fluoro-2-methyl-1H-indol-3-yl]-N-propylacetamide
2-[1-(4-fluorobenzoyl)-5-methoxy-6-fluoro-2-methyl-1H-indol-3-yl]-N-isopropylacetamide
2-[1-(4-fluorobenzoyl)-5-methoxy-6-fluoro-2-methyl-1H-indol-3-yl]-N-butylacetamide
2-[1-(4-fluorobenzoyl)-5-methoxy-6-fluoro-2-methyl-1H-indol-3-yl]-N-sec-butylacetamide
2-[1-(4-fluorobenzoyl)-5-methoxy-6-fluoro-2-methyl-1H-indol-3-yl]-N-(2-hydroxyethyl)acetamide
methyl[1-(4-fluorobenzoyl)-5-hydroxy-6-fluoro-2-methyl-1H-indol-3-yl]acetate
ethyl[1-(4-fluorobenzoyl)-5-hydroxy-6-fluoro-2-methyl-1H-indol-3-yl]acetate
propyl[1-(4-fluorobenzoyl)-5-hydroxy-6-fluoro-2-methyl-1H-indol-3-yl]acetate
butyl[1-(4-fluorobenzoyl)-5-hydroxy-6-fluoro-2-methyl-1H-indol-3-yl]acetate
sec-butyl[1-(4-fluorobenzoyl)-5-hydroxy-6-fluoro-2-methyl-1H-indol-3-yl]acetate
(R)sec-butyl[1-(4-fluorobenzoyl)-5-hydroxy-6-fluoro-2-methyl-1H-indol-3-yl]acetate
(S)sec-butyl[1-(4-fluorobenzoyl)-5-hydroxy-6-fluoro-2-methyl-1H-indol-3-yl]acetate
isopropyl[1-(4-fluorobenzoyl)-5-hydroxy-6-fluoro-2-methyl-1H-indol-3-yl]acetate
tert-butyl[1-(4-fluorobenzoyl)-5-hydroxy-6-fluoro-2-methyl-1H-indol-3-yl]acetate
cyclobutyl[1-(4-fluorobenzoyl)-5-hydroxy-6-fluoro-2-methyl-1H-indol-3-yl]acetate
cyclopentyl[1-(4-fluorobenzoyl)-5-hydroxy-6-fluoro-2-methyl-1H-indol-3-yl]acetate
2-[1-(4-fluorobenzoyl)-5-hydroxy-6-fluoro-2-methyl-1H-indol-3-yl]acetamide
2-[1-(4-fluorobenzoyl)-5-hydroxy-6-fluoro-2-methyl-1H-indol-3-yl]-N-methylacetamide
2-[1-(4-fluorobenzoyl)-5-hydroxy-6-fluoro-2-methyl-1H-indol-3-yl]-N-ethylacetamide
2-[1-(4-fluorobenzoyl)-5-hydroxy-6-fluoro-2-methyl-1H-indol-3-yl]-N-propylacetamide
2-[1-(4-fluorobenzoyl)-5-hydroxy-6-fluoro-2-methyl-1H-indol-3-yl]-N-isopropylacetamide
2-[1-(4-fluorobenzoyl)-5-hydroxy-6-fluoro-2-methyl-1H-indol-3-yl]-N-butylacetamide
2-[1-(4-fluorobenzoyl)-5-hydroxy-6-fluoro-2-methyl-1H-indol-3-yl]-N-sec-butylacetamide
2-[1-(4-fluorobenzoyl)-5-hydroxy-6-fluoro-2-methyl-1H-indol-3-yl]-N-(2-hydroxyethyl)acetamide
methyl[1-(4-bromobenzoyl)-5-hydroxy-6-fluoro-2-methyl-1H-indol-3-yl]acetate
ethyl[1-(4-bromobenzoyl)-5-hydroxy-6-fluoro-2-methyl-1H-indol-3-yl]acetate
propyl[1-(4-bromobenzoyl)-5-hydroxy-6-fluoro-2-methyl-1H-indol-3-yl]acetate
butyl[1-(4-bromobenzoyl)-5-hydroxy-6-fluoro-2-methyl-1H-indol-3-yl]acetate
sec-butyl[1-(4-bromobenzoyl)-5-hydroxy-6-fluoro-2-methyl-1H-indol-3-yl]acetate
(R)sec-butyl[1-(4-bromobenzoyl)-5-hydroxy-6-fluoro-2-methyl-1H-indol-3-yl]acetate
(S)sec-butyl[1-(4-bromobenzoyl)-5-hydroxy-6-fluoro-2-methyl-1H-indol-3-yl]acetate
isopropyl[1-(4-bromobenzoyl)-5-hydroxy-6-fluoro-2-methyl-1H-indol-3-yl]acetate
tert-butyl[1-(4-bromobenzoyl)-5-hydroxy-6-fluoro-2-methyl-1H-indol-3-yl]acetate
cyclobutyl[1-(4-bromobenzoyl)-5-hydroxy-6-fluoro-2-methyl-1H-indol-3-yl]acetate
cyclopentyl[1-(4-bromobenzoyl)-5-hydroxy-6-fluoro-2-methyl-1H-indol-3-yl]acetate
2-[1-(4-bromobenzoyl)-5-hydroxy-6-fluoro-2-methyl-1H-indol-3-yl]acetamide
2-[1-(4-bromobenzoyl)-5-hydroxy-6-fluoro-2-methyl-1H-indol-3-yl]-N-methylacetamide
2-[1-(4-bromobenzoyl)-5-hydroxy-6-fluoro-2-methyl-1H-indol-3-yl]-N-ethylacetamide
2-[1-(4-bromobenzoyl)-5-hydroxy-6-fluoro-2-methyl-1H-indol-3-yl]-N-propylacetamide
2-[1-(4-bromobenzoyl)-5-hydroxy-6-fluoro-2-methyl-1H-indol-3-yl]-N-isopropylacetamide
2-[1-(4-bromobenzoyl)-5-hydroxy-6-fluoro-2-methyl-1H-indol-3-yl]-N-butylacetamide
2-[1-(4-bromobenzoyl)-5-hydroxy-6-fluoro-2-methyl-1H-indol-3-yl]-N-sec-butylacetamide
2-[1-(4-bromobenzoyl)-5-hydroxy-6-fluoro-2-methyl-1H-indol-3-yl]-N-(2-hydroxyethyl)acetamide
methyl[1-(4-bromobenzoyl)-5-methoxy-6-fluoro-2-methyl-1H-indol-3-yl]acetate
ethyl[1-(4-bromobenzoyl)-5-methoxy-6-fluoro-2-methyl-1H-indol-3-yl]acetate
propyl[1-(4-bromobenzoyl)-5-methoxy-6-fluoro-2-methyl-1H-indol-3-yl]acetate
butyl[1-(4-bromobenzoyl)-5-methoxy-6-fluoro-2-methyl-1H-indol-3-yl]acetate
sec-butyl[1-(4-bromobenzoyl)-5-methoxy-6-fluoro-2-methyl-1H-indol-3-yl]acetate
(R)sec-butyl[1-(4-bromobenzoyl)-5-methoxy-6-fluoro-2-methyl-1H-indol-3-yl]acetate
(S)sec-butyl[1-(4-bromobenzoyl)-5-methoxy-6-fluoro-2-methyl-1H-indol-3-yl]acetate
isopropyl[1-(4-bromobenzoyl)-5-methoxy-6-fluoro-2-methyl-1H-indol-3-yl]acetate
tert-butyl[1-(4-bromobenzoyl)-5-methoxy-6-fluoro-2-methyl-1H-indol-3-yl]acetate
cyclobutyl[1-(4-bromobenzoyl)-5-methoxy-6-fluoro-2-methyl-1H-indol-3-yl]acetate
cyclopentyl[1-(4-bromobenzoyl)-5-methoxy-6-fluoro-2-methyl-1H-indol-3-yl]acetate
2-[1-(4-bromobenzoyl)-5-methoxy-6-fluoro-2-methyl-1H-indol-3-yl]acetamide
2-[1-(4-bromobenzoyl)-5-methoxy-6-fluoro-2-methyl-1H-indol-3-yl]-N-methyl acetamide
2-[1-(4-bromobenzoyl)-5-methoxy-6-fluoro-2-methyl-1H-indol-3-yl]-N-ethylacetamide
2-[1-(4-bromobenzoyl)-5-methoxy-6-fluoro-2-methyl-1H-indol-3-yl]-N-propylacetamide
2-[1-(4-bromobenzoyl)-5-methoxy-6-fluoro-2-methyl-1H-indol-3-yl]-N-isopropylacetamide
2-[1-(4-bromobenzoyl)-5-methoxy-6-fluoro-2-methyl-1H-indol-3-yl]-N-butylacetamide
2-[1-(4-bromobenzoyl)-5-methoxy-6-fluoro-2-methyl-1H-indol-3-yl]-N-sec-butylacetamide
2-[1-(4-bromobenzoyl)-5-methoxy-6-fluoro-2-methyl-1H-indol-3-yl]-N-(2-hydroxyethyl)acetamide
methyl[1-(4-bromobenzoyl)-5-hydroxy-2-methyl-1H-indol-3-yl]acetate
ethyl[1-(4-bromobenzoyl)-5-hydroxy-2-methyl-1H-indol-3-yl]acetate
propyl[1-(4-bromobenzoyl)-5-hydroxy-2-methyl-1H-indol-3-yl]acetate
butyl[1-(4-bromobenzoyl)-5-hydroxy-2-methyl-1H-indol-3-yl]acetate
sec-butyl[1-(4-bromobenzoyl)-5-hydroxy-2-methyl-1H-indol-3-yl]acetate
(R)sec-butyl[1-(4-bromobenzoyl)-5-hydroxy-2-methyl-1H-indol-3-yl]acetate
(S)sec-butyl[1-(4-bromobenzoyl)-5-hydroxy-2-methyl-1H-indol-3-yl]acetate
isopropyl[1-(4-bromobenzoyl)-5-hydroxy-2-methyl-1H-indol-3-yl]acetate
tert-butyl[1-(4-bromobenzoyl)-5-hydroxy-2-methyl-1H-indol-3-yl]acetate
cyclobutyl[1-(4-bromobenzoyl)-5-hydroxy-2-methyl-1H-indol-3-yl]acetate
cyclopentyl[1-(4-bromobenzoyl)-5-hydroxy-2-methyl-1H-indol-3-yl]acetate
2-[1-(4-bromobenzoyl)-5-hydroxy-2-methyl-1H-indol-3-yl]acetamide
2-[1-(4-bromobenzoyl)-5-hydroxy-2-methyl-1H-indol-3-yl]-N-methylacetamide
2-[1-(4-bromobenzoyl)-5-hydroxy-2-methyl-1H-indol-3-yl]-N-ethylacetamide
2-[1-(4-bromobenzoyl)-5-hydroxy-2-methyl-1H-indol-3-yl]-N-propylacetamide
2-[1-(4-bromobenzoyl)-5-hydroxy-2-methyl-1H-indol-3-yl]-N-isopropylacetamide
2-[1-(4-bromobenzoyl)-5-hydroxy-2-methyl-1H-indol-3-yl]-N-butylacetamide
2-[1-(4-bromobenzoyl)-5-hydroxy-2-methyl-1H-indol-3-yl]-N-sec-butylacetamide
2-[1-(4-bromobenzoyl)-5-hydroxy-2-methyl-1H-indol-3-yl]-N-(2-hydroxyethyl)acetamide
methyl[1-(4-methylbenzoyl)-5-methoxy-6-fluoro-2-methyl-1H-indol-3-yl]acetate
ethyl[1-(4-methylbenzoyl)-5-methoxy-6-fluoro-2-methyl-1H-indol-3-yl]acetate
propyl[1-(4-methylbenzoyl)-5-methoxy-6-fluoro-2-methyl-1H-indol-3-yl]acetate
butyl[1-(4-methylbenzoyl)-5-methoxy-6-fluoro-2-methyl-1H-indol-3-yl]acetate
sec-butyl[1-(4-methylbenzoyl)-5-methoxy-6-fluoro-2-methyl-1H-indol-3-yl]acetate
(R)sec-butyl[1-(4-methylbenzoyl)-5-methoxy-6-fluoro-2-methyl-1H-indol-3-yl]acetate
(S)sec-butyl[1-(4-methylbenzoyl)-5-methoxy-6-fluoro-2-methyl-1H-indol-3-yl]acetate
isopropyl[1-(4-methylbenzoyl)-5-methoxy-6-fluoro-2-methyl-1H-indol-3-yl]acetate
tert-butyl[1-(4-methylbenzoyl)-5-methoxy-6-fluoro-2-methyl-1H-indol-3-yl]acetate
cyclobutyl[1-(4-methylbenzoyl)-5-methoxy-6-fluoro-2-methyl-1H-indol-3-yl]acetate
cyclopentyl[1-(4-methylbenzoyl)-5-methoxy-6-fluoro-2-methyl-1H-indol-3-yl]acetate
2-[1-(4-methylbenzoyl)-5-methoxy-6-fluoro-2-methyl-1H-indol-3-yl]acetamide
2-[1-(4-methylbenzoyl)-5-methoxy-6-fluoro-2-methyl-1H-indol-3-yl]-N-methylacetamide
2-[1-(4-methylbenzoyl)-5-methoxy-6-fluoro-2-methyl 1H-indol-3-yl]-N-ethylacetamide
2-[1-(4-methylbenzoyl)-5-methoxy-6-fluoro-2-methyl-1H-indol-3-yl]-N-propylacetamide
2-[1-(4-methylbenzoyl)-5-methoxy-6-fluoro-2-methyl-1H-indol-3-yl]-N-isopropylacetamide
2-[1-(4-methylbenzoyl)-5-methoxy-6-fluoro-2-methyl-1H-indol-3-yl]-N-butylacetamide
2-[1-(4-methylbenzoyl)-5-methoxy-6-fluoro-2-methyl-1H-indol-3-yl]-N-sec-butylacetamide
2-[1-(4-methylbenzoyl)-5-methoxy-6-fluoro-2-methyl-1H-indol-3-yl]-N-(2-hydroxyethyl)acetamide
methyl[1-(4-methylbenzoyl)-5-hydroxy-6-fluoro-2-methyl-1H-indol-3-yl]acetate
ethyl[1-(4-methylbenzoyl)-5-hydroxy-6-fluoro-2-methyl-1H-indol-3-yl]acetate
propyl[1-(4-methylbenzoyl)-5-hydroxy-6-fluoro-2-methyl-1H-indol-3-yl]acetate
butyl[1-(4-methylbenzoyl)-5-hydroxy-6-fluoro-2-methyl-1H-indol-3-yl]acetate
sec-butyl[1-(4-methylbenzoyl)-5-hydroxy-6-fluoro-2-methyl-1H-indol-3-yl]acetate
(R)sec-butyl[1-(4-methylbenzoyl)-5-hydroxy-6-fluoro-2-methyl-1H-indol-3-yl]acetate
(S)sec-butyl[1-(4-methylbenzoyl)-5-hydroxy-6-fluoro-2-methyl-1H-indol-3-yl]acetate
isopropyl[1-(4-methylbenzoyl)-5-hydroxy-6-fluoro-2-methyl-1H-indol-3-yl]acetate
tert-butyl[1-(4-methylbenzoyl)-5-hydroxy-6-fluoro-2-methyl-1H-indol-3-yl]acetate
cyclobutyl[1-(4-methylbenzoyl)-5-hydroxy-6-fluoro-2-methyl-1H-indol-3-yl]acetate
cyclopentyl[1-(4-methylbenzoyl)-5-hydroxy-6-fluoro-2-methyl-1H-indol-3-yl]acetate
2-[1-(4-methylbenzoyl)-5-hydroxy-6-fluoro-2-methyl-1H-indol-3-yl]acetamide
2-[1-(4-methylbenzoyl)-5-hydroxy-6-fluoro-2-methyl-1H-indol-3-yl]-N-methylacetamide
2-[1-(4-methylbenzoyl)-5-hydroxy-6-fluoro-2-methyl-1H-indol-3-yl]-N-ethylacetamide
2-[1-(4-methylbenzoyl)-5-hydroxy-6-fluoro-2-methyl-1H-indol-3-yl]-N-propylacetamide
2-[1-(4-methylbenzoyl)-5-hydroxy-6-fluoro-2-methyl-1H-indol-3-yl]-N-isopropylacetamide
2-[1-(4-methylbenzoyl)-5-hydroxy-6-fluoro-2-methyl-1H-indol-3-yl]-N-butylacetamide
2-[1-(4-methylbenzoyl)-5-hydroxy-6-fluoro-2-methyl-1H-indol-3-yl]-N-sec-butylacetamide
2-[1-(4-methylbenzoyl)-5-hydroxy-6-fluoro-2-methyl-1H-indol-3-yl]-N-(2-hydroxyethyl)acetamide
methyl[1-(4-methylthiobenzoyl)-5-methoxy-6-fluoro-2-methyl-1H-indol-3-yl]acetate
ethyl[1-(4-methylthiobenzoyl)-5-methoxy-6-fluoro-2-methyl-1H-indol-3-yl]acetate
propyl[1-(4-methylthiobenzoyl)-5-methoxy-6-fluoro-2-methyl-1H-indol-3-yl]acetate
butyl[1-(4-methylthiobenzoyl)-5-methoxy-6-fluoro-2-methyl-1H-indol-3-yl]acetate
sec-butyl[1-(4-methylthiobenzoyl)-5-methoxy-6-fluoro-2-methyl-1H-indol-3-yl]acetate
(R)sec-butyl[1-(4-methylthiobenzoyl)-5-methoxy-6-fluoro-2-methyl-1H-indol-3-yl]acetate
(S)sec-butyl[1-(4-methylthiobenzoyl)-5-methoxy-6-fluoro-2-methyl-1H-indol-3-yl]acetate
isopropyl[1-(4-methylthiobenzoyl)-5-methoxy-6-fluoro-2-methyl-1H-indol-3-yl]acetate
tert-butyl[1-(4-methylthiobenzoyl)-5-methoxy-6-fluoro-2-methyl-1H-indol-3-yl]acetate
cyclobutyl[1-(4-methylthiobenzoyl)-5-methoxy-6-fluoro-2-methyl-1H-indol-3-yl]acetate
cyclopentyl[1-(4-methylthiobenzoyl)-5-methoxy-6-fluoro-2-methyl-1H-indol-3-yl]acetate
2-[1-(4-methylthiobenzoyl)-5-methoxy-6-fluoro-2-methyl-1H-indol-3-yl]acetamide
2-[1-(4-methylthiobenzoyl)-5-methoxy-6-fluoro-2-methyl-1H-indol-3-yl]-N-methylacetamide
2-[1-(4-methylthiobenzoyl)-5-methoxy-6-fluoro-2-methyl-1H-indol-3-yl]-N-ethylacetamide
2-[1-(4-methylthiobenzoyl)-5-methoxy-6-fluoro-2-methyl-1H-indol-3-yl]-N-propylacetamide
2-[1-(4-methylthiobenzoyl)-5-methoxy-6-fluoro-2-methyl-1H-indol-3-yl]-N-isopropylacetamide
2-[1-(4-methylthiobenzoyl)-5-methoxy-6-fluoro-2-methyl-1H-indol-3-yl]-N-butylacetamide
2-[1-(4-methylthiobenzoyl)-5-methoxy-6-fluoro-2-methyl-1H-indol-3-yl]-N-sec-butylacetamide
2-[1-(4-methylthiobenzoyl)-5-methoxy-6-fluoro-2-methyl-1H-indol-3-yl]-N-(2-hydroxyethyl)acetamide
methyl[1-(4-methylthiobenzoyl)-5-hydroxy-6-fluoro-2-methyl-1H-indol-3-yl]acetate
ethyl[1-(4-methylthiobenzoyl)-5-hydroxy-6-fluoro-2-methyl-1H-indol-3-yl]acetate
propyl[1-(4-methylthiobenzoyl)-5-hydroxy-6-fluoro-2-methyl-1H-indol-3-yl]acetate
butyl[1-(4-methylthiobenzoyl)-5-hydroxy-6-fluoro-2-methyl-1H-indol-3-yl]acetate
sec-butyl[1-(4-methylthiobenzoyl)-5-hydroxy-6-fluoro-2-methyl-1H-indol-3-yl]acetate
(R)sec-butyl[1-(4-methylthiobenzoyl)-5-hydroxy-6-fluoro-2-methyl-1H-indol-3-yl]acetate
(S)sec-butyl[1-(4-methylthiobenzoyl)-5-hydroxy-6-fluoro-2-methyl-1H-indol-3-yl]acetate
isopropyl[1-(4-methylthiobenzoyl)-5-hydroxy-6-fluoro-2-methyl-1H-indol-3-yl]acetate
tert-butyl[1-(4-methylthiobenzoyl)-5-hydroxy-6-fluoro-2-methyl-1H-indol-3-yl]acetate
cyclobutyl[1-(4-methylthiobenzoyl)-5-hydroxy-6-fluoro-2-methyl-1H-indol-3-yl]acetate
cyclopentyl[1-(4-methylthiobenzoyl)-5-hydroxy-6-fluoro-2-methyl-1H-indol-3-yl]acetate
2-[1-(4-methylthiobenzoyl)-5-hydroxy-6-fluoro-2-methyl-1H-indol-3-yl]acetamide
2-[1-(4-methylthiobenzoyl)-5-hydroxy-6-fluoro-2-methyl-1H-indol-3-yl]-N-methylacetamide
2-[1-(4-methylthiobenzoyl)-5-hydroxy-6-fluoro-2-methyl-1H-indol-3-yl]-N-ethylacetamide
2-[1-(4-methylthiobenzoyl)-5-hydroxy-6-fluoro-2-methyl-1H-indol-3-yl]-N-propylacetamide
2-[1-(4-methylthiobenzoyl)-5-hydroxy-6-fluoro-2-methyl-1H-indol-3-yl]-N-isopropylacetamide
2-[1-(4-methylthiobenzoyl)-5-hydroxy-6-fluoro-2-methyl-1H-indol-3-yl]-N-butylacetamide
2-[1-(4-methylthiobenzoyl)-5-hydroxy-6-fluoro-2-methyl-1H-indol-3-yl]-N-sec-butylacetamide
2-[1-(4-methylthiobenzoyl)-5-hydroxy-6-fluoro-2-methyl-1H-indol-3-yl]-N-(2-hydroxyethyl)acetamide
methyl[1-(4-chlorobenzoyl)-5-hydroxy-2-methyl-1H-indol-3-yl]acetate
ethyl[1-(4-chlorobenzoyl)-5-hydroxy-2-methyl-1H-indol-3-yl]acetate
propyl[1-(4-chlorobenzoyl)-5-hydroxy-2-methyl-1H-indol-3-yl]acetate
butyl[1-(4-chlorobenzoyl)-5-hydroxy-2-methyl-1H-indol-3-yl]acetate
sec-butyl[1-(4-chlorobenzoyl)-5-hydroxy-2-methyl-1H-indol-3-yl]acetate
(R)sec-butyl[1-(4-chlorobenzoyl)-5-hydroxy-2-methyl-1H-indol-3-yl]acetate
(S)sec-butyl[1-(4-chlorobenzoyl)-5-hydroxy-2-methyl-1H-indol-3-yl]acetate
isopropyl[1-(4-chlorobenzoyl)-5-hydroxy-2-methyl-1H-indol-3-yl]acetate
tert-butyl[1-(4-chlorobenzoyl)-5-hydroxy-2-methyl-1H-indol-3-yl]acetate
cyclobutyl[1-(4-chlorobenzoyl)-5-hydroxy-2-methyl-1H-indol-3-yl]acetate
cyclopentyl[1-(4-chlorobenzoyl)-5-hydroxy-2-methyl-1H-indol-3-yl]acetate
2-[1-(4-chlorobenzoyl)-5-hydroxy-2-methyl-1H-indol-3-yl]acetamide
2-[1-(4-chlorobenzoyl)-5-hydroxy-2-methyl-1H-indol-3-yl]-N-methylacetamide
2-[1-(4-chlorobenzoyl)-5-hydroxy-2-methyl-1H-indol-3-yl]-N-ethylacetamide
2-[1-(4-chlorobenzoyl)-5-hydroxy-2-methyl-1H-indol-3-yl]-N-propylacetamide
2-[1-(4-chlorobenzoyl)-5-hydroxy-2-methyl-1H-indol-3-yl]-N-isopropylacetamide
2-[1-(4-chlorobenzoyl)-5-hydroxy-2-methyl-1H-indol-3-yl]-N-butylacetamide
2-[1-(4-chlorobenzoyl)-5-hydroxy-2-methyl-1H-indol-3-yl]-N-sec-butylacetamide
2-[1-(4-chlorobenzoyl)-5-hydroxy-2-methyl-1H-indol-3-yl]-N-(2-hydroxyethyl)acetamide
methyl{1-[4-fluorobenzoyl]-6-chloro-5-methoxy-2-methyl-1H-indol-3-yl]acetate
ethyl{1-[4-fluorobenzoyl]-6-chloro-5-methoxy-2-methyl-1H-indol-3-yl]acetate
propyl{1-[4-fluorobenzoyl]-6-chloro-5-methoxy-2-methyl-1H-indol-3-yl]acetate
butyl{1-[4-fluorobenzoyl]-6-chloro-5-methoxy-2-methyl-1H-indol-3-yl]acetate
sec-butyl{1-[4-fluorobenzoyl]-6-chloro-5-methoxy-2-methyl-1H-indol-3-yl]acetate
(R)sec-butyl{1-[4-fluorobenzoyl]-6-chloro-5-methoxy-2-methyl-1H-indol-3-yl]acetate
(S)sec-butyl{1-[4-fluorobenzoyl]-6-chloro-5-methoxy-2-methyl-1H-indol-3-yl]acetate
isopropyl{1-[4-fluorobenzoyl]-6-chloro-5-methoxy-2-methyl-1H-indol-3-yl]acetate
tert-butyl{1-[4-fluorobenzoyl]-6-chloro-5-methoxy-2-methyl-1H-indol-3-yl]acetate
cyclobutyl{1-[4-fluorobenzoyl]-6-chloro-5-methoxy-2-methyl-1H-indol-3-yl]acetate
cyclopentyl{1-[4-fluorobenzoyl]-6-chloro-5-methoxy-2-methyl-1H-indol-3-yl]acetate
2-{1-[4-fluorobenzoyl]-6-chloro-5-methoxy-2-methyl-1H-indol-3-yl]acetamide
2-{1-[4-fluorobenzoyl]-6-chloro-5-methoxy-2-methyl-1H-indol-3-yl]-N-methylacetamide
2-{1-[4-fluorobenzoyl]-6-chloro-5-methoxy-2-methyl-1H-indol-3-yl]-N-ethylacetamide
2-{1-[4-fluorobenzoyl]-6-chloro-5-methoxy-2-methyl-1H-indol-3-yl]-N-propylacetamide
2-{1-[4-fluorobenzoyl]-6-chloro-5-methoxy-2-methyl-1H-indol-3-yl]-N-isopropylacetamide
2-{1-[4-fluorobenzoyl]-6-chloro-5-methoxy-2-methyl-1H-indol-3-yl]-N-butylacetamide
2-{1-[4-fluorobenzoyl]-6-chloro-5-methoxy-2-methyl-1H-indol-3-yl]-N-sec-butylacetamide
2-{1-[4-fluorobenzoyl]-6-chloro-5-methoxy-2-methyl-1H-indol-3-yl]-N-(2-hydroxyethyl)acetamide
methyl{1-[4-fluorobenzoyl]-6-chloro-5-hydroxy-2-methyl-1H-indol-3-yl]acetate
ethyl{1-[4-fluorobenzoyl]-6-chloro-5-hydroxy-2-methyl-1H-indol-3-yl]acetate
propyl{1-[4-fluorobenzoyl]-6-chloro-5-hydroxy-2-methyl-1H-indol-3-yl]acetate
butyl{1-[4-fluorobenzoyl]-6-chloro-5-hydroxy-2-methyl-1H-indol-3-yl]acetate
sec-butyl{1-[4-fluorobenzoyl]-6-chloro-5-hydroxy-2-methyl-1H-indol-3-yl]acetate
(R)sec-butyl{1-[4-fluorobenzoyl]-6-chloro-5-hydroxy-2-methyl-1H-indol-3-yl]acetate
(S)sec-butyl{1-[4-fluorobenzoyl]-6-chloro-5-hydroxy-2-methyl-1H-indol-3-yl]acetate
isopropyl{1-[4-fluorobenzoyl]-6-chloro-5-hydroxy-2-methyl-1H-indol-3-yl]acetate
tert-butyl{1-[4-fluorobenzoyl]-6-chloro-5-hydroxy-2-methyl-1H-indol-3-yl]acetate
cyclobutyl{1-[4-fluorobenzoyl]-6-chloro-5-hydroxy-2-methyl-1H-indol-3-yl]acetate
cyclopentyl{1-[4-fluorobenzoyl]-6-chloro-5-hydroxy-2-methyl-1H-indol-3-yl]acetate
2-{1-[4-fluorobenzoyl]-6-chloro-5-hydroxy-2-methyl-1H-indol-3-yl]acetamide
2-{1-[4-fluorobenzoyl]-6-chloro-5-hydroxy-2-methyl-1H-indol-3-yl]-N-methylacetamide
2-{1-[4-fluorobenzoyl]-6-chloro-5-hydroxy-2-methyl-1H-indol-3-yl]-N-ethylacetamide
2-{1-[4-fluorobenzoyl]-6-chloro-5-hydroxy-2-methyl-1H-indol-3-yl]-N-propylacetamide
2-{1-[4-fluorobenzoyl]-6-chloro-5-hydroxy-2-methyl-1H-indol-3-yl]-N-isopropylacetamide
2-{1-[4-fluorobenzoyl]-6-chloro-5-hydroxy-2-methyl-1H-indol-3-yl]-N-butylacetamide
2-{1-[4-fluorobenzoyl]-6-chloro-5-hydroxy-2-methyl-1H-indol-3-yl]-N-sec-butylacetamide
2-{1-[4-fluorobenzoyl]-6-chloro-5-hydroxy-2-methyl-1H-indol-3-yl]-N-(2-hydroxyethyl)acetamide
methyl{1-[4-bromobenzoyl]-6-chloro-5-methoxy-2-methyl-1H-indol-3-yl]acetate
ethyl{1-[4-bromobenzoyl]-6-chloro-5-methoxy-2-methyl-1H-indol-3-yl]acetate
propyl{1-[4-bromobenzoyl]-6-chloro-5-methoxy-2-methyl-1H-indol-3-yl]acetate
butyl{1-[4-bromobenzoyl]-6-chloro-5-methoxy-2-methyl-1H-indol-3-yl]acetate
sec-butyl{1-[4-bromobenzoyl]-6-chloro-5-methoxy-2-methyl-1H-indol-3-yl]acetate
(R)sec-butyl{1-[4-bromobenzoyl]-6-chloro-5-methoxy-2-methyl-1H-indol-3-yl]acetate
(S)sec-butyl{1-[4-bromobenzoyl]-6-chloro-5-methoxy-2-methyl-1H-indol-3-yl]acetate
isopropyl{1-[4-bromobenzoyl]-6-chloro-5-methoxy-2-methyl-1H-indol-3-yl]acetate
tert-butyl{1-[4-bromobenzoyl]-6-chloro-5-methoxy-2-methyl-1H-indol-3-yl]acetate
cyclobutyl{1-[4-bromobenzoyl]-6-chloro-5-methoxy-2-methyl-1H-indol-3-yl]acetate
cyclopentyl{1-[4-bromobenzoyl]-6-chloro-5-methoxy-2-methyl-1H-indol-3-yl]acetate
2-{1-[4-bromobenzoyl]-6-chloro-5-methoxy-2-methyl-1H-indol-3-yl]acetamide
2-{1-[4-bromobenzoyl]-6-chloro-5-methoxy-2-methyl-1H-indol-3-yl]-N-methylacetamide
2-{1-[4-bromobenzoyl]-6-chloro-5-methoxy-2-methyl-1H-indol-3-yl]-N-ethylacetamide
2-{1-[4-bromobenzoyl]-6-chloro-5-methoxy-2-methyl-1H-indol-3-yl]-N-propylacetamide
2-{1-[4-bromobenzoyl]-6-chloro-5-methoxy-2-methyl-1H-indol-3-yl]-N-isopropylacetamide
2-{1-[4-bromobenzoyl]-6-chloro-5-methoxy-2-methyl-1H-indol-3-yl]-N-butylacetamide
2-{1-[4-bromobenzoyl]-6-chloro-5-methoxy-2-methyl-1H-indol-3-yl]-N-sec-butylacetamide
2-{1-[4-bromobenzoyl]-6-chloro-5-methoxy-2-methyl-1H-indol-3-yl]-N-(2-hydroxyethyl)acetamide
methyl{1-[4-bromobenzoyl]-6-chloro-5-hydroxy-2-methyl-1H-indol-3-yl]acetate
ethyl{1-[4-bromobenzoyl]-6-chloro-5-hydroxy-2-methyl-1H-indol-3-yl]acetate
propyl{1-[4-bromobenzoyl]-6-chloro-5-hydroxy-2-methyl-1H-indol-3-yl]acetate
butyl{1-[4-bromobenzoyl]-6-chloro-5-hydroxy-2-methyl-1H-indol-3-yl]acetate
sec-butyl{1-[4-bromobenzoyl]-6-chloro-5-hydroxy-2-methyl-1H-indol-3-yl]acetate
(R)sec-butyl{1-[4-bromobenzoyl]-6-chloro-5-hydroxy-2-methyl-1H-indol-3-yl]acetate
(S)sec-butyl{1-[4-bromobenzoyl]-6-chloro-5-hydroxy-2-methyl-1H-indol-3-yl]acetate
isopropyl{1-[4-bromobenzoyl]-6-chloro-5-hydroxy-2-methyl-1H-indol-3-yl]acetate
tert-butyl{1-[4-bromobenzoyl]-6-chloro-5-hydroxy-2-methyl-1H-indol-3-yl]acetate
cyclobutyl{1-[4-bromobenzoyl]-6-chloro-5-hydroxy-2-methyl-1H-indol-3-yl]acetate
cyclopentyl{1-[4-bromobenzoyl]-6-chloro-5-hydroxy-2-methyl-1H-indol-3-yl]acetate
2-{1-[4-bromobenzoyl]-6-chloro-5-hydroxy-2-methyl-1H-indol-3-yl]acetamide
2-{1-[4-bromobenzoyl]-6-chloro-5-hydroxy-2-methyl-1H-indol-3-yl]-N-methylacetamide
2-{1-[4-bromobenzoyl]-6-chloro-5-hydroxy-2-methyl-1H-indol-3-yl]-N-ethylacetamide
2-{1-[4-bromobenzoyl]-6-chloro-5-hydroxy-2-methyl-1H-indol-3-yl]-N-propylacetamide
2-{1-[4-bromobenzoyl]-6-chloro-5-hydroxy-2-methyl-1H-indol-3-yl]-N-isopropylacetamide
2-{1-[4-bromobenzoyl]-6-chloro-5-hydroxy-2-methyl-1H-indol-3-yl]-N-butylacetamide
2-{1-[4-bromobenzoyl]-6-chloro-5-hydroxy-2-methyl-1H-indol-3-yl]-N-sec-butylacetamide
2-{1-[4-bromobenzoyl]-6-chloro-5-hydroxy-2-methyl-1H-indol-3-yl]-N-(2-hydroxyethyl)acetamide
methyl{1-[4-trifluoromethylbenzoyl]-6-chloro-5-methoxy-2-methyl-1H-indol-3-yl]acetate
ethyl{1-[4-trifluoromethylbenzoyl]-6-chloro-5-methoxy-2-methyl-1H-indol-3-yl]acetate
propyl{1-[4-trifluoromethylbenzoyl]-6-chloro-5-methoxy-2-methyl-1H-indol-3-yl]acetate
butyl{1-[4-trifluoromethylbenzoyl]-6-chloro-5-methoxy-2-methyl-1H-indol-3-yl]acetate
sec-butyl{1-[4-trifluoromethylbenzoyl]-6-chloro-5-methoxy-2-methyl-1H-indol-3-yl]acetate
(R)sec-butyl{1-[4-trifluoromethylbenzoyl]-6-chloro-5-methoxy-2-methyl-1H-indol-3-yl]acetate
(S)sec-butyl{1-[4-trifluoromethylbenzoyl]-6-chloro-5-methoxy-2-methyl-1H-indol-3-yl]acetate
isopropyl{1-[4-trifluoromethylbenzoyl]-6-chloro-5-methoxy-2-methyl-1H-indol-3-yl]acetate
tert-butyl{1-[4-trifluoromethylbenzoyl]-6-chloro-5-methoxy-2-methyl-1H-indol-3-yl]acetate
cyclobutyl{1-[4-trifluoromethylbenzoyl]-6-chloro-5-methoxy-2-methyl-1H-indol-3-yl]acetate
cyclopentyl{1-[4-trifluoromethylbenzoyl]-6-chloro-5-methoxy-2-methyl-1H-indol-3-yl]acetate
2-{1-[4-trifluoromethylbenzoyl]-6-chloro-5-methoxy-2-methyl-1H-indol-3-yl]acetamide
2-{1-[4-trifluoromethylbenzoyl]-6-chloro-5-methoxy-2-methyl-1H-indol-3-yl]-N-methylacetamide
2-{1-[4-trifluoromethylbenzoyl]-6-chloro-5-methoxy-2-methyl-1H-indol-3-yl]-N-ethylacetamide
2-{1-[4-trifluoromethylbenzoyl]-6-chloro-5-methoxy-2-methyl-1H-indol-3-yl]-N-propylacetamide
2-{1-[4-trifluoromethylbenzoyl]-6-chloro-5-methoxy-2-methyl-1H-indol-3-yl]-N-isopropylacetamide
2-{1-[4-trifluoromethylbenzoyl]-6-chloro-5-methoxy-2-methyl-1H-indol-3-yl]-N-butylacetamide
2-{1-[4-trifluoromethylbenzoyl]-6-chloro-5-methoxy-2-methyl-1H-indol-3-yl]-N-sec-butylacetamide
2-{1-[4-trifluoromethylbenzoyl]-6-chloro-5-methoxy-2-methyl-1H-indol-3-yl]-N-(2-hydroxyethyl)acetamide
methyl{1-[4-trifluoromethylbenzoyl]-6-chloro-5-hydroxy-2-methyl-1H-indol-3-yl]acetate
ethyl{1-[4-trifluoromethylbenzoyl]-6-chloro-5-hydroxy-2-methyl-1H-indol-3-yl]acetate
propyl{1-[4-trifluoromethylbenzoyl]-6-chloro-5-hydroxy-2-methyl-1H-indol-3-yl]acetate
butyl{1-[4-trifluoromethylbenzoyl]-6-chloro-5-hydroxy-2-methyl-1H-indol-3-yl]acetate
sec-butyl{1-[4-trifluoromethylbenzoyl]-6-chloro-5-hydroxy-2-methyl-1H-indol-3-yl]acetate
(R)sec-butyl{1-[4-trifluoromethylbenzoyl]-6-chloro-5-hydroxy-2-methyl-1H-indol-3-yl]acetate
(S)sec-butyl{1-[4-trifluoromethylbenzoyl]-6-chloro-5-hydroxy-2-methyl-1H-indol-3-yl]acetate
isopropyl{1-[4-trifluoromethylbenzoyl]-6-chloro-5-hydroxy-2-methyl-1H-indol-3-yl]acetate
tert-butyl{1-[4-trifluoromethylbenzoyl]-6-chloro-5-hydroxy-2-methyl-1H-indol-3-yl]acetate
cyclobutyl{1-[4-trifluoromethylbenzoyl]-6-chloro-5-hydroxy-2-methyl-1H-indol-3-yl]acetate
cyclopentyl{1-[4-trifluoromethylbenzoyl]-6-chloro-5-hydroxy-2-methyl-1H-indol-3-yl]acetate
2-{1-[4-trifluoromethylbenzoyl]-6-chloro-5-hydroxy-2-methyl-1H-indol-3-yl]acetamide
2-{1-[4-trifluoromethylbenzoyl]-6-chloro-5-hydroxy-2-methyl-1H-indol-3-yl]-N-methylacetamide
2-{1-[4-trifluoromethylbenzoyl]-6-chloro-5-hydroxy-2-methyl-1H-indol-3-yl]-N-ethylacetamide
2-{1-[4-trifluoromethylbenzoyl]-6-chloro-5-hydroxy-2-methyl-1H-indol-3-yl]-N-propylacetamide
2-{1-[4-trifluoromethylbenzoyl]-6-chloro-5-hydroxy-2-methyl-1H-indol-3-yl]-N-isopropylacetamide
2-{1-[4-trifluoromethylbenzoyl]-6-chloro-5-hydroxy-2-methyl-1H-indol-3-yl]-N-butylacetamide
2-{1-[4-trifluoromethylbenzoyl]-6-chloro-5-hydroxy-2-methyl-1H-indol-3-yl]-N-sec-butylacetamide
2-{1-[4-trifluoromethylbenzoyl]-6-chloro-5-hydroxy-2-methyl-1H-indol-3-yl]-N-(2-hydroxyethyl)acetamide
methyl{1-[4-trifluoromethoxybenzoyl]-6-chloro-5-methoxy-2-methyl-1H-indol-3-yl]acetate
ethyl{1-[4-trifluoromethoxybenzoyl]-6-chloro-5-methoxy-2-methyl-1H-indol-3-yl]acetate
propyl{1-[4-trifluoromethoxybenzoyl]-6-chloro-5-methoxy-2-methyl-1H-indol-3-yl]acetate
butyl{1-[4-trifluoromethoxybenzoyl]-6-chloro-5-methoxy-2-methyl-1H-indol-3-yl]acetate
sec-butyl{1-[4-trifluoromethoxybenzoyl]-6-chloro-5-methoxy-2-methyl-1H-indol-3-yl]acetate
(R)sec-butyl{1-[4-trifluoromethoxybenzoyl]-6-chloro-5-methoxy-2-methyl-1H-indol-3-yl]acetate
(S)sec-butyl{1-[4-trifluoromethoxybenzoyl]-6-chloro-5-methoxy-2-methyl-1H-indol-3-yl]acetate
isopropyl{1-[4-trifluoromethoxybenzoyl]-6-chloro-5-methoxy-2-methyl-1H-indol-3-yl]acetate
tert-butyl{1-[4-trifluoromethoxybenzoyl]-6-chloro-5-methoxy-2-methyl-1H-indol-3-yl]acetate
cyclobutyl{1-[4-trifluoromethoxybenzoyl]-6-chloro-5-methoxy-2-methyl-1H-indol-3-yl]acetate
cyclopentyl{1-[4-trifluoromethoxybenzoyl]-6-chloro-5-methoxy-2-methyl-1H-indol-3-yl]acetate
2-{1-[4-trifluoromethoxybenzoyl]-6-chloro-5-methoxy-2-methyl-1H-indol-3-yl]acetamide
2-{1-[4-trifluoromethoxybenzoyl]-6-chloro-5-methoxy-2-methyl-1H-indol-3-yl]-N-methylacetamide
2-{1-[4-trifluoromethoxybenzoyl]-6-chloro-5-methoxy-2-methyl-1H-indol-3-yl]-N-ethylacetamide
2-{1-[4-trifluoromethoxybenzoyl]-6-chloro-5-methoxy-2-methyl-1H-indol-3-yl]-N-propylacetamide
2-{1-[4-trifluoromethoxybenzoyl]-6-chloro-5-methoxy-2-methyl-1H-indol-3-yl]-N-isopropylacetamide
2-{1-[4-trifluoromethoxybenzoyl]-6-chloro-5-methoxy-2-methyl-1H-indol-3-yl]-N-butylacetamide
2-{1-[4-trifluoromethoxybenzoyl]-6-chloro-5-methoxy-2-methyl-1H-indol-3-yl]-N-sec-butylacetamide
2-{1-[4-trifluoromethoxybenzoyl]-6-chloro-5-methoxy-2-methyl-1H-indol-3-yl]-N-(2-hydroxyethyl)acetamide
methyl{1-[4-trifluoromethoxybenzoyl]-6-chloro-5-hydroxy-2-methyl-1H-indol-3-yl]acetate
ethyl{1-[4-trifluoromethoxybenzoyl]-6-chloro-5-hydroxy-2-methyl-1H-indol-3-yl]acetate
propyl{1-[4-trifluoromethoxybenzoyl]-6-chloro-5-hydroxy-2-methyl-1H-indol-3-yl]acetate
butyl{1-[4-trifluoromethoxybenzoyl]-6-chloro-5-hydroxy-2-methyl-1H-indol-3-yl]acetate
sec-butyl{1-[4-trifluoromethoxybenzoyl]-6-chloro-5-hydroxy-2-methyl-1H-indol-3-yl]acetate
(R)sec-butyl{1-[4-trifluoromethoxybenzoyl]-6-chloro-5-hydroxy-2-methyl-1H-indol-3-yl]acetate
(S)sec-butyl{1-[4-trifluoromethoxybenzoyl]-6-chloro-5-hydroxy-2-methyl-1H-indol-3-yl]acetate
isopropyl{1-[4-trifluoromethoxybenzoyl]-6-chloro-5-hydroxy-2-methyl-1H-indol-3-yl]acetate
tert-butyl{1-[4-trifluoromethoxybenzoyl]-6-chloro-5-hydroxy-2-methyl-1H-indol-3-yl]acetate
cyclobutyl{1-[4-trifluoromethoxybenzoyl]-6-chloro-5-hydroxy-2-methyl-1H-indol-3-yl]acetate
cyclopentyl{1-[4-trifluoromethoxybenzoyl]-6-chloro-5-hydroxy-2-methyl-1H-indol-3-yl]acetate
2-{1-[4-trifluoromethoxybenzoyl]-6-chloro-5-hydroxy-2-methyl-1H-indol-3-yl]acetamide
2-{1-[4-trifluoromethoxybenzoyl]-6-chloro-5-hydroxy-2-methyl-1H-indol-3-yl]-N-methylacetamide
2-{1-[4-trifluoromethoxybenzoyl]-6-chloro-5-hydroxy-2-methyl-1H-indol-3-yl]-N-ethylacetamide
2-{1-[4-trifluoromethoxybenzoyl]-6-chloro-5-hydroxy-2-methyl-1H-indol-3-yl]-N-propylacetamide
2-{1-[4-trifluoromethoxybenzoyl]-6-chloro-5-hydroxy-2-methyl-1H-indol-3-yl]-N-isopropylacetamide
2-{1-[4-trifluoromethoxybenzoyl]-6-chloro-5-hydroxy-2-methyl-1H-indol-3-yl]-N-butylacetamide
2-{1-[4-trifluoromethoxybenzoyl]-6-chloro-5-hydroxy-2-methyl-1H-indol-3-yl]-N-sec-butylacetamide
2-{1-[4-trifluoromethoxybenzoyl]-6-chloro-5-hydroxy-2-methyl-1H-indol-3-yl]-N-(2-hydroxyethyl)acetamide
methyl{1-[4-difluoromethoxybenzoyl]-6-chloro-5-methoxy-2-methyl-1H-indol-3-yl]acetate
ethyl{1-[4-difluoromethoxybenzoyl]-6-chloro-5-methoxy-2-methyl-1H-indol-3-yl]acetate
propyl{1-[4-difluoromethoxybenzoyl]-6-chloro-5-methoxy-2-methyl-1H-indol-3-yl]acetate
butyl{1-[4-difluoromethoxybenzoyl]-6-chloro-5-methoxy-2-methyl-1H-indol-3-yl]acetate
sec-butyl{1-[4-difluoromethoxybenzoyl]-6-chloro-5-methoxy-2-methyl-1H-indol-3-yl]acetate
(R)sec-butyl{1-[4-difluoromethoxybenzoyl]-6-chloro-5-methoxy-2-methyl-1H-indol-3-yl]acetate
(S)sec-butyl{1-[4-difluoromethoxybenzoyl]-6-chloro-5-methoxy-2-methyl-1H-indol-3-yl]acetate
isopropyl{1-[4-difluoromethoxybenzoyl]-6-chloro-5-methoxy-2-methyl-1H-indol-3-yl]acetate
tert-butyl{1-[4-difluoromethoxybenzoyl]-6-chloro-5-methoxy-2-methyl-1H-indol-3-yl]acetate
cyclobutyl{1-[4-difluoromethoxybenzoyl]-6-chloro-5-methoxy-2-methyl-1H-indol-3-yl]acetate
cyclopentyl{1-[4-difluoromethoxybenzoyl]-6-chloro-5-methoxy-2-methyl-1H-indol-3-yl]acetate
2-{1-[4-difluoromethoxybenzoyl]-6-chloro-5-methoxy-2-methyl-1H-indol-3-yl]acetamide
2-{1-[4-difluoromethoxybenzoyl]-6-chloro-5-methoxy-2-methyl-1H-indol-3-yl]-N-methylacetamide
2-{1-[4-difluoromethoxybenzoyl]-6-chloro-5-methoxy-2-methyl-1H-indol-3-yl]-N-ethylacetamide
2-{1-[4-difluoromethoxybenzoyl]-6-chloro-5-methoxy-2-methyl-1H-indol-3-yl]-N-propylacetamide
2-{1-[4-difluoromethoxybenzoyl]-6-chloro-5-methoxy-2-methyl-1H-indol-3-yl]-N-isopropylacetamide
2-{1-[4-difluoromethoxybenzoyl]-6-chloro-5-methoxy-2-methyl-1H-indol-3-yl]-N-butylacetamide
2-{1-[4-difluoromethoxybenzoyl]-6-chloro-5-methoxy-2-methyl-1H-indol-3-yl]-N-sec-butylacetamide
2-{1-[4-difluoromethoxybenzoyl]-6-chloro-5-methoxy-2-methyl-1H-indol-3-yl]-N-(2-hydroxyethyl)acetamide
methyl{1-[4-difluoromethoxybenzoyl]-6-chloro-5-hydroxy-2-methyl-1H-indol-3-yl]acetate
ethyl{1-[4-difluoromethoxybenzoyl]-6-chloro-5-hydroxy-2-methyl-1H-indol-3-yl]acetate
propyl{1-[4-difluoromethoxybenzoyl]-6-chloro-5-hydroxy-2-methyl-1H-indol-3-yl]acetate
butyl{1-[4-difluoromethoxybenzoyl]-6-chloro-5-hydroxy-2-methyl-1H-indol-3-yl]acetate
sec-butyl{1-[4-difluoromethoxybenzoyl]-6-chloro-5-hydroxy-2-methyl-1H-indol-3-yl]acetate
(R)sec-butyl{1-[4-difluoromethoxybenzoyl]-6-chloro-5-hydroxy-2-methyl-1H-indol-3-yl]acetate
(S)sec-butyl{1-[4-difluoromethoxybenzoyl]-6-chloro-5-hydroxy-2-methyl-1H-indol-3-yl]acetate
isopropyl{1-[4-difluoromethoxybenzoyl]-6-chloro-5-hydroxy-2-methyl-1H-indol-3-yl]acetate
tert-butyl{1-[4-difluoromethoxybenzoyl]-6-chloro-5-hydroxy-2-methyl-1H-indol-3-yl]acetate
cyclobutyl{1-[4-difluoromethoxybenzoyl]-6-chloro-5-hydroxy-2-methyl-1H-indol-3-yl]acetate
cyclopentyl{1-[4-difluoromethoxybenzoyl]-6-chloro-5-hydroxy-2-methyl-1H-indol-3-yl]acetate
2-{1-[4-difluoromethoxybenzoyl]-6-chloro-5-hydroxy-2-methyl-1H-indol-3-yl]acetamide
2-{1-[4-difluoromethoxybenzoyl]-6-chloro-5-hydroxy-2-methyl-1H-indol-3-yl]-N-methylacetamide
2-{1-[4-difluoromethoxybenzoyl]-6-chloro-5-hydroxy-2-methyl-1H-indol-3-yl]-N-ethylacetamide
2-{1-[4-difluoromethoxybenzoyl]-6-chloro-5-hydroxy-2-methyl-1H-indol-3-yl]-N-propylacetamide
2-{1-[4-difluoromethoxybenzoyl]-6-chloro-5-hydroxy-2-methyl-1H-indol-3-yl]-N-isopropylacetamide
2-{1-[4-difluoromethoxybenzoyl]-6-chloro-5-hydroxy-2-methyl-1H-indol-3-yl]-N-butylacetamide
2-{1-[4-difluoromethoxybenzoyl]-6-chloro-5-hydroxy-2-methyl-1H-indol-3-yl]-N-sec-butylacetamide
2-{1-[4-difluoromethoxybenzoyl]-6-chloro-5-hydroxy-2-methyl-1H-indol-3-yl]-N-(2-hydroxyethyl)acetamide
methyl{1-[4-methylthiobenzoyl]-6-chloro-5-methoxy-2-methyl-1H-indol-3-yl]acetate
ethyl{1-[4-methylthiobenzoyl]-6-chloro-5-methoxy-2-methyl-1H-indol-3-yl]acetate
propyl{1-[4-methylthiobenzoyl]-6-chloro-5-methoxy-2-methyl-1H-indol-3-yl]acetate
butyl{1-[4-methylthiobenzoyl]-6-chloro-5-methoxy-2-methyl-1H-indol-3-yl]acetate
sec-butyl{1-[4-methylthiobenzoyl]-6-chloro-5-methoxy-2-methyl-1H-indol-3-yl]acetate
(R)sec-butyl{1-[4-methylthiobenzoyl]-6-chloro-5-methoxy-2-methyl-1H-indol-3-yl]acetate
(S)sec-butyl{1-[4-methylthiobenzoyl]-6-chloro-5-methoxy-2-methyl-1H-indol-3-yl]acetate
isopropyl{1-[4-methylthiobenzoyl]-6-chloro-5-methoxy-2-methyl-1H-indol-3-yl]acetate
tert-butyl{1-[4-methylthiobenzoyl]-6-chloro-5-methoxy-2-methyl-1H-indol-3-yl]acetate
cyclobutyl{1-[4-methylthiobenzoyl]-6-chloro-5-methoxy-2-methyl-1H-indol-3-yl]acetate
cyclopentyl{1-[4-methylthiobenzoyl]-6-chloro-5-methoxy-2-methyl-1H-indol-3-yl]acetate
2-{1-[4-methylthiobenzoyl]-6-chloro-5-methoxy-2-methyl-1H-indol-3-yl]acetamide
2-{1-[4-methylthiobenzoyl]-6-chloro-5-methoxy-2-methyl-1H-indol-3-yl]-N-methylacetamide
2-{1-[4-methylthiobenzoyl]-6-chloro-5-methoxy-2-methyl-1H-indol-3-yl]-N-ethylacetamide
2-{1-[4-methylthiobenzoyl]-6-chloro-5-methoxy-2-methyl-1H-indol-3-yl]-N-propylacetamide
2-{1-[4-methylthiobenzoyl]-6-chloro-5-methoxy-2-methyl-1H-indol-3-yl]-N-isopropylacetamide
2-{1-[4-methylthiobenzoyl]-6-chloro-5-methoxy-2-methyl-1H-indol-3-yl]-N-butylacetamide
2-{1-[4-methylthiobenzoyl]-6-chloro-5-methoxy-2-methyl-1H-indol-3-yl]-N-sec-butylacetamide
2-{1-[4-methylthiobenzoyl]-6-chloro-5-methoxy-2-methyl-1H-indol-3-yl]-N-(2-hydroxyethyl)acetamide
methyl{1-[4-methylthiobenzoyl]-6-chloro-5-hydroxy-2-methyl-1H-indol-3-yl]acetate
ethyl{1-[4-methylthiobenzoyl]-6-chloro-5-hydroxy-2-methyl-1H-indol-3-yl]acetate
propyl{1-[4-methylthiobenzoyl]-6-chloro-5-hydroxy-2-methyl-1H-indol-3-yl]acetate
butyl{1-[4-methylthiobenzoyl]-6-chloro-5-hydroxy-2-methyl-1H-indol-3-yl]acetate
sec-butyl{1-[4-methylthiobenzoyl]-6-chloro-5-hydroxy-2-methyl-1H-indol-3-yl]acetate
(R)sec-butyl{1-[4-methylthiobenzoyl]-6-chloro-5-hydroxy-2-methyl-1H-indol-3-yl]acetate
(S)sec-butyl{1-[4-methylthiobenzoyl]-6-chloro-5-hydroxy-2-methyl-1H-indol-3-yl]acetate
isopropyl{1-[4-methylthiobenzoyl]-6-chloro-5-hydroxy-2-methyl-1H-in dol-3-yl]acetate
tert-butyl{1-[4-methylthiobenzoyl]-6-chloro-5-hydroxy-2-methyl-1H-indol-3-yl]acetate
cyclobutyl{1-[4-methylthiobenzoyl]-6-chloro-5-hydroxy-2-methyl-1H-indol-3-yl]acetate
cyclopentyl{1-[4-methylthiobenzoyl]-6-chloro-5-hydroxy-2-methyl-1H-indol-3-yl]acetate
2-{1-[4-methylthiobenzoyl]-6-chloro-5-hydroxy-2-methyl-1H-indol-3-yl]acetamide
2-{1-[4-methylthiobenzoyl]-6-chloro-5-hydroxy-2-methyl-1H-indol-3-yl]-N-methylacetamide
2-{1-[4-methylthiobenzoyl]-6-chloro-5-hydroxy-2-methyl-1H-indol-3-yl]-N-ethylacetamide
2-{1-[4-methylthiobenzoyl]-6-chloro-5-hydroxy-2-methyl-1H-indol-3-yl]-N-propylacetamide
2-{1-[4-methylthiobenzoyl]-6-chloro-5-hydroxy-2-methyl-1H-indol-3-yl]-N-isopropylacetamide
2-{1-[4-methylthiobenzoyl]-6-chloro-5-hydroxy-2-methyl-1H-indol-3-yl]-N-butylacetamide
2-{1-[4-methylthiobenzoyl]-6-chloro-5-hydroxy-2-methyl-1H-indol-3-yl]-N-sec-butylacetamide
2-{1-[4-methylthiobenzoyl]-6-chloro-5-hydroxy-2-methyl-1H-indol-3-yl]-N-(2-hydroxyethyl)acetamide
methyl[1-(4-methylbenzoyl)-5-methoxy-6-chloro-2-methyl-1H-indol-3-yl]acetate
ethyl[1-(4-methylbenzoyl)-5-methoxy-6-chloro-2-methyl-1H-indol-3-yl]acetate
propyl[1-(4-methylbenzoyl)-5-methoxy-6-chloro-2-methyl-1H-indol-3-yl]acetate
butyl[1-(4-methylbenzoyl)-5-methoxy-6-chloro-2-methyl-1H-indol-3-yl]acetate
sec-butyl[1-(4-methylbenzoyl)-5-methoxy-6-chloro-2-methyl-1H-indol-3-yl]acetate
(R)sec-butyl[1-(4-methylbenzoyl)-5-methoxy-6-chloro-2-methyl-1H-indol-3-yl]acetate
(S)sec-butyl[1-(4-methylbenzoyl)-5-methoxy-6-chloro-2-methyl-1H-indol-3-yl]acetate
isopropyl[1-(4-methylbenzoyl)-5-methoxy-6-chloro-2-methyl-1H-indol-3-yl]acetate
tert-butyl[1-(4-methylbenzoyl)-5-methoxy-6-chloro-2-methyl-1H-indol-3-yl]acetate
cyclobutyl[1-(4-methylbenzoyl)-5-methoxy-6-chloro-2-methyl-1H-indol-3-yl]acetate
cyclopentyl[1-(4-methylbenzoyl)-5-methoxy-6-chloro-2-methyl-1H-indol-3-yl]acetate
2-[1-(4-methylbenzoyl)-5-methoxy-6-chloro-2-methyl-1H-indol-3-yl]acetamide
2-[1-(4-methylbenzoyl)-5-methoxy-6-chloro-2-methyl-1H-indol-3-yl]-N-methylacetamide
2-[1-(4-methylbenzoyl)-5-methoxy-6-chloro-2-methyl-1H-indol-3-yl]-N-ethylacetamide
2-[1-(4-methylbenzoyl)-5-methoxy-6-chloro-2-methyl-1H-indol-3-yl]-N-propylacetamide
2-[1-(4-methylbenzoyl)-5-methoxy-6-chloro-2-methyl-1H-indol-3-yl]-N-isopropylacetamide
2-[1-(4-methylbenzoyl)-5-methoxy-6-chloro-2-methyl-1H-indol-3-yl]-N-butylacetamide
2-[1-(4-methylbenzoyl)-5-methoxy-6-chloro-2-methyl-1H-indol-3-yl]-N-sec-butylacetamide
2-[1-(4-methylbenzoyl)-5-methoxy-6-chloro-2-methyl-1H-indol-3-yl]-N-(2-hydroxyethyl)acetamide
methyl[1-(4-methylbenzoyl)-5-hydroxy-6-chloro-2-methyl-1H-indol-3-yl]acetate
ethyl[1-(4-methylbenzoyl)-5-hydroxy-6-chloro-2-methyl-1H-indol-3-yl]acetate
propyl[1-(4-methylbenzoyl)-5-hydroxy-6-chloro-2-methyl-1H-indol-3-yl]acetate
butyl[1-(4-methylbenzoyl)-5-hydroxy-6-chloro-2-methyl-1H-indol-3-yl]acetate
sec-butyl[1-(4-methylbenzoyl)-5-hydroxy-6-chloro-2-methyl-1H-indol-3-yl]acetate
(R)sec-butyl[1-(4-methylbenzoyl)-5-hydroxy-6-chloro-2-methyl-1H-indol-3-yl]acetate
(S)sec-butyl[1-(4-methylbenzoyl)-5-hydroxy-6-chloro-2-methyl-1H-indol-3-yl]acetate
isopropyl[1-(4-methylbenzoyl)-5-hydroxy-6-chloro-2-methyl-1H-indol-3-yl]acetate
tert-butyl[1-(4-methylbenzoyl)-5-hydroxy-6-chloro-2-methyl-1H-indol-3-yl]acetate
cyclobutyl[1-(4-methylbenzoyl)-5-hydroxy-6-chloro-2-methyl-1H-indol-3-yl]acetate
cyclopentyl[1-(4-methylbenzoyl)-5-hydroxy-6-chloro-2-methyl-1H-indol-3-yl]acetate
2-[1-(4-methylbenzoyl)-5-hydroxy-6-chloro-2-methyl-1H-indol-3-yl]acetamide
2-[1-(4-methylbenzoyl)-5-hydroxy-6-chloro-2-methyl-1H-indol-3-yl]-N-methylacetamide
2-[1-(4-methylbenzoyl)-5-hydroxy-6-chloro-2-methyl-1H-indol-3-yl]-N-ethylacetamide
2-[1-(4-methylbenzoyl)-5-hydroxy-6-chloro-2-methyl-1H-indol-3-yl]-N-propylacetamide
2-[1-(4-methylbenzoyl)-5-hydroxy-6-chloro-2-methyl-1H-indol-3-yl]-N-isopropylacetamide
2-[1-(4-methylbenzoyl)-5-hydroxy-6-chloro-2-methyl-1H-indol-3-yl]-N-butylacetamide
2-[1-(4-methylbenzoyl)-5-hydroxy-6-chloro-2-methyl-1H-indol-3-yl]-N-sec-butylacetamide and
2-[1-(4-methylbenzoyl)-5-hydroxy-6-chloro-2-methyl-1H-indol-3-yl]-N-(2-hydroxyethyl)acetamide.

The invention also includes useful intermediates such as (4-chloro-5-methoxy-2-methyl-1H-indol-3-yl)acetic acid, (6-chloro-5-methoxy-2-methyl-1H-indol-3-yl)acetic acid, 2-trimethylsilylethyl{6-chloro-5-methoxy-2-methyl-1H-indol-3-yl}acetate and 2-trimethylsilylethyl{6-chloro-1-[4-(difluoromethoxy)benzoyl]-5-methoxy-2-methyl-1H-indol-3-yl}acetate.

The term “suitable leaving group” refers to a group which may be readily displaced by a nucleophile which has a greater affinity for the positively charged carbon atom to which said leaving group is attached than said leaving group. Preferred leaving groups include: chloro, symmetrical or a mixed anhydride, acid fluoride, imidazole amide, activated esters such as a 4-nitrophenyl ester, a thioester, and the like.

An “inert organic solvent” is any organic solvent or combination of solvents that is unreactive in the reaction being conducted and is a solvent for the reactants. Such solvents used in the various reactions of this invention are identified in the discussion of reaction schemes and in the examples. Typical suitable solvents are halogenated compounds such as chloroform or dichloromethane; heterocyclic compounds such as tetrahydrofuran (THF) and 1,4-dioxane; ethers such as dimethoxyethane; dimethylformamide (DMF); dimethylsulfoxide (DMSO), lower alkanols (C1-C6 branched or straight chain alkanols) such as methanol; acetonitrile; and carbocyclic aromatics such as toluene.

A “base” means a metal hydroxide base such as lithium, sodium or potassium hydroxide. A basic organic compound such as pyridine, triethylamine, and the like are also commonly referred to as bases. In addition, potassium fluoride may also be employed as a base in certain reactions.

A “strong base” typically means a non-aqueous base such as a metal hydride, an amide base such as lithium diisopropyl amide, alkyllithiums and the like.

The term “metal hydride” refers to metal hydrides such as lithium, sodium or potassium hydride.

Examples of organic or inorganic aqueous acids include trifluoroacetic acid, HCl, HBr, trichloroacetic acid, methane sulfonic acid, sulfuric acid and the like.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it (i.e., 1, 2, 3, 4, 5, 6, 7, 8, 9, 10, 11, 12) and includes methyl, ethyl, n-propyl, n-butyl, i-propyl, i-butyl, t-butyl, s-butyl, n-pentyl, i-pentyl, t-pentyl, neo-pentyl, n-hexyl, and i-hexyl. The term “haloalkyl” refers to an alkyl in which one or more hydrogen atoms are replaced by halo, and includes alkyl moieties in which all hydrogens have been replaced by halo (e.g., perfluoroalkyl). The terms “arylalkyl” or “aralkyl” refer to an alkyl moiety in which an alkyl hydrogen atom is replaced by an aryl group. Examples of “arylalkyl” or “aralkyl” include benzyl and 9-fluorenyl groups.

The term “alkenyl” refers to a hydrocarbon chain that may be a straight chain or branched chain, containing the indicated number of carbon atoms and at least one double bond. For example, C₁-C₁₂ alkenyl indicates that the group may have from 1 to 12 (inclusive) carbon atoms in it (i.e., 1, 2, 3, 4, 5, 6, 7, 8, 9, 10, 11, 12), e.g., ethenyl, i-propenyl, n-butenyl, i-butenyl, allyl, 1,3-butadienyl. Where there are two double bonds or more than two double bonds they can be conjugated or non-conjugated.

The term “alknyl” refers to a hydrocarbon chain that may be a straight chain or branched chain, containing the indicated number of carbon atoms and at least one triple bond. For example, C₁-C₁₂ alknyl indicates that the group may have from 1 to 12 (inclusive) carbon atoms in it (i.e., 1, 2,3,4, 5,6,7, 8,9, 10, 11, 12).

The terms “alkylamino” and “dialkylamino” refer to —NH(alkyl) and —N(alkyl)₂ radicals respectively. The term “aralkylamino” refers to a —NH(aralkyl) radical. The term “alkoxy” refers to an —O-alkyl radical. The term “mercapto” refers to an SH radical. The term “thioalkoxy” refers to an —S-alkyl radical.

The term “aryl” refers to an aromatic monocyclic, bicyclic, or tricyclic hydrocarbon ring system, wherein any ring atom capable of substitution can be substituted by a substituent. Examples of aryl moieties include, but are not limited to, phenyl, naphthyl, and anthracenyl.

The term “cycloalkyl” as employed herein includes saturated monocyclic, bicyclic, tricyclic, or polycyclic hydrocarbon groups having 3 to 12 carbons, wherein any ring atom capable of substitution can be substituted by a substituent. Examples of cycloalkyl moieties include, but are not limited to, cyclopentyl, norbornyl, and adamantyl.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “oxo” refers to an oxygen atom, which forms a carbonyl when attached to carbon, an N-oxide when attached to nitrogen, and a sulfoxide or sulfone when attached to sulfur.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, alkenyl, alkynyl, alkoxy, acyloxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino (alkyl, aryl, aralkyl), S(O)ₙalkyl (where n is 0-2), S(O)ₙaryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents.

The details of one or more embodiments of the invention are set forth in the accompanying drawings and the description below. Other features, objects, and advantages of the invention will be apparent from the description and drawings, and from the claims. The patents, patent applications, and publications referenced herein are hereby incorporated by reference in their entirety.

### DETAILED DESCRIPTION

Described below are three different general methods (Schemel, Scheme 2, Scheme 3) for the preparation of certain indoles that are COX-2 and/or FAAH inhibitors. The compounds produced by the methods described below are useful in treating pain, inflammation and autoimmune diseases as well as other disorders such as anxiety, sleep disorders and weight disorders.

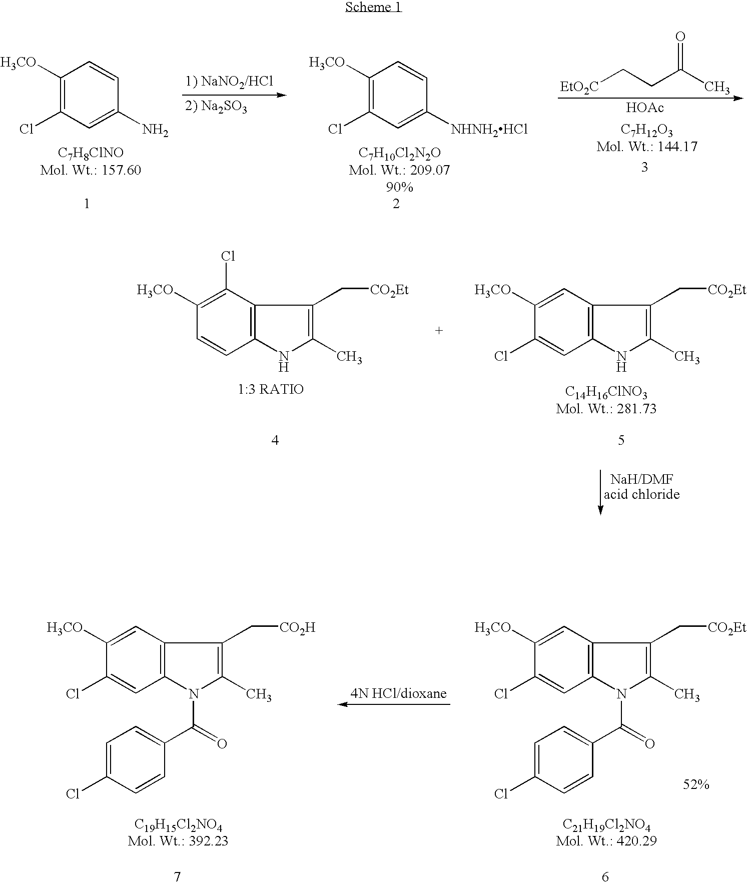

### Step 1. Preparation of (3-chloro-4-methoxyphenyl)hydrazine (2)

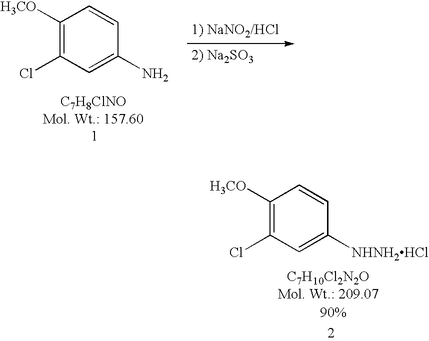

The preparation of phenylhydrazine derivative (2) begins with treatment of commercially available 3-chloro-4-methoxyaniline (1) with nitrous acid, generated from sodium nitrite and concentrated hydrochloric acid, to produce the corresponding diazonium salt. In the same reaction vessel the diazonium salt is treated with sodium sulfite and hydrochloric acid to produce the desired hydrazine hydrochloride (2) in 90% yield. Alternatively, the diazonium salt can be reduced with stannous chloride in hydrochloric acid.

### Step 2. Preparation of ethyl (6-chloro-5-methoxy-2-methyl-1H-indol-3-yl)acetate (5)

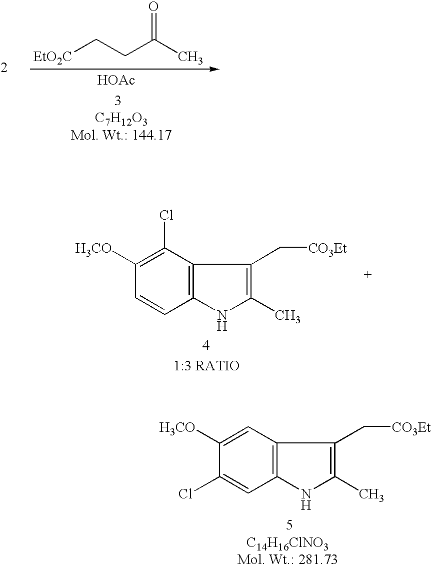

Condensation of hydrazine hydrochloride (2) with ethyl levulinate (3) in acetic acid results in the formation of two regioisomeric indole derivatives 4 and 5 in a 1:3 ratio. The desired regioisomer 5 can be isolated in pure form by crystallization of the reaction mixture from ethanol in 36% yield. The filtrate, containing a mixture of 4 and 5, can then be separated by chromatography on silica gel eluting with a mixture of hexanes and ethyl acetate.

### Step 3. Preparation of ethyl[6-chloro-1-(4-chlorobenzoyl)-5-methoxy-2-methyl-1H-indol-3-yl]acetate (6)

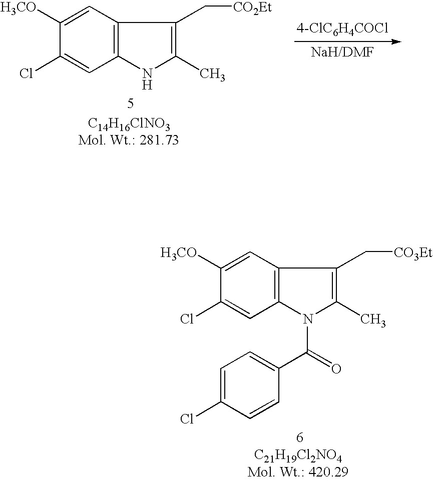

Treatment of the indole ester 5 with sodium hydride in dimethylformamide (DMF) followed by treatment with 4-chlorobenzoyl chloride afforded the acylated indole derivative 6 in 82% yield.

### Step 4. Preparation of [6-chloro-1-(4-chlorobenzoyl)-5-methoxy-2-methyl-1H-indol-3-yl]acetic acid (7)

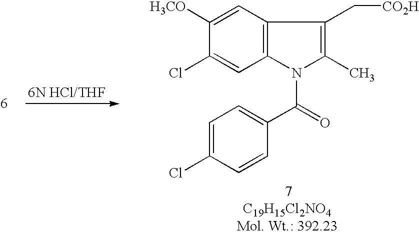

The ethyl ester 6 upon treatment with aqueous hydrochloric acid in tetrahydrofuran gave the corresponding acid 7 in 58% yield.

### Step 5. Preparation of [6-chloro-1-(4-chlorobenzoyl)-5-methoxy-2-methyl-1H-indol-3-yl]acetic acid compound with 2-amino-2-(hydroxymethyl)-1,3-propanediol (8)

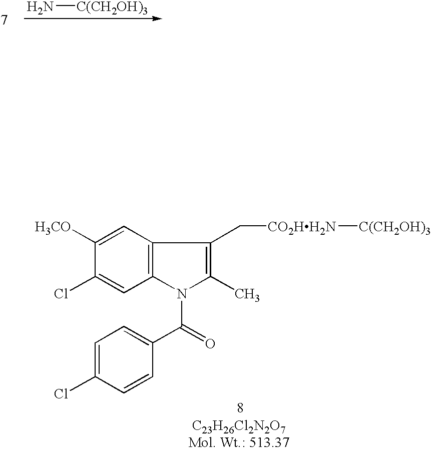

Treatment of the free acid 7 with 2-amino-2-(hydroxymethyl)-1,3-propanediol provided the salt form 8 in greater than 90% yield.

Alternatively, the route outlined in Scheme 2 may be employed to prepare acylated indoles. In the first step the hydrazine (2) is condensed with levulinic acid in acetic acid to afford a mixture of regioisomers 9 and 10 that can then be esterified with 2trimethylsilylethanol in the presence of a suitable dehydrating agent such as 1-(3-dimethylaminopropyl)-3-ethylcarbodiimide hydrochloride (EDCl). The esters are then separated and acylated with an acid chloride (4-chlorobenzoyl chloride in this instance) in the presence of base, to afford the desired product (12) in good yield. Finally, the ester may then be removed to provide the carboxylic acid (7 in the present example) by treatment with trifluoroacetic acid in excellent yield.

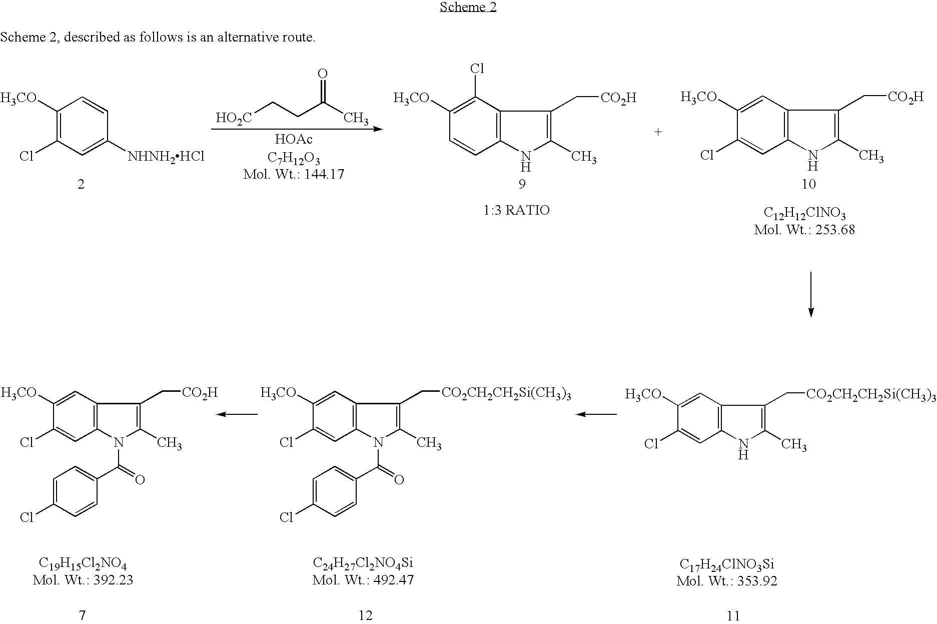

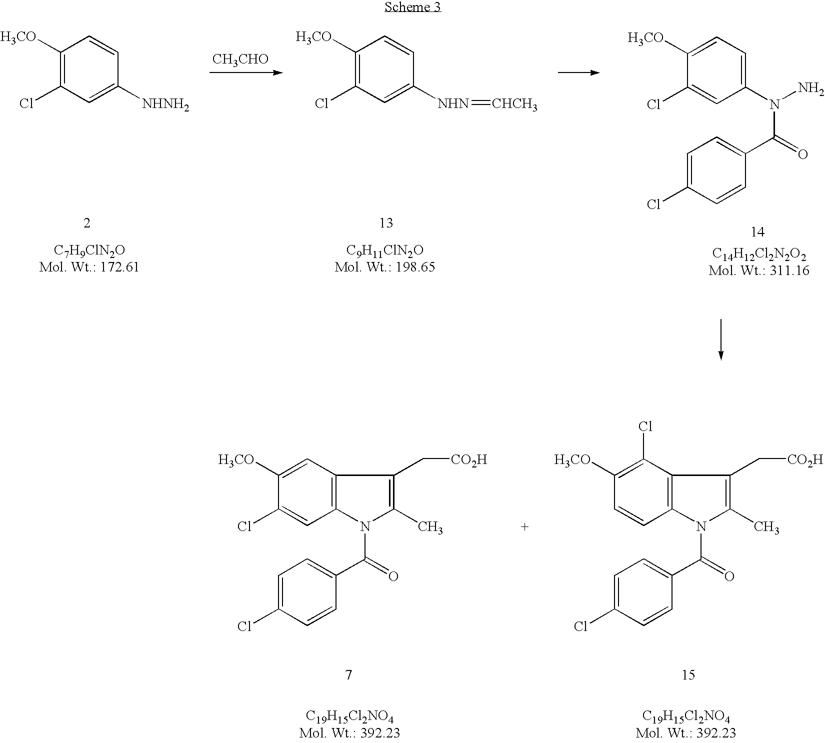

A third method for the preparation of acylated indoles is illustrated in Scheme 3. The route commences with the condensation of phenylhydrazine derivatives such as 2 with acetaldehyde or a different suitable aldehyde or SO₃ to protect the terminal nitrogen of the hydrazine to afford the corresponding hydrazone 13. Acylation of 13 with an acid chloride, in the present example 4-chlorobenzoyl chloride, followed by treatment with gaseous hydrochloric acid in an alcohol such as methanol provides the desired acylated hydrazine 14 after neutralization of the excess acid. Condensation of 14 with levulinic acid provides a mixture of regioisomers that can then be separated to afford acylated indoles, in the present example, 7 and 15.

Exemplery Final Compounds and Intermediates

Regarding Scheme 1, it is evident that various useful intermediate and final compounds include:

Methyl Ester Derivatives:

methyl (4-chloro-5-methoxy-2-methyl-1H-indol-3-yl)acetate 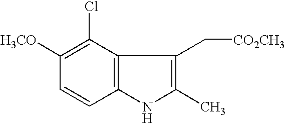
methyl (6-chloro-5-methoxy-2-methyl-1H-indol-3-yl)acetate 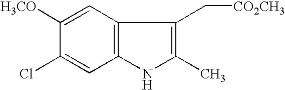
methyl[6-chloro-1-(4-chlorobenzoyl)-5-methoxy-2-methyl-1H-indol-3-yl]acetate 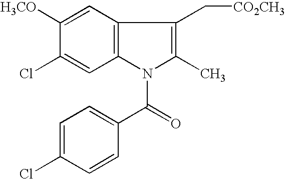
   Benzyl Ester Derivatives:
benzyl(4-chloro-5-methoxy-2-methyl-1H-indol-3-yl)acetate 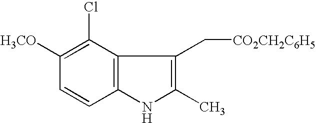
benzyl(6-chloro-5-methoxy-2-methyl-1H-indol-3-yl)acetate 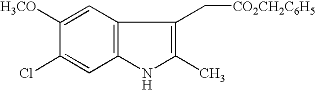
benzyl[6-chloro-1-(4-chlorobenzoyl)-5-methoxy-2-methyl-1H-indol-3-yl]acetate 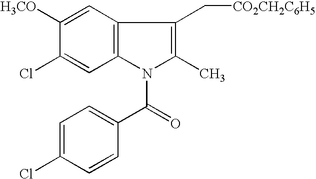
   Allyl Ester Derivatives:
ally(4-chloro-5-methoxy-2-methyl-1H-indol-3-yl)acetate 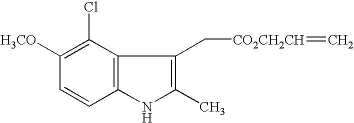
allyl(6-chloro-5-methoxy-2-methyl-1H-indol-3-yl)acetate 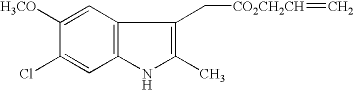
allyl[6-chloro-1-(4-chlorobenzoyl)-5-methoxy-2-methyl-1H-indol-3-yl]acetate 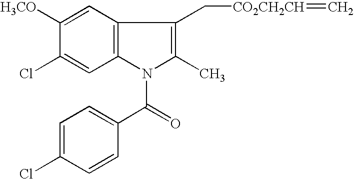
   tert-Butyl Ester Derivatives:
tert-butyl(4-chloro-5-methoxy-2-methyl-1H-indol-3-yl)acetate 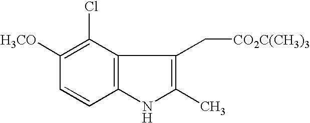
tert-butyl(6-chloro-5-methoxy-2-methyl-1H-indol-3-yl)acetate 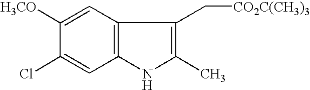
tert-butyl[6-chloro-1-(4-chlorobenzoyl)-5-methoxy-2-methyl-1H-indol-3-yl]acetate 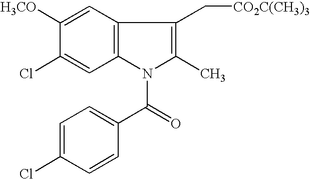
   Fluoro Derivatives:
ethyl(4-fluoro-5-methoxy-2-methyl-1H-indol-3-yl)acetate 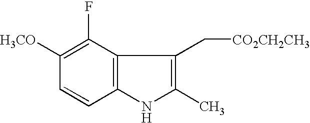
ethyl(6-fluoro-5-methoxy-2-methyl-1H-indol-3-yl)acetate 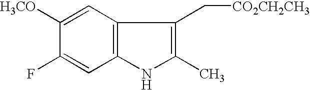
ethyl[6-fluoro-1-(4-chlorobenzoyl)-5-methoxy-2-methyl-1H-indol-3-yl]acetate 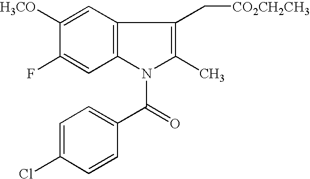
[6-fluoro-1-(4-chlorobenzoyl)-5-methoxy-2-methyl-1H-indol-3-yl]acetic acid 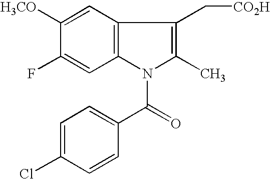
   Methyl Ester Analogues:
methyl(4-fluoro-5-methoxy-2-methyl-1H-indol-3-yl)acetate 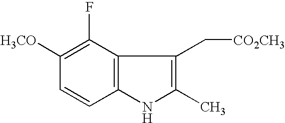
methyl(6-fluoro-5-methoxy-2-methyl-1H-indol-3-yl)acetate 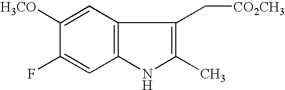
methyl[6-fluoro-1-(4-chlorobenzoyl)-5-methoxy-2-methyl-1H-indol-3-yl]acetate 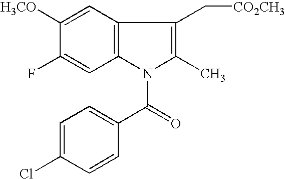
   Allyl Ester Analogues:
allyl(4-fluoro-5-methoxy-2-methyl-1H-indol-3-yl)acetate 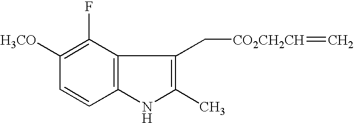
allyl(6-fluoro-5-methoxy-2-methyl-1H-indol-3-yl)acetate 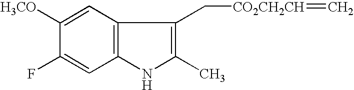
allyl[6-fluoro-1-(4-chlorobenzoyl)-5-methoxy-2-methyl-1H-indol-3-yl]acetate 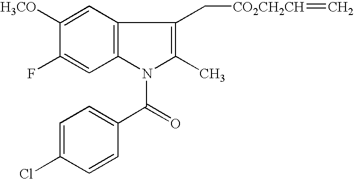
   Benzyl Ester Analogues:
benzyl(4-fluoro-5-methoxy-2-methyl-1H-indol-3-yl)acetate 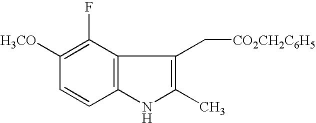
benzyl(6-fluoro-5-methoxy-2-methyl-1H-indol-3-yl)acetate 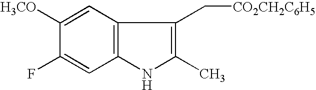
benzyl[6-fluoro-1-(4-chlorobenzoyl)-5-methoxy-2-methyl-1H-indol-3-yl]acetate 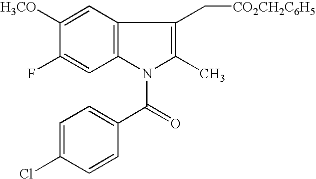
   tert-Butyl Ester Derivatives:
tert-butyl(4-fluoro-5-methoxy-2-methyl-1H-indol-3-yl)acetate 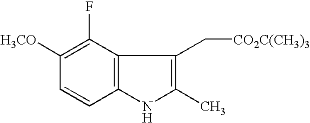
tert-butyl(6-fluoro-5-methoxy-2-methyl-1H-indol-3-yl)acetate 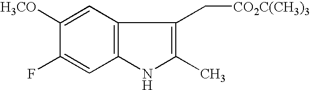
tert-butyl[6-fluoro-1-(4-chlorobenzoyl)-5-methoxy-2-methyl-1H-indol-3-yl]acetate

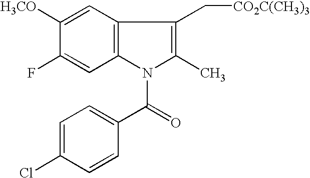

Also useful and synthetic intermediates or final products are the following compounds: 4-fluoro-N-(3-fluoro-4-methoxyphenyl)benzohydrazide 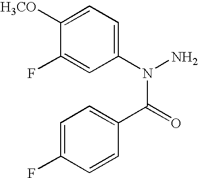
[6-fluoro-1-(4-fluorobenzoyl)-5-methoxy-2-methyl-1H-indol-3-yl]acetic acid 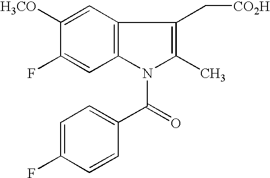
[4-fluoro-1-(4-fluorobenzoyl)-5-methoxy-2-methyl-1H-indol-3-yl]acetic acid 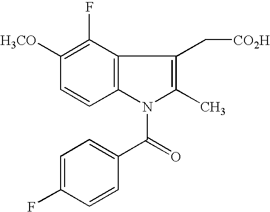
4-fluoro-N-(3-chloro-4-methoxyphenyl)benzohydrazide 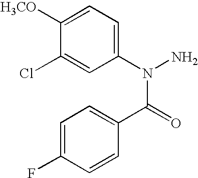
[4-chloro-1-(4-fluorobenzoyl)-5-methoxy-2-methyl-1H-indol-3-yl]acetic acid 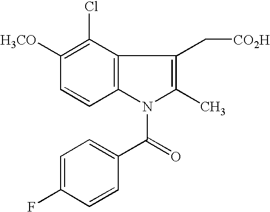
[6-chloro-1-(4-fluorobenzoyl)-5-methoxy-2-methyl-1H-indol-3-yl]acetic acid 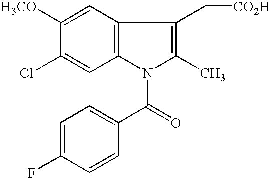
N-(3-chloro-4-methoxyphenyl)benzohydrazide 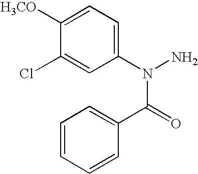
[4-chloro-1-(benzoyl)-5-methoxy-2-methyl-1H-indol-3-yl]acetic acid 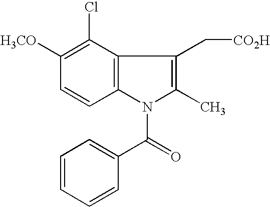
[6-chloro-1-(benzoyl)-5-methoxy-2-methyl-1H-indol-3-yl]acetic acid 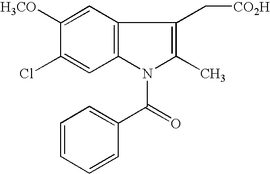
N-(3-fluoro-4-methoxyphenyl)benzohydrazide 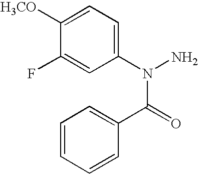
[4-fluoro-1-(benzoyl)-5-methoxy-2-methyl-1H-indol-3-yl]acetic acid 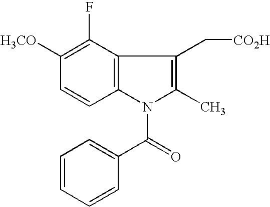
[6-fluoro-1-(benzoyl)-5-methoxy-2-methyl-1H-indol-3-yl]acetic acid 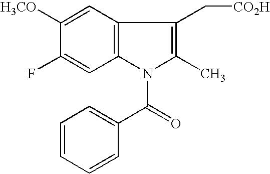
methyl[6-fluoro-1-(benzoyl)-5-methoxy-2-methyl-1H-indol-3-yl]acetate 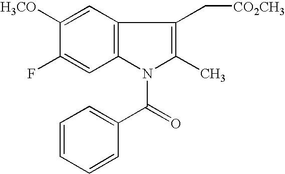
ethyl[6-fluoro-1-(benzoyl)-5-methoxy-2-methyl-1H-indol-3-yl]acetate 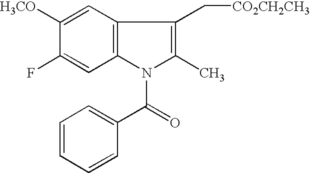
benzyl[6-fluoro-1-(benzoyl)-5-methoxy-2-methyl-1H-indol-3-yl]acetate 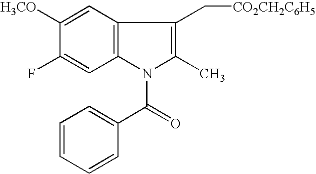
allyl[6-fluoro-1-(benzoyl)-5-methoxy-2-methyl-1H-indol-3-yl]acetate 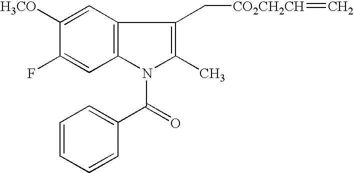
tert-butyl[6-fluoro-1-(benzoyl)-5-methoxy-2-methyl-1H-indol-3-yl]acetate 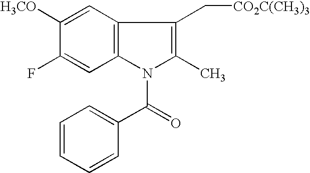
tert-butyl[4-fluoro-1-(benzoyl)-5-methoxy-2-methyl-1H-indol-3-yl]acetate 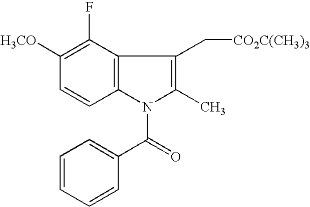
methyl[4-fluoro-1-(benzoyl)-5-methoxy-2-methyl-1H-indol-3-yl]acetate 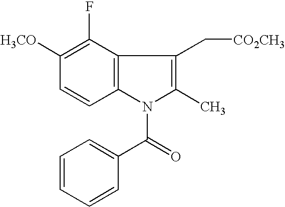
benzyl[4-fluoro-1-(benzoyl)-5-methoxy-2-methyl-1H-indol-3-yl]acetate 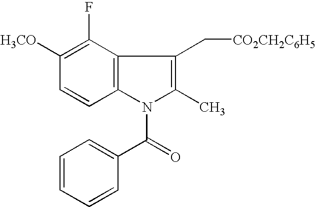
ethyl[4-fluoro-1-(benzoyl)-5-methoxy-2-methyl-1H-indol-3-yl]acetate 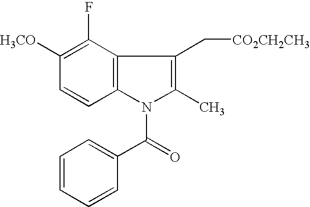
allyl[4-fluoro-1-(benzoyl)-5-methoxy-2-methyl-1H-indol-3-yl]acetate 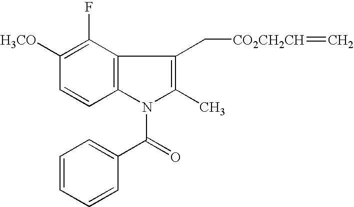
tert-butyl[6-fluoro-1-(4-fluorobenzoyl)-5-methoxy-2-methyl-1H-indol-3-yl]acetate 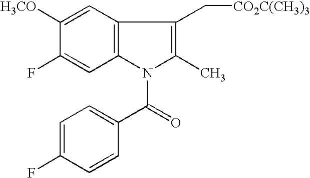
methyl[6-fluoro-1-(4-fluorobenzoyl)-5-methoxy-2-methyl-1H-indol-3-yl]acetate 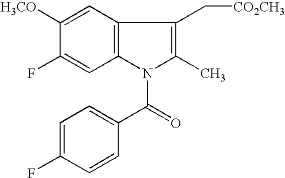
benzyl[6-fluoro-1-(4-fluorobenzoyl)-5-methoxy-2-methyl-1H-indol-3-yl]acetate 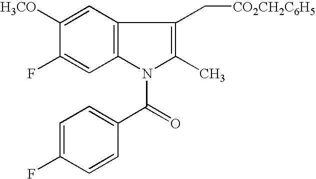
allyl[6-fluoro-1-(4-fluorobenzoyl)-5-methoxy-2-methyl-1H-indol-3-yl]acetate 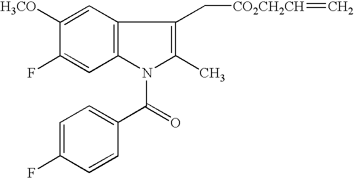
ethyl[6-fluoro-1-(4-fluorobenzoyl)-5-methoxy-2-methyl-1H-indol-3-yl]acetate 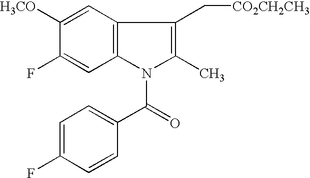
allyl[4-fluoro-1-(4-fluorobenzoyl)-5-methoxy-2-methyl-1H-indol-3-yl]acetate 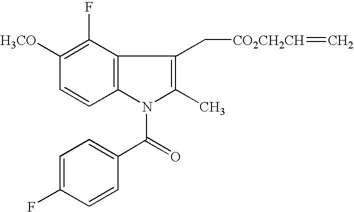
methyl[4-fluoro-1-(4-fluorobenzoyl)-5-methoxy-2-methyl-1H-indol-3-yl]acetate 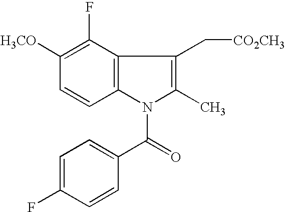
benzyl[4-fluoro-1-(4-fluorobenzoyl)-5-methoxy-2-methyl-1H-indol-3-yl]acetate 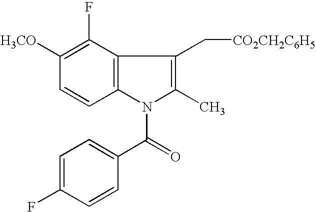
ethyl[4-fluoro-1-(4-fluorobenzoyl)-5-methoxy-2-methyl-1H-indol-3-yl]acetate 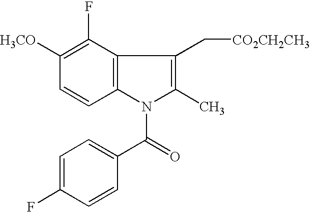
tert-butyl[4-fluoro-1-(4-fluorobenzoyl)-5-methoxy-2-methyl-1H-indol-3-yl]acetate 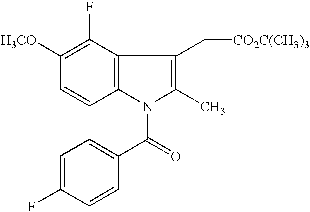
ethyl[6-chloro-1-(4-fluorobenzoyl)-5-methoxy-2-methyl-1H-indol-3-yl]acetate 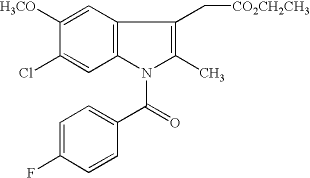
methyl[6-chloro-1-(4-fluorobenzoyl)-5-methoxy-2-methyl-1H-indol-3-yl]acetate 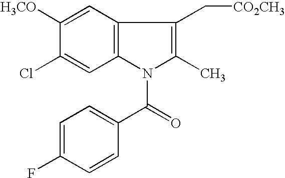
benzyl[6-chloro-1-(4-fluorobenzoyl)-5-methoxy-2-methyl-1H-indol-3-yl]acetate 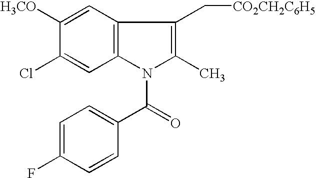
tert-butyl[6-chloro-1-(4-fluorobenzoyl)-5-methoxy-2-methyl-1H-indol-3-yl]acetate 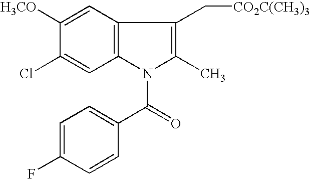
allyl[6-chloro-1-(4-fluorobenzoyl)-5-methoxy-2-methyl-1H-indol-3-yl]acetate 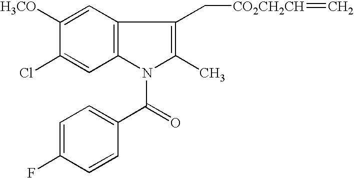
tert-butyl[4-chloro-1-(4-fluorobenzoyl)-5-methoxy-2-methyl-1H-indol-3-yl]acetate 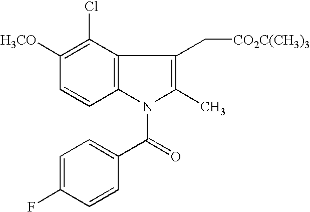
methyl[4-chloro-1-(4-fluorobenzoyl)-5-methoxy-2-methyl-1H-indol-3-yl]acetate 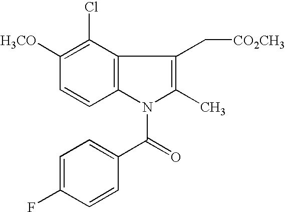
benzyl[4-chloro-1-(4-fluorobenzoyl)-5-methoxy-2-methyl-1H-indol-3-yl]acetate 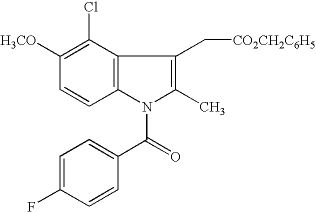
ethyl[4-chloro-1-(4-fluorobenzoyl)-5-methoxy-2-methyl-1H-indol-3-yl]acetate 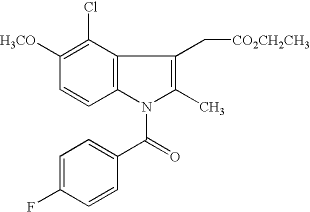
allyl[4-chloro-1-(4-fluorobenzoyl)-5-methoxy-2-methyl-1H-indol-3-yl]acetate 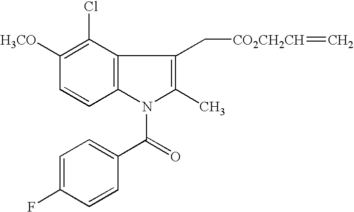
allyl[4-chloro-1-(benzoyl)-5-methoxy-2-methyl-1H-indol-3-yl]acetate 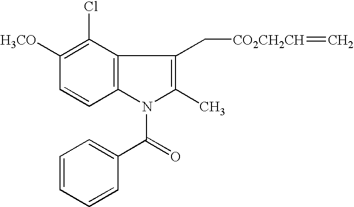
methyl[4-chloro-1-(benzoyl)-5-methoxy-2-methyl-1H-indol-3-yl]acetate 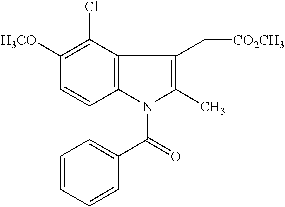
benzyl[4-chloro-1-(benzoyl)-5-methoxy-2-methyl-1H-indol-3-yl]acetate 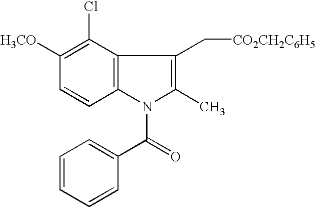
ethyl[4-chloro-1-(benzoyl)-5-methoxy-2-methyl-1H-indol-3-yl]acetate 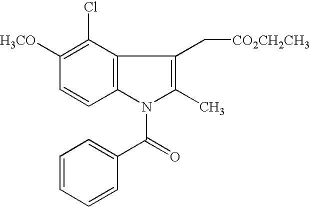
tert-butyl[4-chloro-1-(benzoyl)-5-methoxy-2-methyl-1H-indol-3-yl]acetate 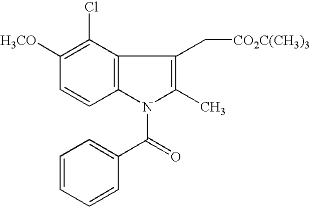
allyl[6-chloro-1-(benzoyl)-5-methoxy-2-methyl-1H-indol-3-yl]acetate 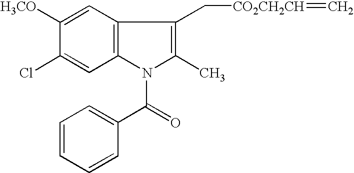
methyl[6-chloro-1-(benzoyl)-5-methoxy-2-methyl-1H-indol-3-yl]acetate 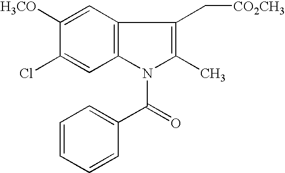
benzyl[6-chloro-1-(benzoyl)-5-methoxy-2-methyl-1H-indol-3-yl]acetate 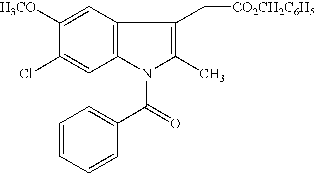
ethyl[6-chloro-1-(benzoyl)-5-methoxy-2-methyl-1H-indol-3-yl]acetate 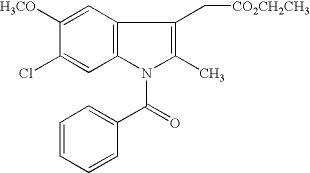
tert-butyl[6-chloro-1-(benzoyl)-5-methoxy-2-methyl-1H-indol-3-yl]acetate

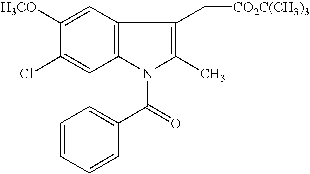

In the context of scheme 3, useful intermediates include: acetaldehyde (3-fluoro-4-methoxyphenyl)hydrazone 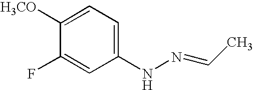
4-chloro-N-(3-fluoro-4-methoxyphenyl)benzohydrazide

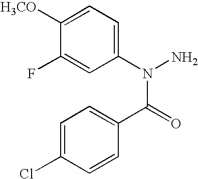

Described below are a number of specific examples of the synthesis of compounds by the above described methods.

### EXAMPLE 1

### (1-Benzoyl-5-hydroxy-2-methyl-1H-indol-3-yl)acetic acid (6) via Method A

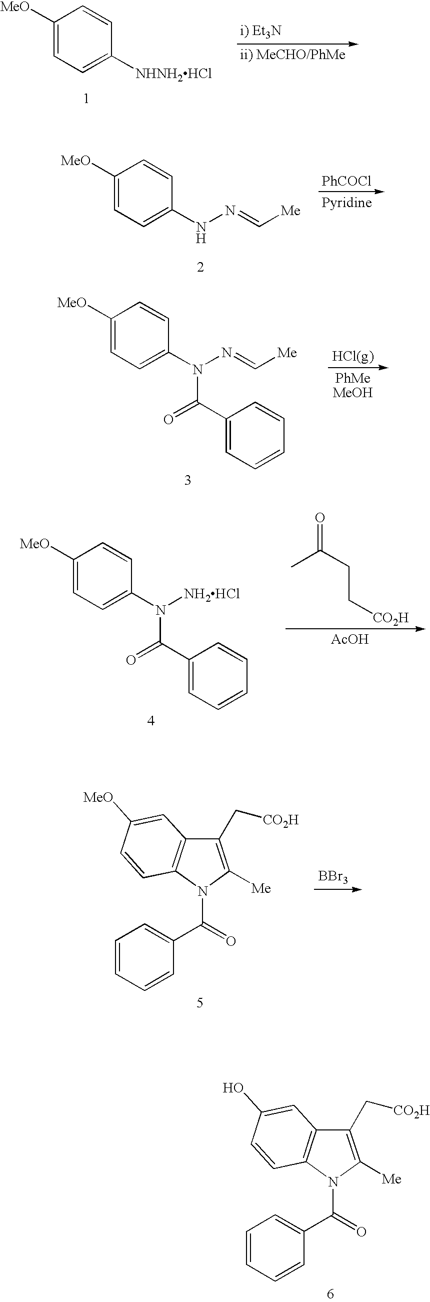

### Step 1. Preparation of acetaldehyde 4-methoxyphenylhydrazone (17)

Triethylamine (45 mL, 32.7 g, 0.323 mol) was added dropwise to a stirred suspension of 1-(4-methoxphenyl)hydrazine hydrochloride (1) (60 g, 0.344 mol) in toluene (400 mL), the mixture was stirred at ambient temperature for 30 min, then it was filtered and dried (MgSO₄). The drying agent was removed by filtration, the stirred solution was cooled to 0° C. and acetaldehyde (29 mL, 22.77 g, 0.517 mol) was added dropwise. The mixture was allowed to warm to ambient temperature then it was stirred under nitrogen for 3 h, filtered through Celite, and the solvent was removed in vacuo. The resulting black oil was subjected to Kugelröhr distillation to give acetaldehyde 4-methoxyphenylhydrazone (2) (22 g, 39%) as a yellow oil, bp 190° C. at 0.1 mmHg; 250 MHz ¹H-NMR (CDCl₃) δ (ppm): 1.75 (2×d, 3H, N═CHCH₃, E and Z isomers), 3.64, 3.65 (2×s, 3H, OCH₃, E and Z isomer), 6.55 (q, 0.5H, N═CHCH₃, one isomer), 6.70-6.98 (m, 5.5H, C₆H₄, NH and N═CHCH₃, one isomer).

### Step 2. Preparation of N′-ethylidene-N-(4-methoxyphenyl)benzohydrazide (3)

Benzoyl chloride (28.7 mL, 34.76 g, 0.247 mol) was added dropwise at 10° C. under nitrogen to a stirred solution of acetaldehyde 4-methoxyphenylhydrazone (2) (22 g, 0.134 mol) in pyridine (70 mL), the mixture was stirred at ambient temperature for 2 h, then it was quenched with water (200 mL). The product was extracted into dichloromethane (2×200 mL), the combined extracts were washed with water (2×100 mL), dried (MgSO₄), and the solvent was removed in vacuo. The residual black oil was purified by chromatography over silica using a 3:7 mixture of ethyl acetate and hexane as eluant. Appropriate fractions were combined and the solvents were removed in vacuo to give N′-ethylidene-N-(4-methoxyphenyl)benzohydrazide (3) (21 g, 58%). as a yellow solid, mp 68-70° C.; 250 MHz ¹H-NMR (CDCl₃) δ (ppm): 1.80 (d, 3H, N═CHCH₃), 3.75 (s, 3H, OCH₃), 6.79 (q, 1H, N═CHCH₃), 6.90-8.10 (m, 9H, C₆H₄ and Ph).

### Step 3. Preparation of N-(4-methoxyphenyl)benzohydrazide hydrochloride (4)

Hydrogen chloride gas was bubbled through a solution of N′-ethylidene-N-(4-methoxyphenyl)benzohydrazide (3) (21 g, 78.3 mmol) in a mixture of toluene (200 mL) and methanol (50 mL) at 0° C. for one h. The solvents were removed in vacuo and the residual solid was triturated with ethyl acetate (200 mL). The resulting solid was collected by filtration and dried in vacuo to give N-(4-methoxyphenyl)benzohydrazide hydrochloride (4) (12.7 g, 58%) as an off white solid, mp 170-172° C.; 250 MHz ¹H-NMR (d₆-DMSO) δ(ppm): 3.81 (s, 3H, OCH₃), 7.00 (d, 2H, 2H in Ph), 7.32-7.55 (m, 7H, C₆H₄ and 3H in Ph). (Note—there was no signal evident down to 12 ppm for —NH₃⁺).

### Step 4. Preparation of (1-benzoyl-5-methoxy-2-methyl-1H-indol-3-yl)acetic acid (5)

Levulinic acid (6.34 g, 54.6 mmol) was added to a stirred solution of N-(4-methoxyphenyl)benzohydrazide hydrochloride (4) (12.7 g, 45.6 mmol) in acetic acid (100 mL), the mixture was heated at 80° C. under nitrogen for 3 h, then it was allowed to cool to ambient temperature and poured onto ice-water (200 mL). The resulting precipitate was collected by filtration and dried in vacuo to give (1-benzoyl-5-methoxy-2-methyl-1H-indol-3-yl)acetic acid (5) (12.7 g, 86%) as a grey solid, mp 158-160° C.; 250 MHz ¹H-NMR (CDCl₃) δ(ppm): 2.31 (s, 3H, CH₃), 3.60 (s, 2H, CH₂COOH), 3.79 (s, 3H, OCH₃), 6.58 (dd, 1H, indole 6-H), 6.79 (d, 1H, indole 7-H), 6.90 (d, 1H, indole 4-H), 7.40-7.68 (m, 5H, C₆H₅), 10.00-12.00 (very br, cannot be integrated, COOH).

### Step 5. Preparation of (1-benzoyl-5-hydroxy-2-methyl-1H-indol-3-yl)acetic acid (6)

Boron tribromide (1 M solution in dichloromethane; 64.9 mL, 64.9 mmol) was added dropwise at −78° C. under nitrogen to a stirred solution of (1-benzoyl-5-methoxy-2-methyl-1H-indol-3-yl)acetic acid (5) (7.0 g, 21.6 mmol) in dichloromethane (200 mL), the mixture was allowed to warm to ambient temperature and stirred for 1 h, then it was poured onto water (200 mL). The resulting solid was collected by filtration and dried in vacuo to give (1-benzoyl-5-hydroxy-2-methyl-1H-indol-3-yl)acetic acid (6) (5.6 g, 83%) as a grey solid, mp 186-188° C.; 250 MHz ¹H-NMR (d₆-DMSO) δ(ppm): 2.12 (s, 3H, CH₃), 3.51 (s, 2H, CH₂COOH), 6.42 (dd, 1H, indole 6-H), 6.75 (m, 2H, indole 4-H and 7-H), 7.42-7.66 (m, 5H, C₆H₅), 9.10 (br, 1H, OH), 12.31 (br, 1H, COOH).

### EXAMPLE 2

### [1-(4-Fluorobenzoyl)-5-hydroxy-2-methyl-1H-indol-3-yl]acetic acid (10)

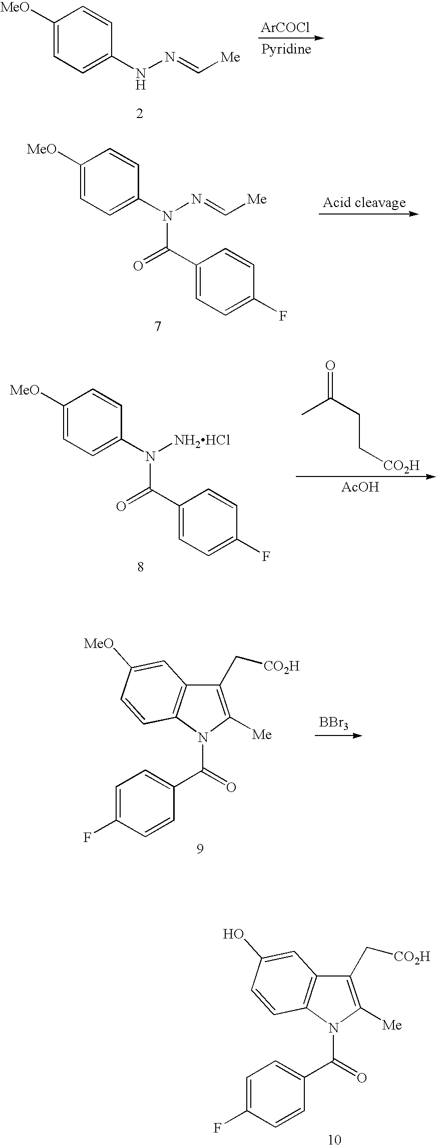

### Step 1. Preparation of N′-ethylidene-4-fluoro-N-(4-methoxyphenyl)benzohydrazide (7)

4-Fluorobenzoyl chloride (14.5 mL, 19.3 g, 0.122 mol) was added dropwise at 10° C. under nitrogen to a stirred solution of acetaldehyde 4-methoxyphenylhydrazone (2) (10 g, 0.061 mol) and pyridine (7.4 mL, 7.27 g, 0.092 mol) in ether (150 mL), the mixture was stirred at ambient temperature for 18 h, then it was added to ice-water (250 mL). The product was extracted into ether (2×200 mL), the combined extracts were dried (MgSO₄) and the solvent was removed in vacuo. The residue was purified by flash chromatography over silica using a 3:7 mixture of ethyl acetate and hexane as eluant. Appropriate fractions were combined and the solvents were removed in vacuo to give NA-ethylidene-4-fluoro-N-(4-methoxyphenyl)benzohydrazide (7) as a yellow oil (3.6 g, 21%) which was used without further purification.

### Step 2. Preparation of 4-fluoro-N-(4-methoxyphenyl)benzohydrazide hydrochloride (8)

Hydrogen chloride gas was bubbled through a solution of N′-ethylidene-4-fluoro-N-(4-methoxyphenyl)benzohydrazide (7) (3.6 g, 12.6 mmol) in a mixture of toluene (100 mL) and methanol (5 mL) at 0° C. for 1 h. The solvents were removed in vacuo and the residual solid was triturated with ethyl acetate (200 mL). The resulting solid was collected by filtration and dried in vacuo to give 4-fluoro-N-(4-methoxyphenyl)benzohydrazide hydrochloride (8) (1.3 g, 35%) as a green solid, mp 148-150° C.; 250 MHz ¹H-NMR (d₆-DMSO) δ(ppm): 3.85 (s, 3H, OCH₃), 6.99 (d, 2H, 2H in Ph), 7.22-7.55 (m, 6H, C₆H₄ and 2H in Ph). (Note—there was no signal evident down to 12 ppm for —NH₃⁺).

### Step 3. Preparation of [1-(4-fluorobenzoyl)-5-methoxy-2-methyl-1H-indol-3-yl]acetic acid (9)

Levulinic acid (0.56 g, 4.85 mmol) was added to a stirred solution of 4-fluoro-N-(4-methoxyphenyl)benzohydrazide hydrochloride (8) (1.2 g, 4.04 mmol) in acetic acid (20 mL), the mixture was heated under reflux under nitrogen for 4 h, then it was allowed to cool to ambient temperature and poured onto ice-water (50 mL). The resulting precipitate was collected by filtration and dried in vacuo to give [1-(4-fluorobenzoyl)-5-methoxy-2-methyl-1H-indol-3-yl]acetic acid (9) (0.9 g, 68%) as a grey solid, mp 130-132° C.; 250 MHz ¹H-NMR (CDCl₃) δ (ppm): 2.31 (s, 3H, CH₃), 3.60 (s, 2H, CH₂COOH), 3.72 (s, 3H, OCH₃), 6.58 (dd, 1H, indole 6-H), 6.79 (d, 1H, indole 7-H), 6.90 (d, 1H, indole 4-H), 7.10 (d, 2H, 2H in Ph), 7.67 (d, 2H, 2H in Ph), 8.00-9.00 (very br, cannot be integrated, COOH).

### Step 4. Preparation of [1-(4-fluorobenzoyl)-5-hydroxy-2-methyl-1H-indol-3-yl]acetic acid (10)

Boron tribromide (1 M solution in dichloromethane; 7.7 mL, 7.7 mmol) was added dropwise at −78° C. under nitrogen to a stirred solution of [1-(4-fluorobenzoyl)-5-methoxy-2-methyl-1H-indol-3-yl]acetic acid (9) (0.84 g, 2.6 mmol) in dichloromethane (10 mL), the mixture was allowed to warm to ambient temperature and stirred for 4 h, then it was poured onto water (15 mL). The resulting solid was collected by filtration and dried in vacuo to give [1-(4-fluorobenzoyl)-5-hydroxy-2-methyl-1H-indol-3-yl]acetic acid (10) (0.5 g, 61%) as an off white solid, mp 180-182° C.; 250 MHz ¹H-NMR (d₆-DMSO) δ (ppm): 2.22 (s, 3H, CH₃), 3.60 (s, 2H, CH₂COOH), 6.50 (d, 1H, indole 6-H), 6.80 (m, 2H, indole 4-H and 7-H), 7.42 (m, 2H, 2H in Ph), 7.78(m, 2H, 2H in Ph), 9.20 (br, 1H, OH), 12.41 (br, 1H, COOH).

### EXAMPLE 3

### {5-Hydroxy-2-methyl-1-[4-(trifluoromethoxy)benzoyl]-1H-indol-3-yl}acetic acid (14)

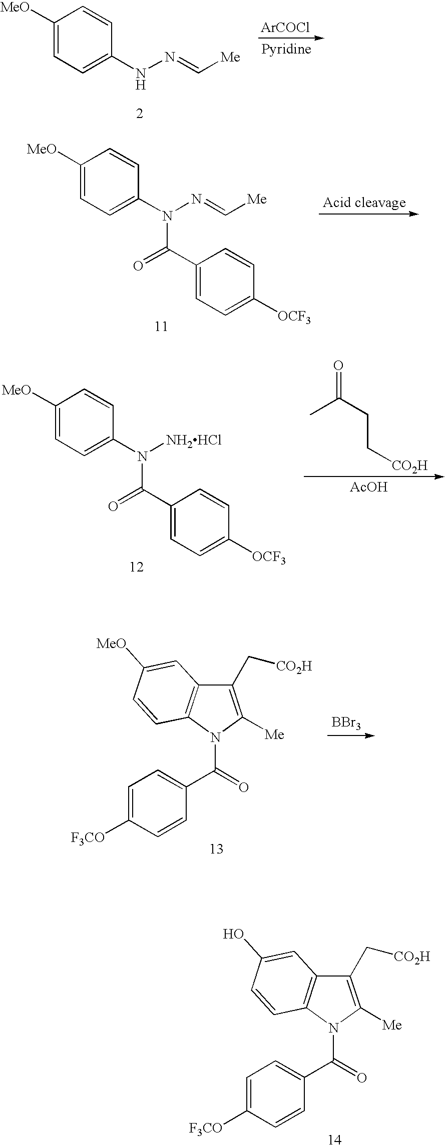

### Step 1. Preparation of N′-ethylidene-N-(4-methoxyphenyl)-4-(trifluoromethoxy)benzohydrazide (11)

4-(Trifluoromethoxy)benzoyl chloride (19.3 mL, 27.4 g, 0.122 mol) was added dropwise at 10° C. under nitrogen to a stirred solution of acetaldehyde 4-methoxyphenylhydrazone (2) (10 g, 0.061 mol) and pyridine (7.4 mL, 7.27 g, 0.092 mol) in ether (150 mL), the mixture was stirred at ambient temperature for 18 h, then it was added to ice-water (250 mL). The product was extracted into ether (2×200 mL), the combined extracts were dried (MgSO₄) and the solvent was removed in vacuo. The residue was purified by flash chromatography over silica using a 3:7 mixture of ethyl acetate and hexane as eluant. Appropriate fractions were combined and the solvents were removed in vacuo to give impure (˜20% by lc-ms) N′-ethylidene-N-(4-methoxyphenyl)-4-(trifluoromethoxy)benzohydrazide (11) as a green oil (21 g) which was used without further purification.

### Step 2. Preparation of N-(4-methoxyphenyl)-4-(trifluoromethoxy)benzohydrazide hydrochloride (12)

Hydrogen chloride gas was bubbled through a solution of crude N′-ethylidene-N-(4-methoxyphenyl)-4-(trifluoromethoxy)benzohydrazide (11) (21 g) in a mixture of toluene (100 mL) and methanol (10 mL) at 0° C. for 1 h. The solvents were removed in vacuo and the residual solid was triturated with ethyl acetate (200 mL). The resulting solid was collected by filtration and dried in vacuo to give N-(4-methoxyphenyl)-4-(trifluoromethoxy)benzohydrazide hydrochloride (12) (2.4 g) as a white solid, mp 154-156° C.; 250 MHz ¹H-NMR (d₆-DMSO) δ (ppm): 3.78 (s, 3H, OCH₃), 6.90 (d, 2H, 2H in Ph), 7.22-7.34 (m, 4H, C₆H₄), 7.56 (m, 2H, 2H in Ph). (Note—there was no signal evident down to 12 ppm for —NH₃⁺).

### Step 3. Preparation of {5-methoxy-2-methyl-1-[4-(trifluoromethoxy)benzoyl]-1H-indol-3-yl}acetic acid (13)

Levulinic acid (0.92 g, 7.9 mmol) was added to a stirred solution of N-(4-methoxyphenyl)-4-(trifluoromethoxy)benzohydrazide hydrochloride (12) (2.4 g, 6.6 mmol) in acetic acid (20 mL), the mixture was heated under reflux under nitrogen for 4 h, then it was allowed to cool to ambient temperature and poured onto ice-water (50 mL). The resulting precipitate was collected by filtration and dried in vacuo to give {5-methoxy-2-methyl-1-[4-(trifluoromethoxy)benzoyl]-1H-indol-3-yl}acetic acid (13) (2.3 g, 89%) as a white solid, mp 136-138° C.; 250 MHz ¹H-NMR (CDCl₃) δ (ppm): 2.30 (s, 3H, CH₃), 3.59 (s, 2H, CH₂COOH), 3.72 (s, 3H, OCH₃), 6.60 (dd, 1H, indole 6-H), 6.79 (d, 1H, indole 7-H), 6.86 (d, 1H, indole 4-H), 7.22 (d, 2H, 2H in Ph), 7.70 (d, 2H, 2H in Ph), 10.0-11.0 (very br, cannot be integrated, COOH).

### Step 4. Preparation of {5-hydroxy-2-methyl-1-[4-(trifluoromethoxy)benzoyl]-1H-indol-3-yl}acetic acid (14)

Boron tribromide (1 M solution in dichloromethane; 15.2 mL, 15.2 mmol) was added dropwise at −78° C. under nitrogen to a stirred solution of {5-methoxy-2-methyl-1-[4-(trifluoromethoxy)benzoyl]-1H-indol-3-yl}acetic acid (13) (2.0 g, 5.1 mmol) in dichloromethane (20 mL), the mixture was allowed to warm to ambient temperature and stirred for 4 h, then it was poured onto water (30 mL). The resulting solid was collected by filtration and dried in vacuo to give {5-hydroxy-2-methyl-1-[4-(trifluoromethoxy)benzoyl]-1H-indol-3-yl}acetic acid (14) (1.6 g, 83%) as an off-white solid, mp 193-195° C.; 250 MHz ¹H-NMR (d₆-DMSO) δ (ppm): 2.19 (s, 3H, CH₃), 3.60 (s, 2H, CH₂COOH), 6.50 (d, 1H, indole 6-H), 6.85 (dd, 2H, indole 4-H and 7-H), 7.50 (d, 2H, 2H in Ph), 7.78(d, 2H, 2H in Ph), 9.20 (s, 1H, OH), 12.38 (s, 1H, COOH).

### EXAMPLE 4

### (1-Benzoyl-6-fluoro-5-hydroxy-2-methyl-1H-indol-3-yl)acetic acid (21)

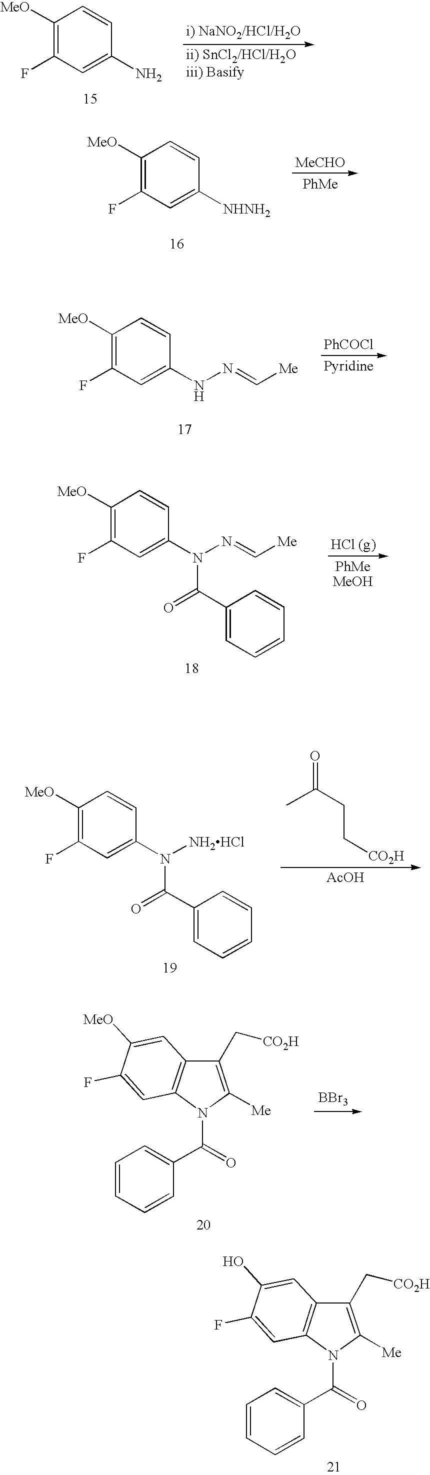

### Step 1. Preparation of (3-fluoro-4-methoxyphenyl)hydrazine (16)

3-Fluoro-4-methoxyaniline (15) (95 g, 0.67 mol) was added to concentrated hydrochloric acid (250 mL), the suspension was stirred at ambient temperature for 18 h, then it was cooled to 0° C. and a solution of sodium nitrite (53.7 g, 0.78 mol) in water (200 mL) was added dropwise at 0-5° C. When the addition was complete, the resulting solution was stirred at 0° C. for 1 h then it was added dropwise at 0-5° C. to a stirred solution of tin(II) chloride dihydrate (638.9 g, 2.83 mol) in concentrated hydrochloric acid (500 mL). The mixture was allowed to warm to ambient temperature then it was stored at 4° C. for 18 h. The resulting precipitate was collected by filtration, washed with water (400 mL), and ether (1000 mL) and dried in vacuo. The solid hydrochloride salt was basified by addition to 10% aqueous sodium hydroxide solution (800 mL), the free base was extracted into ether (2×400 mL), and the combined extracts were dried (MgSO₄) and the solvent removed in vacuo to give (3-fluoro-4-methoxyphenyl)hydrazine (16) (51.9 g, 50%) as a yellow solid, mp 46-50° C.; 250 MHz ¹H-NMR (CDCl₃) δ(ppm): 1.5 (s, 1H, NH—NH₂), 3.85 (s, 3H, OCH₃), 5.0 (s, 2H, NH—NH₂), 6.44 (m, 1H, phenyl 6-H), 6.60 (dd, 1H, phenyl 5-H), 6.79 (t, 1H, phenyl 2-H).

### Step 2. Preparation of acetaldehyde (3-fluoro-4-methoxyphenyl)hydrazone (17)

Acetaldehyde (11.2 mL, 8.8 g, 0.2 mol) was added dropwise at 0° C. under nitrogen to a stirred mixture of (3-fluoro-4-methoxyphenyl)hydrazine (16) (20.8 g, 0.133 mol), magnesium sulfate (80 g) and toluene (200 mL), the mixture was stirred at ambient temperature for 3 h, then it was filtered and the solvent was removed in vacuo. The residue was subjected to Kugelröhr distillation to give acetaldehyde (3-fluoro-4-methoxyphenyl)hydrazone (17) (10.5 g, 43%) as a yellow oil, bp 190° C. at 1 mmHg, which solidified slowly at ambient temperature. This material was used in the next step without further purification.

### Step 3. Preparation of N′-ethylidene-N-(3-fluoro-4-methoxyphenyl)benzohydrazide (18)

Benzoyl chloride (14.0 mL, 16.95 g, 0.121 mol) was added dropwise at 10° C. under nitrogen to a stirred solution of acetaldehyde (3-fluoro-4-methoxyphenyl)hydrazone (17) (11 g, 0.06 mol) in pyridine (100 mL), the mixture was stirred at ambient temperature for 2 h, then it was added to ice-water (250 mL). The product was extracted into dichloromethane (2×200 mL), the combined extracts were dried (MgSO₄) and the solvent was removed in vacuo. The residue was purified by flash chromatography over silica using a 3:7 mixture of ethyl acetate and hexane as eluant. Appropriate fractions were combined and the solvents were removed in vacuo to give N′-ethylidene-N-(3-fluoro-4-methoxyphenyl)benzohydrazide (18) (8 g, 47%) as a yellow solid, mp 110-112° C.; 250 MHz ¹H-NMR (CDCl₃) δ (ppm): 1.85 (d, 3H, N═CHCH₃), 3.85 (s, 3H, OCH₃), 6.8 (q, 1H, N═CHCH₃), 6.90-7.7 (m, 8H, C₆H₃ and Ph).

### Step 4. Preparation of N-(3-fluoro-4-methoxyphenyl)benzohydrazide hydrochloride (19)

Hydrogen chloride gas was bubbled through a stirred solution of N′-ethylidene-N-(3-fluoro-4-methoxyphenyl)benzohydrazide (18) (8 g, 0.028 mol) in a mixture of toluene (200 mL) and methanol (10 mL) at 0° C. for 1 h. The solvents were removed in vacuo, the residue was triturated with ethyl acetate (150 mL) and the resulting solid was collected by filtration and dried in vacuo to give N-(3-fluoro-4-methoxyphenyl)benzohydrazide hydrochloride (19) (5 g, 60%) as a white solid, mp 175-178° C.; 250 MHz ¹H-NMR (d₆-DMSO) δ (ppm): 3.9 (s, 3H, OCH₃), 7.1-7.55 (m, 8H, C₆H₃ and Ph), 8-10 (very br, 3H, —NH₃⁺).

### Step 4. Preparation of (1-Benzoyl-6-fluoro-5-methoxy-2-methyl-1H-indol-3-yl)acetic acid (20)

A stirred mixture of N-(3-fluoro-4-methoxyphenyl)benzohydrazide hydrochloride (19) (4.7 g, 15.8 mmol), levulinic acid (2.2 g, 19 mmol) and acetic acid (20 mL) was heated at 80° C. for 4 h, cooled to ambient temperature and added to ice-water (50 mL). The resulting solid was collected by filtration, dried in vacuo and crystallized from toluene to give a 5:1 mixture of (1-benzoyl-6-fluoro-5-methoxy-2-methyl-1H-indol-3-yl)acetic acid and (1-benzoyl-4-fluoro-5-methoxy-2-methyl-1H-indol-3-yl)acetic acid (3.6 g) as a grey solid. A portion (2.6 g) of the mixture was purified by column chromatography over silica using a 1:1 mixture of ethyl acetate and hexane containing a few drops of acetic acid as eluant. Appropriate fractions were combined and the solvents were removed in vacuo to give a small sample (200 mg) of the required regioisomer, (1-benzoyl-6-fluoro-5-methoxy-2-methyl-1H-indol-3-yl)acetic acid, and a larger sample (1.6 g) which was still a mixture of the two regioisomers. The larger sample (1.6 g) was subjected to a further purification by UV-triggered radial compression Biotage chromatography using gradient elution with 15:85-3:7 mixtures of ethyl acetate and hexane as eluants. Appropriate fractions were combined and the solvents were removed in vacuo to give the required regioisomer, (1-benzoyl-6-fluoro-5-methoxy-2-methyl-1H-indol-3-yl)acetic acid (500 mg) and a larger sample (1.1g) which was still a mixture of the two regioisomers. (1-Benzoyl-6-fluoro-5-methoxy-2-methyl-1H-indol-3-yl)acetic acid (20) (0.7 g, 13%) was thus obtained as a white solid, mp 118-120° C.; 250 MHz ¹H-NMR (CDCl₃) δ (ppm): 2.30 (s, 3H, CH₃), 3.61 (s, 2H, CH₂CO₂H), 3.88 (s, 3H, OCH₃), 6.71 (d, 1H, indole 4-H), 6.90 (d, 1H, indole 7-H), 7.48 (m, 2H, 2H in Ph), 7.59 (m, 3H, 3H in Ph), 10.00-11.00 (very br. COOH).

### Step 4. Preparation of (1-benzoyl-6-fluoro-5-hydroxy-2-methyl-1H-indol-3-yl)acetic acid (21)

Boron tribromide (1 M solution in dichloromethane; 3.51 mL, 3.51 mmol) was added dropwise at −78° C. under nitrogen to a stirred solution of (1-benzoyl-6-fluoro-5-methoxy-2-methyl-1H-indol-3-yl)acetic acid (20) (400 mg, 1.17 mmol) in dichloromethane (10 mL), the mixture was allowed to warm to ambient temperature, then it was stirred for a further 4.5 h. The mixture was added to ice-water (15 mL) and the resulting solid was collected by filtration and dried in vacuo to give (1-benzoyl-6-fluoro-5-hydroxy-2-methyl-1H-indol-3-yl)acetic acid (21) (200 mg, 52%) as a white solid, mp 159-161° C.; 250 MHz ¹H-NMR (d₆-DMSO) δ (ppm): 2.12 (s, 3H, CH₃), 3.59 (s, 2H, CH₂CO₂H)), 6.81 (d, 1H, indole 4-H), 7.01 (d, 1H, indole 7-H), 7.66 (m, 5H, C₆H₅), 9.69 (s, 1H, OH), 12.41 (s, 1H, COOH).

### EXAMPLES 5 AND 6

### [1-(4-Chlorobenzoyl)-6-fluoro-5-hydroxy-2-methyl-1H-indol-3-yl]acetic acid and [1-(4-chlorobenzoyl)-4-fluoro-5-hydroxy-2-methyl-1H-indol-3-yl]acetic acid via Method A

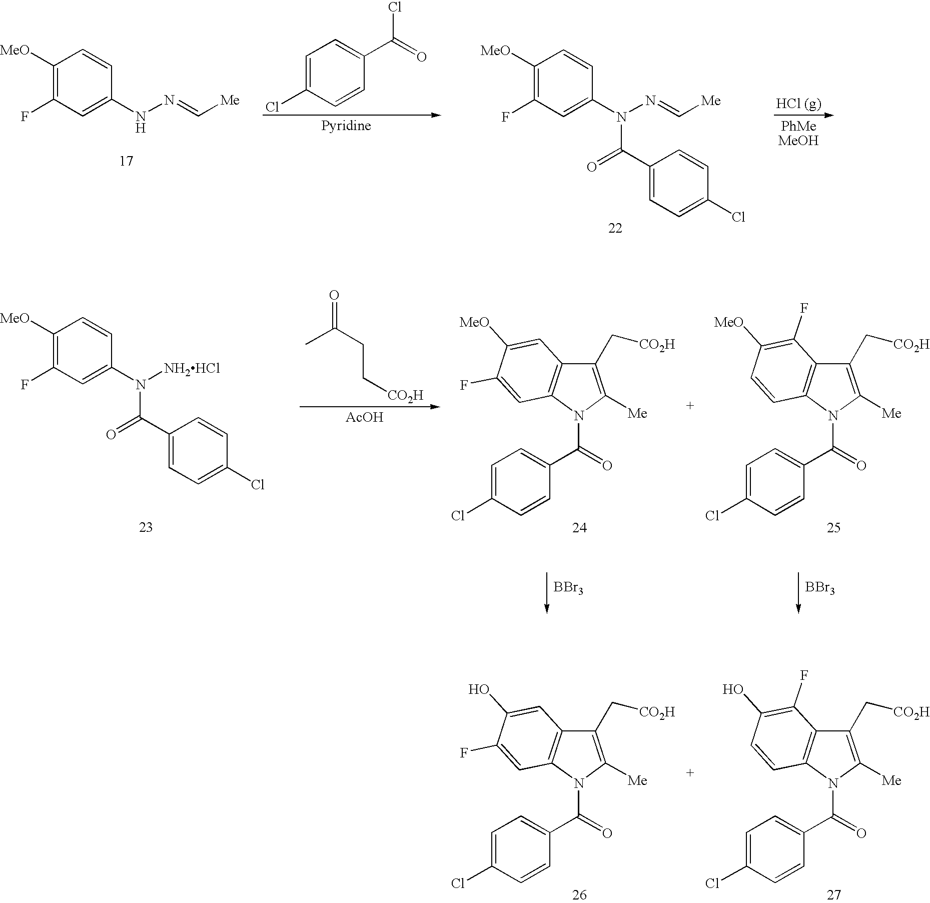

### Step 1. Preparation of 4-chloro-N′-ethylidene-N-(3-fluoro-4-methoxyphenyl) benzohydrazide (22)

4-Chlorobenzoyl chloride (20.4 mL, 28.0 g, 0.16 mol) was added dropwise at 10° C. to a stirred solution of acetaldehyde (3-fluoro-4-methoxyphenyl)hydrazone (17) (14.6 g, 0.08 mol) and pyridine (9.7 mL, 9.49 g, 0.12 mol) in ether (150 mL), the mixture was stirred at ambient temperature for 4 h, then it was quenched with water (200 mL). The product was extracted into ethyl acetate (2×100 mL), the combined extracts were washed with water (2×100 mL), dried (MgSO₄), and the solvent was removed in vacuo. The residual red solid was purified by chromatography over silica using a 3:7 mixture of ethyl acetate and hexane as eluant. Appropriate fractions were combined and the solvents were removed in vacuo to give 4-chloro-N′-ethylidene-N-(3-fluoro-4-methoxyphenyl)benzohydrazide (22) (10.8 g, 42%). as a yellow solid, mp 97-99° C.; 250 MHz ¹H-NMR (CDCl₃) δ (ppm): 1.80 (d, 3H, N═CHCH₃), 3.83 (s, 3H, OCH₃), 6.72-7.20 (m, 4H, C₆H₃ and N═CHCH₃), 7.30 (d, 2H, C₆H₂), 7.68 (d, 2H, C₆H₂).

### Step 2. Preparation of 4-chloro-N-(3-fluoro-4-methoxyphenyl)benzohydrazide hydrochloride (23)

Hydrogen chloride gas was bubbled through a solution of 4-chloro-N′-ethylidene-N-(3-fluoro-4-methoxyphenyl)benzohydrazide (22) (10.8 g, 33.67 mmol) in a mixture of toluene (150 mL) and methanol (10 mL) at 0° C. for 1 h. The resulting white solid was collected by filtration, washed with ethyl acetate (2×200 mL) and dried in vacuo to give 4-chloro-N-(3-fluoro-4-methoxyphenyl)benzohydrazide hydrochloride (23) (7.84 g, 70%) as a white solid, mp 180-182° C. 250 MHz ¹H-NMR (d₆-DMSO) δ (ppm): 3.86 (s, 3H, OCH₃), 7.1-7.55 (m, 7H, C₆H₃ and C₆H₄).

### Step 3. Preparation of [1-(4-chlorobenzoyl)-4-fluoro-5-methoxy-2-methyl-1H-indol-3-yl]acetic acid (25) and [1-(4-chlorobenzoyl)-6-fluoro-5-methoxy-2-methyl-1H-indol-3-yl]acetic acid (24)

A stirred mixture of 4-chloro-N-(3-fluoro-4-methoxyphenyl)benzohydrazide hydrochloride (23) (7.84 g, 23.67 mmol), levulinic acid (3.3 g, 28.4 mmol) and acetic acid (50 mL) was heated at 80° C. for 4 h, cooled to ambient temperature and added to ice-water (100 mL). The resulting solid was collected by filtration, dried in vacuo and crystallized from toluene to give a 5:2 mixture of [1-(4-chlorobenzoyl)-6-fluoro-5-methoxy-2-methyl-1H-indol-3-yl]acetic acid (24) and [1-(4-chlorobenzoyl)-4-fluoro-5-methoxy-2-methyl-1H-indol-3-yl]acetic acid (25) (7.2 g) as an off white solid. The mixture (7.2 g) was purified by column chromatography over silica using a 3:7 mixture of ethyl acetate and hexane as eluant. Appropriate fractions were combined and the solvents were removed in vacuo to give a small sample (300 mg) of the single regioisomer, [1-(4-chlorobenzoyl)-4-fluoro-5-methoxy-2-methyl-1H-indol-3-yl]acetic acid (25), and a larger sample (6.5 g), which was still a mixture of the two regioisomers. The larger sample (6.5 g) was subjected to a further purification by UV-triggered radial compression Biotage chromatography using gradient elution with 15:85-40:60 mixtures of ethyl acetate and hexane as eluants. Appropriate fractions were combined and the solvents were removed in vacuo to give [1-(4-chlorobenzoyl)-6-fluoro-5-methoxy-2-methyl-1H-indol-3-yl]acetic acid (24) 400 mg, [1-(4-chlorobenzoyl)-4-fluoro-5-methoxy-2-methyl-1H-indol-3-yl]acetic acid (25) 300 mg, and a larger sample (4.2 g) which was still a mixture of the two regioisomers.

[1-(4-Chlorobenzoyl)-6-fluoro-5-methoxy-2-methyl-1H-indol-3-yl]acetic acid (24) (400 mg, 5%) was thus obtained as a white solid, mp 145-147° C.; 250 MHz ¹H-NMR (CDCl₃) δ (ppm): 2.29 (s, 3H, CH₃), 3.62 (s, 2H, CH₂CO₂H), 3.86 (s, 3H, OCH₃), 6.76 (d, 1H, indole 4-H), 6.92 (d, 1H, indole 7-H), 7.42 (d, 2H, 2H in Ph), 7.60 (d, 2H, 2H in Ph), 10.00-11.00 (very br. COOH).

[1-(4-Chlorobenzoyl)-4-fluoro-5-methoxy-2-methyl-1H-indol-3-yl]acetic acid (25) (600 mg, 7%) was thus obtained as a yellow solid, mp 170-172° C.; 250 MHz ¹H-NMR (CDCl₃) δ (ppm): 2.27 (s, 3H, CH₃), 3.80 (s, 2H, CH₂CO₂H), 3.81 (s, 3H, OCH₃), 6.66 (m, 2H, indole 6-H and 7-H), 7.40 (d, 2H, 2H in Ph), 7.61 (d, 2H, 2H in Ph), 10.00-11.00 (very br. COOH).

### Step 4. Preparation of [1-(4-chlorobenzoyl)-6-fluoro-5-hydroxy-2-methyl-1H-indol-3-yl]acetic acid (26)

Boron tribromide (1 M solution in dichloromethane; 3.19 mL, 3.19 mmol) was added dropwise at −78° C. under nitrogen to a stirred solution of [1-(4-chlorobenzoyl)-6-fluoro-5-methoxy-2-methyl-1H-indol-3-yl]acetic acid (24) (400 mg, 1.06 mmol) in dichloromethane (10 mL), the mixture was allowed to warm to ambient temperature, then it was stirred for a further 4.5 h and added to ice-water (15 mL). The resulting solid was collected by filtration and dried in vacuo to give [1-(4-chlorobenzoyl)-6-fluoro-5-hydroxy-2-methyl-1H-indol-3-yl]acetic acid (26) (270 mg, 70%) as a white solid, mp 193-195° C.; 250 MHz ¹H—N (d₆-DMSO) δ (ppm): 2.09 (s, 3H, CH₃), 3.52 (s, 2H, CH₂CO₂H)), 6.83 (dd, 2H, indole 4-H and 7-H), 7.61 (s, 4H, C₆H₄), 9.60 (s, 1H, OH), 12.39 (s, 1H, COOH).

### Step 5. Preparation of [1-(4-chlorobenzoyl)-4-fluoro-5-hydroxy-2-methyl-1H-indol-3-yl]acetic acid (27)

Boron tribromide (1 M solution in dichloromethane; 3.99 mL, 3.99 mmol) was added dropwise at −78° C. under nitrogen to a stirred solution of [1-(4-chlorobenzoyl)-4-fluoro-5-methoxy-2-methyl-1H-indol-3-yl]acetic acid (25) (500 mg, 1.33 mmol) in dichloromethane (10 mL), the mixture was allowed to warm to ambient temperature, then it was stirred for a further 4.5 h and added to ice-water (15 mL). The resulting solid was collected by filtration and dried in vacuo to give [1-(4-chlorobenzoyl)-4-fluoro-5-hydroxy-2-methyl-1H-indol-3-yl]acetic acid (27) (410 mg, 70%) as an off white solid, mp 187-189° C.; 250 MHz ¹H-NMR (d₆-DMSO) δ (ppm): 2.15 (s, 3H, CH₃), 3.77 (s, 2H, CH₂CO₂H)), 6.72 (s, 2H, indole 6-H and 7-H), 7.69 (s, 4H, C₆H₄), 9.48 (s, 1H, OH), 12.39 (s, 1H, COOH).

### EXAMPLE 7

### [1-(4-Chlorobenzoyl)-4,6-difluoro-5-hydroxy-2-methyl-1H-indol-3-yl]acetic acid

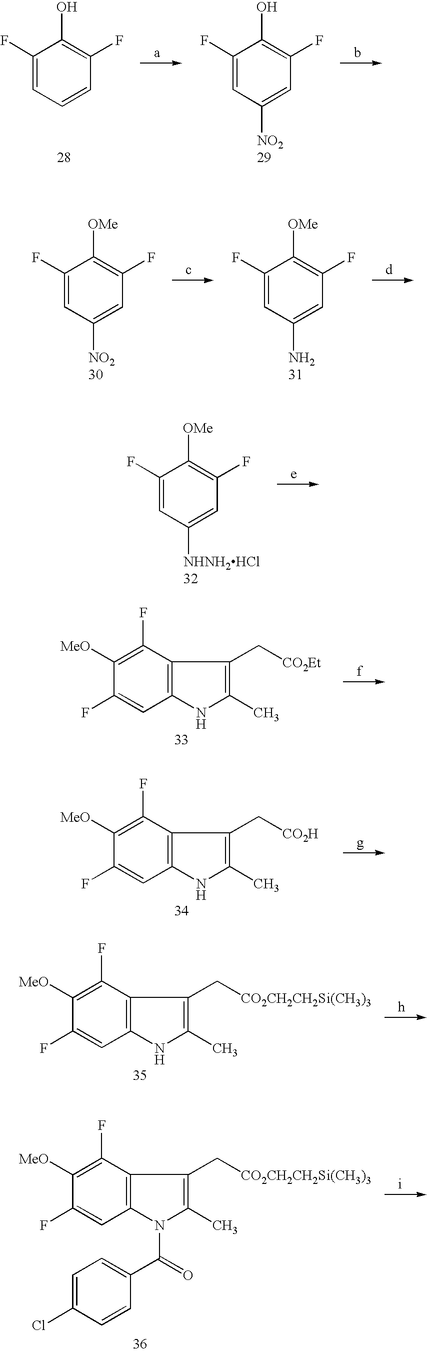

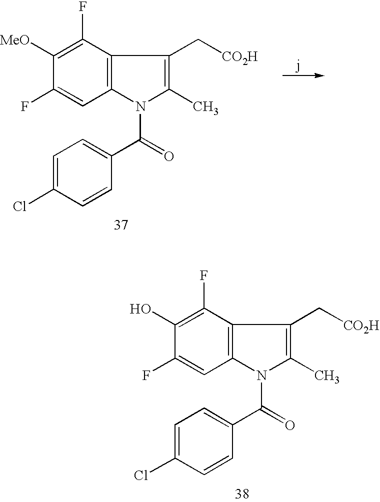

Reagents: a) HNO₃, (b) CH₃I, K₂CO₃, (c) H₂ Pd/C, (d) 1. NaNO₂, HCl 2. SnCl₂, HCl (e) HCl, EtOH, levulinic acid, (f) NaOH, (g) Me₃SiCH₂CH₂OH, EDCl, (h) NaH, ClC₆H₄COCl, DMF, (i) n-Bu₄NF, THF, (J) BBr₃

### Step 1. Preparation of 2,6-difluoro-4-nitrophenol (29)

Nitric acid (39.5 mL, 58.85 g, 0.93 mol) was added dropwise at 5° C. to a stirred solution of 2,6-difluorophenol (28) (97.5 g, 0.75 mol) in acetic acid (250 mL). After addition was complete the mixture was allowed to warm to ambient temperature, then it was stirred for 1 h and poured into ice-water (2 L). The resulting solution was concentrated under reduced pressure to ˜1 L and the product was extracted into dichloromethane (5×250 mL). The combined extracts were washed with water (2×800 mL), dried (MgSO₄) and the solvent was removed in vacuo. The residual oil was heated under reflux with isooctane (1.5 L) for a few min, then the solution was decanted from an insoluble residue and cooled in ice-water. The resulting yellow solid was collected by filtration and dried in vacuo to give 2,6-difluoro-4-nitrophenol (29) (94.6 g, 72%) as a yellow solid, mp 98.4-100.8° C.; 250 MHz ¹H-NMR (CDCl₃) δ (ppm): 7.09 (broad s, 1H, OH), 7.80 (m, 2H, 2×ArH).

### Step 2. Preparation of 1,3-difluoro-2-methoxy-5-nitrobenzene (30)

Iodomethane (125.1 mL, 285.3 g, 2.01 mol) was added dropwise at ambient temperature to a stirred mixture of 2,6-difluoro-4-nitrophenol (29) (116.7 g, 0.67 mol; prepared in a manner similar to that described above), acetone (1200 mL) and potassium carbonate (237.58 g, 1.33 mol), the stirred mixture was heated under reflux for 4 h, then it was allowed to cool to ambient temperature. The mixture was filtered, the solvent was removed in vacuo, and the residual red oil was poured into water (500 mL). The product was extracted into ethyl acetate (4×250 mL), the combined extracts were dried (MgSO₄) and the solvent was removed in vacuo. The residual dark oil was purified by distillation to give 1,3-difluoro-2-methoxy-5-nitrobenzene (30) (117.7 g, 93%) as a yellow oil, bp 72-78° C. @ 0.05 mbar, which solidified on standing at ambient temperature; 250 MHz

¹H-NMR (CDCl₃) δ (ppm): 4.10 (t, J=1.75 Hz, 3H, OCH₃), 7.77 (d, J=8.75 Hz, 2H, 2×ArH).

### Step 3. Preparation of 3,5-difluoro-4-methoxyaniline (31)

A stirred mixture of 10% palladium on carbon catalyst (1.5 g), 1,3-difluoro-2-methoxy-5-nitrobenzene (30) (117 g, 0.62 mol) and ethyl acetate (750 mL) was hydrogenated at 400 psi pressure and ambient temperature until hydrogen uptake ceased, then it was filtered through Celite and dried (MgSO₄). The solvent was removed in vacuo, the residue was triturated with hexane (300 mL) and the resulting solid was collected by filtration and dried in vacuo to give 3,5-difluoro-4-methoxyaniline (31) (94.85 g, 96%) as a yellow solid, mp 75.9-77.4° C.; 250 MHz ¹H-NMR (CDCl₃) δ (ppm): 3.72 (br s, 2H, NH₂), 3.92 (t, J=0.6 Hz, 3H, OCH₃), 6.25 (d, J=10.1 Hz, 2H, 2×ArH).

### Step 4. Preparation of 3,5-difluoro-4-methoxyphenylhydrazine (32)

3,5-Difluoro-4-methoxyaniline (31) (50 g, 0.31 mol) was added to concentrated hydrochloric acid (150 mL), the suspension was stirred at ambient temperature for 30 min, then it was cooled to 0° C. and a solution of sodium nitrite (21.7 g, 0.31 mol) in water (100 mL) was added dropwise at 0-5° C. When the addition was complete, the resulting solution was stirred at 0° C. for 30 min then it was added dropwise at 0-5° C. to a stirred solution of tin(II) chloride dihydrate (209.8 g, 0.93 mol) in concentrated hydrochloric acid (300 mL). The mixture was allowed to warm to ambient temperature then it was stored at 4° C. for 18 h. The resulting precipitated hydrochloride salt was collected by filtration and washed with water (400 mL) and ether (400 mL), then it was basified by addition to 10% aqueous sodium hydroxide solution (600 mL). The free base was extracted into ether (4×300 mL), and the combined extracts were dried (MgSO₄) and the solvent removed in vacuo to give 3,5-difluoro-4-methoxyphenylhydrazine (32) (36.3 g, 67%) as a light brown solid, mp 60.5-62.3° C.; 250 MHz ¹H-NMR (CDCl₃) 8 (ppm): 3.50 (br s, 2H, NH₂), 3.90 (t, 3H, OCH₃), 5.22 (br s, 1H, NH), 6.42 (dd, 2H, 2×ArH).

### Step 5. Preparation of ethyl (4,6-difluoro-5-methoxy-2-methyl-1H-indol-3-yl)acetate (33)

Hydrogen chloride gas was bubbled into a solution of 3,5-difluoro-4-methoxyphenylhydrazine (32) (10.6 g, 60.9 mmol) and levulinic acid (7.77 g, 66.95 mmol) in ethanol (250 mL) at 0° C. until the solution was saturated. The solution was heated under reflux for 72 h, then it was allowed to cool to room temperature and the ethanol was removed in vacuo. The residue was dissolved in water (200 mL) and the product was extracted into dichloromethane (3×100 mL). The combined extracts were washed with water (200 mL) and saturated sodium bicarbonate solution (200 mL), dried (MgSO₄) and the solvent removed in vacuo to give a red oil. Unreacted ethyl levulinate (3.06 g) was removed from the oil by kugelröhr distillation at 80° C. @ 1.0 mbar, then the residue was triturated with hexane (50 mL) and the resulting brown solid was collected by filtration. The solid was purified by column chromatography over silica using dichloromethane as eluant. Appropriate fractions were combined and the solvent was removed in vacuo to give ethyl (4,6-difluoro-5-methoxy-2-methyl-1H-indol-3-yl)acetate (33) (7.1 g, 37%) as a red solid, mp 92.1-92.7° C.; 250 MHz ¹H-NMR (CDCl₃) δ (ppm): 1.21 (t, J=7.1 Hz, 3H, CH₃), 2.13 (s, 3H, CH₃), 3.66 (s, 2H, CH₂), 3.86 (s, 3H, OCH₃), 4.12 (q, J=7.1 Hz, 2H, CH₂), 6.60 (dd, J=1.58 and 10.42 Hz, 1H, ArH), 8.06 (br s, 1H, NH).

### Step 6. Preparation of (4,6-difluoro-5-methoxy-2-methyl-1H-indol-3-yl)acetic acid (34)

A mixture of sodium hydroxide (1.49 g, 37.30 mmol), ethyl (4,6-difluoro-5-methoxy-2-methyl-1H-indol-3-yl)acetate (33) (7.05 g, 24.9 mmol) and ethanol (75 mL) was heated under reflux for 1 h, then it was allowed to cool and the solvent was removed in vacuo. The residue was dissolved in water (150 mL), the solution was washed with ether (3×50 mL), then it was acidified by the addition of dilute (10%) hydrochloric acid. The resulting precipitate was collected by filtration and dissolved in ethyl acetate (300 mL). The solution was dried (MgSO₄) and the solvent was removed in vacuo to give (4,6-difluoro-5-methoxy-2-methyl-1H-indol-3-yl)acetic acid (34) (4.85 g, 76%) as an orange solid, mp 176.3-178.2° C.; 250 MHz ¹H-NMR (d₆-DMSO) δ (ppm) 2.27 (s, 3H, CH₃), 3.62 (s, 2H, CH₂), 3.83 (s, 3H, OCH₃), 6.98 (dd, J=1.42 and 10.74 Hz, 1H, ArH), 11.12 (br s, 1H, NH), 12.16 (br s, 1H, CO₂H).

### Step 7. Preparation of (2-(trimethylsilyl)ethyl (4,6-difluoro-5-methoxy-2-methyl-1H-indol-3-yl)acetate (35)

4-Dimethylaminopyridine (0.23 g, 1.85 mmol) and 1-[3-(dimethylamino)propyl]-3-ethylcarbodiimide hydrochloride (3.91 g, 20.4 mmol) were added at 0° C. to a stirred solution of (4,6-difluoro-5-methoxy-2-methyl-1H-indol-3-yl)acetic acid (4.73 g, 18.53 mmol) and 2-(trimethylsilyl)ethanol (3.98 mL, 3.29 g, 27.80 mmol) in dichloromethane (75 mL), the mixture was allowed to warm to ambient temperature then it was stirred for a further 3.5 h and poured onto water (100 mL). The organic layer was separated, washed with water (2×100 mL), dried (MgSO₄) and the solvent was removed in vacuo. The residue was triturated with hexane (50 mL) and the resulting solid was collected by filtration and dried in vacuo to give 2-(trimethylsilyl)ethyl (4,6-difluoro-5-methoxy-2-methyl-1H-indol-3-yl)acetate (35) (5.32 g, 81%) as a light brown solid, mp 86.9-88.7° C.; 250 MHz ¹H-NMR (CDCl₃) δ (ppm): 0.00 (s, 9H, SiMe₃), 0.97 (m, 2H, CH₂), 2.26 (s, 3H, CH₃), 3.72 (s, 2H, CH₂), 3.93 (s, 3H, OCH₃), 4.19 (m, 2H, CH₂), 6.72 (dd, J=1.58 and 10.43 Hz, 1H, ArH), 7.92 (br s, 1H, NH).

### Step 8. Preparation of 2-(trimethylsilyl)ethyl[1-(4-chlorobenzoyl]-4,6-difluoro-5-methoxy-2-methyl-1H-indol-3-yl]acetate (36)

A solution of 2-(trimethylsilyl)ethyl (4,6-difluoro-5-methoxy-2-methyl-1H-indol-3-yl)acetate (35) (3 g, 8.44 mmol) in dimethylformamide (10 mL) was added dropwise at ambient temperature under nitrogen to a stirred suspension of sodium hydride (60% dispersion in mineral oil, 0.51 g, 12.66 mmol) in dimethylformamide (40 mL). The mixture was stirred under nitrogen at ambient temperature for 30 min, then it was cooled to 0° C. 4-Chlorobenzoyl chloride (1.62 g, 9.28 mmol) was added dropwise, the mixture was allowed to warm to ambient temperature, then it was stirred for 1 h and poured onto ice-water (200 mL). The product was extracted into ethyl acetate (3×100 mL), the combined extracts were washed with water (200 mL) and saturated aqueous sodium chloride solution (200 mL), then they were dried (MgSO₄) and the solvent was removed in vacuo. The residual red oil was purified by column chromatography over silica using a 1:5 mixture of ethyl acetate and hexane as eluant. Appropriate fractions were combined and the solvents were removed in vacuo to give 2-(trimethylsilyl)ethyl[1-(4-chlorobenzoyl]-4,6-difluoro-5-methoxy-2-methyl-1H-indol-3-yl]acetate (36) (2.32 g, 55%) as a yellow oil; 250 MHz ¹H-NMR (CDCl₃) δ (ppm): 0.00 (s, 9H, SiMe₃), 0.97 (m, 2H, CH₂), 2.20 (s, 3H, CH₃), 3.73 (s, 2H, CH₂), 3.93 (s, 3H, OCH₃), 4.20 (m, 2H, CH₂), 6.72 (dd, J=1.58 and 11.22 Hz, 1H, ArH), 7.47 (d, J=8.53 Hz, 2H, 2×ArH), 7.63 (d, J=8.85 Hz, 2H, 2×ArH).

### Step 9. Preparation of [1-(4-chlorobenzoyl)-4,6-difluoro-5-methoxy-2-methyl-1H-indol-3-yl]acetic acid (37)

Tetrabutylammonium fluoride (1.0 M solution in tetrahydrofuran, 5.0 mL, 5.07 mmol) was added at 0° C. under nitrogen to a stirred solution of 2-(trimethylsilyl)ethyl[1-(4-chlorobenzoyl]-4,6-difluoro-5-methoxy-2-methyl-1H-indol-3-yl]acetate (36) (2.28 g, 4.62 mmol) in tetrahydrofuran (20 mL), the mixture was allowed to warm to ambient temperature, then it was stirred for 3 h, and the solvent was removed in vacuo. The residual orange oil was dissolved in water (50 mL), the solution was acidified to pH 1 by the addition of 2M aqueous citric acid solution, and the product was extracted into ethyl acetate (3×50 mL). The combined extracts were dried (MgSO₄) and the solvent was removed in vacuo to give a sticky solid. The solid was triturated with hot dichloromethane (50 mL), the resulting suspension was filtered to remove an insoluble white solid (1.45 g), then the filtrate was concentrated in vacuo. The residue was purified by column chromatography over silica using a 1:1 mixture of ethyl acetate and hexane as eluant. Appropriate fractions were combined and the solvents were removed in vacuo to give [1-(4-chlorobenzoyl)-4,6-difluoro-5-methoxy-2-methyl-1H-indol-3-yl]acetic acid (37) (0.070 g, 4%) as an impure yellow gum which was used without further purification. The maJor product isolated was (4,6-difluoro-5-methoxy-2-methyl-1H-indol-3-yl)acetic acid (0.92 g, 56%).

### Step 10. Preparation of [1-(4-chlorobenzoyl)-4,6-difluoro-5-hydroxy-2-methyl-1H-indol-3-yl]acetic acid (38)

Boron tribromide (1 M solution in dichloromethane; 0.53 mL, 0.53 mmol) was added dropwise at −78° C. under nitrogen to a stirred solution of [1-(4-chlorobenzoyl)-4,6difluoro-5-methoxy-2-methyl-1H-indol-3-yl]acetic acid (37) (0.07 g, 0.18 mmol) in dichloromethane (5 mL), the-mixture was allowed to warm to ambient temperature, then it was stirred for a further 3 h. The mixture was added to ice-water (50 mL) and the resulting precipitate was collected by filtration and dried in vacuo to give [1-(4-chlorobenzoyl)-4,6-difluoro-5-hydroxy-2-methyl-1H-indol-3-yl]acetic acid (38) (7 mg, 10%) as a brown solid; 250 MHz ¹H-NMR (d₆-DMSO) δ (ppm): 2.10 (s, 3H, CH₃), 3.70 (s, 2H, CH₂), 6.87 (dd, J=1.26 and 11.53 Hz, 1H, ArH), 7.69 (s, 4H, 4×ArH), 9.83 (s, 1H, OH), 12.46 (br s, 1H CO₂H).

### EXAMPLE 8

### [1-(3-Chlorobenzoyl)-5-hydroxy-2-methyl-1H-indol-3-yl]acetic acid

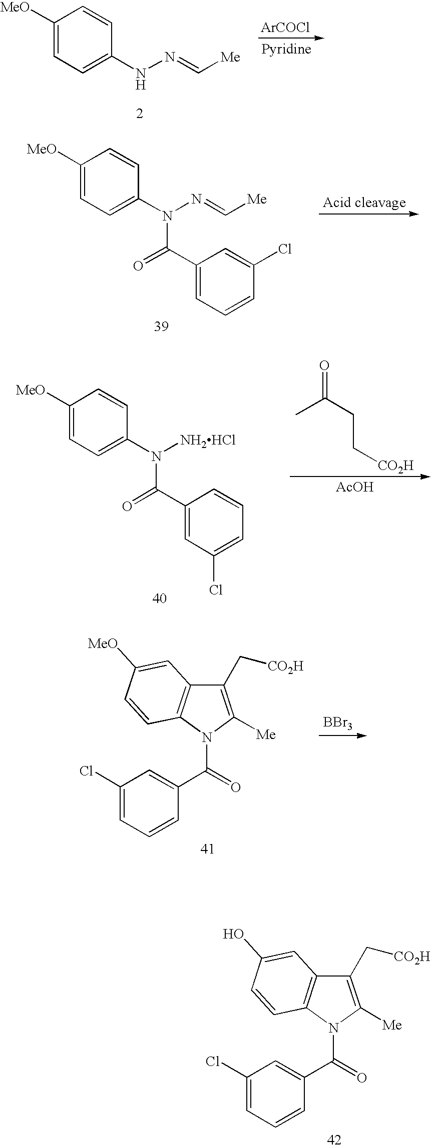

### Step 1. Preparation of 3-chloro-N′-ethylidene-N-(4-methoxyphenyl)benzohydrazide (39)

3-Chlorobenzoyl chloride (15.0 g, 0.086 mol) was added dropwise at 10° C. to a stirred solution of acetaldehyde 4-methoxyphenylhydrazone (7 g, 0.043 mol) in pyridine (60 mL), the mixture was stirred at ambient temperature for 4 h, then it was quenched with water (200 mL). The product was extracted into ethyl acetate (2×100 mL), the combined extracts were washed with water (2×100 mL), dried (MgSO₄), and the solvent was removed in vacuo. The residual red solid was purified by chromatography over silica using a 3:7 mixture of ethyl acetate and hexane as eluant. Appropriate fractions were combined and the solvents were removed in vacuo to give 3-chloro-N′-ethylidene-N-(4-methoxyphenyl)benzohydrazide (39) (6.3 g) as a yellow oil which was used without further purification.

### Step 2. Preparation of 3-chloro-N-(4-methoxyphenyl)benzohydrazide hydrochloride (40)

Hydrogen chloride gas was bubbled through a solution of crude 3-chloro-N′-ethylidene-N-(4-methoxyphenyl)benzohydrazide (39) (6.3 g) in a mixture of toluene (100 mL) and methanol (10 mL) at 0° C. for 1 h. The solvents were removed in vacuo and the residual solid was triturated with ethyl acetate (200 mL). The resulting solid was collected by filtration and dried in vacuo to give 3-chloro-N-(4-methoxyphenyl)benzohydrazide hydrochloride (40) (2.5 g) as a white solid, mp 155-157° C.; 250 MHz ¹H-NMR (d₆-DMSO) δ (ppm): 3.76 (s, 3H, OCH₃), 6.90 (d, 2H, 2H in Ph), 7.22-7.55 (m, 6H, C₆H₄ and 2H in Ph). (Note—there was no signal evident down to 12 ppm for —NH₃⁺).

### Step 3. Preparation of [1-(3-chlorobenzoyl)-5-methoxy-2-methyl-1H-indol-3-yl]acetic acid (41)

Levulinic acid (1.11 g, 9.6 mmol) was added to a stirred solution of 3-chloro-N-(4-methoxyphenyl)benzohydrazide hydrochloride (40) (2.5 g, 8.0 mmol) in acetic acid (20 mL), the mixture was heated under reflux under nitrogen for 4 h, then it was allowed to cool to ambient temperature and poured onto ice-water (50 mL). The resulting precipitate was collected by filtration and dried in vacuo to give [1-(3-chlorobenzoyl)-5-methoxy-2-methyl-1H-indol-3-yl]acetic acid (41) (2.0 g, 73%) as an off white solid, mp 90-92° C.; 250 MHz ¹H-NMR (CDCl₃) δ (ppm): 2.30 (s, 3H, CH₃), 3.59 (s, 2H, CH₂COOH), 3.72 (s, 3H, OCH₃), 6.60 (dd, 1H, indole 6-H), 6.79 (d, 1H, indole 7-H), 6.82 (d, 1H, indole 4-H), 7.34 (t, 1H, 1H in Ph), 7.50 (m, 2H, 2H in Ph), 7.62 (s, 1H, 1H in Ph), 10.00-11.00 (very br, cannot be integrated, COOH).

### Step 4. Preparation of [1-(3-chlorobenzoyl)-5-hydroxy-2-methyl-1H-indol-3-yl]acetic acid (42)

Boron tribromide (1 M solution in dichloromethane; 13.9 mL, 13.9 mmol) was added dropwise at −78° C. under nitrogen to a stirred solution of [1-(3-chlorobenzoyl)-5-methoxy-2-methyl-1H-indol-3-yl]acetic acid (41) (1.6 g, 4.65 mmol) in dichloromethane (20 mL), the mixture was allowed to warm to ambient temperature and stirred for 4 h, then it was poured onto water (30 mL). The resulting solid was collected by filtration and dried in vacuo to give [1-(3-chlorobenzoyl)-5-hydroxy-2-methyl-1H-indol-3-yl]acetic acid (42) (1.4 g, 91%) as a grey solid, mp 158-160° C.; 250 MHz ¹H-NMR (d₆-DMSO) δ (ppm): 2.19 (s, 3H, CH₃), 3.60 (s, 2H, CH₂COOH), 6.50 (d, 1H, indole 6-H), 6.85 (dd, 2H, indole 4-H and 7-H), 7.60 (m, 2H, 2H in Ph), 7.78 (m, 2H, 2H in Ph), 9.20 (br, 1H, OH), 12.00-13.00 (br, 1H, COOH).

### EXAMPLE 9

### [1-(3,4-Difluorobenzoyl)-5-hydroxy-2-methyl-1H-indol-3-yl]acetic acid (46)

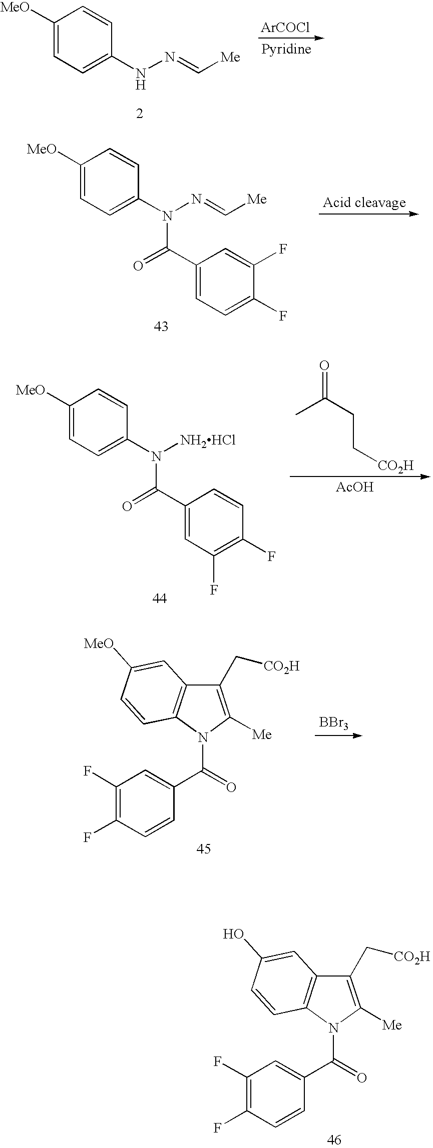

### Step 1. Preparation of N′-ethylidene-3,4-difluoro-N-(4-methoxyphenyl)benzohydrazide (43)

3,4-Difluorobenzoyl chloride (12.3 ml, 17.3 g, 0.098 mol) was added dropwise at 10° C. under nitrogen to a stirred solution of acetaldehyde 4-methoxyphenylhydrazone (8 g, 0.049 mol) in pyridine (40 mL), the mixture was stirred at ambient temperature for 18 h, then it was added to ice-water (250 mL). The product was extracted into dichloromethane (2×200 mL), the combined extracts were dried (MgSO₄) and the solvent was removed in vacuo. The residue was purified by flash chromatography over silica using a 3:7 mixture of ethyl acetate and hexane as eluant. Appropriate fractions were combined and the solvents were removed in vacuo to give N′-ethylidene-3,4-difluoro-N-(4-methoxyphenyl)benzohydrazide (43) (11 g) as a brown oil which was used without further purification.

### Step 2. Preparation of 3,4-difluoro-N-(4-methoxyphenyl)benzohydrazide hydrochloride (44)

Hydrogen chloride gas was bubbled through a solution of crude NY-ethylidene-3,4-difluoro-N-(4-methoxyphenyl)benzohydrazide (43) (11 g) in a mixture of toluene (100 mL) and methanol (10 mL) at 0° C. for 1 h. The solvents were removed in vacuo and the residual solid was triturated with ethyl acetate (200 mL). The resulting solid was collected by filtration and dried in vacuo to give 3,4-difluoro-N-(4-methoxyphenyl)benzohydrazide hydrochloride (44) (2.0 g) as a white solid, mp 164-166° C.; 250 MHz ¹H-NMR (d₆-DMSO) δ (ppm): 3.76 (s, 3H, OCH₃), 6.99 (m, 3H, 3H in Ph), 7.35-7.49 (m, 4H, C₆H₄), 8.0-9.0 (br, —NH₃⁺).

### Step 3. Preparation of [1-(3,4-difluorobenzoyl)-5-methoxy-2-methyl-1H-indol-3-yl]acetic acid (45)

Levulinic acid (0.88 g, 7.62 mmol) was added to a stirred solution of 3,4-difluoro-N-(4-methoxyphenyl)benzohydrazide hydrochloride (44) (2.0 g, 6.35 mmol) in acetic acid (20 mL), the mixture was heated under reflux under nitrogen for 4 h, then it was allowed to cool to ambient temperature and poured onto ice-water (50 mL). The resulting precipitate was collected by filtration and dried in vacuo to give [1-(3,4-difluorobenzoyl)-5-methoxy-2-methyl-1H-indol-3-yl]acetic acid (45) (1.8 g, 82%) as a grey solid, mp 113-115° C.; 250 MHz ¹H-NMR (CDCl₃) δ (ppm): 2.35 (s, 3H, CH₃), 3.60 (s, 2H, CH₂COOH), 3.72 (s, 3H, OCH₃), 6.58 (d, 1H, indole 6-H), 6.80 (d, 1H, indole 7-H), 6.90 (d, 1H, indole 4-H), 7.10-7.60 (m, 3H, 3H in Ph), 11.00 (very br, cannot be integrated, COOH).

### Step 4. Preparation of [1-(3,4-difluorobenzoyl)-5-hydroxy-2-methyl-1H-indol-3-yl]acetic acid (46)

Boron tribromide (1 M solution in dichloromethane; 13 mL, 13 mmol) was added dropwise at −78° C. under nitrogen to a stirred solution of [1-(3,4-difluorobenzoyl)-5-methoxy-2-methyl-1H-indol-3-yl]acetic acid (45) (1.6 g, 4.6 mmol) in dichloromethane (20 mL), the mixture was allowed to warm to ambient temperature and stirred for 4 h, then it was poured onto water (30 mL). The resulting solid was collected by filtration and dried in vacuo to give [1-(3,4-difluorobenzoyl)-5-hydroxy-2-methyl-1H-indol-3-yl]acetic acid (46) (1.6 g, 98%) as a grey solid, mp 143-145° C.; 250 MHz ¹H-NMR (d₆-DMSO) δ (ppm): 2.22 (s, 3H, CH₃), 3.60 (s, 2H, CH₂COOH), 6.65 (m, 1H, indole 6-H), 6.88 (m, 2H, indole 4-H and 7-H), 7.60-7.89 (m, 3H, 3H in Ph). (Note—there was no signal evident for OH and COOH).

### EXAMPLE 10

### [1-(3,4-Dichlorobenzoyl)-5-hydroxy-2-methyl-1H-indol-3-yl]acetic acid (50)

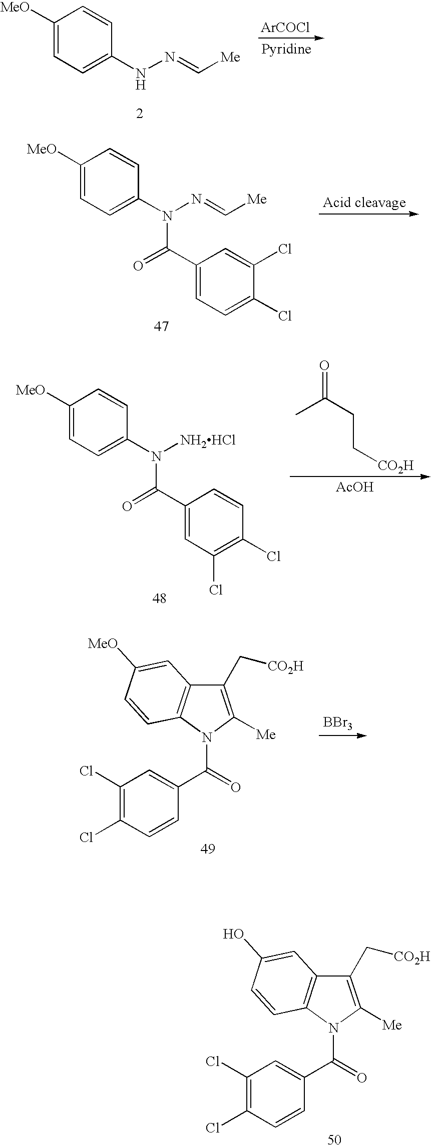

### Step 1. Preparation of 3,4-dichloro-N′-ethylidene-N-(4-methoxyphenyl)benzohydrazide (47)

3,4-Dichlorobenzoyl chloride (25.68 g, 0.123 mol) was added dropwise at 10° C. to a stirred solution of acetaldehyde 4-methoxyphenylhydrazone (2) (10 g, 0.613 mol) and pyridine (7.4 mL, 7.27 g, 0.092 mol) in ether (150 mL), the mixture was stirred at ambient temperature for 18 h, then it was quenched with water (200 mL). The product was extracted into ethyl acetate (2×100 n-L), the combined extracts were washed with water (2×100 mL), dried (MgSO₄), and the solvent was removed in vacuo. The residual red solid was purified by chromatography over silica using a 3:7 mixture of ethyl acetate and hexane as eluant. Appropriate fractions were combined and the solvents were removed in vacuo to give 3,4-dichloro-N′-ethylidene-N-(4-methoxyphenyl)benzohydrazide (47) (6.0 g) as a yellow oil which was used without further purification.

### Step 2. Preparation of 3,4-dichloro-N-(4-methoxyphenyl)benzohydrazide hydrochloride (48)

Hydrogen chloride gas was bubbled through a solution of crude 3,4-dichloro-N′-ethylidene-N-(4-methoxyphenyl)benzohydrazide (47) (6.0 g) in a mixture of toluene (100 mL) and methanol (10 mL) at 0° C. for 1 h. The solvents were removed in vacuo and the residual solid was triturated with ethyl acetate (200 mL). The resulting solid was collected by filtration and dried in vacuo to give 3,4-dichloro-N-(4-methoxyphenyl)benzohydrazide hydrochloride (48) (3.7 g) as a white solid, mp 177-179° C.; 250 MHz ¹H-NMR (d₆-DMSO) δ (ppm): 3.76 (s, 3H, OCH₃), 6.90 (d, 2H, 2H in Ph), 7.22-7.55 (m, 5H, C₆H₃ and 2H in Ph). (Note—there was no signal evident down to 12 ppm for —NH₃⁺).

### Step 3. Preparation of [1-(3,4-dichlorobenzoyl)-5-methoxy-2-methyl-1H-indol-3-yl]acetic acid (49)

Levulinic acid (1.48 g, 12.8 mmol) was added to a stirred solution of 3,4-dichloro-N-(4-methoxyphenyl)benzohydrazide hydrochloride (48) (3.7 g, 10.6 mmol) in acetic acid (40 mL), the mixture was heated under reflux under nitrogen for 4 h, then it was allowed to cool to ambient temperature and poured onto ice-water (50 mL). The resulting precipitate was collected by filtration and dried in vacuo to give [1-(3,4-dichlorobenzoyl)-5-methoxy-2-methyl-1H-indol-3-yl]acetic acid (49) (3.8 g, 95%) as a grey solid, mp 95-97° C.; 250 MHz ¹H-NMR (CDCl₃) δ (ppm): 2.31 (s, 3H, CH₃), 3.59 (s, 2H, CH₂COOH), 3.72 (s, 3H, OCH₃), 6.60 (dd, 1H, indole 6-H), 6.79 (d, 1H, indole 7-H), 6.82 (d, 1H, indole 4-H), 7.44 (m, 2H, 2H in Ph), 7.71 (s, 1H, 1H in Ph), 11.00 (very br, cannot be integrated, COOH).

### Step 4. Preparation of [1-(3,4-dichlorobenzoyl)-5-hydroxy-2-methyl-1H-indol-3-yl]acetic acid (50)

Boron tribromide (1 M solution in dichloromethane; 23.7 mL, 23.7 mmol) was added dropwise at −78° C. under nitrogen to a stirred solution of [1-(3,4-dichlorobenzoyl)-5-methoxy-2-methyl-1H-indol-3-yl]acetic acid (49) (3 g, 7.93 mmol) in dichloromethane (50 mL), the mixture was allowed to warm to ambient temperature and stirred for 4 h, then it was poured onto water (50 mL). The resulting solid was collected by filtration and dried in vacuo to give give [1-(3,4-dichlorobenzoyl)-5-hydroxy-2-methyl-1H-indol-3-yl]acetic acid (50) (1.8 g, 62%) as grey solid, mp 177-179° C.; 250 MHz ¹H-NMR (d₆-DMSO) δ (ppm): 2.10 (s, 3H, CH₃), 3.52 (s, 2H, CH₂COOH), 6.41 (d, 1H, indole 6-H), 6.80 (dd, 2H, indole 4-H and 7-H), 7.50 (d, 1H, 1H in Ph), 7.72 (d, 1H, 1H in Ph), 7.85 (s, 1H, 1H in Ph), 9.20 (br, 1H, OH), 12.3 (br, 1H, COOH).

### EXAMPLE 11

### [1-(4-Chlorobenzoyl)-6-fluoro-5-hydroxy-2-methyl-1H-indol-3-yl]acetic acid (26) via Method B

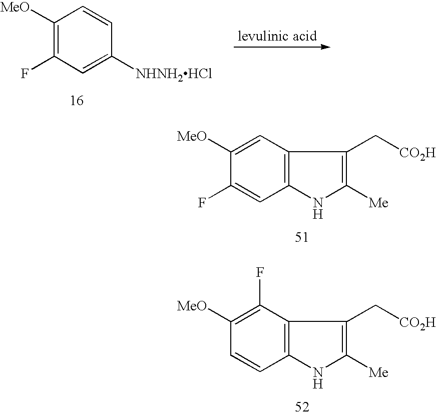

### Step 1. Preparation of (6-fluoro-5-methoxy-2-methyl-1H-indol-3-yl)acetic acid (51) and (4-fluoro-5-methoxy-2-methyl-1H-indol-3-yl)acetic acid (52)

Levulinic acid (38 mL, 354 mmol) and 3-fluoro-6-methoxy-phenylhydrazine hydrochloride (16) (67.5 g, 350 mmol) were combined and 150 mL of glacial acetic acid added and the slurry was stirred at 80° C. for 4 h. The reaction was cooled to room temperature and added to ice water (500 mL). The resulting aqueous solution was extracted with dichloromethane (3×500 mL) and the organics dried (MgSO₄) and concentrated to afford a thick semi-solid. Water (450-500 mL) was added and the slurry was stirred vigorously overnight while manually breaking up large solid pieces with a spatula. The fine tan solid that results is isolated by filtration and dried to afford a mixture of indoles 56.3 grams, 67% yield, ˜93% pure by HPLC (7/1 ratio of (6-fluoro-5methoxy-2-methyl-1H-indol-3-yl)acetic acid (51) and (4-fluoro-5-methoxy-2-methyl-1H-indol-3-yl)acetic acid (52) of by NM). MaJor isomer 300 MHz ¹H-NMR (CDCl₃) δ 2.27 (s, 3H), 3.82 (s, 2H), 3.84 (s, 3H), 6.92-6.97 (m, 2H, ArH).

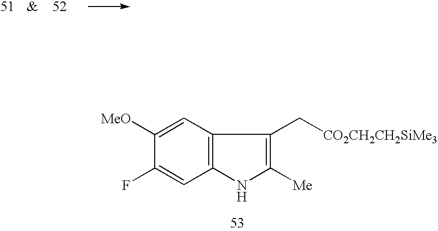

### Step 2. Preparation of 2-trimethylsilylethyl (6-fluoro-5-methoxy-2-methyl-1H-indol-3-yl)acetate (53)

The indoles from Step 1 (56.3 g, 238 mmol) were combined with 2-trimethylsilylethanol (41 mL, 1.25 eq.) and DMAP (4 g) in dichloromethane (600 mL) and cooled to 0° C. 1-[3-(Dimethylamino)propyl)]-3-ethylcarbodiimide hydrochloride (EDCl) (50.2 g, 1.1 eq.) was added in portions and the reaction was stirred for 30 min at 0° C. and then allowed to warm to room temperature and stir overnight. The reaction mixture was diluted with dichloromethane (600 mL) and washed with water (2×200 mL), dried and concentrated to give a thick orange syrup which after triturating with hexanes induced solid formation, the solid was recrystallized from hexane-ethyl acetate to afford tan needles of 2-trimethylsilylethyl (6-fluoro-5-methoxy-2-methyl-1H-indol-3-yl)acetate (53), 52 g, 65% yield, >98% pure; 300 MHz ¹H-NMR (CDCl₃) δ 0.16 (s, 9H), 0.98 (m, 2H), 2.37 (s, 3H), 3.61 (s, 2H), 3.93 (s, 3H), 4.12 (m, 2H), 7.00-7.05 (m, 2H, ArH).

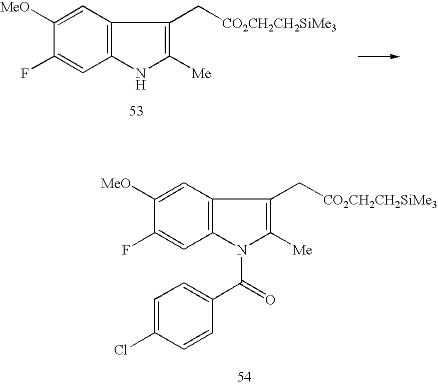

### Step 3. Preparation of 2-trimethysilylethyl[1-(4-chlorobenzoyl)-6-fluoro-5-methoxy-2-methyl-1H-indol-3-yl]acetate (54)

In a dry flask 2-trimethylsilylethyl (6-fluoro-5-methoxy-2-methyl-1H-indol-3-yl)acetate (53) (5 g, 14.8 mmol) was dissolved in THF (50 mL) and HMPA (5 mL) and cooled to −78° C. Potassium bis(trimethylsilyl)amide 0.5M in toluene (32.5 mL) was added and the reaction was stirred for 30 min. 4-Chlorobenzoyl chloride (2.1 ML) was added and the reaction was stirred for 4 h, and then treated with saturated ammonium chloride (50 mL) and the reaction extracted with ethyl acetate (3×100 mL), dried over MgSO₄ and concentrated to give a thick oil which was purified by chromatography to afford 2-trimethysilylethyl[1-(4-chlorobenzoyl)-6-fluoro-5-methoxy-2-methyl-1H-indol-3-yl]acetate (54) (4.0 g, 8.4 mmol, 57%, >99% pure) as light yellow glassy prisms; 300 MHz ¹H-NMR (CDCl₃) δ 0.01 (s, 9H), 0,98 (m, 2H), 2.33 (s, 3H), 3.63 (s, 2H), 3.94 (s, 3H), 4.20 (m, 2H), 6.86-7.06 (m, 2H), 7.45-7.67 (m, 4H).

### Step 4. Preparation of [1-(4-chlorobenzoyl)-6-fluoro-5-hydroxy-2-methyl-1H-indol-3-yl]acetic acid (26)

To a solution of 2-trimethysilylethyl[1-(4-chlorobenzoyl)-6-fluoro-5-methoxy-2-methyl-1H-indol-3-yl]acetate (54) (4.0 g, 8.4 mmol) in dichloromethane (30 mL) cooled to −78° C. was added BBr₃ (10 g, 5 eqv) and the reaction was stirred for 15 min then allowed to warm to room temperature. After 3 h the reaction was poured into ice water (150 mL) with stirring and then filtered and the solid washed with dichloromethane (50 mL) to afford [1-(4-chlorobenzoyl)-6-fluoro-5-hydroxy-2-methyl-1H-indol-3-yl]acetic acid (26) (>99% pure, 2.3 g, 76%); 300 MHz ¹H-NMR (DMSO-d₆) δ 2.12 (s, 3H), 3.58 (s, 2H), 6.89-7.04 (m, 2H, ArH), 7.66 (m, 4H, ArH), 9.44 (br, 1H, CO₂H); mp 204.7° C.

### EXAMPLE 12

### [6-chloro-1-(4-chlorobenzoyl)-5-methoxy-2-methyl-1H-indol-3-yl]acetic acid (60) via Method C

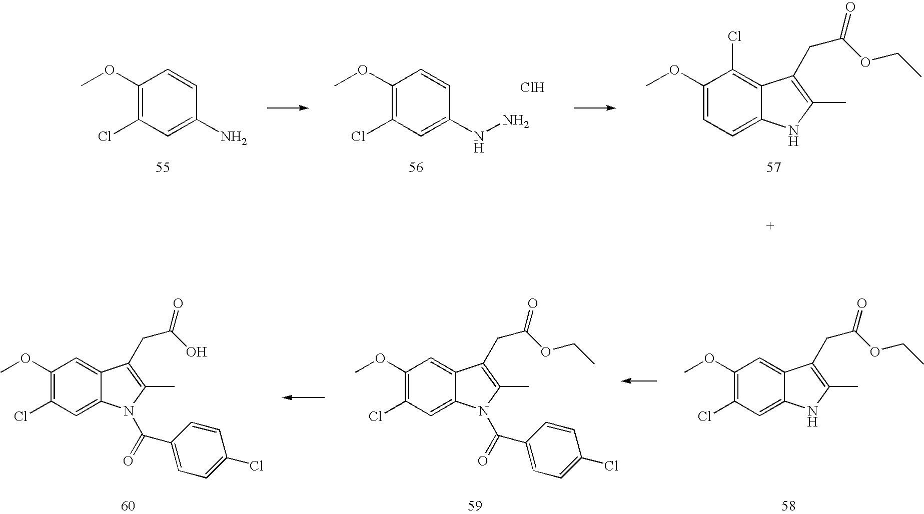

### Step 1. Preparation of 3-chloro-4-methoxyphenylhydrazine hydrochloride (56)

A 2 L flask was charged with 540 mL of 4N HCl, 3-chloro-4-methyoxyaniline 97% (115.6 g, 0.711 mol), and treated with a solution of sodium nitrite [51.5 g, 0.747 mol in 200 mL of H₂O (5N)] at such a rate that the temperature was maintained 0° C. and maintained at that temperature for an additional 1 h. A second flask equipped with over-head stirrer was charged with sodium sulfite (358.6 g, 2.845 mol) and water (1000 mL) and then the contents of the first flask, cooled to ca 10° C., were rapidly added causing the solution to become a bright orange-red color. The solution was stirred at room temperature for 45 min whereupon the solution acquired a yellow color. The solution was finally heated and stirred at 70° C. for 2 h. Finally, the mixture was treated with 770 mL of conc. HCl at 70° C. for 5 h to give brown colored solution and precipitate. Reaction was stirred at 4° C. for 12 h and the precipitate isolated by filtration to afford a crude light cream colored solid; 3-chloro-4-methoxyphenylhydrazine hydrochloride (56). The product was recrystallized by dissolving crude product in 850 mL of water at 90° C. in 1˜2 hours, filtered through pad of diatomaceous earth and washed with 250 mL of water. To the filtrate was added 300 mL of conc. HCl, cooled to 0° C., filtered and dried under vacuum to give pure 3-chloro-4-methoxyphenylhydrazine hydrochloride (56).

### Step 2. Preparation of ethyl (6-chloro-5-methoxy-2-methyl-1H-indol-3-yl) acetate (58) and ethyl (4-chloro-5-methoxy-2-methyl-1H-indol-3-yl) acetate (57)

A flask was charged with ethanol (320 mL) and sulfuric acid (24 mL) and warmed to 45° C. 3-Chloro-4-methoxyphenylhydrazine HCl salt (50 g, 239 mmol), and ethyl levulinate (34.4 g, 239 mmol) were added and heated at 70° C. for 2.5 h. The reaction mixture was cooled to room temperature and concentrated to 1/4 volume. Water (500 mL) was added and the mixture extracted with dichloromethane (2×400 mL). The combined organic layer was washed with brine (10 mL), dried over sodium sulfate and concentrated to afford the crude product as a purple solid (58 g) as a mixture of isomers (−4/1 ratio). Recrystallization was accomplished by dissolving the crude product in ethyl acetate (˜200 mL) with warming followed by addition of hexane (˜100 ML). The single regioisomer, ethyl (6-chloro-5-methoxy-2-methyl-1H-indol-3-yl) acetate (58), ¹H NMR (CDCl₃/300 MHz) 8.02 (brs, 1H), 7.08 (s, 1H), 7.01 (s, 1H), 4.13 (q, 2H, J=6.9 Hz), 3.62 (s, 2H), 2.26 (s, 3H), 1.24 (t, 3H, J=6.9 Hz), was obtained as light tan crystals (23 g, 34% yield, >98% pure). The other regioisomer, ethyl (4-chloro-5-methoxy-2-methyl-1H-indol-3-yl) acetate (57), ¹H NMR (CDCl₃/300 MHz) 8.26 (brs, 1H), 6.89 (d, 1H, J=8.7 Hz), 6.73 (d, 1H, J=8.7), 4.20 (q, 2H, J=6.9 Hz), 3.91 (s, 2H), 3.87 (s, 3H), 2.10 (s, 3H), 1.28 (t, 3H, J=6.9 Hz), was obtained by concentration of the filtrate and purification by chromatography on silica gel eluting with ethyl acetate/hexanes.

### Step 3. Preparation of ethyl[6-chloro-1-(4-chlorobenzoyl)-5-methoxy-2-methyl-1H-indol-3-yl]acetate (59)

A mixture of sodium hydride (116 mg, 4.6 mmol) in 10 mL of dimethylformamide was treated dropwise with a solution of ethyl (6-chloro-5-methoxy-2-methyl-1H-indol-3-yl) acetate (58) (1.18 g, 4.17 mmol) in 3 mL of dimethylformamide (3 mL) at 0° C. After 10 min, the solution was treated with 4-chlorobenzoyl chloride (693 mg, 3.96 mmol) and the temperature allowed to warm to room temperature over 30 min. The reaction mixture was then diluted with saturated ammonium chloride and extracted with ethyl acetate (3×30 mL). The combined extracts were washed with brine, dried over sodium sulfate, concentrated in vacuo to afford a yellow solid that was recrystallized from ethyl acetate hexanes to afford 1.00 g, 57%, of pure ethyl[6-chloro-1-(4-chlorobenzoyl)-5-methoxy-2methyl-1H-indol-3-yl]acetate (59), mp 148-149° C., ¹H NMR (CDCl₃/300 MHz) 7.65 (d, 2H, J=8.7 Hz), 7.49 (d, 2H, J=8.7), 7.19 (s, 1H), 7.01 (s, 1H), 4.16 (q, 2H, J=6.9 Hz), 3.94 (s, 3H), 3.64 (s, 2H), 2.31 (s, 3H), 1.26 (t, 3H, J=6.9 Hz).

### Step 4. Preparation of [6-chloro-1-(4-chlorobenzoyl)-5-methoxy-2-methyl-1H-indol-3-yl]acetic acid (60)

A pressure bottle was charged with ethyl[6-chloro-1-(4-chlorobenzoyl)-5-methoxy-2-methyl-1H-indol-3-yl]acetate (59) (4.6 g, 10.94 mmol) and THF (46 mL). This solution was heated to 55° C. and treated with 6N HCl (23 mL) and the tube was sealed and heated at 55° C. for 3.5 h. The reaction mixture was cooled to 0° C. and diluted with ethyl acetate (250 mL) and then washed with water (3×150 mL) and brine (150 mL), dried over sodium sulfate, and concentrated to afford the crude product as a brown oil. This crude product was redissolved in ethyl acetate and concentrated (2×). White solids were formed when ethyl acetate was added to this brown residue. The solids were isolated by filtration and washed with 30% ethyl acetate in hexanes solution to give 1.7 g of [6-chloro-1-(4-chlorobenzoyl)-5-methoxy-2-methyl-1H-indol-3-yl]acetic acid (60) as a white solid. The filtrate was concentrated and dissolved in methanol (70 mL) and cooled in refrigerator for 3 h. White solids formed and filtered to give 500 mg mixture of product and starting material in a ratio of 70:30. The filtrate was concentrated and crystallized from EA/Hex (˜100 mL/150 mL) to afford 800 mg product as a yellow solid. The total yield of [6-chloro-1-(4-chlorobenzoyl)-5-methoxy-2-methyl-1H-indol-3-yl]acetic acid (60) was 2.5 g, 58%, TLC on SiO₂ 10% MeOH/CH₂Cl₂, R_{f}=0.39, mp 215° C., ¹H NMR (CDCl₃/300 MHz) 7.65 (d, 2H, J=8.4 Hz), 7.50 (d, 2H, J=8.4 Hz), 7.71 (s, 1H), 6.98 (s, 1H), 3.93 (s, 3H), 3.70 (s 2H), 2.31 (s, 3H).

### EXAMPLES 13-50

The following compounds were made by Method B from the appropriate starting materials. The 5-hydroxy derivatives resulted from demethylation of the corresponding 5-methoxy analogs by treatment with boron tribromide in dichloromethane.

### EXAMPLE 13

### [1-(4-ethylbenzoyl)-5-methoxy-2-methyl-1H-indol-3 yl]acetic acid

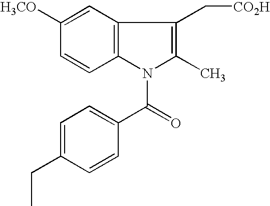

mp 136-137° C.

¹H NMR (CDCl₃/300 MHz) 7.64 (d, 2H, J=8.0 Hz), 7.30 (d, 2H, J=8.0 Hz), 6.97 (d, 1H, J=1.8 Hz), 6.92 (d, 1H, J=8.8 Hz), 6.67 (dd, 1H, J=8.8, 1.8 Hz), 3.82 (s, 3H), 3.70 (s, 2H), 2.75 (q, 2H, J=7.8 Hz), 2.38 (s, 3H), 1.29 (t, 3H, J=7.8 Hz).

### EXAMPLE 14

### [5-hydroxy-2-methyl-1-(4-methylbenzoyl)-1H-indol-3-yl]acetic acid

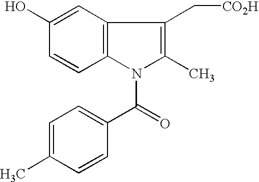

mp 225-227° C.

¹H NMR (CDCl₃/300 MHz) 7.73 (d, 2H, J=9.0 Hz), 7.29 (d, 2H, J=9.0 Hz), 6.86 (d, 1H, J=2.4 Hz), 6.79 (d, 1H, J=8.7 Hz), 6.56 (dd, 1H, J=8.7, 2.4 Hz), 3.58 (s, 2H), 3.43 (s, 3H), 2.33 (s, 3H).

### EXAMPLE 15

### [1-(2,4-dichlorobenzoyl)-5-hydroxy-2-methyl-1H-indol-3-yl]acetic acid

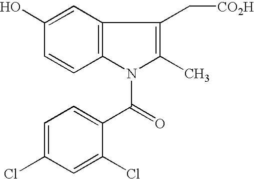

¹H NMR (DMSO-d₆/300 MHz) 7.65 (m, 1H), 7.56 (m, 2H), 7.22 (d, 1H, J=9.0 Hz), 6.87 (d, 1H, J=2.1 Hz), 6.60 (dd, 1H, J=9.0, 2.1 Hz), 3.59 (s, 2H), 2.14 (s, 3H).

### EXAMPLE 16

### {5-hydroxy-2-methyl-1-[4-(trifluoromethyl)benzoyl]-1H-indol-3-yl}acetic acid

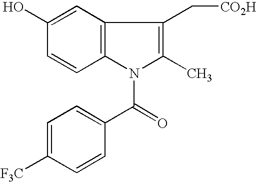

mp 192-194° C.

¹H NMR (CD₃OD/300 MHz) 7.87 (s, 4H), 6.90-6.86 (m, 2H), 6.56 (dd, 1H, J=9.0, 2.4 Hz), 3.65 (s, 2H), 2.27 (s, 3H).

### EXAMPLE 17

### [1-(2,3-dichlorobenzoyl)-5-methoxy-2-methyl-1H-indol-3-yl]acetic acid

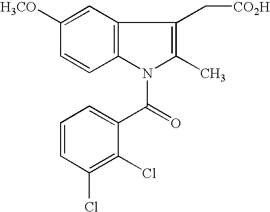

mp 120-122° C.

¹H NMR (CDCl₃/300 MHz) 7.65 (m, 1H), 7.37 (m, 2H), 7.27 (d, 1H, J=9.0 Hz), 6.93 (d, 1H, J=2.4 Hz), 6.74 (dd, 1H, J=9.0, 2.4 Hz), 3.83 (s, 3H), 3.66 (s 2H), 2.21 (s 3H).

### EXAMPLE 18

### [1-(2,3-dichlorobenzoyl)-5-hydroxy-2-methyl-1H-indol-3-yl]acetic acid

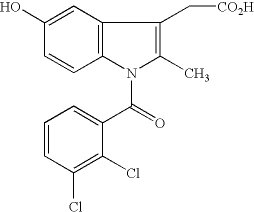

mp 151-152° C.

¹H NMR (CD₃OD/300 MHz) 7.76 (m, 1H), 7.50 (m, 2H), 7.23 (d, 1H, J=9.3 Hz), 6.86 (d, 1H, J=2.4 Hz), 6.60 (dd, 1H, J=9.3, 2.4 Hz), 3.61 (s, 2H), 2.12 (s, 3H).

### EXAMPLE 19

### {6-fluoro-5-methoxy-2-methyl-1-[4-(trifluoromethyl)benzoyl]-1H-indol-3-yl}acetic acid

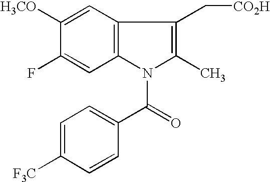

mp of tris salt 177-178° C.

¹H NMR (DMSO-d₆/300 MHz) 7.95 (d, 2H, J=8.4 Hz), 7.86 (d, 2H, J=8.4 Hz), 7.28 (d, 1H, J=8.7 Hz), 7.06 (m, 1H), 3.86 (s, 3H), 3.68 (s, 2H), 2.06 (s, 3H).

### EXAMPLE 20

### {6-fluoro-5-hydroxy-2-methyl-1-[4-(trifluoromethyl)benzoyl]-1H-indol-3-yl}acetic acid

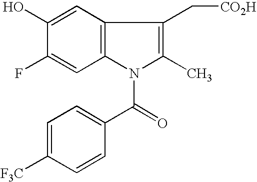

mp 203° C.

¹H NMR (DMSO-d₆/300 MHz) 7.97 (d, 2H, J=8.1 Hz), 7.88 (d, 2H, J=8.1 Hz), 7.06 (m, 2H), 3.58 (s, 2H), 2.06 (s, 3H).

### EXAMPLE 21

### {6-fluoro-5-methoxy-2-methyl-1-[4-(trifluoromethoxy)benzoyl]-1H-indol-3-yl}acetic acid

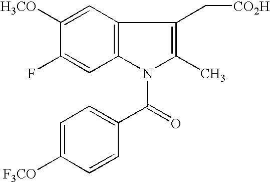

mp 148-150° C.

¹H NMR (DMSO-d₆/300 MHz) 7.82 (d, 2H, J=8.7 Hz), 7.59 (d, 2H, J=8.7 Hz), 7.30 (d, 1H, J=8.4 Hz), 6.97 (d, 1H, J=12.3 Hz), 3.86 (s, 3H), 3.69 (s, 2H), 2.12 (s, 3H).

### EXAMPLE 22

### {6-fluoro-5-hydroxy-2-methyl-1-[4-(trifluoromethoxy)benzoyl]-1H-indol-3-yl}acetic acid

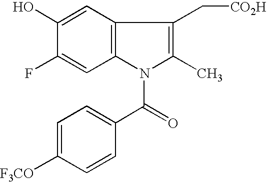

mp 190° C.

¹H NMR (DMSO-d₆/300 MHz) 7.80 (d, 2H, J=8.4 Hz), 7.57 (d, 2H, J=8.4 Hz), 7.01 (d, 1H, J=9.0 Hz), 6.91 (d, 1H, J=12.3 Hz), 3.58 (s, 2H), 2.10 (s, 3H).

### EXAMPLE 23

### {6-fluoro-5-methoxy-2-methyl-1-(4-methylbenzoyl)-1H-indol-3-yl}acetic acid

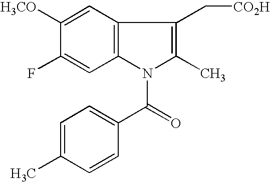

mp 137° C.

¹H NMR (DMSO-d₆/300 MHz) 7.55 (d, 2H, J=8.1 Hz), 7.39 (d, 2H, J=8.1 Hz), 7.26 (d, 1H, J=8.4 Hz), 6.84 (d, 1H, J=12.3 Hz), 3.86 (s, 3H), 3.69 (s, 2H), 2.50 (s, 3H), 2.12 (s, 3H).

### EXAMPLE 24

### [6-fluoro-5-hydroxy-2-methyl-1-(4-methylbenzoyl)-1H-indol-3-yl]acetic acid

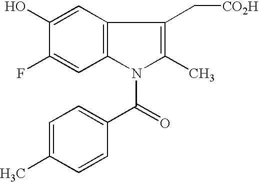

mp 198-199° C.

¹H NMR (DMSO-d₆/300 MHz) 7.55 (d, 2H, J=7.8 Hz), 7.39 (d, 2H, J=7.8 Hz), 6.99 (d, 1H, J=9.0 Hz), 6.79 (d, 1H, J=12.0 Hz), 3.58 (s, 2H), 2.50 (s, 3H), 2.15 (s, 3H).

### EXAMPLE 25

### {1-[4-(difluoromethoxy)benzoyl]-6-fluoro-5-methoxy-2-methyl-1H-indol-3-yl}acetic acid

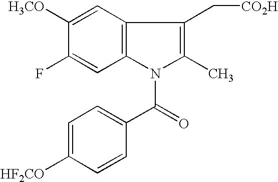

mp 124° C.

¹H NMR (CDCl₃/300 MHz) 7.73 (d, 2H, J=8.7 Hz), 7.22 (d, 2H, J=8.7 Hz), 6.99 (d, 1H, J=8.1 Hz), 6.84 (d, 1H, J=12.0 Hz), 6.95 (t, 1H, J=73.6 Hz), 3.91 (s, 3H), 3.68 (s, 2H), 2.32 (s, 3H).

### EXAMPLE 26

### [1-(4-bromobenzoyl)-6-fluoro-5-hydroxy-2-methyl-1H-indol-3-yl]acetic acid

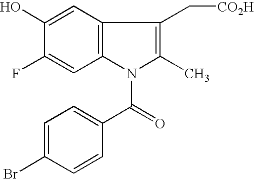

mp of tris salt 180-182° C.

¹H NMR (DMSO-d₆/300 MHz) 7.80 (d, 2H, J=8.4 Hz), 7.59 (d, 2H, J=8.4 Hz), 7.00 (d, 1H, J=9.0 Hz), 6.92 (d, 1H, J=12.3 Hz), 3.58 (s, 2H), 2.11 (s, 3H).

### EXAMPLE 27

### [1-(4-bromobenzoyl)-6-fluoro-5-methoxy-2-methyl-1H-indol-3-yl]acetic acid

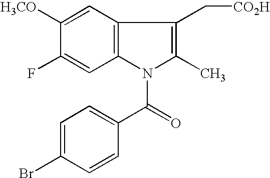

mp 154° C.

¹H NMR (DMSO-d₆/300 MHz) 7.81 (d, 2H, J=8.7 Hz), 7.61 (d, 2H, J=8.7 Hz), 7.29 (d, 1H, J=8.7 Hz), 7.00 (d, 1H, J=12.3 Hz), 3.88 (s, 3H), 3.70 (s, 2H), 2.14 (s, 3H).

### EXAMPLE 28

### {6-fluoro-5-methoxy-2-methyl-1-[4-(methylthio)benzoyl]-1H-indol-3-yl}acetic acid

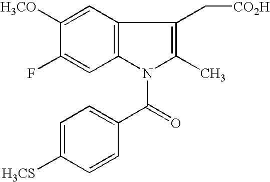

¹H NMR (DMSO-d₆/300 MHz) 7.57 (d, 2H, J=8.4 Hz), 7.41 (d, 2H, J=8.4 Hz), 7.27 (d, 1H, J=8.4 Hz), 6.91 (d, 1H, J=12.3 Hz), 3.86 (s, 3H), 3.69 (s, 2H), 2.56 (s, 3H), 2.17 (s, 3H).

### EXAMPLE 29

### {6-fluoro-5-hydroxy-2-methyl-1-[4-(methylthio)benzoyl]-1H-indol-3-yl}acetic acid

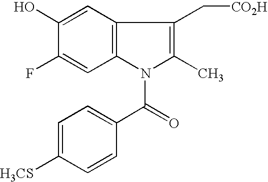

mp 185° C.

¹H NMR (DMSO-d₆/300 MHz) 9.62 (brs, 1H), 7.57 (d, 2H, J=8.4 Hz), 7.41 (d, 2H, J=8.4 Hz), 7.00 (d, 1H, J=8.7 Hz), 6.85 (d, 1H, J=12.3 Hz), 3.58 (s, 2H), 2.56 (s, 3H), 2.17 (s, 3H).

### EXAMPLE 30

### [6-fluoro-1-(4-fluorobenzoyl)-5-methoxy-2-methyl-1H-indol-3-yl]acetic acid

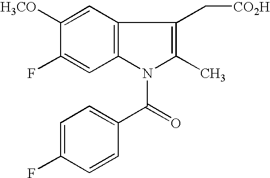

¹H NMR (DMSO-d₆/300 MHz) 7.74 (dd, 2H, J=9.0, 8.7 Hz), 7.24 (dd, 2H, J=9.0, 8.7 Hz), 7.27 (d, 1H, J=8.1 Hz), 6.92 (d, 1H, J=12.6 Hz), 3.86 (s, 3H), 3.69 (s, 2H), 2.14 (s, 3H).

### EXAMPLE 31

### [6-fluoro-1-(4-fluorobenzoyl)-5-hydroxy-2-methyl-1H-indol-3-yl]acetic acid

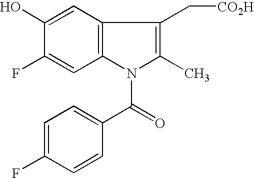

mp 151-152° C.

¹H NMR (DMSO-d₆/300 MHz) 9.65 (brs, 1H), 7.73 (dd, 2H, J=8.5, 7.5 Hz), 7.42 (dd, 2H, J=8.5, 7.5 Hz), 7.00 (d, 1H, J=8.7 Hz), 6.85 (d, 1H, J=12.9 Hz), 3.58 (s, 2H), 2.13 (s, 3H).

### EXAMPLE 32

### {6-fluoro-5-methoxy-2-methyl-1-[4-(1,1,2,2-tetrafluoroethoxy)benzoyl]-1H-indol-3-yl}acetic acid

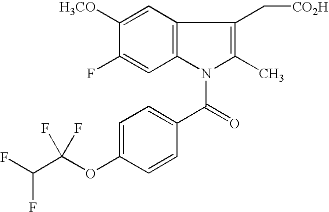

mp 141-142° C.

¹H NMR (DMSO-d₆/300 MHz) 7.77 (d, 2H, J=8.4 Hz), 7.49 (d, 2H, J=8.4 Hz), 7.27 (d, 1H, J=8.4 Hz), 6.94 (d, 1H, J=12.3 Hz), 6.82 (t, 1H, J=51.6 Hz), 3.86 (s, 3H), 3.69 (s, 2H), 2.13 (s, 3H).

### EXAMPLE 33

### {6-fluoro-5-hydroxy-2-methyl-1-[4-(1,1,2,2-tetrafluoroethoxy)benzoyl]-1H-indol-3-yl}acetic acid

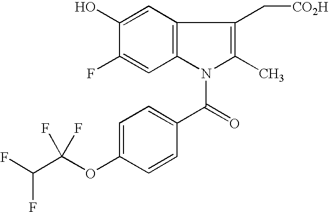

mp 181-182° C.

¹H NMR (DMSO-d₆/300 MHz) 9.65 (brs, 1H), 7.77 (d, 2H, J=8.7 Hz), 7.49 (d, 2H, J=8.7 Hz), 7.00 (d, 1H, J=8.4 Hz), 6.88 (d, 1H, J=12.3 Hz), 6.88 (t, 1H, J=53.4 Hz), 3.58 (s, 2H), 2.12 (s, 3H).

### EXAMPLE 34

### {1-[4-(difluoromethoxy)benzoyl]-6-fluoro-5-hydroxy-2-methyl-1H-indol-3-yl}acetic acid

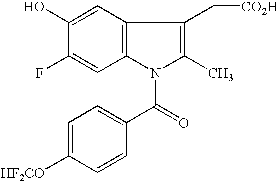

mp 182-183° C.

¹H NMR (CD₃OD/300 MHz) 7.75 (d, 2H, J=8.7 Hz), 7.30 (d, 2H, J=8.7 Hz), 7.05 (t, 1H, J=73.2 Hz), 7.00 (d, 1H, J=8.4 Hz), 6.89 (d, 1H, J=12.3 Hz), 6.95 (t, 1H, J=73.6 Hz), 3.64 (s, 2H), 2.32 (s, 3H).

### EXAMPLE 35

### {1-[4-(difluoromethoxy)benzoyl]-5-methoxy-2-methyl-1H-indol-3-yl}acetic acid

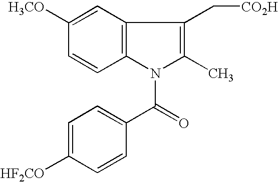

¹H NMR (CDCl₃/300 MHz) 7.75 (d, 2H, J=8.7 Hz), 7.20 (d, 2H, J=8.7 Hz), 6.95 (d, 1H, J=2.5 Hz), 6.86 (d, 1H, J=9.0 Hz), 6.67 (dd, 1H, J=9.0, 2.5 Hz), 6.65 (t, 1H, J=72.9 Hz), 3.83 (s, 3H), 3.71 (s, 2H), 2.40 (s, 3H).

### EXAMPLE 36

### {1-[4-(difluoromethoxy)benzoyl]-5-hydroxy-2-methyl-1H-indol-3-yl}acetic acid

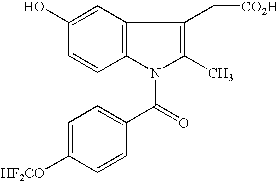

mp 195-196° C.

¹H NMR (CD₃OD/300 MHz) 7.75 (d, 2H, J=8.7 Hz), 7.29 (d, 2H, J=8.7 Hz), 7.03 (t, 1H, J=73.5 Hz), 6.87 (d, 1H, J=2.4 Hz), 6.84 (d, 1H, J=8.7 Hz), 6.54 (dd, 1H, J=8.7, 2.4 Hz), 3.65 (s, 2H), 2.30 (s, 3H).

### EXAMPLE 37

### [4-chloro-1-(4-chlorobenzoyl)-5-methoxy-2-methyl-1H-indol-3-yl]acetic acid

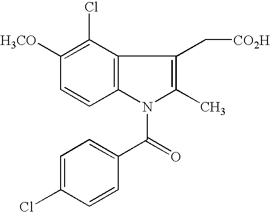

mp 209° C. (dec)

¹H NMR (CDCl₃/300 MHz) 7.60 (d, 2H, J=8.7 Hz), 7.48 (d, 2H, J=8.7 Hz), 6.96 (d, 1H, J=9.0 Hz), 6.74 (d, 1H, J=9.0 Hz), 4.06 (s, 2H), 3.88 (s, 3H), 2.30 (s, 3H).

### EXAMPLE 38

### [6-chloro-1-(4-chlorobenzoyl)-5-hydroxy-2-methyl-1H-indol-3-yl]acetic acid

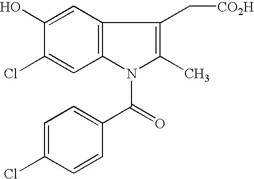

mp 203° C.

¹H NMR (CD₃OD/300 MHz) 7.69 (d, 2H, J=8.4 Hz), 7.58 (d, 2H, J=8.4 Hz), 7.14 (s, 1H), 7.00 (s, 1H), 3.64 (s, 2H), 2.23 (s, 3H).

### EXAMPLE 39

### {6-chloro-1-[4-(difluoromethoxy)benzoyl]-5-methoxy-2-methyl-1H-indol-3-yl}acetic acid

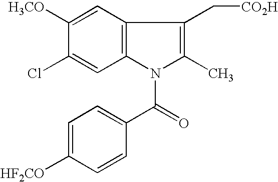

mp of tris salt 106° C.

¹H NMR (CDCl₃/300 MHz) 7.74 (d, 2H, J=8.7 Hz), 7.23 (d, 2H, J=8.7 Hz), 7.13 (s, 1H), 6.98 (s, 1H), 6.64 (t, 1H, J=72.6 Hz), 3.93 (s, 3H), 3.70 (s, 2H), 2.33 (s, 3H).

### EXAMPLE 40

### {1-[(5-chlorothien-2-yl)carbonyl]-5-hydroxy-2-methyl-1H-indol-3-yl}acetic acid

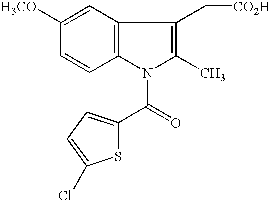

mp 195° C.

¹H NMR (CDCl₃/300 MHz) 7.43 (d, 1H, J=4.2 Hz), 7.13-7.10 (m, 2H), 6.87 (d, 1H, J=2.1 Hz), 6.61 (dd, 1H, J=8.7, 2.1 Hz), 3.66 (s, 2H), 2.38 (s, 3H).

### EXAMPLE 41

### {1-[(5-chlorothien-2-yl)carbonyl]-6-fluoro-5-methoxy-2-methyl-1H-indol-3-yl}acetic acid

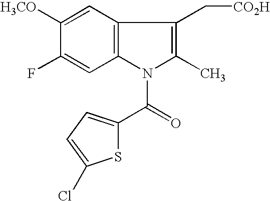

mp 169° C.

¹H NMR (CDCl₃/300 MHz) 7.35 (d, 1H, J=4.0 Hz), 7.09 (d, 1H, J=11.7 Hz), 7.00 (d, 1H, J=7.2 Hz), 6.98 (d, 1H, J=4.0 Hz), 3.93 (s, 3H), 3.70 (s, 2H), 2.42 (s, 3H).

### EXAMPLE 42

### {1-[(5-chlorothien-2-yl)carbonyl]-6-fluoro-5-hydroxy-2-methyl-1H-indol-3-yl}acetic acid

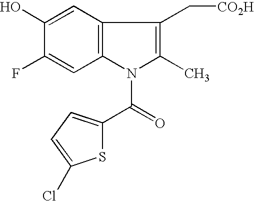

mp 174° C.

¹H NMR (CDCl₃/300 MHz) 7.34 (d, 1H, J=3.9 Hz), 7.13 (d, 1H, J=11.1 Hz), 7.07 (d, 1H, J=8.4 Hz), 6.98 (d, 1H, J=3.9 Hz), 3.66 (s, 2H), 2.39 (s, 3H).

### EXAMPLE 43

### [6-fluoro-5-methoxy-2-methyl-1-(thien-2-ylcarbonyl)-1H-indol-3-yl]acetic acid

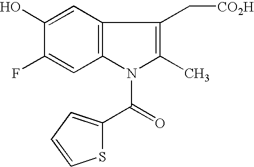

mp 137° C.

¹H NMR (CDCl₃/300 MHz) 7.77 (dd, 1H, J=5.0, 1.2 Hz), 7.54 (dd, 1H, J=3.9, 1.2 Hz), 7.15 (dd, 1H, J=5.0, 3.9 Hz), 7.01 (d, 1H, J=12.0 Hz), 7.00 (d, 1H, J=8.1 Hz), 3.92 (s, 3H), 3.69 (s, 2H), 2.41 (s, 3H).

### EXAMPLE 44

### {6-fluoro-5-methoxy-2-methyl-1-[(5-methylthien-2-yl)carbonyl]-1H-indol-3-yl}acetic acid

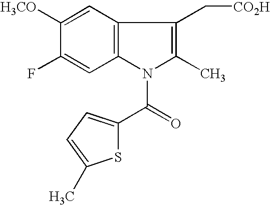

mp 152° C.

¹H NMR (CDCl₃/300 MHz) 7.35 (d, 1H, J=3.9 Hz), 7.06 (d, 1H, J=12.3), 6.99 (d, 1H, J=8.1 Hz), 6.81 (d, 1H, J=3.9 Hz), 3.92 (s, 3H), 3.68 (s, 2H), 2.60 (s, 3H), 2.42 (s, 3H).

### EXAMPLE 45

### {6-fluoro-5-hydroxy-2-methyl-1-[(5-methylthien-2-yl)carbonyl]-1H-indol-3-yl}acetic acid

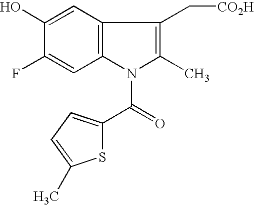

mp 197° C.

¹H NMR (CD₃OD/300 MHz) 7.40 (d, 1H, J=4.0 Hz), 6.99 (d, 1H, J=8.7 Hz), 6.98 (d, 1H, J=11.7 Hz), 6.93 (d, 1H, J=4.0 Hz), 3.64 (s, 2H), 2.62 (s, 3H), 2.34 (s, 3H).

### EXAMPLE 46

### [6-fluoro-5-hydroxy-2-methyl-1-(thien-2-ylcarbonyl)-1H-indol-3-yl]acetic acid

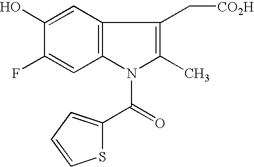

mp 219° C.

¹H NMR (CD₃OD/300 MHz) 7.97 (dd, 1H, J=5.1, 1.2 Hz), 7.59 (dd, 1H, J=3.9, 1.2 Hz), 7.22 (dd, 1H, J=5.1, 3.9 Hz), 7.00 (d, 1H, J=8.7 Hz), 6.94 (d, 1H, J=12.0 Hz), 3.65 (s, 2H), 2.32 (s, 3H).

### EXAMPLE 47

### {6-Chloro-5-hydroxy-2-methyl-1-[4-(trifluoromethoxy)benzoyl]-1H-indol-3-yl}acetic acid

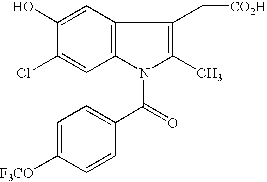

mp 160° C.

¹H NMR (DMSO-d₆/300 MHz) 9.96 (brs, 1H), 7.79 (d, 2H, J=8.4 Hz), 7.61 (d, 2H, J=8.4 Hz), 7.08 (s, 1H), 7.02 (s, 1H), 3.58 (s, 2H), 2.12 (s, 3H).

### EXAMPLE 48

### [6-chloro-1-(4-fluorobenzoyl)-5-methoxy-2-methyl-1H-indol-3-yl]acetic acid

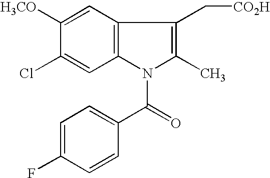

mp 201° C.

¹H NMR (CDCl₃/300 MHz) 7.73 (dd, 2H, J=8.7, 5.4 Hz), 7.20 (dd, 2H, J=8.7, 8.7 Hz), 7.10 (s, 1H), 6.98 (s, 1H), 3.93 (s, 3H), 3.70 (s, 2H), 2.34 (s, 3H).

### EXAMPLE 49

### (1-benzoyl-6-chloro-5-methoxy-2-methyl-1H-indol-3-yl)acetic acid

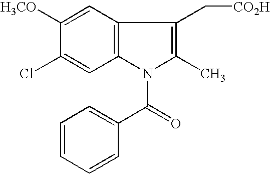

mp 204° C.

¹H NMR (CDCl₃/300 MHz) 7.68 (m, 3H), 7.51 (m, 2H), 7.10 (s, 1H), 6.97 (s, 1H), 3.93 (s, 3H), 3.70 (s, 2H), 2.33 (s, 3H).

### EXAMPLE 50

### Ethyl[1-(4-chlorobenzol)-5-hydroxy-2-methyl-1H-indol-3-yl]acetate

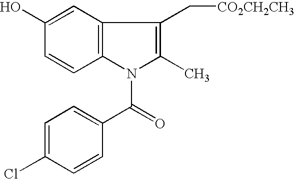

The various esters described herein can be prepared from the corresponding acid using methods similar to the following preparation of The thionyl chloride (0.026 mL, 0.3499 mmol) was added dropwise to the solution of [1-(4-chlorobenzol)-5-hydroxy-2-methyl-1H-indol-3-yl]acetic acid (100 mg, 0.2915 mmol) in anhydrous ethanol (5 mL) at room temperature under an atmosphere of dry nitrogen. The reaction mixture was stirred for 5 h at room temperature until the TLC showed that all the starting acid had been consumed. The reaction mixture was cooled to 0° C., diluted with water (10 mL), extracted with dichloromethane (3×30 mL), the combined organic extracts were washed with brine, dried over sodium sulfate, filtered, concentrated and purified by chromatography (12 g silica gel, 10% to 100% ethyl acetate-hexanes) to afford ethyl[1-(4-chlorobenzol)-5hydroxy-2-methyl-1H-indol-3-yl]acetic acid (85 mg, 80% yield) as a white solid; mp 154.7° C.; R_{f} 0.6 (1:1 ethyl acetate-hexane); ¹H NMR (CDCl₃/300 MHz) 7.60 (d, 2H, J=8.7 Hz), 7.41 (d, 2H, J=8.7), 6.84 (d, 1H, J=2.4 Hz), 6.76 (d, 1H, J=8.7 Hz), 6.55 (dd, 1H, J=8.7, 2.4 Hz), 4.17 (q, 2H, J=6.6 Hz), 3.57 (s, 2H), 2.30 (s, 3H), 1.21 (t, 3H, J=6.6 Hz).

### EXAMPLES 51-73

The esters in these Examples were prepared by the general method described above.

### EXAMPLE 51

### Methyl[1-(4-chlorobenzoyl)-5-hydroxy-2-methyl-1H-indol-3-yl]acetate

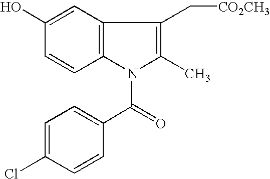

mp 159° C.

¹H NMR (CDCl₃/300 MHz) 7.66 (d, 2H, J=8.1 Hz), 7.47 (d, 2H, J=8.1), 6.91 (d, 1H, J=2.4 Hz), 6.84 (d, 1H, J=8.7 Hz), 6.57 (dd, 1H, J=8.7, 2.4 Hz), 3.70 (s, 3H), 3.64 (s, 2H), 2.37 (s, 3H).

### EXAMPLE 52

### Methyl[1-(4-chlorobenzoyl)-6-fluoro-5-hydroxy-2-methyl-1H-indol-3-yl]acetate

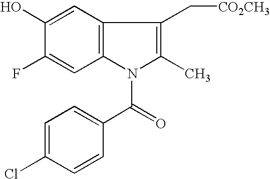

mp 166° C.

¹H NMR (DMSO-d₆/300 MHz) 9.66 (brs, 1H), 7.66 (s, 4H), 6.98 (d, 1H, J=8.7 Hz), 6.93 (d, 1H, J=12.3 Hz), 3.70 (s, 2H), 3.62 (s, 3H), 2.12 (s, 3H).

### EXAMPLE 53

### Butyl[1-(4-chlorobenzoyl)-5-hydroxy-2-methyl-1H-indol-3-yl]acetate

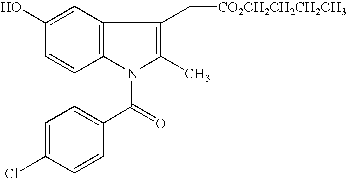

mp 101.5° C.

¹H NMR (CDCl₃/300 MHz) 7.64 (d, 2H, J=8.7 Hz), 7.45 (d, 2H, J=8.7), 6.90 (d, 1H, J=2.4 Hz), 6.83 (d, 1H, J=8.7 Hz), 6.55 (dd, 1H, J=8.7, 2.4 Hz), 5.05 (brs, 1H), 4.10 (t, 2H, J=6.6 Hz), 3.62 (s, 2H), 2.35 (s, 3H), 1.60 (m, 2H), 1.32 (m, 2H), 0.90 (t, 3H, J=7.2 Hz).

### EXAMPLE 54

### Propyl[1-(4-chlorobenzoyl)-5-hydroxy-2-methyl-1H-indol-3-yl]acetate

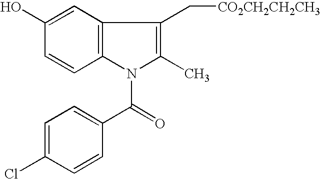

mp 110° C.

¹H NMR (CDCl₃/300 MHz) 7.64 (d, 2H, J=8.1 Hz), 7.45 (d, 2H, J=8.1), 6.91 (d, 1H, J=2.4 Hz), 6.82 (d, 1H, J=8.7 Hz), 6.55 (dd, 1H, J=8.7, 2.4 Hz), 5.38 (brs, 1H), 4.06 (q, 2H, J=6.9 Hz), 3.62 (s, 2H), 2.36 (s, 3H), 1.63 (td, 2H, J=7.2, 6.9 Hz), 1.28 (t, 3H, J=6.9 Hz).

### EXAMPLE 55

### Ethyl[1-(4-chlorobenzoyl)-6-fluoro-5-hydroxy-2-methyl-1H-indol-3-yl]acetate

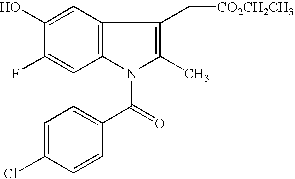

mp 150.4° C.

¹H NMR (CDCl₃/300 MHz) 7.64 (d, 2H, J=8.4 Hz), 7.46 (d, 2H, J=8.4), 7.03 (d, 1H, J=8.4 Hz), 6.92 (d, 1H, J=11.7 Hz), 5.47 (brs, 1H), 4.16 (q, 2H, J=7.2 Hz), 3.60 (s, 2H), 2.26 (s, 3H), 1.26 (t, 3H, J=7.2 Hz).

### EXAMPLE 56

### Propyl[1-(4-chlorobenzoyl)-6-fluoro-5-hydroxy-2-methyl-1H-indol-3-yl]acetate

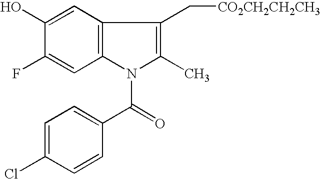

mp 92.0° C.

¹H NMR (CDCl₃/300 MHz) 7.63 (d, 2H, J=8.7 Hz), 7.46 (d, 2H, J=8.7), 7.04 (d, 1H, J=8.4 Hz), 6.92 (d, 1H, J=12.0 Hz), 5.57 (brs, 1H), 4.06 (t, 2H, J=8.1 Hz), 3.60 (s, 2H), 2.26 (s, 3H), 1.63 (td, 2H, J=8.1, 6.9 Hz), 0.91 (t, 3H, J=6.9 Hz).

### EXAMPLE 57

### Isopropyl[1-(4-chlorobenzoyl)-6-fluoro-5-hydroxy-2-methyl-1H-indol-3-yl]acetate

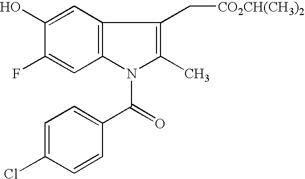

mp 120° C.

¹H NMR (CDCl₃/300 MHz) 7.62 (d, 2H, J=8.7 Hz), 7.46 (d, 2H, J=8.7), 7.02 (d, 1H, J=8.4 Hz), 6.92 (s, 1H, J=12.0 Hz), 5.72 (brs, 1H), 5.01 (sept, 1H, J=6.3 Hz), 3.56 (s, 2H), 2.24 (s, 3H), 1.23 (d, 3H, J=6.3 Hz).

### EXAMPLE 58

### sec-Butyl[1-(4-chlorobenzoyl)-6-fluoro-5-hydroxy-2-methyl-1H-indol-3-yl]acetate

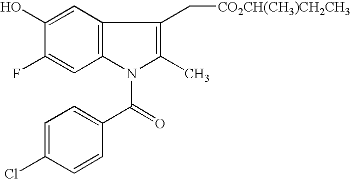

mp oil

¹H NMR (CDCl₃/300 MHz) 7.63 (d, 2H, J=8.4 Hz), 7.46 (d, 2H, J=8.4), 7.05 (d, 1H, J=8.4 Hz), 6.94 (s, 1H, J=11.7 Hz), 5.39 (brs, 1H), 4.83 (m, 1H), 3.58 (s, 2H), 2.25 (s, 3H), 1.55 (m, 2H), 1.20 (d, 3H, J=6.6 Hz), 0.84 (t, 3H, J=7.2 Hz).

### EXAMPLE 59

### Methyl[1-(4-chlorobenzoyl)-6-fluoro-5-methoxy-2-methyl-1H-indol-3-yl]acetate

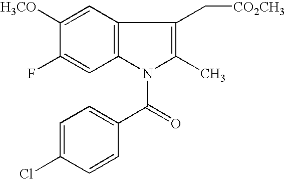

mp 137.8° C.

¹H NMR (CDCl₃/300 MHz) 7.65 (d, 2H, J=8.7 Hz), 7.49 (d, 2H, J=8.7), 7.01 (d, 1H, J=8.1 Hz), 6.86 (d, 1H, J=12.3 Hz), 3.92 (s 3H), 3.71 (s, 3H), 3.66 (s, 2H), 2.32 (s, 3H).

### EXAMPLE 60

### Methyl[6-chloro-1-(4-chlorobenzoyl)-5-methoxy-2-methyl-1H-indol-3-yl]acetate

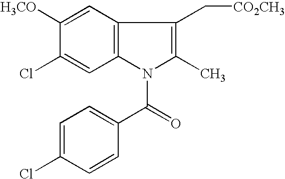

mp 123° C.

¹H NMR (CDCl₃/300 MHz) 7.65 (d, 2H, J=8.7 Hz), 7.49 (d, 2H, J=8.7), 7.17 (s, 1H), 6.99 (s, 1H), 3.94 (s, 3H), 3.70 (s, 3H), 3.66 (s, 2H), 2.30 (s, 3H), 1.63 (m, 2H), 0.90 (t, 3H, J=7.5 Hz).

### EXAMPLE 61

### Propyl[6-chloro-1-(4-chlorobenzoyl)-5-methoxy-2-methyl-1H-indol-3-yl]acetate

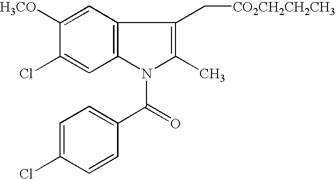

mp 132° C.

¹H NMR (CDCl₃/300 MHz) 7.64 (d, 2H, J=8.7 Hz), 7.48 (d, 2H, J=8.7), 7.19 (s, 1H), 7.00 (s, 1H), 4.06 (t, 2H, J=6.6 Hz), 3.94 (s, 3H), 3.65 (s, 2H), 2.30 (s, 3H), 1.63 (m, 2H), 0.90 (t, 3H, J=7.5 Hz).

### EXAMPLE 62

### Ethyl{6-chloro-1-[4-(difluoromethoxy)benzoyl]-5-methoxy-2-methyl-1H-indol-3-yl}acetate

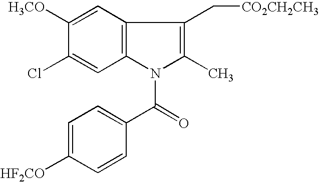

mp 120° C.

¹H NMR (CDCl₃/300 MHz) 7.74 (d, 2H, J=8.7 Hz), 7.23 (d, 2H, J=8.7), 7.16 (s, 1H), 7.00 (s, 1H), 6.64 (t, 1H, J=72.6 Hz), 4.15 (q, 2H, J=7.2 Hz), 3.94 (s, 3H), 3.65 (s, 2H), 2.32 (s, 3H), 1.26 (t, 3H, J=7.2 Hz).

### EXAMPLE 63

### sec-Butyl[6-chloro-1-(4-chlorobenzoyl)-5-methoxy-2-methyl-1H-indol-3-yl]acetate

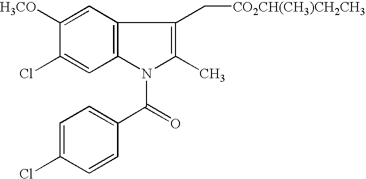

mp 105° C.

¹H NMR (CDCl₃/300 MHz) 7.63 (d, 2H, J=8.7 Hz), 7.48 (d, 2H, J=8.7), 7.21 (s, 1H), 7.00 (s, 1H), 4.83 (m, 1H), 3.92 (s, 3H), 3.62 (s, 2H), 2.29 (s, 3H), 1.55 (m, 2H), 1.17 (d, 3H, J=6.6 Hz), 0.83 (t, 3H, J=7.2 Hz).

### EXAMPLE 64

### sec-Butyl{6-chloro-1-[4-(difluoromethoxy)benzoyl]-5-methoxy-2-methyl-1H-indol-3-yl}acetate

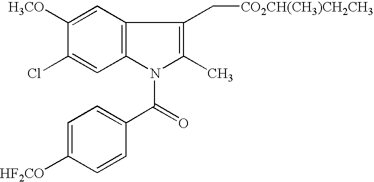

mp 90° C.

¹H NMR (CDCl₃/300 MHz) 7.72 (d, 2H, J=8.7 Hz), 7.22 (d, 2H, J=8.7), 7.17 (s, 1H), 7.01 (s, 1H), 6.93 (t, 1H, J=72.6 Hz), 4.83 (m, 1H), 3.96 (s, 3H), 3.63 (s, 2H), 2.31 (s, 3H), 1.54 (m, 2H), 1.19 (d, 3H, J=6.6 Hz), 0.84 (t, 3H, J=7.2 Hz).

### EXAMPLE 65

### Ethyl N-{[1-(4-chlorobenzoyl)-6-fluoro-5-hydroxy-2-methyl-1H-indol-3-yl]acetyl}glycinate

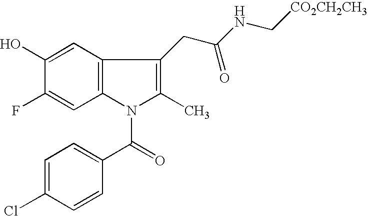

¹H NMR (DMSO-d₆/300 MHz) 9.59 (brs, 1H), 8.32 (t, 1H, J=6.0 Hz), 7.70 (d, 2H, J=8.7 Hz), 7.65 (d, 2H, J=8.7 Hz), 7.08 (d, 1H, J=8.7 Hz), 6.96 (d, 1H, J=12.0 Hz), 4.07 (q, 2H, J=7.2 Hz), 3.80 (d, 2H, J=6.0 Hz), 3.50 (s, 2H), 2.12 (s, 3H), 1.15 (t, 3H, J=7.2 Hz).

### EXAMPLE 66

### Methyl N-{[1-(4-chlorobenzoyl)-6-fluoro-5-hydroxy-2-methyl-1H-indol-3-yl]acetyl}-β-alaninate

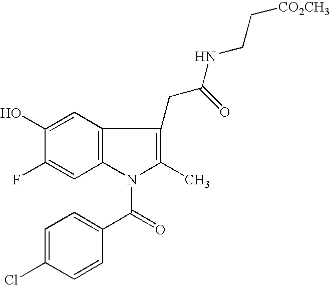

mp 165° C.

¹H NMR (DMSO-dr/300 MHz) 9.58 (s, 1H), 8.05 (t, 1H, J=3.9 Hz), 7.67-7.66 (m, 4H), 7.04 (d, 1H, J=9.0 Hz), 6.95 (d, 1H, J=12.3 Hz), 3.57 (s, 3H), 3.42 (s, 2H), 3.28 (m, 2H), 2.49 (m, 2H), 2.09 (s, 3H).

### EXAMPLE 67

### Ethyl4-({[1-(4-chlorobenzoyl)-6-fluoro-5-hydroxy-2-methyl-1H-indol-3-yl]acetyl}amino)butanoate

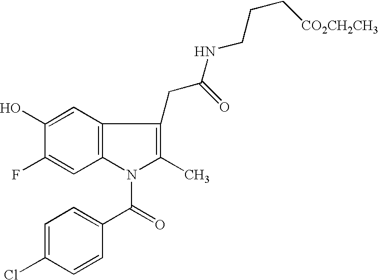

mp 189° C.

¹H NMR (DMSO-d₆/300 MHz) 9.60 (s, 1H), 7.97 (t, 1H, J=5.7 Hz), 7.71-7.63 (m, 4H), 7.05 (d, 1H, J=8.7 Hz), 6.96 (d, 1H, J=12.3 Hz), 4.03 (q, 2H, J=7.2 Hz), 3.42 (s, 2H), 3.06 (m, 2H), 2.71 (t, 2H, J=7.5 Hz), 2.10 (s, 3H), 1.64 (m, 2H), 1.16 (t, 3H, J=7.2 Hz).

### EXAMPLE 68

### 2-[1-(4-Chlorobenzoyl)-6-fluoro-5-hydroxy-2-methyl-1H-indol-3-yl]acetamide

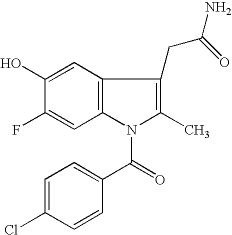

mp 205° C.

¹H NMR (CDCl₃/300 MHz) 7.52 (d, 2H, J=8.4 Hz), 7.36 (d, 2H, J=8.4), 6.82 (d, 1H, J=8.4 Hz), 6.71 (d, 1H, J=11.7 Hz), 3.41 (s, 2H), 2.14 (s, 3H).

### EXAMPLE 69

### 2-[1-(4-Chlorobenzoyl)-5-methoxy-2-methyl-1H-indol-3-yl]-N-(2-hydroxyethyl)acetamide

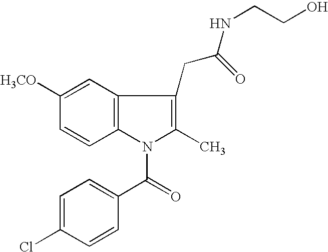

mp 158° C.

¹H NMR (CDCl₃/300 MHz) 7.67 (d, 2H, J=8.4 Hz), 7.47 (d, 2H, J=8.4), 6.90 (d, 1H, J=2.4 Hz), 6.87 (d, 1H, J=9.0 Hz), 6.69 (dd, 1H, J=9.0, 2.4 Hz), 6.05 (brt, 1H), 3.82 (s, 3H), 3.65 (s, 2H), 3.65 (m, 2H), 3.37 (m, 2H), 2.39 (s, 3H).

### EXAMPLE 70

### Ethyl N-[1-(4-chlorobenzoyl)-5-methoxy-2-methyl-1H-indol-3-yl]acetylglycinate

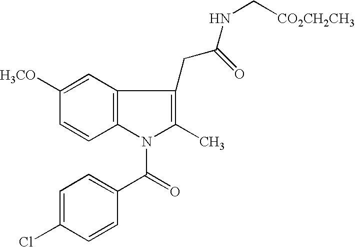

mp 179° C.

¹H NMR (CDCl₃/300 MHz) 7.65 (d, 2H, J=8.4 Hz), 7.46 (d, 2H, J=8.4), 6.95 (d, 1H, J=2.4 Hz), 6.93 (d, 1H, J=9.0 Hz), 6.70 (dd, 1H, J=9.0, 2.4 Hz), 6.16 (brt, 1H, J=5.4 Hz), 4.15 (q, 2H, J=7.2 Hz), 3.96 (d, 2H, J=5.4 Hz), 3.83 (s, 3H), 3.68 (s, 2H), 2.36 (s, 3H), 1.23 (t, 3H, J=7.2 Hz).

### EXAMPLE 71

### ({[1-(4-Chlorobenzoyl)-5-methoxy-2-methyl-1H-indol-3-yl]acetyl}amino)acetic acid

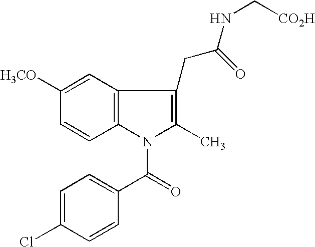

mp 194.2° C.

¹H NMR (DMSO-d₆/300 MHz) 7.71 (d, 2H, J=8.7 Hz), 7.56 (d, 1H, J=8.7 Hz), 7.05 (d, 1H, J=2.7 Hz), 6.95 (d, 1H, J=9.0 Hz), 6.66 (dd, 1H, J=9.0, 2.7 Hz), 3.92 (s, 2H), 3.82 (s, 3H), 3.69 (s, 2H), 2.31 (s, 3H).

### EXAMPLE 72

### Ethyl N-{[6-chloro-1-(4-chlorobenzoyl)-5-methoxy-2-methyl-1H-indol-3-yl]acetyl}glycinate

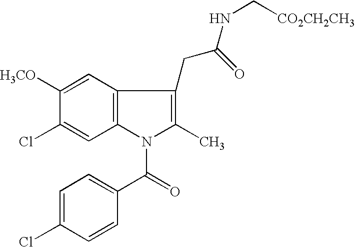

mp 191.9° C.

¹H NMR (CDCl₃/300 MHz) 7.67 (d, 2H, J=8.7 Hz), 7.50 (d, 2H, J=8.7), 7.25 (s, 1H), 6.99 (s, 1H), 6.08 (t, 1H, J=5.4 Hz), 4.17 (q, 2H, J=7.5 Hz), 3.98 (d, 2H, J=5.4 Hz), 3.97 (s, 3H), 3.68 (s, 2H), 2.31 (s, 3H), 1.25 (t, 3H, J=7.5 Hz).

### EXAMPLE 73

### ({[6-Chloro-1-(4-chlorobenzoyl)-5-methoxy-2-methyl-1H-indol-3-yl]acetyl}amino)acetic acid

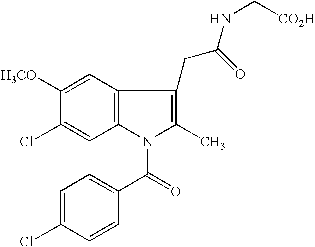

mp 219.1° C.

¹H NMR (DMSO-d₆/300 MHz) 7.72 (d, 2H, J=8.7 Hz), 7.58 (d, 1H, J=8.7 Hz), 7.23 (s, 1H), 7.20 (s, 1H), 3.93 (s, 3H), 3.92 (s, 2H), 3.69 (s, 2H), 2.24 (s, 3H).

1. A method for preparing a compound having the formula: 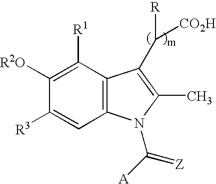
wherein:
each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen;
m is 1, 2, 3 or 4;
R¹ is H or a halogen;
R² is H; a C₁ to C₆ alkyl, optionally independently substituted with one or more halogen; 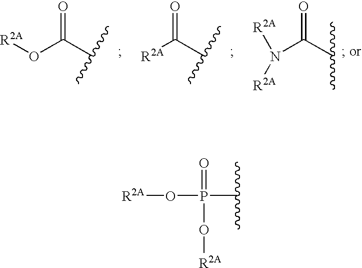
wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂;
R³ is H or a halogen;
A is 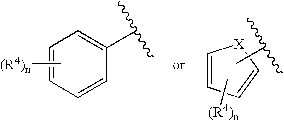
wherein each R⁴ is independently H, a halogen, —CN, —SH; —OH; a C₁ to C₃ alkyl, optionally, independently substituted with one or more halogen; —OR^{4A}; —SR^{4A}; SOR^{4A}; —S(O)₂R^{4A}, wherein R^{4A} is a C₁ to C₃ alkyl, optionally independently substituted with one or more halogen;
X is S, O, NH or NR⁵, wherein R⁵ is a C₁ to C₁₀ alkyl;
n=1,2,3, 4 or 5 when A is 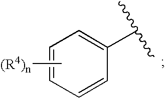
n=1, 2, 3 or 4 when A is; 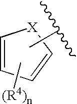
and Z is O or S,
the method comprising,
reacting a compound having the formula: 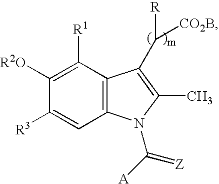
wherein A, R, R¹, R², R³ and Z are as defined above and B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃ and wherein B is optionally independently substituted with one or more halogen, —OH, —C(O)OH, or —NH₂,
with an organic acid or an inorganic acid in the presence of an organic solvent to prepare a compound having the formula:

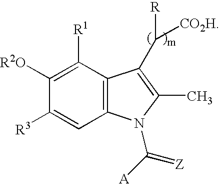

2. The method of claim 1 wherein A is

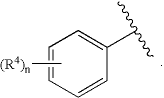

3. The method of claim 1 wherein Z is 0.

4. The method of claim 1 wherein m is 1.

5. The method of claim 1 wherein R is H.

6. The method of claim 1 wherein R is H and m is 1.

7. The method of claim 1 wherein B is —CH₂CH₂Si(CH₃)₃.

8. The method of claim 1 wherein R⁴ is halogen.

9. The method of claim 8 wherein R⁴ is Cl.

10. The method of claim 1 wherein n is 1.

11. The method of claim 1 wherein R¹ is H.

12. The method of claim 1 wherein A is

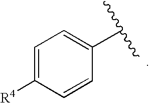

13. A method for preparing a compound having the formula: 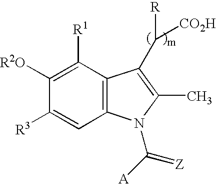
wherein:
each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen;
m is 1,2, 3 or 4;
R¹ is H or a halogen;
R² is H; a C₁ to C₆ alkyl, optionally independently substituted with one or more halogen; 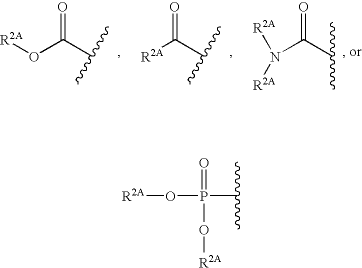
wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂;
R³ is H or a halogen; A is 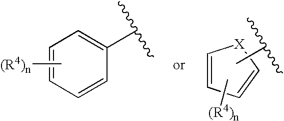
wherein each R⁴ is independently H, a halogen, —CN, —SH; —OH; a C₁ to C₃ alkyl, optionally, independently substituted with one or more halogen; —OR^{4A}; —SR^{4A}; —SOR^{4A}; —S(O)₂R^{4A}, wherein R^{4A} is a C₁ to C₃ alkyl, optionally independently substituted with one or more halogen;
X is S, O, NH or NR⁵, wherein R⁵ is a C₁ to C₁₀ alkyl;
n=1,2,3, 4 or 5 when A is 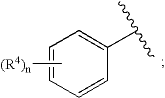
n=1,2, 3 or 4 when A is 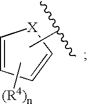
and Z is O or S;
the method comprising:
(a) reacting a compound having the formula: 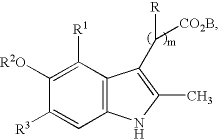
wherein m, R, R¹, R², and R³ are as defined above, and B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃ and wherein B is optionally independently substituted with one or more halogen, —OH, —C(O)OH, or —NH₂ with a strong base in an organic solvent;
(b) treating with a compound of the formula 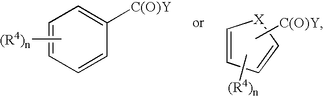
wherein R⁴, n and X are described above and Y is a suitable leaving group to generate a compound having the formula 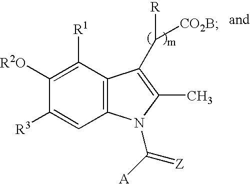
(c) treating with an organic or inorganic aqueous acid in an organic solvent to prepare a compound having the formula:
formula:

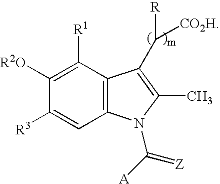

14. The method of claim 13 wherein A is

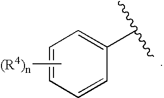

15. The method of claim 13 wherein Z is O.

16. The method of claim 13 wherein m is 1.

17. The method of claim 13 wherein R is H.

18. The method of claim 13 wherein R is H and m is 1.

19. The method of claim 13 wherein B is —CH₂CH₂Si(CH₃)₃.

20. The method of claim 13 wherein R⁴ is halogen.

21. The method of claim 20 wherein R⁴ is Cl.

22. The method of claim 13 wherein n is 1.

23. The method of claim 13 wherein R¹ is H.

24. The method of claim 13 wherein A is R⁴

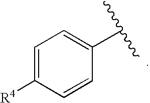

25. The method of claim 13 wherein Y is a halogen.

26. The method of claim 13 wherein 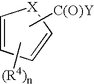
is

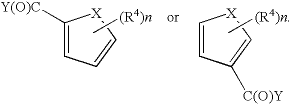

27. A compound having the formula: 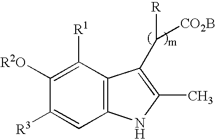
wherein, each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen;
m is 1, 2, 3 or 4;
R¹ is H or a halogen;
R² is H; a C₁ to C₆ alkyl, optionally independently substituted with one or more halogen; 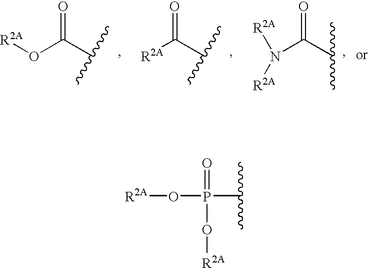
wherein each R^{2A} is independently: H, a C₁ to C₆ alkyl, a C₆ to C₁₀ alkenyl, a C₆ to C₁₀ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂;
R³ is H or a halogen; and
B is a C₁ to C₆ alkyl, a C₁ to C₆ alkenyl, a C₁ to C₆ alkynyl, aryl, cycloalkyl, or arylalkyl wherein one carbon is optionally replaced by Si(CH₃)₃ and wherein B is optionally independently substituted with one or more halogen, —OH, —C(O)OH, or —NH₂.

28. The compound of claim 27 wherein R is H; m is 1; R¹ is H or a halogen;
R² is H; a C₁ to C₆ alkyl, optionally independently substituted with one or more halogen;
R³ is H or a halogen; and
B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃.

29. The compound of claim 27 or claim 28 wherein R² is H; a C₁ to C₃ alkyl, optionally independently substituted with one or more halogen.

30. The compound of claim 27 or claim 28 wherein B is —CH₂CH₃, —CH₃, —CH₂C₆H₅, —CH₂CH═CH₂, or —C(CH₃)₃, optionally substituted with one or more halogen.

31. The compound of claim 27 or claim 28 wherein B is —CH₂CH₂Si(CH₃)₃.

32. A compound having the formula 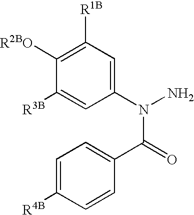
wherein R^{1B} is H or a halogen; R^{2B} is H or a C₁ to C₆ alkyl, optionally independently substituted with one or more halogen; R^{3B} is H or a halogen; and R^{4B} is H or a halogen.

33. The compound of claim 32 wherein RIB is H, Cl or F; R^{3B} is H, Cl or F; and R^{4B} is H, Cl or F.

34. The compound of claim 32 or claim 33 wherein R^{2B} is H or a C₁ to C₃ alkyl.

35. A compound having the formula: 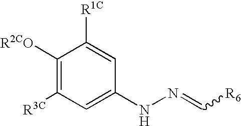
wherein R^{2C} is H or a C₁ to C₆ alkyl, optionally independently substituted with one or more halogen; R^{1C} and R^{3C} are independently H or a halogen; and R⁶ is a C₁ to C₁₀ alkyl, a C₆ to C₂₀ arylalkyl or a C₂ to C₂₀ alkenyl group

36. The compound of claim 35 wherein R^{2C} is methyl or ethyl.

37. The compound of claim 35 or claim 36 wherein R^{1C} and R^{3C} are independently Cl or F.

38. The compound of claim 35 or claim 36 wherein R^{1C} and/or R^{3C} are Cl.

39. The compound of claim 35 or claim 36 wherein R^{1C} and/or R^{3C} are F.

40. The compound of claim 35 wherein R⁶ is a C₁ to C₁₀ alkyl, optionally substituted with a phenyl.

41. A method for preparing a compound having the formula: 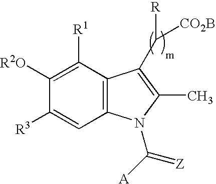
wherein:
B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein B is optionally independently substituted with one or more halogen, —OH, —C(O)OH, or —NH₂;
R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen;
m is 1,2, 3 or 4;
R¹ is H or a halogen;
R² is H; a C₁ to C₆ alkyl, optionally independently substituted with one or more halogen; 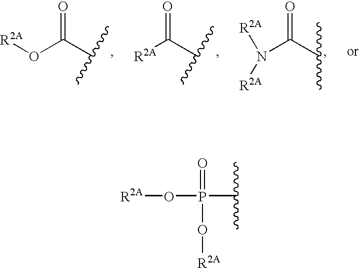
wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂;
R³ is H or a halogen;
A is 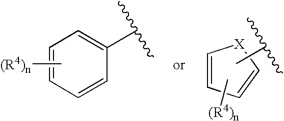
wherein each R⁴ is independently H, a halogen, —CN, —SH; —OH; a C₁-C₃ alkyl, optionally, independently substituted with one or more halogen; —OR^{4A}; —SR^{4A}; —SOR^{4A}; —S(O)₂R^{4A}, wherein R^{4A} is a C₁ to C₃ alkyl, optionally independently substituted with one or more halogen;
X is S, O, NH or NR⁵, wherein R⁵ is a C₁ to C₁₀ alkyl;
n=1, 2,3, 4 or 5 when A is 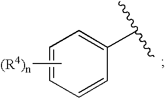
n=1, 2, 3 or 4 when A is 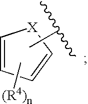
and Z is O or S:
the method comprising:
(a) reacting a compound having the formula: 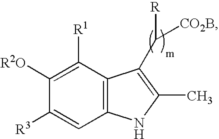
wherein m, R, R¹, R², B and R³ are as defined above with a strong base in an organic solvent;
(b) adding 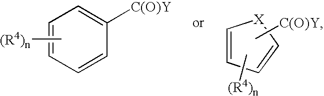
wherein R⁴, n and X are described above and Y is a suitable leaving group to prepare a compound having the formula:

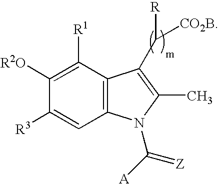

42. The method of claim 41 wherein A is

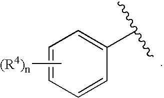

43. The method of claim 41 wherein Z is O.

44. The method of claim 41 wherein m is 1.

45. The method of claim 41 wherein R is H.

46. The method of claim 41 wherein R is H and m is 1.

47. The method of claim 13 or claim 41 wherein 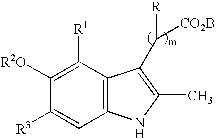
in which B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃ and wherein B is optionally independently substituted with one or more halogen, —OH, —C(O)OH, or —NH₂;
R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen;
m is 1, 2, 3 or 4;
R¹ is H or a halogen;
R² is H; a C₁ to C₆ alkyl, optionally independently substituted with one or more halogen; 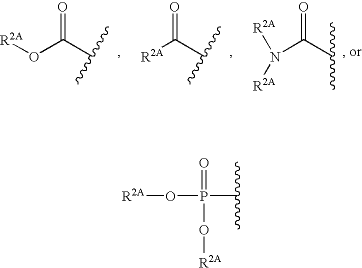
wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂; and
R³ is H or a halogen, is provided by reacting: 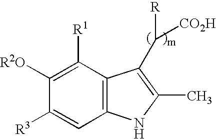
with BOH, wherein B is as defined above.

48. The method of claim 1 wherein 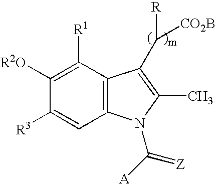
is provided by:
(a) treating 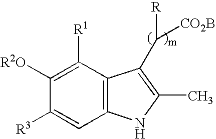
wherein A, Z, m, R, R¹, R², R³ and B are as defined in claim 1 with a strong base in an organic solvent; and
(b) adding 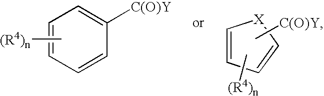
wherein R⁴, n and X are as defined in claim 1 and Y is a suitable leaving group.

49. A method for preparing a compound having the formula: 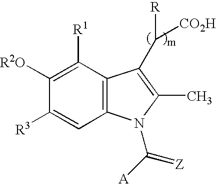
wherein:
each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen;
m is 1, 2, 3 or 4;
R¹ is H or a halogen;
R² is H; a C₁ to C₆ alkyl, optionally independently substituted with one or more halogen; 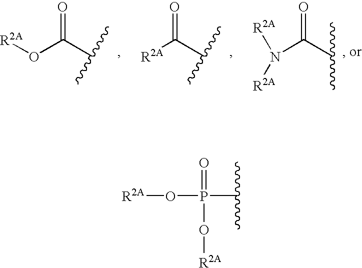
wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂;
R³ is H or a halogen;
A is 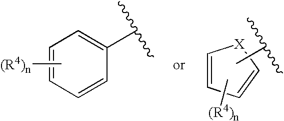
wherein each R⁴ is independently H, a halogen, —CN, —SH; —OH; a C₁ to C₃ alkyl, optionally, independently substituted with one or more halogen; —OR^{4A}; —SR^{4A}; —SOR^{4A}, —S(O)₂R^{4A}, wherein R^{4A} is a C₁ to C₃ alkyl, optionally independently substituted with one or more halogen;
X is S, O, NH or NR⁵, wherein R⁵ is a C₁ to C₁₀ alkyl;
n=1, 2, 3, 4 or 5 when A is 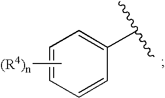
n=1, 2, 3 or 4 when A is 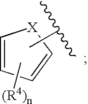
and Z is O or S:
the method comprising:
(a) reacting 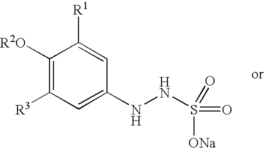 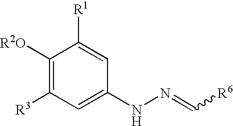
wherein R¹, R² and R³ are as defined above and R⁶ is a C₁ to C₁₀ alkyl, alkenyl or alkynl with 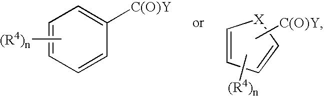
wherein R⁴, n and X are described above and Y is a suitable leaving group;
(b) treating with an acid in an alcohol and then neutralizing the acid to yield 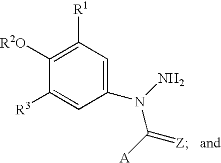
(c) reacting the resulting compound with 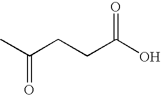
to produce a compound having the formula:

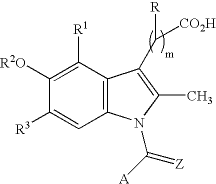

50. A method for preparing a compound having the formula: 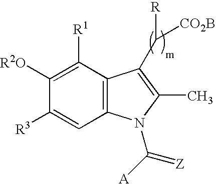
wherein:
B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃ and wherein B is optionally independently substituted with one or more halogen, —OH, —C(O)OH, or —NH₂;
Each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen;
m is 1, 2, 3 or 4;
R¹ is H or a halogen;
R² is H; a C₁ to C₆ alkyl, optionally independently substituted with one or more halogen; 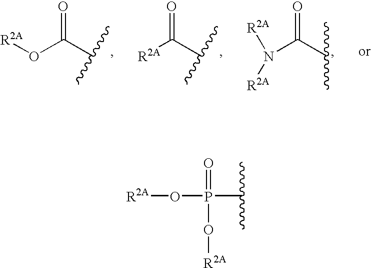
wherein each R is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂;
R³ is H or a halogen;
A is 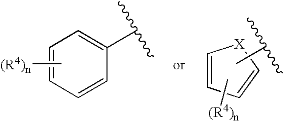
wherein each R⁴ is independently H, a halogen, —CN, —SH; —OH; a C₁ to C₃ alkyl, optionally, independently substituted with one or more halogen; —OR^{4A}; —SR^{4A}; —SOR^{4A}, —S(O)₂R^{4A}, wherein R^{4A} is a C₁ to C₃ alkyl, optionally independently substituted with one or more halogen;
X is S, O, NH or NR⁵, wherein R⁵ is a C₁ to C₁₀ alkyl;
n=1,2,3, 4 or 5 when A is 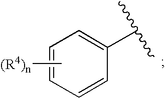
n==2, 3 or 4 when A is 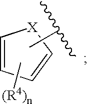
and Z is O or S:
the method comprising:
(a) reacting 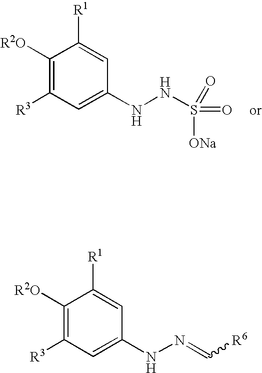
wherein R¹, R² and R³ are as defined above and R⁶ is a C₁ to C₁₀ alkyl, alkenyl or alkynl with 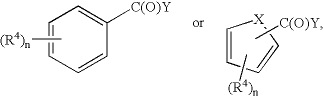
wherein R⁴ n and X are described above and Y is a suitable leaving group;
(b) treating with an acid in an alcohol and then neutralizing the acid to yield 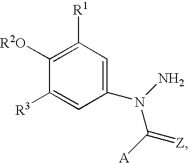
(c) reacting the resulting compound with 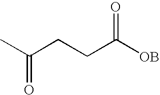
wherein B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein B is optionally independently substituted with one or more halogen, —OH, —C(O)OH, or —NH₂ to produce a compound having the formula:

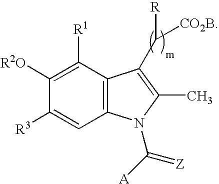

51. A method for preparing a compound having the formula: 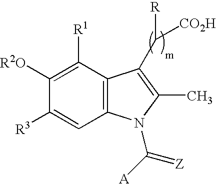
wherein:
B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃ and wherein B is optionally independently substituted with one or more halogen, —OH, —C(O)OH, or —NH₂;
Each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen;
m is 1, 2, 3 or 4;
R¹ is H or a halogen;
R² is H; a C₁ to C₆ alkyl, optionally independently substituted with one or more halogen; 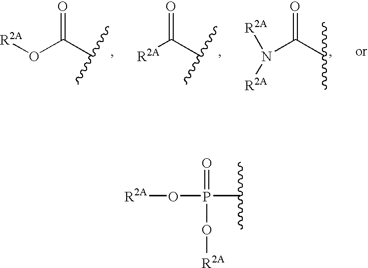
wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, (?or) —NH₂;
R³ is H or a halogen;
A is 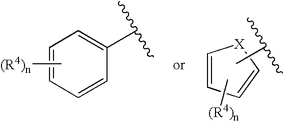
wherein each R⁴ is independently H, a halogen, —CN, —SH; —OH; a C₁ to C₃ alkyl, optionally, independently substituted with one or more halogen; —OR^{4A}; —SR^{4A}; —SOR^{4A}, —S(O)₂R^{4A}, wherein R^{4A} is a C₁ to C₃ alkyl, optionally independently substituted with one or more halogen;
X is S, O, NH or NR⁵, R⁵ is a C₁ to C₁₀ alkyl;
n=1, 2, 3, 4 or 5 when A is 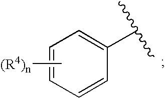
n=1, 2, 3 or 4 when A is 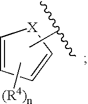
and Z is O or S:
the method comprising:
(a) reacting 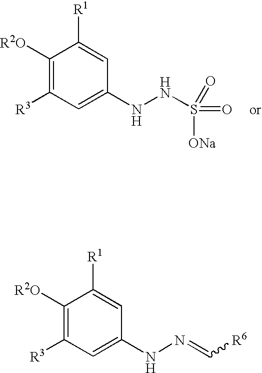
wherein R¹, R² and R³ are as defined above and R⁶ is a C₁ to C₁₀ alkyl, alkenyl or alkynl with 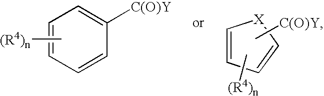
wherein R⁴, n and X are described above and Y is a suitable leaving group;
(b) treating with an acid in an alcohol and then neutralizing the acid to yield 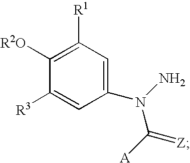
(c) reacting the resulting compound with 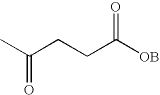
to produce a compound having the formula: 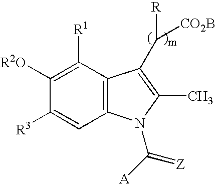
and treating with an organic or inorganic acid to generate a compound having the formula:

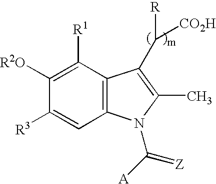

52. A compound having the formula: 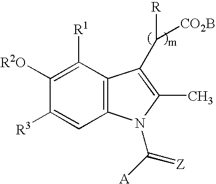
wherein each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen;
m is 1,2, 3 or 4;
R¹ is H or a halogen;
R² is H; a C₁ to C₆ alkyl, optionally independently substituted with one or more halogen; 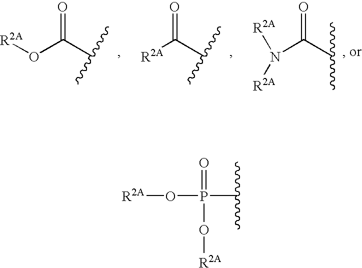
wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, (?or) —NH₂;
R³ is H or a halogen;
A is 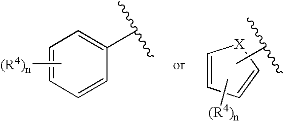
wherein each R⁴ is independently H, a halogen, —CN, —SH; —OH; a C₁ to C₃ alkyl, optionally, independently substituted with one or more halogen; —OR^{4A}; —SR^{4A}; —SOR^{4A}; —S(O)₂R^{4A}, wherein R^{4A} is a C₁ to C₃ alkyl, optionally independently substituted with one or more halogen;
X is S, O, NH or NR⁵, wherein R⁵ is a C₁ to C₁₀ alkyl; n=1, 2, 3, 4 or 5 when A is 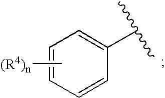
n=1,2, 3 or 4 when A is 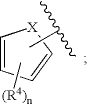
Z is O or S;
and B is a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl wherein one carbon is optionally replaced by Si(CH₃)₃ and wherein B is optionally independently substituted with one or more halogen, —OH, —C(O)OH, or —NH₂.

53. A compound having the formula: 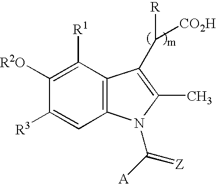
wherein each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen;
m is 1, 2, 3 or 4;
R¹ is H or a halogen;
R² is H; a C₁ to C₆ alkyl, optionally independently substituted with one or more halogen; 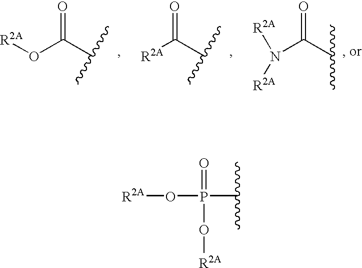
wherein each R^{2A} is independently: H, a C₁ to C₆ alkyl, a C₁ to C₆ alkenyl, a C₁ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂;
R³ is H or a halogen;
A is 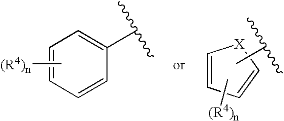
wherein each R⁴ is independently H, a halogen, —CN, —SH; —OH; a C₁ to C₃ alkyl, optionally, independently substituted with one or more halogen; —OR^{4A}; —SR^{4A}; —SOR^{4A}; —S(O)₂R^{4A}, wherein R^{4A} is a C₁ to C₃ alkyl, optionally independently substituted with one or more halogen;
X is S, O, NH or NR⁵, wherein R⁵ is a C₁ to C₁₀ alkyl;
n=1, 2, 3, 4 or 5 when A is 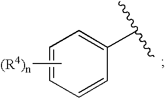
n=1, 2, 3 or 4 when A is 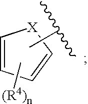
Z is O or S.

54. A compound having the formula: 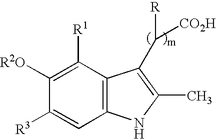
wherein, each R is, independently, H; a C₁ to C₆ alkyl, optionally independently substituted with one or more —OH, —NH₂, or halogen; or a C₁ to C₆ alkenyl optionally independently substituted with one or more —OH, —NH₂ or halogen;
m is 1,2, 3 or 4;
R¹ is H or a halogen;
R² is H; a C₁ to C₆ alkyl, optionally independently substituted with one or more halogen; 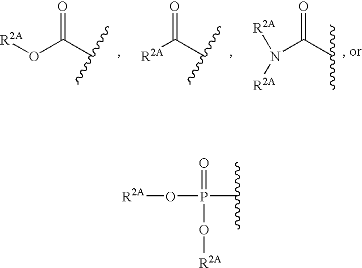
wherein each R^{2A} is independently: H, a C₁ to C₆ alkyl, a C₆ to C₁₀ alkenyl, a C₆ to C₁₀ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂; and
R³ is H or a halogen.
